(19) United States
(12) Patent Application Publication
(10) Pub. No.: US 2022/0073565 A1
(43) Pub. Date: Mar. 10, 2022

(54) DE NOVO DESIGN OF PROTEIN SWITCHES FOR TUNABLE CONTROL OF PROTEIN DEGRADATION

(71) Applicants: University of Washington, Seattle, WA (US); The Regents of the University of California, Oakland, CA (US)

(72) Inventors: Robert LANGAN, Seattle, WA (US); Andrew NG, Oakland, CA (US); Scott BOYKEN, Seattle, WA (US); Marc LAJOIE, Seattle, WA (US); Hana EL-SAMAD, Oakland, CA (US); David BAKER, Seattle, WA (US)

(21) Appl. No.: 17/419,413

(22) PCT Filed: Jan. 6, 2020
(86) PCT No.: PCT/US2020/012372

§ 371 Date: Jun. 29, 2021

Related U.S. Application Data
(60) Provisional application No. 62/850,336, filed on May 20, 2019.
(60) Provisional application No. 62/789,351, filed on Jan. 7, 2019.

(51) Int. Cl. C07K 14/00

(52) U.S. Cl. CPC C07K 14/001; C07K 2319/00

(57) ABSTRACT

Disclosed herein are non-naturally occurring cage polypeptides, kits and degron LOCKRs including the cage polypep-tides, and uses thereof, wherein the cage polypeptides include (a) a helical bundle, comprising between 2 and 7 alpha-helices, wherein the helical bundle includes: (i) a structural region; and (ii) a latch region, wherein the latch region composes a degron located within the latch region, wherein the structural region interacts with the latch region to prevent activity of the degron; and (b) amino acid linkers connecting each alpha helix

### CROSS REFERENCE

This application claims priority to U.S. Provisional Patent Application Ser. Nos. 62/789,351 filed Jan. 7. 2019 and 62/850,336 filed May 20, 2019, each incorporated by reference herein in its entirety.

### FEDERAL FUNDING STATEMENT

This invention was made with government support under Grant No. HR0011-16-2-0045, awarded by the Defense Advanced Research Projects Agency. The government has certain rights in the invention.

### REFERENCE TO SEQUENCE LISTING SUBMITTED ELECTRONICALLY VIA EFS-WEB

The content of the electronically submitted sequence listing in ASCII text file (Name: 18-1783-PCT_Sequence-Listing_ST25.txt; Size: 34,384 kB kb; and Date of Creation: Jan. 6, 2020) filed with the application is herein incorporated by reference in its entirety.

### BACKGROUND

There has been considerable progress in the de novo design of stable protein structures based on the principle that proteins fold into their lowest free energy state. These efforts have focused on maximizing the free energy gap between the desired folded structure and all other structures. Designing proteins that can switch conformations is more challenging, as multiple states must have sufficiently low free energies to be populated relative to the unfolded state, and the free energy differences between the states must be small enough that the state occupancies can be toggled by an external input. The de novo design of a protein system which switches conformational state in the presence of an external input has not been achieved.

### SUMMARY

In one aspect, the disclosure provides non-naturally occurring cage polypeptides comprising:

(a) a helical bundle, comprising between 2 and 7 alpha-helices, wherein the helical bundle comprises:
(i) a structural region; and
(ii) a latch region, wherein the latch region comprises a degron located
   within the latch region, wherein the structural region interacts with the latch region to prevent activity of the degron; and

(b) amino acid linkers connecting each alpha helix.

In one embodiment, the latch region is C-terminal to the structural region, and the degron is located within about 100, 95, 90, 85, 80, 75, 70, 65, 60, 55, 50, 45, 40, 35, 30, 25, 20, 15, 10, 5, 4, 3, 2, 1, or 0 amino acid residues of the C-terminus of the latch region, and/or within about 100, 95, 90, 85, 80, 75, 70, 65, 60. 55, 50, 45, 40, 35, 30, 25, 20, 15, 10, 5, 4, 3, 2, 1, or 0 amino acid residues of the C-terminus of the cage polypeptide.

In one embodiment, the degron comprises a CA dipeptide located between 10-30 residues from the C-terminus of the cage polypeptide; in various embodiments, the degron comprises the peptide MSCAQES (SEQ ID NO: 28468) and/or L(X)MSCAQES (SEQ ID NO: 28467), wherein X can be any amino acid residue, wherein X is optionally not proline. In another embodiment, the degron comprises an amino acid residue or peptide selected from the group consisting of

(a) GG; RG; KG; QG; WG; PG; AG; RxxG; EE; R; Rxx; Vx; Ax; A, wherein “x” can be any amino acid residue, and wherein the degron is within 10-30 amino acids of a terminus of the latch region, and/or within 10-30 amino acids of a terminus of the cage polypeptide:

(b) an amino acid residue or peptide that recruits an ubiquitin ligase that ubiquitilates the cage polypeptide and/or the operably linked functional polypeptide;

(c) a proteolysis-targeting chimeric molecule (PROTAC); and

(d) any other degron described herein.

In a further embodiment, the latch region is N-terminal to the structural region, and wherein the degron is located within about 100, 95. 90, 85, 80. 75, 70, 65, 60, 55, 50, 45, 40, 35. 30, 25, 20, 15, 10, 5, 4, 3, 2, 1, or 0 amino acid residues of the N-terminus of the latch region, and/or within about 100, 95. 90, 85, 80, 75, 70, 65, 60, 55, 50, 45. 40, 35, 30, 25, 20, 15, 10, 5, 4, 3, 2, 1, or 0 amino acid residues of the N-terminus of the cage polypeptide.

In other embodiments, the degron comprises a peptide selected from the group consisting of the following (residues within brackets are optional):

| (SEQ ID NO: 28469) | |
|---|---|
| | [[KTRGVEEVAEGVVLL]]RRRG [[NK(FAM)KKK]], |
| (SEQ ID NO: 28470) | |
| | [[KPFLNGGPY]] HSREQSTDSG [[LGLGSYK(FAM)KKK]], |
| (SEQ ID NO: 28471) | |
| | ASADLDLEALAPYIPADDDFQLRK(FAM)KKK, |
| (SEQ ID NO: 28472) | |
| | [[K-(PEG)-KEEK]] DINNN [[VKKTK(FAM)KKK]], |
| (SEQ ID NO: 28487) | |
| | [[K-(PEG)]] DVQKADVSST [[GQGIDSK(FAM)KKK]], |
| (SEQ ID NO: 28473) | |
| | KAAEEEEVSLASTPTDVRDVDIK(FAM)KKK, |
| (SEQ ID NO: 28474) | |
| | [[KKYSSQTSQ]] DSGNYS [[NK(FAM)KKK]], |
| (SEQ ID NO: 28475) | |
| | KPLSSSVPSQKTYQGSYGFRLGK(FAM)KKK, |
| (SEQ ID NO: 28476) | |
| | [[KAWQQQSYL]] DSGIHSG [[ATTTAPK(FAM)KKK]], |
| (SEQ ID NO: 28488) | |
| | [[KTRGVEEVAEGVVLL]]RRRG[[NKKKK]], |
| (SEQ ID NO: 28489) | |
| | [[KPFLNGGPY]]HSREQSTDSG[[LGLGSYKKKK]], |
| (SEQ ID NO: 28490) | |
| | ASADLDLEALAPYIPADDDFQLRKKKK, |
| (SEQ ID NO: 28491) | |
| | [[KKEEK]]DINNN[[VKKTKKKK]], |
| (SEQ ID NO: 28492) | |
| | [[K]]DVQKADVSST[[GQGIDSKKKK]], |
| (SEQ ID NO: 28493) | |
| | KAAEEEEVSLASTPTDVRDVDIKKKK, |
| | (SEQ ID NO: 28494) |
| | [[KKYSSQTSQ]]DSGNYS [[NKKKK]], |
| (SEQ ID NO: 28495) | |
| | KPLSSSVPSQKTYQGSYGFRLGKKKK, |
| (SEQ ID NO: 28496) | |
| | [[KAWQQQSYL]]DSGIHSG[[ATTTAPKKKK]], |
| (SEQ ID NO: 28497) | |
| | RRRG; |
| (SEQ ID NO: 28498) | |
| | HSREQSTDSG; |
| (SEQ ID NO: 28499) | |
| | DINNN; |
| (SEQ ID NO: 28500) | |
| | DVQKADVSST; |
| (SEQ ID NO: 28501) | |
| | DSGNYS; |
| | and |
| (SEQ ID NO: 28502 | |
| | DSGIHSG. |

In a further embodiment, the cage polypeptides further comprise one or more functional polypeptide domains. In various embodiments, the one or more functional polypeptide domains may be located at the same terminus or different termini of the cage polypeptide as the latch region. In one embodiment, the one or more functional polypeptide domains are located at the N-terminus of the cage polypeptide and the latch region is located C-terminal to the structural region. In another embodiment, the one or more functional polypeptide domains are located at the C-terminus of the cage polypeptide and the latch region is located N-terminal to the structural region. In a further embodiment, the latch region comprises one or more additional bioactive peptides besides the degron, wherein the structural region interacts with the latch region to prevent activity of the one or more additional bioactive peptides.

In one embodiment, the cage polypeptide comprises the amino acid sequence having at least 45%, 50%, 55%, 60%, 65%, 70%. 75%. 80%, 85%, 90%. 91%, 92%, 93%, 94%, 95%, 96%, 97%, 98%, 99%, or 100% sequence identity, not including optional residues, along the full length of the amino acid sequence of a cage polypeptide selected from the group consisting of (a) SEQ ID NO: 27359-28465 or a cage polypeptide listed in Table 7 (in (a) embodiments, the degron is included within the polypeptide sequence), and (b) SEQ ID NOS:1-49, 51-52, 54-59, 61, 65, 67-91, 92.-2033, SEQ ID NOS:2034-14317, 27094-27117, 27120-27125, 27,278 to 27,321, and cage polypeptides listed in Table 2 (polypeptides with an even-numbered SEQ ID NO between SEQ ID NOS: 27126 and 27276), Table 3, and/or Table 4 ((i.e.: in (b) embodiments, the degron is not included in the amino acid sequence and would be added within the latch region, including but not limited to those degron amino acid sequences disclosed herein.

In another aspect, the disclosure provides kits or degron LOCKR switches comprising:

(a) the cage polypeptide of any embodiment or combination of embodiments disclosed herein; and

(b) a key polypeptide capable of binding to the cage polypeptide structural region, thereby displacing the latch region and activating the one or more degron.

In one embodiment of the kits or degron LOCKR switches, the key polypeptide comprises an amino acid sequence having at least 45%, 50%, 55%, 60%, 65%, 70%, 75%, 80%, 85%, 90%, 91%, 92%, 93%, 94%, 95%, 96%, 97%, 98%, 99%, or 100% sequence identity, not including optional residues, along the full length of the amino acid sequence of a key protein disclosed herein, or a key polypeptide selected from the group consisting of SEQ ID NOS: 26602-27050, and 27,322 to 27,358, and 28477-28486. In another embodiment of the kits or degron LOCKR switches, the cage polypeptide comprises an amino acid sequence having at least 45%, 50%, 55%, 60%, 65%, 70%, 75%, 80%, 85%. 90%, 91%, 92%, 93%, 94%, 95%, 96%, 97%, 98%, 99%, or 100% sequence identity, not including optional residues, along the full length of the amino acid sequence selected from the group consisting of SEQ ID NO: 27359-28465 or a cage polypeptide listed in Table 7. In a further embodiment of the kits or degron LOCKR switches the one or more cage polypeptide and the one or more key polypeptide comprise at least one cage polypeptide and at least one key polypeptide comprising an amino acid sequence having at least 40%, 45%. 50%, 55%, 60%, 65%, 70%. 75%, 80%, 85%. 90%, 91%, 92%, 93%. 94%, 95%, 96%, 97%, 98%, 99%, or 100% sequence identity, not including optional residues, along the full length of a cage polypeptide and a key polypeptide, respectively, the in the same row of Table 2, 3, 4, 5, 6, or 7.

In various aspects, the disclosure provides nucleic acids encoding the cage polypeptide of any embodiment or combination of embodiments of the disclosure; expression vectors comprising the nucleic acid operatively linked to a promoter; kits comprising

(a) one or more nucleic acids encoding the polypeptide of embodiment or combination of embodiments of the disclosure; and

(b) one or more nucleic acids encoding one or more key polypeptides capable of binding to the cage polypeptide structural region; kits comprising

(a) one or more expression vectors of the disclosure; and

(b) one or more expression vectors comprising one or more nucleic acids encoding one or more key polypeptides capable of binding to the cage polypeptide structural region, wherein the one or more nucleic acids encoding one or more key polypeptides are operatively linked to a promoter; and host cells comprising one or more nucleic acids encoding the polypeptide of any embodiment or combination of embodiments of the disclosure, and/or one or more of the expression vectors of the disclosure, optionally further comprising one or more nucleic acids encoding one or more key polypeptides capable of binding to the cage polypeptide structural region. In one embodiment, the one or more nucleic acids encoding the one or more key polypeptides comprise an amino acid sequence having at least 45%, 50%, 55%, 60%, 65%, 70%, 75%. 80%, 85%, 90%, 91%. 92%, 93%, 94%, 95%, 96%, 97%, 98%, 99%, or 100% sequence identity, not including optional residues, along the full length of the amino acid sequence of a key protein disclosed herein, or a key polypeptide selected from SEQ ID NOS: 26602-27050, 27,322-27.358, and 28477-28486, in particular SEQ ID NOS: 28477-28486. In a further embodiment of the kits or host cells, the one or more nucleic acids encoding the cage polypeptide of any embodiment or combination of embodiments, encodes a polypeptide that comprise an amino acid sequence having at least 45%, 50%, 55%, 60%, 65%, 70%, 75%. 80%, 85%, 90%, 91%, 92%, 93%, 94%, 95%, 96%, 97%, 98%, 99%, or 100% sequence identity, not including optional residues, along the full length of the amino acid sequence of a cage polypeptide selected from the group consisting of SEQ ID NOS: 27359-28465 or a cage polypeptide listed in Table 7. In a further embodiment of the kits and host cells, the one or more cage polypeptide and the one or more key polypeptide comprise at least one cage polypeptide and at least one key polypeptide comprising an amino acid sequence having at least 40%, 45%, 50%. 55%, 60%, 65%, 70%, 75%, 80%. 85%, 90%, 91%, 92%, 93%, 94%. 95%, 96%, 97%, 98%, 99%, or 100% sequence identity, not including optional residues, along the full length of a cage polypeptide and a key polypeptide, respectively, the in the same row of Table 2, 3, 4, 5, 6, or T in particular Table 6 or Table 7.

In another aspect, the disclosure provides uses of the cage polypeptides, LOCKR switches, nucleic acids, expression vectors, or host cells disclosed herein to sequester a degron in the cage polypeptide until a key is expressed and activates the cage polypeptide, and the degron targets the cage polypeptide and any functional peptide fused to it for degradation.

### DESCRIPTION OF THE FIGURES

FIGS. 1a-1f. Design of the LOCKR switch system. FIG. 1a shows thermodynamic model describing our design goal. The structural region and latch region in cage form the switch with some equilibrium in the. open and closed states. The key can bind the cage to promote the open state to allow target binding to the latch. FIG. 1b shows plots from the model in (a) for two values of K_{LT} showing how fraction target bound is affected by addition of key (K_{CK}=1 nM); the different colored curves show the effect of log-decreasing values of Kₒₚₑₙ=[open]/[closed]. FIG. 1c shows loops were added to homotrimer 5L6HC3_1⁵ to form monomeric five- and six-helix frameworks; double mutant V217S/1232S weakens the Latch allowing it to be displaced by key, resulting in a LOCKR system able to bind an exogenous key. FIG. 1d shows chemical denaturation with guanidinium chloride (Gdm) monitoring mean residue ellipticity (MRE) at 222 nm. FIG. 1e shows small-angle x-ray scattering (SAXS) shows that the monomeric frameworks exhibit spectra that are in close agreement to each other and the original homotrimer. FIG. 1f. Pulldown assay showing that Key binds to the truncated five-helix framework and LOCKR (V217S/I232S), but not the six-helix monomer; free GFP-Key was added to monomeric frameworks immobilized onto a plate via a hexahistidine tag; after a series of wash steps, binding was measured by GFP fluorescence (n=2, error bars indicate standard deviation).

FIGS. 2a-2d. BimLOCKR design and activity. FIG. 2a. The free energy of the latch-cage interface was tuned through sub-optimal Bim-cage interactions (left, shown as altered hydrophobic packing and a buried hydrogen bond) and by exposing hydrophobic residues at the end of the interface (right) as a toehold. FIG. 2b. Introduction of the toehold allows activation of 250 nM BimLOCKR with addition of 5 μM key (‘on’ bar) via Bio-laver interferometry. FIG. 2c. Bio-layer interferometry shows key-dependent binding to Bcl2 with 250 nM BimLOCKR. Association from 0-500 s. then dissociation from 500-1700 s. FIG. 2d. Each point is a result of fitting data in I and extracting the response at equilibrium. The curves show similar data with shorter keys demonstrating the ability to tune K_{CK} of LOCKR and effect its range of activation.

FIG. 3a-c. Design and validation of orthogonal BimLOCKR. FIG. 3a. Left: LOCKR in cartoon representation. Cage with three different latches superimposed and hydrogen bond networks marked by markers. Right: Hydrogen-bond networks across the orthogonal LOCKR interfaces. FIG. 3b. BimLOCKR binding to Bcl2 in response to its cognate key on Octet. One replicate. FIG. 3c. Response on Octet after 500 seconds for each switch (250 nM) and key (5 μM) pair. Average of two replicates.

FIG. 4a-b. Experimentally determined x-ray crystal structures of asymmetrized LOCKR switch designs. FIG. 4a. Crystal structure of design 1-fix-short-BIM-t0, which contains the encoded BIM peptide. FIG. 4b. Crystal structure of design 1fix-short-noBim(AYYA)-t0 is in very close agreement with the design model with respect to (left) Backbone, (middle) hydrogen bond network, and (right) hydrophobic packing; the region of the Latch where Bim and Gfp11 would be encoded is shown; the electron density map is shown for the network and hydrophobic cross-sections (middle and right).

FIG. 5a-d. LOCKR switch that can prevent split GFP11 from complementing GFP1-10 in the absence of Key. FIG. 5a. Crystal structure of GFP (pdb 2y0g) with strand 11 shown. FIG. 5b. Crystal structure of prototype switch with GFP 11 stabilized as a helix (mesh is electron density). FIG. 5c. The computational design model matches the crystal structure with a root-mean-square deviation of 0.87 Å. Experimentally determined x-ray crystal structure of designed LOCKR switch 1fix-short-GFP-t0, showing the encoded the 11^{th} strand of GFP (GFP11) is an alpha helix and in very close agreement to the design model.

FIG. 5d. GFP fluorescence is only observed in the presence of the Key peptide, demonstrating the switch is function (OFF in the absence of Key, and ON in the presence of Key).

FIG. 6a-c. Designed GFP11-LOCKR switch from FIG. 4, tuned to be colocalization-dependent. FIG. 6a. Schematic of test system, where colocalization-dependence is controlled by linked Spycatcher™/Spytag™ fusions. In this model, the Key should only activate the LOCKR switch (yield fluorescence) when fused to Spytag, which will colocalizing the Key to the Cage (right). When Key alone is added, it should not be able to activate the LOCKR switch (middle). FIG. 6b-c. Fluorescence data demonstrating colocalization-dependence of designed LOCKR switches following the schematic in FIG. 6a. Designs 1fix-latch and 1fix-short fused to Spycatcher™ show more activation when mixed with their cognate Keys fused to Spytag™; Keys lacking Spytag™ show markedly less activation.

FIG. 7a-b. Caged intein LOCKR switches. FIG. 7a. Designed LOCKR switch with Cage component encoding the V MAc Intein shows successful activation when mixed with designed Key fused to sfGFP and VMAn Intein. FIG. 7b. The SDS-PAGE shows successful VMAc-VMAn reaction, with bands corresponding to the correct molecular weight of the expected spliced protein products.

FIG. 8. Multiple sequence alignment (MSA) comparing the original LOCKR_a Cage scaffold design to its asymmetrized (1fix-short-noBim(AYYA)-t0) and orthogonal (LOCKRb-f) design counterparts. Only 150 (40.8%) of the sites are identical across the MSA, with a pairwise % identity of 69.4%. The Latch regions (the C-terminal region starting at position labeled 311 in this MSA) have very little sequence identity/similarity (from top to bottom SEQ ID NOs: 17, 39, 7, 8, 9, 10, 11).

FIG. 9. Superposition of the crystal structure (white) of 1fix-short-noBim(AYYA)-t0 (FIG. 4B) onto the x-ray crystal structure of the base scaffold 5L6HC3_1⁵ (dark) used to make LOCKRa (FIG. 1) demonstrates that the asymmetrizing mutations (variable positions shown in FIG. 8 MSA) do not affect the three-dimensional structure of the protein. The backbone RMSD between the two proteins is 0.85 Angstroms (from superposing of all backbone atoms between chains A).

FIG. 10: GFP Plate assay to find mutations for LOCKR. Different putative LOCKR constructs were adhered via 6x-His tag to a Ni coated 96-well plate, Key-GFP was applied, and excess washed. Resulting fluorescence represents Key-GFP bound to LOCKR constnicts. The truncation was used as a positive control, since the key binds to the open interface. The monomer as a negative control since it does not bind the key. Error bars represent the standard deviation of three replicates.

FIG. 11a-b: Orthogonal LOCKR GFP assays. FIG. 11a. The latch was truncated from the 6x-His tagged cage in the five redesigned LOCKR constructs (b through f). The corresponding keys were GFP tagged. Key-Cage binding was measured by Ni pulldown of the cage and measuring the resulting GFP fluorescence. Error bars are standard deviation of three replicates. FIG. 11b. Each full LOCKR construct that binds key from (a) was given a nine-residue toehold and tested for binding against all four functional keys (a through d) in the GFP pulldown assay. Error bars are standard deviation of five replicates. Key a is suspected to be promiscuous binding, but not activating, due to the pseudosymmetric generation of LOCKR from a homotrimer. LOCKRb shows no binding to its own key, which is attributable to latch strength given results from (a) and FIG. 3B.

FIG. 12a-d: Designed Mad1-SID LOCKR switches for key-dependent transcriptional repression. FIG. 12a. Crystal structure of the interaction between Mad1-SID domain (white) and the PAH2 domain of the mSin3A transcriptional repressor (black) (PDB ID: 1E91). Caging of the Mad1-SID domain should enable key-dependent recruitment of the transcriptional repressor mSin3A enabling precise epigenetic regulation. FIG. 12b. Designed Mad1-LOCKR switches, with Cage component encoding the Mad1-SID sequence at different positions (dark gray). FIG. 12c. SDS-PAGE gel showing successfully purified 1fix302Mad1 (1) , 1fix_309_Mad1 (2) and MBP_Mad1 (3). FIG. 12d. Biolayer interferometry analysis of key-activated binding of the Mad1-LOCKR switches to the purified rnSin3A-PAH2 domain. MBP-Mad1 is a positive control for mSin3a-PAH2 binding. 1fix_309Mad1 (309) shows successful activation when mixed with designed Keyₐ. 1fix_302_Mad1 (302) shows very tight caging of the Mad1-SID domain, but no activation in presence of Keyₐ. Kinetic assays were performed by immobilizing 0.1μg of Biotin-mSin3A-PAH2 protein on Streptavidin biosensor tips (ForteBio). Protein cages were tested at 50 nM in presence or absence of 500 nM Keyₐ.

FIG. 13a-d. Caged STREPII-tag LOCKR switches; demonstration of new 2plus1 and 3plus1 LOCKR switches. FIG. 13a. Designed 2+1. (left) and 3+1 (middle) LOCKR switches were designed to encode the STREPII sequence WSHPQFEK. (SEQ ID NO: 63). FIG. 13b-d. Biolayerinterferometry (Octet) Data demonstrating function of the STREPII-LOCKR designs: anti-strep antibody is immobilized onto Anti-mouse FC tips to assess binding of the STREPII tag: FIG. 13b. The designed proteins show less binding than positive control suggesting the STREPII has been at least partially sequestered as intended. FIG. 13c. Activation of design STREPII-3plus1_Lock_3 by 3plus1_Key_3: The curve is 250 nM of cage with no Key, compared to 250 nM Cage in the presence of increasing concentration of Key ranging from 121 nM to 6000 nM. FIG. 13d. 250 nM STREPII-3plus1_Lock_3 in the presence of Key at 370 nM, 1111 nM, and 3333 nM; 250 nM of Cage with no Key is 250 nM, and the other plots are Key at the same concentrations (370 nM, 1111 nM, and 3333 nM) but in the absence of Cage. In all Octet plots, the left half is the association (binding) step, and the right half is the dissociation step.

FIG. 14. 3plus1 LOCKR switches activate GFP fluorescence in response to expression of Key. LOCKR switches were designed in which 3plus1 Cages were used to sequester strand 11 of GFP (GFP11) in an inactive conformation, thereby preventing reconstitution of split GFP (comprised of GFP1-10 and GFP11), resulting in fluorescence. Expression plasmids were prepared for inducibly expressing the Cage (p15a origin of replication, spectinomycin resistance, arabinose-inducible promoter controlling expression of GFP1-10 and LOCKR-Caged. GFP11) and Key (colE1 origin of replication, kanamycin resistance, and IPTG-inducible promoter). Chemically competent E. coli Stellar cells (Takarabio) were transformed according to manufacturer's protocols either with the Cage plasmid alone or with both the Cage and Key plasmids. These transformations were grown overnight at 37 C in liquid LB media supplemented with spectinomycin (Cage alone) or spectinomycin±kanamycin (Cage and Key). The resulting cultures were diluted 1/100 into fresh LB media supplemented with appropriate antibiotics and either arabinose only (induce expression of Cage and GFP1-10) or both arabinose and IPTG (induce expression of Cage, GFP1-10, and Key), then allowed to grow at 37 C for 16 hours, 200 uL of each expression culture was washed once in 200 uL PBS. resuspended in 200 uL PBS, and transferred to a black-walled 96-well plate. GFP fluorescence was evaluated on a Biotek Synergy H1MF plate reader (excitation/emission 479/520 nm). Fluorescence was minimal for Cage alone, confirming that LOCKR proteins prevented activation of split GFP in the absence of Key. Induction of Key expression resulted in a large increase in fluorescence for SEQ ID NOs 27192, 27198, 27194, 27202, 27206, and 27210. These results demonstrate that the 3plus1 LOCKR architecture is able to control the function of bioactive peptide GFP11.

FIG. 15a-e. Design and in vivo testing of degronLOCKR. FIG. 15a. Schematic of dual inducible system used in S. cerevisiae to test functionality of degronLOCKR. Progesterone (Pg) induces production of Key-BFP, and estradiol (E2) induces production of YFP-degronSwitch. FIG. 15b. Heatmaps of YFP fluorescence as a function of E2 (0-50 nM) and Pg (0-100 nM) for full length Key (left) and a Key that was truncated by 12 residues (right) as measured by flow cytometry. FIG. 15c. Line plot comparing the fluorescence of the YFP-degronSwitchₐ (SEQ ID No: 27362) and Keyₐ-BFP (SEQ ID No: 28477) at a max dose of E2 (black rectangle in (b) as a function of Pg induction. YFP fluorescence was normalized to the no Pg value and BFP fluorescence was normalized to the maximum Pg value. Error bars represent s.d. of three biological replicates. FIG. 15d. Dynamic measurements of active degronLOCKR using an automated flow cytometry platform. E2 was induced to activate expression of YFP-degronSwitchₐ, and Pg was induced at t₄ₕᵣₛ to activate expression of Keyₐ-BFP. Measurements were taken every 24 minutes FIG. 15e. Coexpression of orthogonal LOCKRs in the same cell. YFP-degronSwitchₐ (SEQ ID No: 27362) and RFP-degronSwitch_{c} (SEQ ID No: 27376) were expressed using constitutive promoters and either Keyₐ-BFP (left) or Key_{c}-BFP (right) were expressed using the pZ3 inducible promoter. Normalized fluorescence of YFP-degronSwitchₐ(SEQ ID No: 27362), RFP-degronSwitch_{c} (SEQ ID No: 27376) and either Keyₐ-BFP (SEQ ID No: 28477) or Key_{c}-BFP (28483) are plotted as a function of Pg induction. Error bars represent s.d. of biological replicates.

FIG. 16a-f. Controlling gene expression using degronLOCKR. FIG. 16a. Schematic of dual induction assay to determine the effect of degronLOCKRₐ on a synthetic transcription factor (SynTF). Pg induces expression of Keyₐ-BFP, and E2 induces expression of SynTF-RFP-degronSwitchₐ fusion. The pSynTF promoter is activated by SynTF and expresses YFP. FIG. 16b. Heatmaps of YFP and RFP fluorescence as a function of E2 (0-125 nM) and Pg (0-100 nM) measured by flow cytometry. FIG. 16c. Line plot comparing the fluorescence of YFP, SynTF-RFP-degronSwitchₐ and Keyₐ-BFP at 31.25 nM E2 (black rectangle in 5b) as a function of Pg induction. YFP and RFP fluorescence was normalized to the no Pg value, and BFP fluorescence was normalized to the maximum Pg value. Error bars represent s.d. of three biological replicates. FIG. 16d. Schematic of dual induction assay to determine the effect of degronLOCKRₐ on a dCas9-VP64 targeted to the pTet7x promoter via a constitutively expressed sgRNA (not shown). Pg induces expression of Keyₐ-BFP, and E2 induces expression of dCas9-VP64-RFP-degronSwitchₐ fusion. The pTet7x promoter is activated by dCas9-VP64 and expresses YFP. FIG. 16e. Heatmaps of YFP and RFP fluorescence as a function of E2 (0-125 nM) and Pg (0-100 nM) measured by flow cytometry. FIG. 16f. Line plot comparing the fluorescence of YFP, dCas9-VP64-RFP-degronSwitchₐ and Keyₐ-BFP at 31.25 nM E2 (black rectangle in 5d) as a function of Pg induction. YFP and RFP fluorescence was normalized to the no Pg value, and BFP fluorescence was normalized to the maximum Pg value. Error bars represent s.d. of three biological replicates. Error bars represent s.d. of three biological replicates.

FIG. 17a-b. Caging cODC sequences. FIG. 17a. Three variations of the cODC degron to Cage (cODC Full is SEQ ID NO: 28466; and cODC noPro is SEQ ID NO: 28467). Variations meant to tune Kₒₚₑₙ by removing the destabilizing proline (noPro) and minimizing mutations to the Latch (CA only). FIG. 17b. Predicted models of the full and noPro cODC sequences (orange) threaded onto the Latch (dark blue). Thread position chosen such that the cysteine residue needed for degradation is sequestered against the Cage (light blue). Proline highlighted in red in the full cODC mutated to an isoleucine in the noPro variant.

FIG. 18. Comparing the stability of YFP fused to cODC variants caged in Switchₐ to an empty Switchₐ and to bimSwitchₐ. The dual inducible system from FIG. 15a was used to express the various YFP-Switchₐ fusions (solid lines and dots) via pGal1 and E2, and Keyₐ-BFP via pZ3 and Pg. YFP (Venus) alone, YFP fused to the WT cODC (cODC) or YFP fused to the proline removed cODC (cODC noPro), were also expressed using pGal1 and E2 (dashed lines). Cells were induced with a saturating dose of E2 (50 nM) and Pg was titrated in from 0-100 nM. Fluorescence was measured at steady-state using a flow cytometer and error bars represent s.d. of biological replicates. A moderate decrease in YFP fluorescence was observed as a function of Pg for the full cODC variant, whereas only a small decrease was observed for the proline removed and CA only. No decrease in fluorescence was observed as a function of Key induction for YFP alone, empty Switchₐ, or bimSwitchₐ.

FIG. 19a-b. Tuning toehold lengths of degronLOCKRₐ. The dual inducible system from FIG. 15a was used to express the various YFP-Switchₐ fusions via pGal1 and E2, and Keyₐ-BFP via pZ3 and Pg. YFP fused to the proline removed cODC (cODC no Pro) was also expressed using pGal1 and E2 (dashed line). Cells were induced with a saturating dose of E2 (50 nM) and Pg was titrated in from 0-100 nM. Fluorescence was measured at steady-state using a flow cytometer and error bars represent s.d. of biological replicates. FIG. 19a. cODC variants from FIG. 17 alone to show dynamic range of Full cODC. FIG. 19b. Extending toehold on proline removed version from 9 to 12 and 16aa. Proline removed with 12aa toehold shows the greatest dynamic range of all the switches tested.

FIG. 20a-b. BFP expression corresponding to FIG. 15b. E2 and Pg were used to induce expression of YFP-degronSwitchₐ and Keyₐ (Full length FIG. 20a or truncated FIG. 20b)-BFP, respectively. Fluorescence was measured at steady-state using a flow cytometer. BFP expression was not dependent on expression of the Switch, suggesting the Key does not co-degrade with the Switch.

FIG. 21a-b. Expression of orthogonal YFP-degronSwitch and Key-CFP. Four different switches (FIG. 21a) and Keys (A, B, C, D) (FIG. 21b) were expressed using the strong pTDH3 promoter. Fluorescence was measured at steady-state using a flow cytometer and error bars represent s.d. of biological replicates.

FIG. 22. degronLOCKR_{a-d} (SEQ ID NOs: 27376, 27374, 27376. 27383 for degronSwitches and SEQ ID NOs: 28477, 28482, 28483, 28484 for Keys) orthogonality. All combinations of pTD3-YFP-degronSwitch and pTDH3-Key-CFP were tested. Fluorescence was measured at steady-state using a flow cytometer. Percentage degradation was calculated by subtracting the YFP-degronSwitch fluorescence with the given Key-CFP coexpressed from the YFP-degronSwitch fluorescence without any Key expressed and normalizing by the YFP-degronSwitch fluorescence without any Key expressed. degronSwitchₐ (SEQ ID NO: 27376) is activated strongly by Keyₐ (SEQ ID NO: 28477) and weakly by Key_{b} (SEQ ID NO: 28482). degronSwitch_{c} (SEQ ID NO: 27376) is activated strongly by Key_{c} (SEQ ID NO: 28483) and weakly by Key_{b} (SEQ ID NO: 28482). Because degronSwitchₐ and degronSwitch_{c} are not activated by Key_{c} and Keyₐ respectively, we consider these two to be an orthogonal pair.

FIG. 23a-b. Individual degronLOCKR controls for FIG. 15e. FIG. 23a. YFP-degronSwitchₐ was expressed using the pTEF1 constitutive promoter and FIG. 23b. RFP-degronSwitch_{c} was expressed using the pTEF1 constitutive promoter. The respective Keys fused to BFP were expressed using pZ3 and Pg. Fluorescence was measured at steady-state using a flow cytometer and error bars represent s.d. of biological replicates.

FIG. 24 Exemplary cODC Variants encoded into the Latch summarized in a sequence logo.

FIG. 25a-b provides a comparison of different degronSwitch variants in HEK293T cells. degronSwitcha and the asymmetric 1fix-short_cODC switch each fused to mCherry™ RFP were expressed in human HEK293T cells, and RFP fluorescence was measured in the presence (blue) and absence (red) of Key. The length of the toehold is indicated (t5, t8, t9, t12). FIG. 25a. Mean fluorescence intensity of the LOCKR-mCherry™ RFP fusion protein. FIG. 25b. Raw mCherry™ RFP histograms used to generate the bar plot in panel A.

FIG. 26a-b detail an exemplary degronSwitch variant in human primary CD4+ T cells. FIG. 26a. Fluorescence histograms of tagBFP translationally fused to the Key. FIG. 26b. Fluorescence histograms of mCherry™ translationally fused to the asymmetric short scaffold degronSwitch with a t8 toehold and cODC degron embedded in the latch (1fix-short_cODC_t8 (SEQ ID NO: 27,372)). This data indicates that the Key is able to trigger the degronSwitch and activate degradation of mCherry™.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique. 2^{nd} Ed. (R. I. Freshney. 1987. Liss, Inc. New York, N.Y.), Gene Transfer and Expression Protocols. pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I), leucine (Leu; L), lysine (Lys; K), methionine (Met; M), phenylalanine (Phe; F), proline (Pro: P), serine (Ser; S), threonine (Thr, T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant an will recomize.

In a first aspect, the disclosure provides non-naturally occurring cage polypeptides, comprising:
(a) a helical bundle, comprising between 2 and 7 alpha-helices, wherein the helical bundle comprises:
   (i) a structural region; and
   (ii) a latch region, wherein the latch region comprises a degron, wherein the structural region interacts with the latch region to prevent activity of the degron; and
   (b) amino acid linkers connecting each alpha helix.

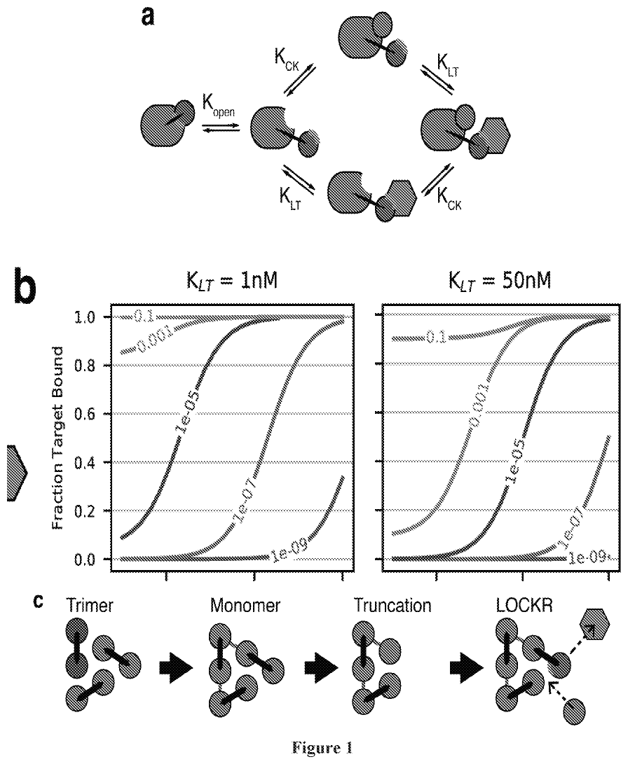

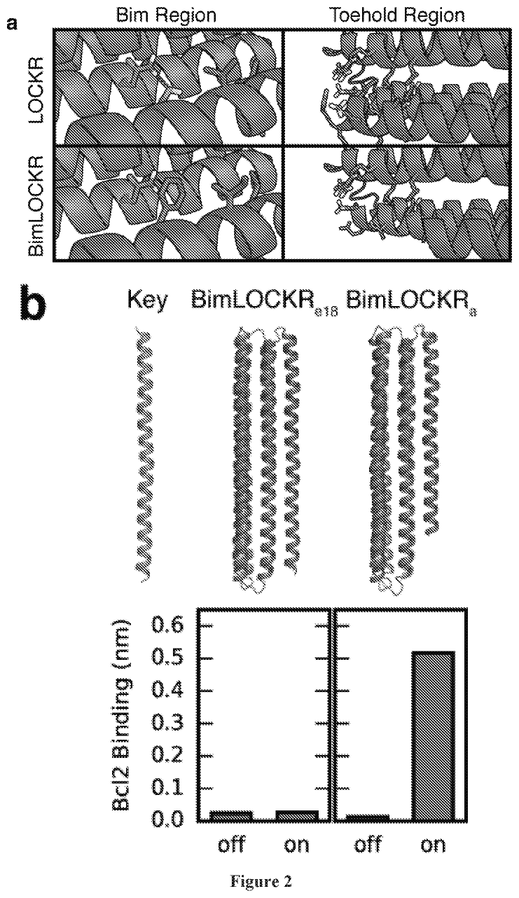

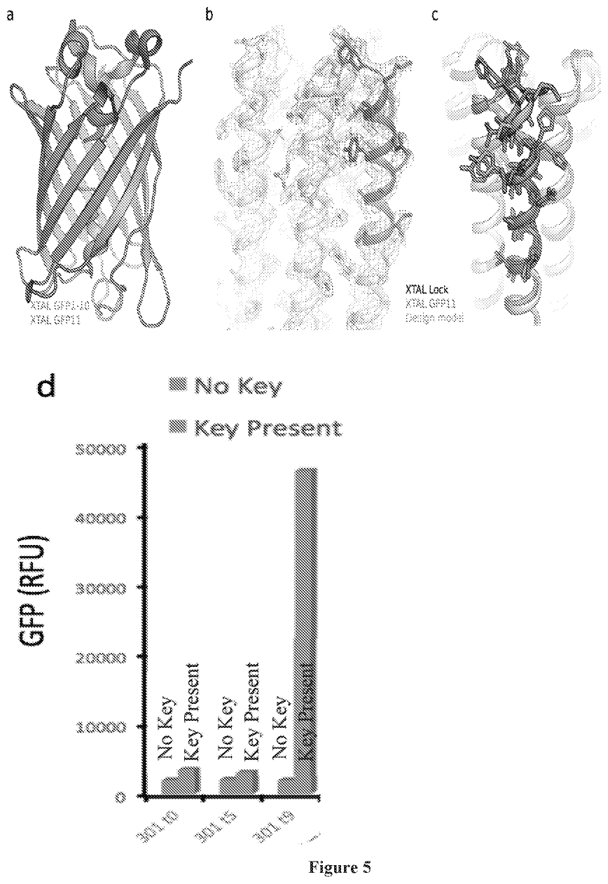

The non-naturally occurring cage polypeptides of this first aspect of the disclosure (which may also be referred to here as the “lock”) can be used, for example, as a component of the novel protein switches disclosed in detail herein. The protein switches can be used, for example, to sequester bioactive peptides in the cage polypeptide, holding them in an inactive (“off”) state, until combined with a second component (the “key” polypeptide) of the novel protein switches disclosed herein; the key polypeptide induces a conformational change that activates (“on”) the bioactive peptide (see FIG. 1a). The polypeptides described herein comprise the first ever de novo designed polypeptides that can undergo conformational switching in response to protein binding. Furthermore, there are no known natural proteins that can switch in such a modular, tunable manner as the polypeptides disclosed herein. The combined use of the cage and key polypeptides is described in more detail herein in the examples that follow, and is referred to as a LOCKR switch. LOCKR stands for Latching Orthogonal Cage-Key pRotiens; each LOCKR design consists of a cage polypeptide and a key polypeptide, which are two separate polypeptide chains. Orthogonal LOCKR designs (see FIG. 3) are denoted by lowercase letter subscripts: LOCKRₐ consists of Cageₐ and Keyₐ, and LOCKR_{b} consists of Cage_{b} and Key_{b}, etc. such that Cageₐ is only activated by Keyₐ, and Cage_{b}is only activated by Key_{b}, etc. Prefixes in the polypeptide and LOCKR names denote the functional group that is encoded and controlled by the LOCKR switch. For example, BimLOCKR refers to designed switches that encode the Bim peptide, and GFP11-LOCKR refers to designed switches that encode GFP11 (the 11th strand of GFP). See FIG. 8 for a sequence alignment comparing the original LOCKR_a Cage scaffold design to its asymmetrized (1fix-short-noBim(AYYA)-t0) and orthogonal (LOCKRb-f) design counterparts. In another embodiment, the nomenclature for the cage is identified by 1fix-short and 1fix-latch, indicating similar, yet distinct, embodiments of Cageₐ as defined above. They are all activated by Keyₐ. The functional group encoded in the latch is identified by the third portion of the name while the suffix indicates the presence of a toehold. For example, 1fix-short-Bim-t0 encodes Bim on the 1fix-short scaffold with no toehold. In another example, 1fix-latch_Mad1SID_T0_2 indicates that the 1fix-latch scaffold was used to encode Mad1SID with no residues. The suffix 2 indicates that there are two versions where the functional sequence is encoded in different locations on the latch region.

As used herein, a “degron” is a single amino acid or peptide capable of targeting the cage polypeptide and any functional polypeptide domain fused for degradation. For example, degrons may target polypeptides for degradation through targeting to the proteasome (including ubiquitin-dependent degrons (ubiquitin protein is enzymatically attached to a protein, which marks it for degradation/targeting to proteasome), and ubiquitin-independent degrons (a degron that targets a protein to the proteasome without ubiquitin), targeting to lysosomes, or recruitment of protease enzymes.

As demonstrated in the examples that follow, when a key is expressed and activates the cage polypeptide by interacting with the structural region, the degron targets the cage polypeptide, and any functional polypeptide domains and/or additional bioactive domain fused to the cage polypeptide, for degradation. In this way, a functional polypeptide domain of interest fused to the cage polypeptide having a degron can be conditionally degraded in a titratable manner via expression of the key. This is sometimes referred to herein as degronLOCKR.

The polypeptides are “non-naturally occurring” in that the entire polypeptide is not found in any naturally occurring polypeptide. It will be understood that components of the polypeptide may be naturally occurring, including but not limited to bioactive peptides that may be included in some embodiments.

The cage polypeptides comprise a helical bundle comprising between 2 and 7 alpha-helices. In various embodiments, the helical bundle comprises 3-7, 4-7, 5-7, 6-7, 2-6, 3-6, 4-6, 5-6, 2-5, 3-5, 4-5, 2-4, 3-4, 2-3, 2, 3, 4, 5, 6, or 7 alpha helices.

Design of the helical bundle cage polypeptides of the disclosure may be carried out by any suitable means. In one non-limiting embodiment, a BundleGridSampler™ in the Rosetta™ program may be used to generate backbone geometry based on the Crick expression for a coiled-coil and allows efficient. parallel sampling of a regular grid of coiled-coil expression parameter values, which correspond to a continuum of peptide backbone conformations. This may be supplemented by design for hydrogen bond networks using any suitable means, including but not limited to as described in Boyken et. al., (Science 352, 680-687 (2016)), followed by Rosetta™ sidechain design. In a further non-limiting embodiment, best scoring designs, based on total score, number of unsatisfied hydrogen bonds, and lack of voids in the core of the protein may be selected for helical bundle cage polypeptide design.

Each alpha helix may be of any suitable length and amino acid composition as appropriate for an intended use. In one embodiment, each helix is independently 38 to 58 amino acids in length. in various embodiments, each helix is independently between 18-60, 18-55, 18-50, 18-45, 22-60, 22-55, 22-50, 22-45, 25-60, 25-55, 25-50, 25-45, 28-60, 28-55, 28-50, 28-45, 32-60, 32-55, 32-50, 32-45, 35-60, 35-55, 35-50, 35-45, 38-60, 38-55, 38-50, 38-45, 40-60, 40-58, 40-55, 40-50, or 40-45 amino acids in length.

In some aspects, one or more linkers are used to link two or more polypeptides, e.g., alpha helices, structural region, latch region, degron, or any combination thereof. The amino acid linkers connecting each alpha helix can be of any suitable length or amino acid composition as appropriate for an intended use. In one non-limiting embodiment, each amino acid linker is independently between 2 and 10 amino acids in length, not including any further functional sequences that may be fused to the linker. In various non-limiting embodiments, each amino acid linker is independently 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, 7-8, 2-7, 3-7, 4-7, 5-7, 6-7, 2-6, 3-6, 4-6, 5-6, 2-5, 3-5, 4-5, 2-4, 3-4, 2-3, 2, 3. 4, 5, 6, 7, 8, 9, or 10 amino acids in length. In all embodiments, the linkers may be structured or flexible (e.g. poly-GS). These linkers may encode further functional sequences, including but not limited to protease cleavage sites or one half of a split intein system (see sequences below). As described herein linkers may further comprise one or more functional polypeptide domains in this embodiment, the linkers may be of any size suitable to include the one or more functional polypeptide domains, while maintaining the ability of the structural region and the latch region to interact.

Suitable linkers can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary linkers include glycine polymers (G)n, glycine-serine polymers (including, for example, (GS)n, (GSGGS)n (SEQ TD NO: 28503) and (GGGS)n (SEQ ID NO: 28504), where n is an integer of at least one), glycinealanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO: 28505), GGSGG (SEQ ID NO: 28506), GSGSG (SEQ ID NO: 28507), GSGGG (SEQ ID NO: 28508), GGGSG (SEQ ID NO: 28509), GSSSG (SEQ ID NO: 28510), GSEGSE (SEQ ID NO: 28547), GSGSE (SEQ ID NO: 28548), GGGSGSE (SEQ ID NO: 28549), and the like.

The polypeptides of this first aspect include a region, termed the “latch region”, for insertion of a bioactive peptide. The cage polypeptide thus comprises a latch reg ion and a structural region (i.e.: the remainder of the cage polypeptide that is not the latch region). When the latch region is modified to include one or more bioactive peptides, the structural region of the cage polypeptide interacts with the latch region to prevent activity of the bioactive peptide. Upon activation by key polypeptide, the latch region dissociates from its interaction with the structural region to expose the bioactive peptide, allowing the peptide to function.

The latch region may be present near either terminus of the cage polypeptide. In one embodiment, the latch region is placed at the C-terminal helix so as to position the bioactive peptide for maximum burial of the functional residues that need to be sequestered to maintain the bioactive peptide in an inactive state while simultaneously burying hydrophobic residues and promoting solvent exposure/compensatory hydrogen bonds of polar residues.

In various embodiments, the latch region may comprise a part or all of a single alpha helix at the N-terminal or C-terminal portions of the cage polypeptide. In various other embodiments, the latch region may comprise a part or all of a first, second, third, fourth, fifth, sixth, or seventh alpha helix in the cage polypeptide. In other embodiments, the latch region may comprise all or part of two or more different alpha helices in the cage polypeptide; for example, a C-terminal part of one alpha helix and an N-terminal portion of the next alpha helix, or all of two consecutive alpha helices. In one embodiment, the latch region comprises a single alpha helix that interacts with the alpha-helices of the structural region in the absence of a key polypeptide; in one such embodiment, the structural region comprises five alpha-helices and the interaction with the latch region results in a six helix bundle cage polypeptide. In one embodiment, the alpha helices of the structural region and the latch region may interact with each other via a combination of hydrophobic contacts and hydrogen bond networks formed between helical interfaces.

Any suitable degron may be used as is deemed appropriate for an intended use based on the disclosure herein. Degrons include portions of proteins that signal and/or target for degradation (or otherwise increase the degradation rate of) the protein to which the degron is attached or otherwise associated (e.g., grafted onto). Non-limiting examples of degrons include short amino acid sequences, structural motifs, exposed amino acids, and the like. Degrons may be prokaryote or eukaryote derived and may be employed in naturally occurring or non-naturally occurring (i.e., recombinant) forms. Degrons may be post-translationally modified to target a protein for degradation where such post-translational modifications include but are not limited to e.g., ubiquitination, proteolytic cleavage, phosphorylation, methylation, ADP-ribosylation, ampylation, lipidation, alkylation, nitrosylation, succinylation, sumoylation, neddylation, isgylation, etc. Useful degrons include ubiquitin-dependent degrons and ubiquitin-independent degrons. For example, in some instances, a protein may be targeted for ubiquitin-independent proteasomal degradation by attachment of an ornithine decarboxylase (ODC) degron, including but not limited to e.g., a mammalian ODC such as e.g., a rodent ODC, including but not limited to e.g., the c-terminal mouse ODC (cODC). In some instances, useful degrons include those described in Takeuchi et al., Biochem. J (2008) 410:401-407 and/or Matsuzawa et al., PNAS (2005) 102(42):14982-7; the disclosures of which are incorporated herein by reference in their entirety. In some instances, a protein may be targeted for ubiquitin-independent proteasomal degradation by post-translational modification (including but not limited to e.g., proteolytic cleavage, phosphorylation, methylation, ADP-ribosylation, ampylation, lipidation, alkylation, nitrosylation, succinylation, sumoylation, neddylation, isgylation, etc.) of a degron, where such modification leads, directly or indirectly, to partial or complete unfolding of the protein or other mechanisms that lead to degradation of the protein.

The degron is present within the latch region. The latch region may be present near either terminus of the cage polypeptide. Thus, in various embodiments, the latch region may be C-terminal to the structural region or N-terminal to the structural region. Thus, in some embodiments, the degron may be present within about 100. 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 amino acids from either the N-terminus or the C-terminus of the latch region, and/or within about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 amino acids from either the N-terminus or the C-terminus of the cage polypeptide. In some embodiments where the latch region is at the terminus of the cage polypeptide, the recited distance in amino acids of the degron from that terminus and from the terminus of the latch region may both be met. In other embodiments, such as where one or more polypeptide functional domains are added to the N-terminus or C-terminus of the cage polypeptide (as described below), the degron may be within the recited distance in amino acids from the terminus of the latch region but not from the terminus of the cage polypeptide.

In one embodiment, the latch region is N-terminal to the structural region, and the degron may be located within about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 amino acid residues of the N-terminus of the latch region. In one such embodiment, the degron may be located within about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 amino acid residues of the N-terminus of the cage polypeptide.

In another embodiment, the latch region is C-terminal to the structural region, and the degron may be located within about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 amino acid residues or less of the C-terminus of the latch region. In one such embodiment, the degron may be located within about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34. 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 amino acid residues of the C-terminus of the cage polypeptide. In one embodiment, the degron may comprise a ubiquitin-independent degradation signal. In one embodiment, the degron comprises a CA dipeptide located between 10-30 residues from the C-terminus of the cage polypeptide; in this embodiment. the “C” residue in the CA dipeptide is between 10-30 residues from the C-terminus of the cage polypeptide. The CA dipeptide is the minimal domain for degradation activity of the murine ornithine decarboxylase (cODC) degron, as described in Example 2 below. In other embodiments of the cODC degron, the degron comprises the peptide MSCAQES (SEQ ID NO: 28468) or L(X)MSCAQES (SEQ ID NO: 28467) (“cODC noPro”), wherein X can be any amino acid residue, wherein X is optionally not proline.

In other non-limiting embodiments, the degron may comprise an amino acid residue or peptide selected from the group consisting of -GG; -RG; -KG; -QG; -WG; -PG; -AG; -RxxG; -EE; -R; -Rxx; -Vx; -Ax; -A, wherein “x” can be any amino acid residue. In one such embodiment, the degron may be located within about 10-30 amino acid residues, or within about 20 amino acid residues, of the C-terminus of the cage polypeptide.

In other non-limiting embodiments, the degron may comprise or consist of a peptide selected from the group consisting of the following (residues within brackets are optional):

| (SEQ ID NO: 28469) | |
|---|---|
| | [[KTRGVEEVAEGVVLL]]RRRG [[NK(FAM)KKK]], |
| (SEQ ID NO: 28470) | |
| | [[KPFLNGGPY]] HSREQSTDSG [[LGLGSYK(FAM)KKK]], |
| (SEQ ID NO: 28471) | |
| | ASADLDLEALAPYIPADDDFQLRK(FAM)KKK, |
| (SEQ ID NO: 28472) | |
| | [[K-(PEG)-KEEK]] DINNN [[VKKTK(FAM)KKK]], |
| (SEQ ID NO: 28487) | |
| | [[K-(PEG)]]DVQKADVSST [[GQGIDSK(FAM)KKK]] |
| (SEQ ID NO: 28473) | |
| | KAAEEEEVSLASTPTDVRDVDIK(FAM)KKK, |
| (SEQ ID NO: 28474) | |
| | [[KKYSSQTSQ]] DSGNYS [[NK(FAM)KKK]], |
| (SEQ ID NO: 28475) | |
| | KPLSSSVPSQKTYQGSYGFREGK(FAM)KKK, |
| (SEQ ID NO: 28476) | |
| | [[KAWQQQSYL]]DSGIHSG[[ATTTAPK(FAM)KKK]], |
| (SEQ ID NO: 28488) | |
| | [[KTRGVEEVAEGVVLL]]RRRG[[NKKKK]], |
| (SEQ ID NO: 28489) | |
| | [[KPFLNGGPY]]HSREQSTDSG[[LGLGSYKKKK]], |
| (SEQ ID NO: 28490) | |
| | ASADLDLEALAPYIPADDDFQLRKKKK, |
| (SEQ ID NO: 28491) | |
| | [[KKEEK]]DINNN[[VKKTKKKK]], |
| (SEQ ID NO: 28492) | |
| | [[K]]DVQKADVSST]]GQGIDSKKKK]], |
| (SEQ ID NO: 28493) | |
| | KAAEEEEVSLASTPTDVRDVDIKKKK, |
| (SEQ ID NO: 28494) | |
| | [[KKYSSQTSQ]]DSGNYS [[NKKKK]], |
| (SEQ ID NO: 28495) | |
| | KPLSSSVPSQKTYQGSYGFRLGKKKK, |
| (SEQ ID NO: 28496) | |
| | [[KAWQQQSYL]]DSGIHSG[[ATTTAPKKKK]]; |
| (SEQ ID NO: 28497) | |
| | RRRG; |
| (SEQ ID NO: 28498) | |
| | HSREQSTDSG; |
| (SEQ ID NO: 28499) | |
| | DINNN; |
| (SEQ ID NO: 28500) | |
| | DVQKADVSST; |
| (SEQ ID NO: 28501) | |
| | DSGNYS; |
| | and |
| (SEQ ID NO: 28502) | |
| | DSGIHSG. |

In other non-limiting embodiments, the degron may comprise a polypeptide sequence that recruits an ubiquitin ligase. Such degrons (e.g., proteolysis-targeting chimeric molecules. PROTACs) have been previously described by Sakamoto et al. (2001) PNAS (15) 8554-8559 and Schneekloth et al. (2004) JACS 126(12):3748-54.

Useful degrons include ubiquitin-dependent degrons and ubiquitin-independent degrons. For example, in some instances, a protein may be targeted for ubiquitin-independent proteasomal degradation by attachment of an ornithine decarboxylase (ODC) degron, including but not limited to e.g., a mammalian ODC such as e.g., a rodent ODC, including but not limited to e.g., the c-terminal mouse ODC (cODC). In some instances, useful degrons include those described in Takeuchi et al., Biochem. J (2008) 410:401-407 and/or Matsuzawa et al., PNAS (2005) 102(42):14982-7; the disclosures of which are incorporated herein by reference in their entirety. In some instances, a protein may be targeted for ubiquitin-independent proteasomal degradation by post-translational modification (including but not limited to e.g., proteoly tic cleavage, phosphorylation, methylation, ADP-ribosylation, ampylation, lipidation, alkylation, nitrosylation, succinylation, sumoylation, neddylation, isgylation, etc.) of a degron, where such modification leads, directly or indirectly, to partial or complete unfolding of the protein or other mechanisms that lead to degradation of the protein.

In some aspects, a degron may include a ubiquitin-independent degradation signal, where such signals may vary. For example, in some instances, a ubiquitin-independent degradation signal may include a dipeptide motif, such as, e.g., a cysteine-alanine (i.e., CA) dipeptide motif. In some instances, a ubiquitin-independent degradation signal may include only a dipeptide motif. In some instances, a ubiquitin UCSF independent degradation signal may include amino acid residues in addition to a dipeptide motif, such as but not limited to e.g., a LXMSCAQE (SEQ ID NO: 28,511) motif, where X may be any amino acid or a LXMSCAQES (SEQ ID NO: 28,467) motif, where X may be any amino acid. In some instances, a LXMSCAQE (SEQ ID NO: 28,511) motif or a LXMSCAQES (SEQ 11) NO: 28,467) motif may include where X is any amino acid except proline.

Accordingly, in some instances, a degradation signal of a degron can include a sequence selected from: LPMSCAQES (SEQ ID NO: 28,466) where the final S is present or absent, LAMSCAQES (SEQ ID NO: 28,512) where the final S is present or absent, LVMSCAQES (SEQ ID NO: 28,513) where the final S is present or absent, LSMSCAQES (SEQ ID NO: 28,514) where the final S is present or absent, LEMSCAQES (SEQ ID NO: 28,515) where the final S is present or absent, and LKMSCAQES (SEQ ID NO: 28,516) where the final S is present or absent. In some instances, a degradation signal of a degron may include a MSCAQE (SEQ ID NO: 28,517) sequence or a MSCAQES (SEQ ID NO: 28468) sequence.

Ubiquitin-dependent degrons include, but are not limited to, e.g., PEST (SEQ ID NO: 28,518) (proline (P). glutamic acid (E), serine (S), and threonine (T)) sequence-containing degrons, as well as those degrons described in Melvin et al. (PLoS One. (2013) 29;8(10):e78082); the disclosure of which is incorporated herein by reference in its entirety, including degrons identified as Bonger and those described as derived from TAZ, HIF-1α, iNOS, SRC3, Cyclin D1, IFNAR1, p53, and β-Catenin.

Useful degrons may also include E3 ubiquitin ligase domains. Such degrons are often defined as the substrate site that is recognized by E3 ubiquitin ligases and a variety of such degrons, including short peptide motifs and specific structural elements, have been characterized. Non-limiting examples of E3 ligase/degrons and the corresponding motif patterns include:
APC/C (DBOX), primary motif R.L.[LIVM].; (SEQ ID NO: 28,519)
APC/C (KEN), primary motif .KEN.; (SEQ ID NO: 28,520)
APC/C (ABBA), primary motif [FIVL].[ILMVP][FHY].[DE]. {0,3}[DEST]; (SEQ ID NO: 28,521)
APCC_TPR_1, primary motif [ILM]R$; (SEQ ID NO: 28,522)
CBL (PTK), primary motif [DN].Y[ST]..P; (SEQ ID NO: 28,523)
CBL (MET), primary motif DYR; (SEQ ID NO: 28,524)
COP1, primary motif [DE][DE].{2,3}VP[DE]; (SEQ II) NO: 28,525)
CRL4_CDT2_1, primary motif
[NQ]{0,1}..[ILMV][ST][DEN][FY][FY].{2,3}[KR]{2,3}[\hat{ }DE]; (SEQ ID NO: 28,526)
CRL4_CDT2_2, primary motif
[NQ]{0,1}..[ILMV]T[DEN][HMFY][FMY]. {2,3}[KR]{2,3}[\hat{ }DE]; (SEQ ID NO: 28,527)
Kelch_KEAP1_1, primary motif [DNS].[DES][TNS]GE; (SEQ ID NO: 28,528)
Kelch_KEAP1_2, primary motif QD.DLGV; (SEQ ID NO: 28,529)
Kelch_actinfilin, primary motif [AP]P[MV][IM]V; (SEQ ID NO: 28,530)
Kelch_KLHL3, primary motif E.EE.E[AV]DQH; (SEQ ID NO: 28,531)
MDM2_SWIB, primary motif F[\hat{ }P]{3}W[\hat{ }P]{2,3}[VIL]; (SEQ ID NO: 28,532)
Nend_Nbox_1, primary motif \hat{ }M{0, 1}[FYLIW][\textcircled{ }P]; (SEQ ID NO: 28,533)
Nend_UBRbox_1, primary motif \textcircled{ }M{0,1}[RK][\textcircled{ }P].; (SEQ ID NO: 28,534)
Nend_UBRbox_2, primary motif \textcircled{ }M{1,1}([ED]).; (SEQ ID NO: 28,535)
Nend_UBRbox_3, primary motif \textcircled{ }M{0,1}([NQ]).; (SEQ ID NO: 28,536)
Nend_UBRbox_4, primary motif \textcircled{ }M{0,1}(C).; (SEQ ID NO: 28,537)
ODPH_VHL_1, primary motif[IL]A(P). {6,8}[FLIVM].[FLIVM]; (SEQ ID NO: 28,538)
SCF_COI1_1, primary motif..[RK][RK].SL..F[FLM].[RK]R[HRK].[W].; (SEQ ID NO: 28,539)
SCF_FBW7_1, primary motif [LIVMP].{0,2}(T)P..([ST]); (SEQ NO: 28,540)
SCF_FBW7_2, primary motif [LIVMP]. {0,2}(T)P..E; (SEQ ID NO: 28,541)
SCF_SKP2-CKS1_1, primary motif..[DE].(T)P.K; (SEQ ID NO: 28,542)

SCF_TIR1_1, primary motif .[VLIA][VLI]GWPP[VLI] . . . R.; (SEQ ID NO: 28,543)
SCF-TRCP1, primary motif D(S)G.{2,3}([ST]), (SEQ ID NO: 28,544)
SIAH, primary motif .P.A.V.P[\textcircled{ }P](SEQ ID NO: 28,545); or
SPOP, primary motif [AVP].[ST][ST][ST] (SEQ ID NO: 28,546);

In all cases above, ‘.’ specifies any amino acid type. ‘[X]’ specifies the allowed amino acid type(s) at that position, ‘\textcircled{ }X’ at the beginning of the pattern specifies that the sequence starts with amino acid type X, ‘[\textcircled{ }X]’ means that the position can have any amino acid other than type X, numbers specified as the following ‘X{x,y}’, where x and y specify the minimum and maximum number of ‘X’ amino acid type required at that position. ‘$’ sign implies the C-terminal of the protein chain. Degrons that include E3 ubiquitin ligase domains are described in Guharoy et al., Nature Communications (2016) 7:10239: the disclosure of which is incorporated herein by reference in its entirety.

In some aspects, useful degrons can include those degrons that contain signals for ER-associated degradation (ERAD), including but not limited to e.g., those described in Maurer et al.. Genes Genomes & Genetics (2016) 6:1854-1866; the disclosure of which is incorporated herein by reference in its entirety. In some instances, useful degrons may also include drug-inducible degrons, such as but not limited to e.g., the auxin inducible degron (AID) which utilizes a specific E3 ubiquitin ligase (e.g., as described in Nishimura et al., Nature Methods (2009) 6(12):917-922, the disclosure of which is incorporated herein by reference in its entirety). As will be readily understood, degrons that include E3 ubiquitin ligase domains will vary and may not be limited to use of those E3 ubiquitin degrons specifically described herein.

Other useful examples of degrons that can be employed in inducible degradation strategies adapted for uses in the present disclosure include but are not limited to e.g., N-end degrons (such as but not limited to e.g., those described in Tasaki & Kwon, Trends in Biochemical Sciences (2007) 32(10:520-528, the disclosure of which is incorporated herein by reference in its entirety); unstructured regions (such as but not limited to e.g., those described in Chung et al., Nat Chem Biol. 2015; 11(9): 713-720, the disclosure of which is incorporated herein by reference in its entirety); ligand induced degradation (LID) and destabilization domain (DD) domains (such as but not limited to e.g., those described in Bonger et al., Nat Chem Biol. 2012; 7(8): 531-537; Grimley et al.. Bioorg. Med, Chem. Lett (2008) 18: 759-761; and Chu et al. Bioorg. Med. Chem. Lett. (2008) 18: 5941-5944; Iwamoto et al., Chemistry &. Biology (2010) 17: 981-988; the disclosures of which are incorporated herein by reference in their entirety); prokaryotic proteasome recognition sequences such as, e.g., ssrA and mf-Lon (such as those described in Cameron et al., (2014) Nature biotechnology 32(12): 1276-1281, the disclosure of which is incorporated herein by reference in its entirety); and the like.

In another embodiment. the cage polypeptide may further comprise one or more functional polypeptide domains, wherein the functional polypeptide domain may be fused to the N-terminus, the C-terminus, or inserted into a linker of the cage polypeptide. As noted above, when a key is expressed and activates the cage polypeptide by interacting with the structural region, the degron targets the cage polypeptide, and any functional polypeptide domains fused to the cage polypeptide, for degradation through, for example, targeting to lysosomes. targeting to the proteasome, or recruitment of protease enzymes. In this way, the functional polypeptide domain of interest fused to the cage polypeptide having a degron can be conditionally degraded in a titratable manner via expression of the key. In various embodiments:
one or more functional polypeptide domains may be located at the N-terminus of the cage polypeptide and the latch region may be located C-terminal or N-terminal to the structural region;
one or more functional polypeptide domains may be located at the C-terminus of the cage polypeptide and the latch region may be located N-terminal or C-terminal to the structural region; and/or
one or more functional polypeptide domains may be located in an amino acid linker and the latch region may be located N-terminal or C-terminal to the structural region.

Any functional polypeptide of interest, or domain thereof, can be expressed as a fusion protein with the cage polypeptide such that it can be conditionally degraded in a titratable manner via expression of the key. In non-limiting embodiments, the one or more functional polypeptide domains may include, but are not limited to metabolic enzymes, transcription factors, kinases, phosphatases, Chimeric Antigen Receptor (CAR), T Cell Receptor (TCR), SynNotch, TCR mimics, cytokine receptors, G-protein coupled receptors (GPCR), co-stimulatory receptors (including but not limited to CD28, CTLA-4, ICOS), co-inhibitory receptors (e.g. PD-1), endogenous signaling domains (including but not limited to Pleckstrin Homology (PH), Src Homology 2 (SH2), Src Homology 3 (SH3), WW, C1, PDZ, CARD, phosphotyrosine-binding, proline-rich region, coiled-coil, and pseudokinase domains), synthetic receptors or synthetic signaling proteins comprising one or more signaling domain (including but not limited to Pleckstrin Homology, (PH), Src Homology 2 (SH2), Src Homology 3 (SH3), WW, Cl, PDZ, CARD, phosphotyrosine-binding, proline-rich region, coiled-coil, and pseudokinase domains), engineered or endogenous receptors containing ITAM or ITIM motifs, JAK/STAT binding motifs, DNA binding domains (including but not limited to Cas9, dCas9, TALEs, and Zinc Fingers), vesicular trafficking domains, protein degradation domains (including but not limited to ubiquitin recruitment domains and proteasomal-targeting domains), cell death domains (including but not limited to those involved in the apoptosis, necroptosis, and pyroptosis), fluorescent proteins, de novo designed proteins, a second cage polypeptide described herein, such as one that binds to a key polypeptide different than a key polypeptide bound by the cage polypeptide), a key polypeptide described herein, such as one that does not bind to the cage polypeptide, and active domains thereof As used herein, a “domain” refers to a portion of a polypeptide that retains one or more particular functions. The functional polypeptide domains may be synthetic or naturally occurring, and may comprise a full protein or a domain of a protein that possesses a specific function.

In another embodiment, the cage polypeptide may further comprise one or more additional bioactive peptides besides the degron, wherein the structural region interacts with the latch region to prevent activity of the one or more additional bioactive peptides. As used herein, a “bioactive peptide” is any peptide of any length or amino acid composition that is capable of selectively binding to a defined target (i.e.: capable of binding to an “effector” polypeptide). Such bioactive peptides may comprise peptides of all three types of secondary structure in an inactive conformation: alpha helix, beta strand, and loop. The polypeptides of this aspect can be used to control the activity of a wide range of functional peptides. The ability to harness these biological functions with tight, inducible control is useful, for example, in engineering cells (inducible activation of function, engineering complex logic behavior and circuits, etc.), developing sensors, developing inducible protein-based therapeutics. and creating new biomaterials.

As will be understood by those of skill in the art, the degron is a “bioactive peptide”. Thus, this embodiment refers to the inclusion of one or more additional bioactive peptides in the latch region, as described above in earlier aspects of the disclosure. When the latch region is modified to include one or more additional bioactive peptides, the structural region of the cage polypeptide interacts with the latch region to prevent activity of the one or more additional bioactive peptides. Upon activation by key polypeptide, the latch region dissociates from its interaction with the structural region to expose the one or more additional bioactive peptides, allowing the one or more additional bioactive peptides to function. Thus, in embodiments in which there are no additional bioactive peptides, the degron only is activated upon key polypeptide binding to the cage polypeptide. In embodiments where there are one or more additional bioactive peptides in the latch, the degron and the one or more additional bioactive peptides are activated by binding of the key polypeptide to the cage polypeptide—in one exemplary such embodiment, the degron can act by modifying the one or more additional bioactive peptides by, for example, inducing degradation of the additional bioactive peptides and thus turning off their function. This embodiment can be particularly useful, for example, to pulse the function of the one or more additional bioactive peptides, and then rapidly degrade the one or more additional bioactive peptides so that the function is transient, or to make degradation of the one or more additional bioactive peptides dependent on binding of an effector protein.

In this embodiment, the one or more additional bioactive peptide(s) may replace one or more amino acids in the latch region, or may be added to the latch region without removal of any amino acid residues from the latch region. In various non-limiting embodiments, the bioactive peptides may comprise the amino acid sequence of SEQ ID NO: 50, 60, 62-64, 66, 27052-27093, and 27118-27119, or variants thereof:

GFP11 fluorescence peptide and binding peptide to
GFP1-10:
(SEQ ID NO: 27052)
RDHMVLHEYVNAAGIT.
BIM binding peptide and apoptotic peptide to
BCL-2:
(SEQ ID NO: 50)
IxxxLRxIGDxFxxxY,
where x is any amino acid;
in one embodiment, the peptide is
(SEQ ID NO: 60)
EIWIAQELRRIGDEFNAYYA
Designed peptide for binding to BCL-2:
(SEQ ID NO: 62)
KMAQELIDKVRAASLQINGDAFYAILRAL
StreptagII binding peptide to streptactin or an
antibody:
(SEQ ID NO: 63)
(N)WSHPQFEK
TEV protease cleavage site:
(SEQ ID NO: 64)
ENLYFQ(G)-X,
wherein (G) can also be S, last position, -X can
be anything except Proline
Thrombin protease cleavage site:
(SEQ ID NO: 66)
LVPRGS
Cathepsin cleavage site:
(SEQ ID NO: 27053)
RLVGFE
Spytag covalent crosslinking peptide to
spycatcher:
(SEQ ID NO: 27054)
AHIVMVDAYK(PTK)
NLS peptide to target the protein to the nucleus:
(SEQ ID NO: 27055)
ANNKRARTS
NES1 peptide to exclude the protein from the
nucleus:
(SEQ ID NO: 27056)
LALKLAGLDIN
NES2 peptide to exclude the protein from the
nucleus:
(SEQ ID NO: 27057)
ELAEKLAGLDIN
NES3 peptide to exclude the protein from the
nucleus:
(SEQ ID NO: 27058)
ELAEKLPAGLDIN
EZH2 binding peptide to recruit DNA-methylases:
(SEQ ID NO: 27059)
TMFSSNRQKILEPTETLNQEWKQRRIQ
MDM2 binding peptide to recruit p53:
(SEQ ID NO: 27060)
ETFSDLWKLL
C25 binding peptide:
(SEQ ID NO: 27061)
GELDELVYLLDGPGYDPIHSDVVTRGGSHLFNF
9aaTAD1 for transcriptional activation:
(SEQ ID 27062)
TMDDVYNYLFDD
9aaTAD2 for transcriptional activation:
(SEQ ID NO: 27063)
LLTGLEVQYLFDD
9aaTAD3 for transcriptional activation:
(SEQ ID NO: 27064)
DDAVVESFFSS
9aaTAD4 for transcriptional activation:
(SEQ ID NO: 27065)
GDFLSDLFD
9aaTAD5 for transcriptional activation:
(SEQ ID NO: 27066)
GDVLSDLVD
Mad1-SID - epigenetic modification:
(SEQ ID NO: 27067)
NIQMLLEAADYLE
Mad1-SID (3A mutant) - epigenetic modification:
(SEQ ID NO: 27068)
NIAMLLAAAAYLE
RHIM Domain 1 from ZBP1:
(SEQ ID NO: 27069)
IQIG
RHIM Domain 2 from ZBP1:
(SEQ ID NO: 27070)
VQLG
nanoBit Split Luciterase:
(SEQ ID NO: 27071)
VSGWPLFKKIS
CC-A:
(SEQ ID NO: 27072)
GLEQEIAALEKENAALEWEIAAEQGG
CC-B:
(SEQ ID NO: 27073)
GLKQKIAALKYKNAALKKKIAALKQGG
GCN4:
(SEQ ID NO: 27074)
RMKQLEDKVEELLSKNYHLENEVARLKKLVGER
CC-Di:
(SEQ ID NO: 27075)
GEIAALKQEIAALKKENAALKWEIAALKQG
Membrane-disrupting/cell-penetrating peptides:
GALA for membrane disruption:
(SEQ ID NO: 27076)
WEAALAFALNEALAEHLAEALAEALEALAA
Aurein 1.2:
(SEQ ID NO: 27077)
GLFDIIKKIAESF
Magainin-1:
(SEQ. ID NO: 27078)
GIGKFLHSAGKFGKAFVGEIMKS
Magainin-2:
(SEQ ID NO: 27079)
GIGKFLKSAKKFGKAFVGEIMNS
Melittin:
(SEQ ID NO: 27080)
GIGAVLKVETTGLPALISWINPKRQQ
Mastoparan X:
(SEQ ID NO: 27081)
INWKGIAAMAKKLL
Cecropin A:
(SEQ ID NO: 27082)
KWKLEKKIEKVGQNIRDGIIKAGPAVAVVGQATQIAK
Cecropin P1:
(SEQ ID NO: 27083)
SWLSKTAKKLENSAKKRISEGIAIAIQGGPR
Citropin 1.1:
(SEQ ID NO: 27084)
GLFDVIKKVASVIGGL
Temporin-1Lb:
(SEQ ID NO: 27085)
NFLGTLINLAKKIL
H2V33 L2 peptide:
(SEQ ID NO: 27086)
SYFILRRRRKRFPYFFTDVRVAA
Adenovirus pVI membrane fusion domain:
(SEQ ID NO: 27087)
AFSWGSLWSGIKNFGSTVKNY
Gamma-1 peptide from flock house virus:
(SEQ ID NO: 27088)
ASMWERVKSIIKSSLAAASNI
Poliovirus 2B pore-forming peptide:
(SEQ ID NO: 27089)
VTSTITEKLLKNLIKIISSLVIITRNYEDTTTVLATLALLGCDASPWQWL
Rhinovirus pore-forming peptide:
(SEQ ID NO: 27090)
IAQNPVENYIDEVLNEVLVVPNIN
Influenza HA2 pore-forming peptide:
(SEQ ID NO: 27091)
FLGIAEAIDIGNGWEGMEFG
Influenza HA2 derivative:
(SEQ ID NO: 27092)
GLFGAIAGFIENGWEGMIDG
HA-derived INF6:
(SEQ ID NO: 27093)
GLFGAIAGFIENGWEGMIDGWYG.
KRAB domain - epigenetic modification:
(SEQ ID NO: 27118)
MDAKSLTAWSRTLVTFKDVFVDFTREEWKLLDTAQQIVYRNVMLENYKNL
VSLGYQLTKPDVILRLEKGEEPWLV
Minimal Krab domain (KOX 11-55) - epigenetic
modification:
(SEQ ID NO: 21119)
RTLVTFKDVFVDFTREEWKLLDTAQQIVYRNVMLENYKNLVSLGY

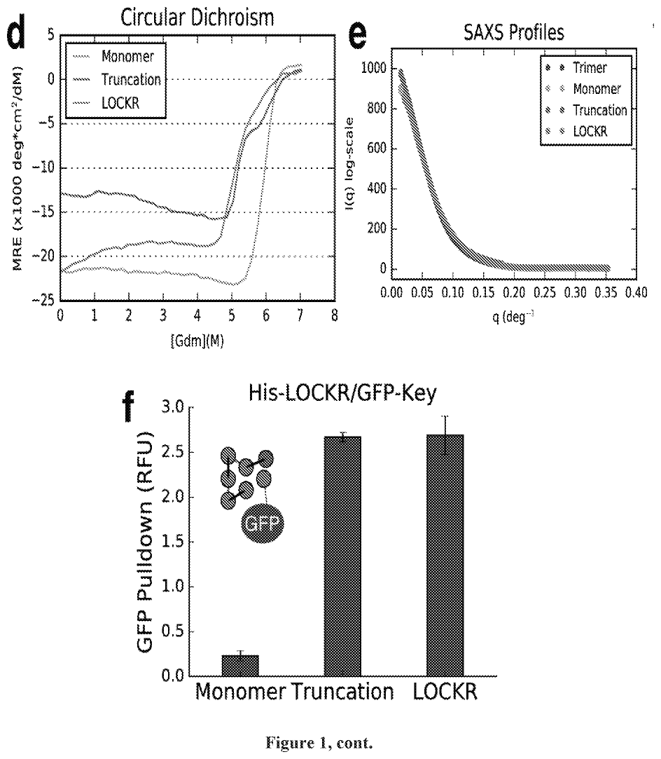

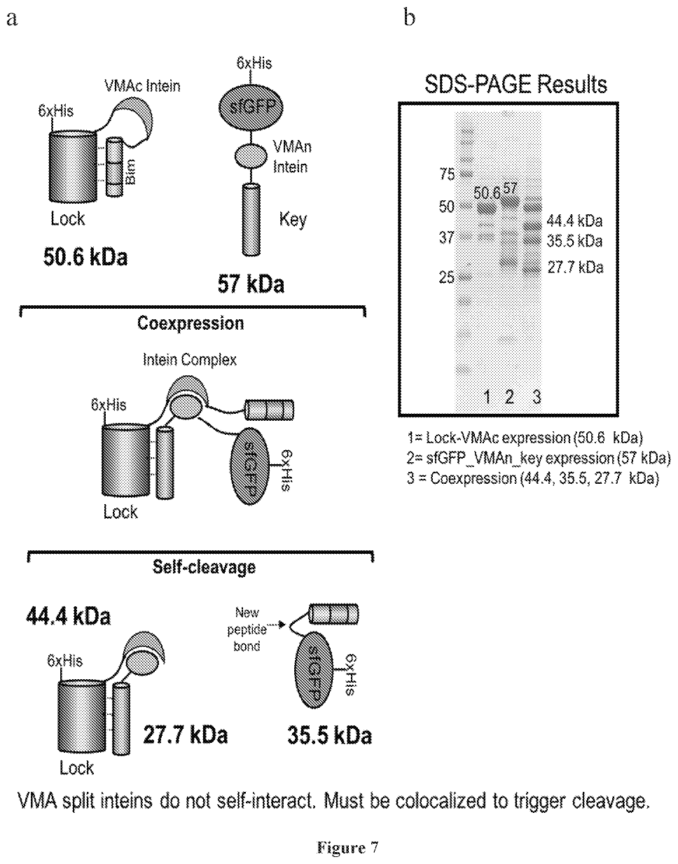

In one embodiment, the dynamic range of activation by key polypeptides can be tuned by truncating the latch region length to be shorter than the alpha-helices in the structural region, simultaneously weakening the cage polypeptide-latch region interaction and opening an exposed region on the cage polypeptide that the key polypeptide can bind to as a “toehold” (FIG. 2). Similarly, the dynamic range of activation by key polypeptides can also be tuned in a similar manner by designing mutations into the Latch that weaken the cage polypeptide-latch region interaction (FIGS. 1-2, and 10). In other embodiments, the latch region can be one or more helices totaling in length between 18-150 amino acids, between 18-100 amino acids, between 18-58 amino acids, or any range encompassed by these ranges. In other embodiments the latch region could consist of helical secondary structure, beta strand secondary structure, loop secondary structure, or combinations thereof.

In another embodiment, the cage polypeptide comprises the amino acid sequence having at least 4.5%, 50%, 55%, 60%. 65%, 70%, 75%, 80%, 85%, 90%, 91%, 92%, 93%, 94%, 95%, 96%, 97%, 98%, 99%, or 100% sequence identity along the length of the amino acid sequence of a cage polypeptide selected from the group consisting of (a) SEQ ID NO: 27359-28465 or a cage polypeptide listed in Table 7 (in (a) embodiments, the degron is included within the polypeptide sequence), and (b) SEQ ID NOS: 1-49, 51-52, 54-59.61, 65, 67-91, 92 -2033, SEQ ID NOS: 2034-14317, 27094-27117, 27120-27125, 27,278 to 27,321, and cage polypeptides listed in Table 2 or 5 (polypeptides with an even-numbered SEQ ID NO between SEQ ID NOS: 27126 and 27276), Table 3, and/or Table 4 ((i.e.: in (b) embodiments, the degron is not included in the amino acid sequence and would be added within the latch region, including but not limited to those degron amino acid sequences disclosed herein.

Amino Acid Sequences of deGronLOCKR Switches
DeGron sequence underlined
Latch indicated by [brackets]
>degonLOCKR_a_327
(SEQ ID NO: 27359)
SKEAVTKZQALNIKLAEKLLEAVTKLQALNIKLAEKLLEALAPLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AALKAESERIIPEGSGSGDPDVARLQELNIELARELLRDVARLQELNIELARELLRAAAELQELNIKLVELASEL
TDP[DEARKAIARVKRESKRIVEDAERLPMSCAQESEKISREAERLIREAA]
>degonLOCKR_a_327_noPro
(SEQ ID NO: 27360)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARRAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDP[DEARKAIARVKRESKRIVEDAERLAMSCAQESEKISREAERLIREAA]
>degonLOCKR_a_CAonly
(SEQ ID NO: 27361)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLNELLLEAIAYLQELNIKLVELLTKLTDPATIREAIRKVKEDSERIVAEAERLIAAAEAESERIIREAERLI
AAAKAESERIIREGSGSGDPDVARLQELNIELARELLRDVARLQELNIELARELLRAAAELQELNIKLVELASEL
TDP[DEARKAIARVKRESKRIVEDAERLIRECAAASEKISREAERLIREAA]
>degonLOCKR_a_324_t12
(SEQ ID NO: 27362)
SKEAVTKLQALNIKLAEKLLEAVTKLQALNIKLAEKLLEALARLQELNIALVYLAVELTDPKRIADEIKKVKDKS
KEIVEPAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLPDVARLQELNIELARELLRAAAELQELNIKLVELASEL
TDP[DEARKAIARVKRESKRIVEDLIMSCAQESAASEKISREAEPLTR]
>degonLOCKR_a_320_t16
(SEQ ID NO: 27363)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDP[DKARKAIARVKRESKRLVMSCAQESREAAAASEKISREAE]
>degron-miniLOCKRa_l_t9
(SEQ ID NO: 27364)
NKEDATEAQKKAIRAAEELLKDVTRIQERAIRFAEKALERLARVQEEAIRRVYEAVESKNKEELKKVKEEIEELL
RRLKRELDELEREIRELLKEIKEKADRIEKEIRDLIERIRRDRNASDEVVPRLARLNEELIRELREDVRRLAELN
KELLPELERAARELARLNEKLLELADRVETE[EFARKAIARVKRESKRIVEDAERLAMSCAQESEKISPEAERLI
REAA]
>degron-miniLOCKRa_1_t12
(SEQ ID NO: 27365)
NKEDATEAQKKAIRAAEELLKDVTRIQERAIRENEKALERLARVQEEAIRRVYEAVESKNKEELKKVKEEIEELL
RRLKRELDELEREIRELLKEIKEKADRLEKEIRDLIERIRRDRNASDEVVTRLARLNEELIRELREDVRRLAELN
KELLPELERAARELARLNEKLLELADRVETE[EEARKAIARVKRESKRIVEDLIMSCAQESAASEKISREAERLI
R]
>degron-miniLOCKRa_2_t9
(SEQ ID NO: 2736)
DERLKRLNERLADELDKDLERLLRLNEELARELTRAAEELRELNEKLVELAKKLQGGRSREVAERAEKEREKIRR
KLEEIKKEIKEDADRIKKRADELRRRLEKTLEDAARELEKIKRERRTEELKRKATELQKEAIRRAEELLKEVTDV
QRRAIERAEELLEKLARLQEEAIRTVYLLVELNKV[DRARKAIARVKRESKRIVEDAERLAMSCAQESEKISREA
ERLIREAA]
>degron-miniLOCKRa_t12
(SEQ ID NO: 27367)
DERLKRLNERLADELDKDLEPLLRLNEELARELTRAAEELPELNEKLVELAKKLOGGRSREVAERAEKEREKTRR
KLEEIKKEIKEDADRIKKRADELRRRLEKTLEDAARELEKLKREPRTEELKRKATELQKENIRRAEELLKEVTDV
QRRNIERAEELLEKLARLQEENIRTVYLLVELNKV[DRARKAIARVKRESKRIVEDLIMSCAQESAASEKISREA
ERLIR]
>lfix-short_cODC_mut
(SEQ ID NO: 21368)
SKEAAKKLQDLNIELARKLLEASTKLQRLNIRLAEALLEAIARLQELNLELVYLAVELTDPKRIRDEIKEVKDKS
KEIIRRAEKEIDDAAKESKKILEEARKAIRDAAEESRKILEEGSGSGSDALDELQKLNLELAKLLLKAIAETQDL
NLRAAKAFLEAAAKLQELNIRAVELLVKLTDPATIRRALEHAKRRSKEIIDEAERAIRAAKRESERIIEEARRLI
EKAKEESERIIREGSGSGDPDIKKLQDLNIELARELLRAHAQLQRLNLELLRELLRALAQLQELNLDLLRLASEL
TDPDEARKAIARVKPESKRIVEDAERLSPEAAALSMSCAQESERSIREAAAASEKISRE]
>lfix-short_cODC_mut_t6
(SEQ ID NO: 27369)
SKEAAKKLQDLNIELARKLLEASTKLQRLNIRLAEALLEAIARLQELNLELVYLAVELTDPKRIRDEIKEVKDKS
KEIIRRAEKEIDDAAKESKKILEEARKAIPDAAEESRKILEEGSGSGSDALDELQKLNLELAKLLLKAIAETQDL
NLRAAKAELEAAAKLQELNIRAVELLVKLTDRATIRPALEHAKRRSKEIIDEAERAIRAAKRESERIIEEARRLI
EKAKEESERIIREGSGSGDRDIKKLQDLNIELARELLRAHAQLQRLNLELLRELLRALAQLQELNLDLLRLASEL
TDP[DEARKAIARVKRESKRIVEDAERLSMSCAQESEKISREAERSIRKAAAS]
>lfix-short_cODC
(SEQ ID NO: 27370)
SELARKLLEASTKLQRLNIPLAEALLFAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIPAAKRESERIIEEARRLIEKGSGSGSELARELLRAHAQLQRLNLELLRELLRALA
QLQELNLDLLRLASELTDP[DEARKAIARVKRESKRIVEDLEMSCAQESAASEKISREAERLIR]
>lfix-short_cODC_t5
(SEQ ID NO: 27371)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAIARVKRESKRLVMSCAQESREAAAASEKISREA]
>lfix-short_cODC_t8
(SEQ ID NO: 27372)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAELKAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAIARVKRLSMSCAQESERLIREAAAASEKIK]
>lfix-short_cODC_t11
(SEQ ID NO: 21373)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKDQELNIRAVELLVKLTDPATIRE
ALEHAKRRSKEIIDEAERAIRAAKRESERIIEEARPLIEKGSGSGSELARELLRAHAQLQRLNLELLRELLRALA
QLQELNLDLLRLASELTDP[DEARKAIARLKMSCAQESEDAERLIREAAAASE]
>degonOCKRb
(SEQ ID NO: 27374)
SHAAVIKLSDLNIRLLDYLLQAVIKLTELNAELNRKLIEALQRLFDLNVALVHLAAELTDPKRIADEIKKVKDKS
KEIVERAEEEIARAAAESKKILDEAEEEIARAAAESKKILDEGSGSGSDAVAELQALNLKLAELLLEAVAELQAD
NLKLAELLLEAIAKLQELNIKLVELLTKLTDPATIREAIRKVKEDSERIVAEAERLIAAAKAESERIIREAERLI
AAAKAESERIIREGSGSNDPQVAQNQETFIELARDALRLVAENQEAFIEVARLTLRAAALAQEVAIKAVEAASEG
GSGSGP[NKEEIEKLAKEAREKLKKAEKEHKMSCAQERKKNKKAREDLKKKNDK]
>degonLOCKRb_t13
(SEQ ID NO: 27375)
SHAAVIKLSDLNIRLLDKLLQAVIKLTELNAELNRKLIEALQRLFDLNVALVHLAAELTDPKRIADEINKVKDKS
KEIVERNEEEIARAAAESKKILDEAEEEIARAAAESKKILDEGSGSGSDAVAELQALNLKLAELLLEAVAELQAL
NLKLAELLLEATAKLQELNIKLVELLTKLTDPATIPEAIRKVKEDSERIVAEAERLIAAAKAESERIIREAERLI
AAAKAESERIIREGSGSNDPQVAQNQETFIELARDALRLVAENQEAFIEVARLTLRAAALAQEVAIKAVEAASEG
GSCSGP[NKEEIEKLAKEAPEKLKKAEMSCAQEHDKDRKKNKKAREDLKK]
>degonLOCKRc_t9
(SEQ ID NO: 27376)
SLEAVLKLAELNLKLSDKLAEAVQKLAALLNKLLEKLSEALQRLFELNVALVTLAIELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSNDPLVARLQELLIEHARELLRLVATSQEIFIELAPAFLANAAQLQEAAIKAVEAASEN
GSGSGP[SSEKVRRELKESLKENHKQNQKLLMSCAQEQEKLNRELEELKKKHKK]
>degonLOCKRc_t13
(SEQ ID NO: 27377)
SLEAVLKLAELNLKLSDKLAEAVQKLAALLNKLLEKLSEALQRLFELNVALVTLAIELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSNDPLVARLQELLIEHARELLRLVATSQEIFIELARAFLANAAQLQEAAIKAVEAASEN
GSGSGP[SSEKVRRELKESLKENHKQNMSCAQEHKRAQEKLNRELEELKK]
>degron-miniLOCKRc_1_t9
(SEQ ID NO: 27378)
LIERLTRLEKEHVRELKRLLDTSLEILPPLVEAFETNLRQLKEALKRALEAANLHNEEVEEVLRKLEEDLRRLEE
ELRKTLDDVRKEVKRLKEELDKRIKEVEDELRKIKEKLKKGDKNEKRVLEEILRLAEDVLKKSDKLAKDVQERAR
ELNEILEELSRKLQELFERVVEEVTRNVPTTE[RIEKVRRELKESLKENHKQNQKLLMSCAQEQEKLNRELEELK
KKHKK]
>degron-miniLOCKRc_1_t13
(SEQ ID NO: 27379)
LIERLTRLEKEHVRELKRLLDTSLEILRRLVEAFETNLRQLKEALKRALEAANLHNEEVEEVLRKLEEDLRRLEE
ELRKTLDDVRKEVKRLKEELDKRIKEVEDELRKIKEKLKKGDKNEKRVLEEILRLAEDVLKKSDKLAKDVQERAR
ELNEILEELSRKLQELFERVVEEVTRNVPTTE[RIEKVRRELKESLKENHKLNMSCAQEHKRAQEKDNRELEELK
K]
>degron-miniLOCKRc_2_t9
(SEQ ID NO: 27380)
SEERVLELAEEALRLSDEAAKEIQELARRLNEELEKDSKELQDLFERIVETVTRLIDADEETLKRAAEEIKKRLE
DARKKAKEAADKAREELDRARKKLKELVDEIRKKAKDALEKAGADEELVARLLRLLEEHARELERLLRTSARIIE
RLLDAFRRNLEQLKEAADKAVEAAEEAVRRVEDVRV[WSEKVRRELKESLKENHKQNQKLLMSCAQEQEKLNREL
EELKKKHKK]
>dearon-miniLOCKRc_t13
(SEQ ID NO: 27381)
SEERVLELAEEALRLSDEAAKEIQELARRDNEELEKLSKELQDDFERIVETVTRLIDADEETLKRAAEEIKKRLE
DARKKAKEAADKAREELDRARKKLKELVDEIRKKAKDALEKAGADEELVARLLRLLEEHARELERLLRTSARIIE
RLLDAFRRNLEQLKEAADKAVEAAEEAVRRVEDVRV[WSEKVRRELKESLKENHKLNMSCAQEHKRAQEKLNREL
EELKK]
>degronLOCKRc_1fix_t13
(SEQ ID NO: 27382)
SLEAALKLAELNLKLSDKLAEASQKLAALLNKLLEKLSEAIQRLFELNLALVTLAIELTDPKRIADEIKKVKDKS
KEIIERAEEEIARAAAESKKILDEAEEEIARAAAESKKILDEGSGSGSDALAELQALNLKLAELLLEAIAETQAL
NLKAAEAFLEAAAKLQELNIKAVELLVKLTDPATIREALRKAKEDSERIIAEAERAIAAAKAESERIIREAERLI
AAAKAESERIIREGSGSNDPLIARLQELLIEHARELLRLHATSQEIFVELLRAFLANLAQLQEAALKALEAASEN
GSGSGP[SSEKVRRELKESLKENHKQNQKLLMSCAQEQEKLNREDELEELKKKHKK]
>degonLOCKRd
(SEQ ID NO: 27383)
SLEAVLKLFELNHKLSEKLLEAVLKLHALNQKLSQKLLEALARLLELNVALVELAIELTDPKRIADEIKKVKDKS
KEIVERAEEEIARAAAESKKILDEAEEEIARAAAESKKTLDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPEVARLQEAFIEQAREILRNVAAAQEALIEQARRLLALAALAQEAAIKAVELASEH
GSGSGP[DTVKRILEELRRRFEKLAKDLDDIAMSCAQEHKKHNKELKDKQRKIK]

Other exemplary cage polypeptides (see also SEQ ID NOS: 92-14317, 27094-27117, 27120-27125, 27728-27321, and cage polypeptides listed in Table 2, Table 3, Table 4, and/or Table 5). In these embodiments, the degron is not included in the amino acid sequence and would be added within the latch region, including but not limited to those degron amino acid sequences disclosed herein.
1) Exemplary reference cage polypeptides; latch regions denoted by brackets [ ]
6His-MBP-TEV, 6His-TEV, and flexible linker sequences are underlined text
fused functional domains (DARPins, components of the split intein, and fluorescent proteins) are bolded text
Functional peptide is italicized underlined text
Exemplary positions that have been mutated to any amino acid to tune responsiveness are underlined bolded text. These positions are exemplary, and not an exhaustive list of residues able to tune responsiveness.
C-terminal sequences that can be removed to tune responsiveness are contained within brackets. A range from one (1) to all residues encompassed within the brackets may be removed, starting from the C-terminus and removing successive residues therein.
All sequences in parentheses are optional

>SB76L
(SEQ ID NO: 1)
(MGSSHHHHHHSSGLVPRGSHM)SKEAVTKLQALNIKLASKLLFALARLQELNIALVYLAVELTDPKRIADEIKK
VKDKSKEIVERAEEEIARAAAESKKILDEGSGSGSDAVAELQALNLKLAELLLEAIAKLQELNIKLVELLTKLTD
PATIREAIRKVKEDSERIVAEAERLIAAAKAESERIIREGSGSGDPDVARLQELNIELARELLRAAAELQELNIK
LVELASELTDP[DFARKAIARVKRESKRIVEDAERLIREAAAASEKISRE]
>SB76L_17
(SEQ ID NO: 2)
(MGSSHHHHHHSSGLVPRGSHM)GSKEAVTKLMALNLKLAEKLLEAIARLQELNIALVYLATELTDPERIREEIR
KVKEESARIVEEAEEEIRRAAARSEDILREGSGSGSDAVAELQRLNLELAELLLRAAAKLQELNIDLVRLLTELT
DFKTIRDAIERVKAESERIVREAERLIREAKADSERILREG5G5GDPDVARLQELFIELARELLEALARLQELNI
DLVRLASELTDP[DTIRDAIRRVKEESARIVEDARRLIKEAAEEAEKISRE]
>SB76L_18
(SEQ ID NO: 3)
(MGSSHHHHHHSSGLVPRGSHM)GSKRAVTELQKLNIELARKLLRALAELMELNIALVYLAVELTDPRRIREEIR
KVKEKSDEIVKRAEDEIRKAAAESEKILREGSGSGSDAVAELQRLNLELAKLLLEAIAKLQALNIDLVRLLTELT
DPETIRRAIKRVKDESARIVEEAEKLIRAAKDKAREIIDKGSGSGDPDVARLQELNIELAPELLEAAARLQELFI
DLVRLASELTDP[DEARKAIERVKREAERIVREAERLIREAKRASKEISDE]
>LOCKR_extendb
(SEQ ID NO: 4)
(MGSSHHHHHHSSGLVPRGSHM)KLLEAVTKLQALNIKLAEKLLEALARLQELNIALVYLAVELTDPKRIADEIK
KVKDKSKEIVERAEEEIARAAAESKKILDEAEEEGSGSGSELLLEAVAELQALNLKLAELLLEAIAKLQELNIKL
VELLTKLTDPATIREAIRKVKEDSERIVAEAERLIAAAKAESERIIREAERLAGSGSGSRELLRDVARLQELNIE
LARELLRAAAELQELNIKLVELASELTDP[DEARKAIARVKRESKRIVEDAERLIREAAAASEKISREAERLI]
>LOCKR_extends
(SEQ ID NO: 5)
(MGSSHHHHHHSSGLVPRGSHM)KLAEKLLEAVTKLQALNIKLAEKLLEALARLQELNIALVYLAVELTDPKRIA
DEIKKVKDKSKEIVERAEEEIARAAAESKKILDEAEEEIARAGSGSGSLKLAELLLEAVAELQALNLKLAELLLE
AIAKLQELNIKLVELLTKLTDPATIREAIRKVKEDSERIVAEAERLIAAAKAESERIIREAERLIAAAAGSGSGS
IELARELLRDVARLQELNIELARELLRAAAELQELNIKLVELASELTDP[DEARKAIARVKRESKRIVEDAERLI
REAAAASEKISREAERLIREAA]
>LOCKR_excend18
(SEQ ID NO: 6)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVEPAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDP[DEARKAIARVKRESKRIVEDAERLIREAAAASEKISREAERLIREAAA
ASEKISRE]
>LOCKRb
(SEQ ID NO: 7)
(MGSSHHHHHHSSGLVPRGSHM)SHAAVIKLSDLNIRLLDKLLQAVIKLTELNAELNRKLIEALQRLFDLNVALV
HLAAELTDPKRIADEIKKVKDKSKEIVERAEEEIARAAAESKKILDEAEEEIARAAAESKKILDEGSGSGSDAVA
ELQALNLKIAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSNDPQVAQNQETFIELARDALRLVAENQEAPIEVAR
LTLPAAALAQEVAIKAVEAASEGGSGSG[NKEEIEKLAKEAREKLKKAEKEHKEIHDKLRKKNKKAREDLKKKAD
ELRETNKRVN]
>LOCKRc
(SEQ ID NO: 8)
(MGSSHHHHHHSSGLVPRGSHM)SLEAVLKLAELNLKLSDKLAEAVQKLAALLNKLLEKLSEALQRLFELNVALV
TLAIELTDPKRIADEIKKVKDKSKEIVERAEEEIARAAAESKKI1DEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSNDPLVAPLQELLIEHARELLRLVATSQEIFIELAR
AFLANAAQLQEAAIKAVEAASENGSGSG[SSEKVRRELKSSLKENHKQNQKLIKDHKRAQEKLNRELEELKKKHK
KTLDDIRRES]
>LOCKRd
(SEQ ID NO: 9)
(MGSSHHHHHHSSGLVPRGSHM)SLEAVLKLFELNHKLSEKLLEAVLKLHALNQKLSQKLLEALARLLELNVALV
ELAIELTDPKRIADEIKKVKDKSKEIVERAEEEIARAAAESKKILDEAEEEIAPAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDFEVARLQEAFIEQAREILRNVAAAQEALIEQAR
RLLALAALAQEAAIKAVELASEHGSGSG[DTVKRILEELRRRFEKLAKDLDDIARKLLEDKKKHNKELKDKQRKI
KKEADDAARS]
>LOCKRe
(SEQ ID NO: 10)
(MGSSHHHHHHSSGLVPRGSHM)SLEAVLKLQDLNSKLSEKLSEAQLKLQALNNKLLRKLLEALLRLQDLNQALV
NLALQLTDPKRIADEIKKVKDKSKEIVERASEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIRECSGSGDPDVAKSQEHLIEHARELLRQVAKSQELFIELAR
QLLRLAAKSQELAIKAVELASEAGSGSG[DDVERRLRKANKESKKEAEELTEEAKKANEKTKEDSKELTKENRKT
NKTIKDEARS]
>LOCKRf
(SEQ ID NO: 11)
(MGSSHHHHHHSSGLVPRGSHM)SREAVEKLAELNHKLSHKLQQAQQKLQALNLKLLQKLLEALDRLQDLNNALV
KLAQRLTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQSLNIKLVELLTKLTDPATIREAIRKVKEDSERIVAE
AERLIAAAKAESERIIREAERLIAAAKAESERIIREGSGSGDPDVARQQETLIEQARRLLRNVAESQELFIEAAR
TVLRLAAKLQEINIKQVELASEAGSGSGIDDEERRSEKTVQDAKREIKKVEDDLQRLNEEQKKKVKKQEDENQKT
LKKHKDDARS|
>miniLOCKRa_1
(SEQ ID NO: 12)
(MGSSHHHHHHSSGLVPRGSHM)NKEDATEAQKKAIRAAEELLKDVTRIQEPAIREAEKALERLARVQEEAIRRV
YEAVESKNKEELKKVKEEIEELLRRLKRELDELEREIRELLKEIKEKADRLEKEIRDLIERIRRDRNASDEVVTR
LARLNEELIRELREDVRRLAELNKELLRELERAARELARLNEKLLELADRVETEIEEARKAIARVKRESKRIVED
AERLIREAAAASEKISREAERLIREAAAASEKISRE]
>miniLOCKRa_2
(SEQ ID NO: 13)
(MGSSHHHHHHSSGLVPRGSHM)DERLKRLNERLADELDKDLERLLRLNEELARELTRAAEELRELNEKLVELAK
KLQGGRSREVAERAEKSRRKIRRKLEEIKKEIKEDADRIKKRADELRRRLEKTLEDAARELEKLKREPRTEELKR
KATELQKFAIRPAEELLKEVTDVQRRAIERAEELLEKLARLQEEAIRTVYLLVELNKV[DRARKAIARVKRESKR
IVEDAERLIREAAAASSKISREAERLIREAAAASEKISRE]
>miniLOCKRc_1
(SEQ ID NO: 14)
(MGSSHHHHHHSSGLVPRGSHM)LIERLTRLEKEHVRELKRLLDTSLEILRRLVEAFETNLPQLKEALKRALEAA
NLHNEEVEEVLRKLEEDLRRLEEELRKTLDDVRKEVKRLKEELDKRIKEVEDELRKIKEKLKKGDKNEKRVLEEI
LRLAEDVLKKSDKLAKDVQERARELNEILEELSRKLQELFERVVEEVTRNVPT[TERIEKVRRELKESLKENHKQ
NQKLLKDHKRAQEKLNRELEELKKKHKKTLDDIRRES]
>miniLOCKRc_2
(SEQ ID NO: 15)
(MGSSHHHHHHSSGLVPRGSHM)SEERVLELAEEALRLSDEAAKEIQELARRLNEELEKLSKELQDLFERIVETV
TRLIDADEETLKRAAEEIKKRLEDARKKAKEAADKAREELDRARKKLKELVDEIRKKAKDALEKAGADEELVARL
LRLLEEHARELERLLRTSARIIERLLDAFRRNLEQLKEAADKAVEAAEEAVRRVED[VRVWSEKVRRELKESEKE
NHKQNQKLLKDHKRAQEKLNEELEELKKKHKKTLDDIRRSS]
>lfix-short-noBim-t0
(SEQ ID NO: 16)
(MGSHHHHHHGSGSENLYFQGSGG)SELARKLLEASTKLQRLNIRLAEALLEAIARLQELNLELVYLAVELTDPK
RIRDEIKEVKDKSKEIIRRAEKEIDDAAKESEKILEEAREAISGSGSELAKLLLKAIAETQDLNLRAAKAFLEAA
AKEQELNIRAVELLVKLTDPATIREALEHAKRRSKEIIDEAERAIRAAKRESERIIEEARRLIEKGSGSGS[ELA
RELLRAHAQLQRLNLELLRELLRALAQLQELNLDLLR1A5ELTDPDEARKAIARVKRESKRIVEDAERLIREAAA
ASEKISREAERLIR]
>1fix-short-noBim(AYYA)-10
(SEQ ID NO: 17)
(MGSHHHHHHGSGSENLYFQGSGG)SELARKLLEASTKLQRLNIRLAEALLEAIARLQELNLELVYLAVELTDPK
RIRDEIKEVKDKSKEIIRRAEKEIDDAAKESEKILEEAREAISGSGSELAKLLLKAIAETQDLNLRAAKAFLEAA
AKLQELNIRAVELLVKLTDPATIREALEHAKRRSKEIIDEAERA1RAAKRESERIIEEARRLIEKGSGSGS[ELA
RELLRAHAQLQRLNLELLRELLRALAQLQELNLDLLPLASELTDPDEARKAIARVKRESNAYYADAERLIREAAA
ASEKISREAERLIR]
“(3) Functional LOCKR Cage designs with bioactive peptides encoded into the Latch”,
>aBc12L0CKR
(SEQ ID NO: 18)
(MGSSHHHHHHSSGLVPRGSHM)GSKFAVTKLQALNIKLAEKLLFALARLQELNIALVYIAVELTDPKRIADEIK
KVKDKSKEIVERAEEEIARAAAESKKILDEGSGSGSDAVAELQALNLKLAELLLEAIAKLQELNIKLVELLTKLT
DPATIREAIRKVKEDSERIVAEAERLIAAAKAESERIIREGSGSGDPDVARLQELNIELARELLRAAAELQELNI
KLVELASELT(GSGSGSG)[DPKMAQEL [symbol] DKVRAASLQI [symbol] GDAFYA [symbol] LRALAASEKLSKE]
>pBimLOCKR
(SEQ ID NO: 19)
(MGSSHHHHHHSSGLVPRGSHM)KEAVTKLQALNIKLAEKLLEALARLQELNIALVYLAVELTDRKRIADEIKKV
KDKSKEIVERAEEEIARAAAESKKILDEGSGSGSDAVASLQALNLKLAELLLEAIAKLQELNIKLVELLTKLTDP
ATIREAIRKVKEDSERIVAEAERLIAAAKAESERIIREGSGSGDPDVARLQELNIELARELLRAAAELQELNIKL
VELASEGSGSGS[EIAEALRAIGDV FNESYRIVEDAERLIREAAAASEKISRE]
> BimLOCKR_extend5
(SEQ ID NO: 20)
(MGSSHHHHHHSSGLVPRGSHM)KLLEAVTKLQALNIKLAEKLLEALARLQELNIALVYLAVELTDPKRIADEIK
KVKDKSKEIVERAEEEIARAAAESKKILDEAEEEGSGSGSELLLFAVAELQALNLKLAELLLEAIAKLQELNIKL
VELLTKLTDPATIEEAIRKVKEDSERIVAEAERLIAAAKAE3ERIIREAERLAGSGSGSRELLRDVARLQELNIE
LARELLRAAAELQELNIKLVELASELTD[EIWIAQELRRIGDEFNAYYADAERLIREAAAASEKISREAERLI]
>BimLOCKR_extend9
(SEQ ID NO: 21)
(MGSSHHHHHHSSGLVPRGSHM)KLAEKLLEAVTKLQALNIKLAEKLLEALARLQELNIALVYLAVELTDPKRIA
DEIKKVKDKSKELVERAEEEIARAAAESKKILDEAEEEIARAGSGSGSLKLAELLLEAVAELQALNLKLAELLLE
AIAKLQELNIKLVELLTKLTDPATIREAIRKVKEDSERIVAEAERLIAAAKAESERIIREAERLIAAAAGSGSGS
IELARELLRDVARLQELNIELARELLRAAAELQELNIKLVELASELTD[EIWIAQELRRIGDEFNAYYADAERLI
REAAAASEKISREAERLIRFAA]
>BimLOCKR_extend18
(SEQ ID NO: 22)
(MGSSHHHHHHSSGLVPRGSHM)SKEAVTKLQALNIKLAEKLLEAVTKLQALNIKLAEKLLFALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTD[EIWIAQELRRIGDEFNAYYADAERLIREAAAASEKISREAERLIREAAA
ASEKISRE]
>BimLOCKRb
(SEQ ID NO: 23)
(MGSSHHHHHHSSGLVPRGSHM)SHAAVIKLSDLNIRLLDKLLQAVIKLTELNAELNRKLIEALQRLFDLNVALV
HLAAELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSNDPQVAQNQETFIELARDALRLVAENQEAFIEVAR
LTLRAAALAQEVAIKAVEAASEGGSGSG[NEIWIAQELRRIGDEFNAYYAEHKEIHDKLRKKNKKAREDLKKKAD
ELRETNKRVN]
>BimLOCKRc
(SEQ ID NO: 24)
(MGSSHHHHHHSSGLVPRGSHM)SLEAVLKLAELNLKLSDKLAEAVQKLAALLNKLLEKLSEALQRLFELNVALV
TLAIELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSNDPLVARLQELLIEHARELLRLVATSQEIFIELAR
AFLANAAQLQEAAIKAVEAASENGSG[EIWIAQELRRIGDEFNAYYAQNQKLLKDHKPAQEKLNRELEELKKKHK
KTLDDIRRES]
>BimLOCKRd
(SEQ ID NO: 25)
(MGSSHHHHHHSSGLVPRGSHM)SLEAVLKLFELNHKLSEKLLEAVLKLHALNQKLSQKLLEALARLLELNVALV
ELAIELTDPKRIADEIKKVKDKSKEIVERAEEEIARAAAESKKILDEAEEEIAPAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPEVARLQEAFIEQAREILRNVAAAQEALIEQAR
KLLALAALAQEAAIKAVELASEHGSGS[EIWIAQELRRIGDEFNAYYADLDDIARKLLEDHKKHNKELKDKQRKI
KTIKDEARS]
>StrepLOCKRa_300
(SEQ ID NO: 26)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELAS(GG)[NWSHPQFEKKAIARVKRESKRIVEDAERLIREAAAASEKISREAERLIR
EAAAASEKISPE]
>strepLOCKRa_306
(SEQ ID NO: 27)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[ENWSHPQFEKRESKRIVEDAERLIREAAAASEKISREAERLIREAAA
ASEKISRE]
>strepLOCKRa_309
(SEQ ID NO: 28)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKNWSHPQFEKKRIVEDAERLIREAAAASEKISREAERLIREAAA
ASEKISRE]
>strepLOCKRa_312
(SEQ ID NO: 29)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIANWSHPQFEKEEDAERLIREAAAASEKISREAERLIREAAA
ASEKISRE]
>strepLOCKRa_313
(SEQ ID NO: 30)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARNWSHPQFEKEDAERLIREAAAASEKISREAERLIREAAA
ASEKISRE]
>strepLOCKRa_317
(SEQ ID NO: 31)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKRENWSHPQFEKRLIREAAAASEKISREAERLIREAAA
ASEKISRE]
>strepLOCKRa_320
(SEQ ID NO: 32)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKRESKRNWSHPQFEKREAAAASEKISREAERLIREAAA
ASEKISRE]
>strepLOCKRa_323
(SEQ ID NO: 33)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKRESKRIVENWSHPQFEKAAASEKISREAERLIREAAA
ASEKISRE]
>strepLOCKRa_329
(SEQ ID NO: 34)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKRESKRIVEDAERLINWSHPQFEKISREAERLIREAAA
ASEKISRE]
>SB13_LOCKR
(SEQ ID NO: 35)
(MGSSHHHHHHSSGLVPRGSHM)GSKEAVTKLQALNIKLAEKLLEALARLQELNIALVYLAVELTDPKRIADEIK
KVKDKSKEIVERAEEEIARAAAESKKILDEGSGSGSDAVAELQALNLKLAELLLEAIAKLQELNIKLVELLTKLT
DPATIREAIRKVKEDSERIVAEAERLIAAAKAESERIIREGSGSGDPDVARLQELNIELARELLRAAAELQELNI
KLVELASEGSGSGSG[YELRRALEELEKALRELKKSLDEI [symbol] RSLEEL [symbol] KNPSEDALVENNRLNVENNKIIVEVLR
IIAEVLKINAS]
>ZCX12_LOCKR
(SEQ ID NO: 36)
(MGSSHHHHHHSSGLVPRGSHM)GSKEAVTKLQALNIKLAEKLLEALARLQELNIALVYLAVELTDPKRIADEIK
KVKDKSKEIVERAEEEIARAAAESKKILDEGSGSGSDAVAELQALNLKLAELLLEAIAKLQELNIKLVELLTKLT
DPATIREAIRKVKEDSERIVAEAERLIAAAKAESERIIREGSGSGDPDVARLQELNIELARELLRAAAELQELNI
KLVELASEGSGSGSG[KKLVEEVERALRELLKTSEDLVRKV [symbol] KALRELLELIRRGGTKDKIEEKIPRVLEEIKRE
LERQKRKIEDVLRQIKEELYRS]
>SB13_LOCKR_extend18
(SEQ ID NO: 37)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASEGSGSGSG[YELRRALEELEKALRELKKSLDELERSLEELEKNFSEDALVENNR
LNVENNKIIVEVLRIIAEVIKLNAKS]
>ZCX12_LOCKR_extend18
(SEQ ID NO: 38)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASEGSGSGSG[KKLVEEVERALRELLKTSEDLVRKVEKALRELLELIRRGGTKDKI
EEKIRRVLEEIKRELERQKRKIEDVLRQIKEELYRS]
>fretLOCKRa
(SEQ ID NO: 39)
(GHHHHHHHHHHGVSKGEELFTGVVPILVELDGDVNGHKFSVSGEGEGDATYGKLTLKFICTTGKLPVPWPTLVT
TLSWGVQCFARYPDHMKQHDFFKSAMPEGYVQERTIFFKDDGNYKTRAEVKFEGDTLVNRIELKGIDFKEDGNIL
GHKLEYNYFSDNVYITADKQKNGIKANFKIRHNIEDGGVQIADHYQQNTPIGDGPVLLPDNHYLSTQSKLSKDPN
EKRDHMVLLEFVTAAGITLGMDELYKGSGCSLQGM)SKEAVTKLQALNIKLAEKLLEAVTKLQALNIKLAEKLLE
ALARLQELNIALVYLAVELTDPKRIADEIKKVKDKSKEIVERAEEEIARAAAESKKILDEAEEEIARAAAESKKI
LDEGSGSGSDAVAELQALNLKLAELLLEAVAELQALNLKLAELLLEAIAKLQELNIKLVELLTKLTDPATIREAI
RKVKEDSERIVAEAERLIAAAKAESERIIREAERLIAAAKAESERIIREGSGSGDPDVARLQELNIELARELLRD
VARLQELNIELARELLRAAAELQELNIKLVELASELTDPD[EARKAIARVKRESKRIVEDAERLIREAAAASEKI
SREAERLIREAAAASEKISRE](VSKGEELFTGVVPILVELDGDVNGHKFSVSGEGEGDATYGKLTLKLICTTGK
LPVPWPTLVTTLGYGVQCFARYPDHMKQHDFFKSAMPEGYVQERTIFFKDDGNYKTRAEVKFEGDTLVNRIELKG
IDFKEDGNILGHKLEYNYNSHNVYITADKQKNGIKANFKIRHNIEDGGVQLADHYQQNTPIGDGPVLLPDNHYLS
YQSKLSKDPNEKRDHMVLLEFVTAAGITLGMDELYK)
>fretLOCKRb
(SEQ ID NO: 40)
(MGHHHHHHHHKHGVSKGEELFTGVVPILVELDGDVNGHKFSVSGEGEGDATYGKLTLKFICTTGKLPVPWPTLV
TTLSWGVQCFARYPDHMKQHDFFKSAMPEGYVQERTIFFKDDGNYKTRAEVKFEGDTLVNRIELKGIDFKEDGNI
LGHKLEYNYFSDNVYITADKQKNGIKANFKIRHNIEDGGVQLADHYQQNTPIGDGPVLLPDNHYLSTQSKLSKDP
NEKRDHMVLLEFVTAAGITLGMDELYKGSGCSLQGM)SHAAVIKLSDLNIRLLDKLLQAVIKLTELNAELNRKLI
EALQRLFDLNVALVHLAAELTDPKRIADEIKKVKDKSKEIVERAEEEIARAAAESKKILDEAEEELAPAAAESKK
ILDEGSGSGSDAVAELQALNLKLAELLLEAVAELQALNLKLAELLLEAIAKLQELNIKLVELLTKLTDPATIREA
IRKVKEDSERIVAEAERLIAAAKAESERIIREAERLIAAAKAESERIIREGSGSNDPQVAQNQETFIELARDALR
LVAENQEAFIEVARLTLRAAALAQEVAIKAVEAASEGGSGSGINKEEIEKLAKEAREKLKKAEKEHKEIHDKLRK
KNKKAREDLKKKADELRETNKRVN](VSKGEELFTGVVPILVELDGDVNGHKFSVSGEGEGDATYGKLTLKLICT
TGKLPVPWPTLVTTLGYGVQCFARYPDHMKQHDFFKSAMPEGYVQERTIFFKDDGNYKTRAEVKFEGDTLVNRIE
LKGIDFKEDGNILGHKLEYNYNSHNVYITADKQKNGIKANFKIRHNIEDGGVQLADHYQQNTPIGDGPVLLPDNH
YLSYQSKLSKDPNEKRDHMVLLEFVTAAGITLGMDELYK)
>fretLOCKRc
(SEQ ID NO: 41)
(GHHHHHHHHHHGVSKGEELFTGVVPILVELDGDVNGHKFSVSGEGEGDATYGKLTLKFICTTGKLPVPWPTLVT
TLSWGVQCFARYPDHMKQHDFFKSAMPEGYVQERTIFFKDDGNYKTRAEVKFEGDTLVNRIELKGIDFKEDGNIL
GHKLEYNYFSDNVYITADKQKNGIKANFKIRHNIEDGGVQLADHYQQNTPIGDGPVLLPDNHYLSTQSKLSKDPN
EKRDHMVLLEFVTAAGITLGMDELYKGSGCSLQGM)SLEAVLKLAELNLKLSDKLAEAVQKLAALLNKLLEKLSE
ALQRLFELNVALVTLAIELTDPKRIADEIKKVKDKSKEIVERAEEEIARAAAESKKILDEAEEEIARAAAESKKI
LDEGSGSGSDAVAELQALNLKLAELLLEAVAELQALNLKLAELLLEAIAKLQELNIKLVELLTKLTDPATIREAI
RKVKEDSERIVAEAERLIAAAKAESERIIREAERLIAAAKAESERIIREGSGSNDPLVARLQELLIEHARELLRL
VATSQEIFIELARAFLANAAQLQEAAIKAVEAASENGSGSGS[SEKVRRELKESLKENHKQNQKLLKDHKFAQEK
LNRELEELKKKHKKTLDDIRRES](VSKGEELFTGVVPILVELDGDVNGHKFSVSGEGEGDATYGKLTLKLICTT
GKLPVPWPTLVTTLGYGVQCFARYPDHMKQHDFFKSAMPEGYVQERTIFFKDDGNYKTRAEVKFEGDTLVNRIEL
KGIDFKEDGNILGHKLEYNYNSHNVYITADKQKNGIKANFKIRHNIEDGGVQLADHYQQNTPIGDGPVLLPDNHY
LSYQSKLSKDPNEKRDHMVLLEFVTAAGITLGMDELYK)
>fretLOCKRd
(SEQ ID NO: 42)
(GKHKHHHHHHHGVSKGEELFTGVVPILVELDGDVNGHKFSVSGEGEGDATYGKLTLKFICTTGKLPVPNPTLVT
TLSWGVQCFARYPDHMKQHDFFKSAMPEGYVQERTIFFKDDGNYKTRAEVKFEGDTLVNRIELKGIDFKEDGNIL
GHKLEYNYFSDNVYITADKQKNGIKANFKIRHNIEDGGVQLADHYQQNTPIGDGPVLLPDNHYLSTQSKLSKDPN
EKRDHMVLLEFVTAAGITLGMDELYKGSGCSLQGM)SLEAVLKLFELNHKLSEKLLEAVLKLKALNQKLSQKLLE
ALARLLELNVALVELAIELTDPKRIADEIKKVKDKSKEIVERAEEEIARAAAESKKILDEAEEEIARAAAESKKI
LDEGSGSGSDAVAELQALNLKLAELLLFAVAELQALNLKLAELLLEAIAKLQELNIKLVELLTKLTDPATIREAI
RKVKEDSERIVAEAERLIAAAKAESERIIREAERLIAAAKAESERIIREGSGSGDPEVARLQEAFIEQAREILRN
VAAAQEALIEQARRLLALAALAQEAAIKAVELASEHGSGSGIDTVKRILEELRRRFEKLAKDLDDIARKLLEDHK
KHNKELKDKQRKIKKEADDAARS](VSKGEELFTGVVPILVELDGDVNGHKFSVSGEGEGDATYGKLTLKLICTT
GKLPVPWPTLVTTLGYGVQCFARYPDHMKQHDFFKSAMPEGYVQERTIFFKDDGNYKTRAEVKFEGDTLVNRIEL
KGIDFKEDGNILGHKLEYNYNSHNVYITADKQKNGIKANFKIRHNIEDGGVQLADHYQQNTPIGDGPVLLPDNHY
LSYQSKLSKDPNEKRDHMVLLEFVTAAGITLGMDELYK)
>tevLOCKR
(SEQ ID NO: 43)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKRESKRIVEDAEENLYFQGAASEKISREAERLIREAAA
ASEKISRE]
>spyLOCKR
(SEQ ID NO: 44)
(MGSSHHHHHHSSGLVPRGSHM)SKEAVTKLQALNIKLLEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARAHIVMVDAYKKRIVEDAERLIREAAAASEKISREAERLIREAAA
ASEKISRE]
>1_nesLOCKR
(SEQ ID NO: 45)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDPD[EARKAIARVKRESKRIVEDLALKLAGLDINSEKISREAERLIREAAAASEKISRE]
>2_nesLOCKR
(SEQ ID NO: 46)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDPD[EARKAIARVKRESKRIVEDAERLIRELAEKLAGLDINAERLIREAAAASEKISRE]
>3_nesLOCKR
(SEQ ID NO: 47)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDPD[EARKAIARVKRESKELAEKLRAGLDLNAAAASEKISREAERLIREAAAASEKISRE]
>nlsLOCKR
(SEQ ID NO: 48)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDPD[EARKAIARVKRESKAAAKRARTSIREAAAASEKISREAERLIREAAAASEKISRE]
>ezh2LOCKR
(SEQ ID NO: 49)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKTMFSSNRQKILERTETLNQEWKQRRIQAERLIREAAA
ASEKISRE]
>1fix_VMAc_C_BIMlatcht9
(SEQ ID NO: 51)
(MGSHHHHHHGSGSENLYFQG)SKEAAKKLQDLNIELARKLLEASTKLQRLNIRLAEALLEAIARLQELNLELVY
LAVELTDPKRIRDEIKEVKDKSKEIIRRAEKEIDDAAKESKKILEEARKAIRDAAEESRKILEEGSGSGSDALDE
LQKLNLELAKLLLKAIAETQDLNLRAAKAFLEAAAKLQELNIRAVELLVKLTDPATIRRALEHAKRRSKEIIDEA
ERAIRAAKRESERIIEEARRLIEKAKEESERIIREGSGSGDPDIKKLQDLNIELARELLRAHAQLQRLNLELLRE
LLRALAQLQELNLDLLRLASELTGGSGGSGGS(VLLNVLSKCAGSKKFRPAPAAAFARECRGFYFELQELKEDDY
YGITLSDDSDHQFLLANQVVVHNC)GGSGGS[DEIWIAQELRRIGDEFNAYYADAERLIREAAAASEKISREAER
LIREAA]
>sfGFP_VMAn_1fix_BIM_t0_latch
(SEQ ID NO: 52)
(MGSHHKHHHGSGSENLYFQG)HMSKGEELFTGVVPILVELDGDVNGHKFSVRGEGEGDATNGKLTLKFICTTGK
LPVPWPTLVTTLTYGVQCFARYPDHMKQHDFFKSAMPEGYVQERTISFKDDGTYKTRAEVKFEGDTLVNRIELKG
IDFKEDGNILGHKLEYNFNSHNVYITADKQKNGIKANFKIRHNVEDGSVQLADHYQQNTPIGDGPVLLPDNHYLS
TQSVLSKDPNEKRDHMVLLEFVTAAGITHGMDELYKSGSGSGCFAKGTNVLMADGSIECIENIEVGNKVMGKDGR
PREVIKLPRGRETMYSVVQKSQHRAHKSDSSREVPELLKFTCNATHELVVRTPRSVRRLSRTIKGVEYFEVITFE
MGQKKAPDGRIVELVKEVSKSYPISEGPERANELVESYRKASNKAYFEWTIEARDLSLLGSHVRKATYQTYAPIL
YGGSGGSGGGGSGGSGSKEAAKKLQDLNIELARKLLEASTKLQRLNIRLAEALLEAIARLQELNLELVYLAVELT
DPKRIRDEIKEVKDKSKEIIRRAEKEIDDAAKESKKILEEARKAIRDAAEESPKILEEGSGSGSDALDELQKLNL
ELAKLLLKAIAETQDLNLRAAKAFLEAAAKLQELNIRAVELLVKLTDPATIRRALEHAKRRSKEIIDEAERAIRA
AKRESERIIEEARRLIEKAKEESERIIREGSGSGDPDIKKLQDLNIELARELLRAHAQLQRLNLELLRELLRALA
QLQELNLDLLRLASELT[DEIWIAQELRRIGDEFNAYYADAERLSREAAAASEKISREAERSIREAAAASEKISR
E]
Asymmetrized functional Cages encoding Bim and GFP11 (i.e.: bioactive
peptides)
(6His-MBP-TEV, 6HIs-TEV, and flexible linker sequences are underlined text)
(Co-localization domain is bolded text)
(Functional peptide is italicized underlined text)
(Positions that can be mutated to any amino acid to tune responsiveness are
underlined bolded text)
(C-terminal sequences that can be removed to tune responsiveness are
italicized text)
(all sequences in parentheses are optional)
> 1fix-long-BIM-t0
(SEQ ID NO: 54)
(MGSHHHHHHGSGSENLYFQG)SKEAAKKLQDLNIELARKLLEASTKLQRLNIRLAEALLEAIARLQELNLELVY
LAVELTDPKRIRDEIKEVKDKSKEIIRRAEKEIDDAAKESKKILEEARKAIRDAAEESRKILEEGSGSGSDALDE
LQKLNLELAKLLLKAIAETQDLNLRAAKAFLEAAAKLQELNIRAVELLVKLTDPATIRRALEHAKRRSKEIIDEA
ERAIRAAKRESERIIEEARRLIEKAKEESERIIREGSGSGDPDIKKLQDLNIELARELLRAHAQLQRLNLELLRE
LLRALAQLQELNLDLLRLASEL(TD[EIWIAQELRRIGDEFNAYYA)DAERLIREAAAASEKISREAERLIREAA
AASEKISRE]
>1fix-long-GFP-t0
(SEQ ID NO: 55)
(MGSHHHHHHGSGSENLYFQG)SKEAAKKLQDLNIEIARKLLEASTKLQRLNIRLAEALIEAIARLQELNLELVY
LAVELTDPKRIRDEIKEVKDKSKEIIRRAEKEIDDAAKESKKIIEEARKAIRDAAEESRKILEEGSGSGSDALDE
LQKLNLELAKLLLKAIAETQDLNLRAAKAFLEAAAKLQELNIRAVELLVKLTDPATIRRALEHAKRRSKEIIDEA
ERAIRAAKRESERIIEEARRLIEKAKEESERIIREGSGSGDPDIKKLQDLNIELARELLRAHAQLQRLNLELLRE
LLRALAQLQELNLDLLRLASEL[(RDHMVLHEYVNAAGITFNAYYA)DAERLIREAAAASEKISREAERLIREAA
AASEKISRE]
>1fix-short-BIM-t0
(SEQ ID NO: 56)
(MGSHHHHHHGSGSENLYFQGSGG)SELARKLLEASTKLQRLNIRLAEALLEAIARLQELNLELVYLAVELTDPK
RIRDEIKEVKDKSKEIIRRAEKEIDDAAKESEKILEEAREAISGSGSELAKLLLKAIAETQDLNLRAAKAFLEAA
AKLQELNIRAVELLVKLTDPATIREALEHAKRRSKEIIDEAERAIRAAKRESERIIEEARRLIEKGSGSGSELAR
ELLRAHAQLQRLNLELLRELLRALAQLQELNLDLLRLASEL(TD[EIWIAQELRRIGDEFNAYYA)DAERLIREA
AAASEKISREAERLIR]
>1fix-short-GFP-t0
(SEQ ID NO: 57)
(MGSHHHHHHGSGSENLYFQGSGG)SELARKLLEASTKLQRLNIRLAEALLEAIARLQELNLELVYLAVELTDPK
RIRDEIKEVKDKSKEIIRRAEKEIDDAAKESEKILEEAREAISGSGSELAKLLLKAIAETQDLNLRAAKAFLEAA
AKLQELNIRAVELLVKLTDPATIREALEHAKRRSKEIIDEAERAIRAAKRESERIIEEARRLIEKGSGSGSELAR
ELLRAHAQLQRLNLELLRELLRALAQLQELNLDLLRLASEL[(RDHMVLHEYVNAAGITFNAYYA)DAERLIREA
AAASEKISREAERLIR]
>Spycatcher-1fix-long-GFP-t0
(SEQ ID NO: 58)
(MGSHHHHHHGSGSENLYFQGS)AMVDTLSGLSSEQGQSGDMTIEEDSATHIKFSKRDEDGKELAGATMELRDSS
GKTISTWISDGQVKDFYLYPGKYTFVETAAPDGYEVATAITFTVNEQGQVTVNGKATKGSGGSKEAAKKLQDLNI
ELARKLLEASTKLQRLNIRLAEALLEAIARLQELNLELVYLAVELTDPKRIRDEIKEVKDKSKEIIRRAEKEIDD
AAKESKKILEEARKAIRDAAEESRKILEEGSGSGSDALDELQKLNLELAKLLLKAIAETQDLNLRAAKAFLEAAA
KLQELNIRAVELLVKLTDPATIRRALEHAKRRSKEIIDEAERAIAAAKRESERIIEEARRLIEKAKEESERIIRE
GSGSGDPDIKKLQDLNIELARELLRAHAQLQRLNLELLRELLRALAQLQELNLDLLRLASELL(RDHMVLHEYVN
AAGITFNAYYA)DAERLIREAAAASEKISREAERLIREAAAASEKISRE]
>Spycatcher-1fix-short-GFP-t0
(SEQ ID NO: 59)
(MGSHHHHHHGSGSENLYFQGS)AMVDTLSGLSSEQGQSGDMTIEEDSATHIKFSKRDEDGKELAGATMELRDSS
GKTISTWISDGQVKDFYLYPGKYTFVETAAPDGYEVATAITFTVNEQGQVTVNGKATKGSGGSELARKLLEASTK
LQRLNIRLAEALLEAIARLQELNLELVYLAVELTDPKRIRDEIKEVKDKSKEIIRRAEKEIDDAAKESEKILEEA
REAISGSGSELAKLLLKAIAETQDLNLRAAKAFLEAAAKLQELNIRAVELLVKLTDPATIREALEHAKRRSKEII
DEAERAIRAAKRESERIIEEARRLIEKGSGSGSELARELLRAHAQLQRLNLELLRELLRALAQLQELNLDLLRLA
SEL[(RDHMVLHEYVNAAGITFNAYYA)DAERLIREAAAASEKISREAERLIR]
>1fix-latch_Mad1SID_t0_1
(SEQ ID NO: 61)
(MGSHHHHHHGSGSENLYFQG)SKEAAKKLQDLNIELARKLLEASTKLQRLNIRLAEALLEAIARLQELNLELVY
LAVELTDPKRIRDEIKEVKDKSKEIIRRAEKEIDDAAKESKKILEEARKAIRDAAEESRKILEEGSGSGSDALDE
LQKLNLELAKLLLKAIAETQDLNLRAAKAFLEAAAKLQELNIRAVELLVKLTDPATIRRALEHAKRRSKEIIDEA
ERAIRAAKRESERIIEEARRLIEKAKEESERIIREGSGSGDPDIKKLQDLNIELARELLRAHAQLQRLNLELLRE
LLRALAQLQELNLDLLRLASELT](NIQMLLEAADYLE)RESKRIVEDAERLIREAAAASEKISREAERSIREAA
AASEKISRE]
>1fix-latch_Mad1SID_T0_2
(SEQ ID NO: 65)
(MGSHHHHHHGSGSENLYFQG)SKEAAKKLQDLNIELARKLLEASTKLQRLNIRLAEALLEAIARLQELNLELVY
LAVELTDPKRIRDEIKEVKDKSKEIIRRAEKEIDDAAKESKKILEEARKAIRDAAEESRKILEEGSGSGSDALDE
LQKLNLELAKLLLKAIAETQDLNLRAAKAFLEAAAKLQELNIRAVELLVKLTDPATIRRALEHAKRRSKEIIDEA
ERAIRAAKRESERIIEEARRLIEKAKEESERIIREGSGSGDPDIKKLQDLNIELARELLRAHAQLQRLNLELLRE
LLRALAQLQELNLDLLRLASELTDP[DEARK(NIQMLLEAADYLE)EDAERLIREAAAASEKISREAERLIREAA
SEKISRE]
>1fix-short-Bim-t0-relooped
(SEQ ID NO: 67)
[MDEARKAIARVKRESKRI(EIWIAQELRRIGDEFNAYYA)EAEKIAP]DELWHRLLEASTKLQRLNIRLAEALL
EAIARLQELNLELVYLAVELTDPKRIRDEIKEVKDKSKEIIRRAEKEIDDAAKESEKILEEAREAISGSGSELAK
LLLKAIAETQDLNLRAAKAFLEAAAKLQELNIRAVELLVKLTDPATIREALEHAKRRSKEIIDEAERAIRAAKRE
SERIIEEARRLIEKGSGSGSELARELLRAHAQLQRLNLELLRELLRALAQLQELNLDLLRLASE
>1fix-short-spyzag-t0_2
(SEQ ID NO: 68)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDPD[EAR(AHIVMVDAYK)KRIVEDAERLIREAAA
ASEKISREAERLIR]
>1fix-shorz-spyrag-t0_8
(SEQ ID NO: 69)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDPD[EARKAIARVKRESK(AHIVMVDAYK)REAAA
ASEKISREAERLIR]
>1fix-short-TEV-t0_1
(SEQ ID NO: 70)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEAR(ENLYFQGS)ESKRIVEDAERLIREAAA
ASEKISREAERLIR]
>1fix-short-TEV-t0_6
(SEQ ID NO: 71)
(MGSSHHHHHHSSGLVPRGSHM)SELARKLLEASTKLQRLNIPLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLPLASELTDP[DEARKAIARVKRESKRIV(ENLYFQGS)EAAA
ASEKISREAERLIR]
>1fix-short-nanoBit-t0_1
(SEQ ID NO: 72)
(MGSSHHHHHHSSGLVPRGSHM)SELARKLLEASTKLQRLNIPLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEAR(VSGWRLFKKIS)RIVEDAERLIREAAA
ASEKISREAERLIR]
>1fix-short-nanoBit-t0_3
(SEQ ID NO: 73)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIARVKRESK(VSGWRLFKKIS)EAAA
ASEKISREAERLIR]
>1fix-short-RHIM-10_8
(SEQ ID NO: 74)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAI(IQIG)RESKRIVEDAERLIREAAA
ASEKIS(VQLG)RLIR)
>1fix-short-RHJLM-t0_19
(SEQ ID NO: 75)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIARVKRESKRIV(IQIG)RLI(VQLG
)AASEKISREAERLIR]
>1fix-short-RHIM-t0_22
(SEQ ID NO: 76)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIARVKRESKRIV(IQIG)RLIREAAA
ASEKIS(VQLFG)RLIR]
>1fix-short-gcn4-t0_4
(SEQ ID NO: 77)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DESVKE(LEDKVEELLSKNYHLEMEVARLKKL
VGER)SREAERLIR]
>1fix-short-ccDi-t0_6
(SEQ ID NO: 78)
(MGSSHHHHHHSSGLVPRGSHM)SELARKLLEASTKLQRLNIRLAEALLEAIARLQELNLELVYLAVELTDPKRI
RDEIKEVKDKSKEIIRRAEkEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIA(GEIAALKQEIAALKKENAALKHE
IAALKQG)AERLIR]
>1fix-short-cc-a-t0_6
(SEQ ID NO: 79)
(MGSSHHHHHHSSGLVPRGSHM)SELARKLLEASTKLQRLNIRLAEALLEAIARLQELNLELVYLAVELTDPKRI
RDEIKEVKDKSKEIIRRAEkEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIARVKR(GLKQEIAALEKSNAALEWE
IAALEQGG)EPLIR]
>1fix-short-cc-b-t0_6
(SEQ ID NO: 80)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIARVKR(GLKQKIAALKYKNAALKKK
IAALKQGG)ERLIR
STREPII-LOCKR functional Cages:
>STREPII-2plus1_DOCK_1
(SEQ ID NO: 81)
SRVEEIIEDLRRLLEEIRKENADSIRASKELLDRVKEINDTIIAELERLLKD1EKEVREKGSESEEVKKALRRVL
EELEKLLRRVAEINEEVLRRNSKLVEEDARRNAEVLKELKRLVEELMREIGDED[KVRKVAEVAEKVLRDIDKLD
R(WSHPQFEK)TNGEISKLDEDTRRVAERVKKAIEDLAK]
>STREPII-2plus1_LOCK_2
(SEQ ID NO: 82)
[SEVDEIIADNERALDEVRREVEEIDKENAERLGE(WSHPQFEK)GDPLAKALEEIRK]GVRSRLVDELERAIRE
VEEVIRRVLERVRRLIEEVSKIITDVLREVERLHEEVTKELRKVEDGNSREALDALRPLIEKVVEDSARLIKKVD
EALKAVNKE1EDLSREVADLVRAVAEELDARVK
>STREPII-2plus1_LOCK_3
(SEQ ID NO: 83)
SSDEVLKEIEEIIRRDEAEVRRVNAEVNASTEDLAREVEEVLRATNELIEELERRVTGTEELKRVIDELRDRDRK
VRRRVERVIEESAKRDDESRKRLTRAVEKLRADLKKLADDGVPE[EALSKAIKDVRDIVKKVKDELKE(WSHPQF
EK)VDRLSEELKEWLKDVERVLKELTDKDR]
>STREPII-2plus1_LOCK_4C
(SEQ ID NO: 84)
SDAEELLKRVADLLKASIESLEKILRDSKELMDRWRKKLEDLLRESEELVDRAEKILRRGGSDKEVLDKIAEEVR
RTNDDSRRLDEELHRLSRDTLRKLEENLRRTEKEVREMDKRAAERGIVDERVREELKKLLTRVE(WSHFQFEK)G
DKKILKEAHKESKEVNDRRRELLERLEESVR]
>STREPII-2plus1_LOCK_4N
(SEQ ID NO: 85)
[SPASELLKRVADLLKASLESLEKILRDSKELMDR(WSHPQEEK)LGESEELVDPAEKILRR]GGSDKEVLDKIA
EEVRRTNDDSRRLDEELHRLSRDTLRKLEEMLRRTEKEVREMDKRAAERGVDERVREELKKLLTRVEEEHRKVLE
TDKKILKEAHKESKEVNDRDRELLERLEESVR
>STREPII-3plus1_LOCK_1
(SEQ ID NO: 86)
SEAEDLLERVKRVLDELIEIVDRNHELNARVVETSARLVERLLEEVERALETLEREIPGRELLDKAIKDLRDVLR
RVAEKVKRSIEELKEVLEESRRVLEEVVRALAEVIDRVRRLVEKGVDLRDLIRELKRVLEEAVSLIERLVRLNTR
AAEKDNESLRELVRAIKEALKRAVDMVRADGL[PSRLVKKLDEIVKEVAKKLEDVVRANEEL(WSHPQFEK)GSS
VARLPFAVSRVARDLEETAR]
>STREPII-3plus1_LOCK_2
(SEQ ID NO: 87)
[SDEERLEKVVKDVIEKVRRILEK(WSHPQFEK)GSELRRILEEWEKIIREVLDKVRR]GSGSADALVEVLEEIL
RLAEELSKRVEEVLREILKLAKALSDELVKVLAEIVEAAKRISRDDELRKAVEDVARELEDLAAKDRKILDDVRE
ALERIAKEDKDILREAEETLRRLADEMRRSGVDERLLKRVVDILARLLELNATTIERLLRILEELLKLNKELAER
VIRVLEKLLEEIKR
>STREPII-3plus1_LOCK_3
(SEQ ID NO: 88)
SVLETVKKALEDSSEKIERIVEEDERVAKESSDRIRRLVEEDKPVADEILDLIEKGGDTDTLAKLVEEWSRTSKK
LLDDVLKLHKDWSDDSRRLLEEILRVHEELIRAVKEILDRGGKPEEVVRELEKVLKESLDTLEEIIRRLDEANAR
TVKRVADVIRELEDANAKVLEEIERKGD[DKDAVIKVIEELIRANAAV(WSHPQFEK)GDLVRVNKTVWKELLRV
NEKLARDLERWK]
>STREPII-3plus1_LOCK_4
(SEQ ID NO: 89)
[SLVDELRKSLERNVRVSEEVARRLKFALGR(WSHPQFEK)GGDLIRLNEDVVRVVEKV]GVDESAIERVRRIIE
ELNRALDAVLKKNEDLVRRLTELLDKLLEENRRLVEELDEDLKRRGGTEEVIDTILELIERSIERLKRLLDELLR
IVREALKDNARVADENLKALKEILDELRKDGVSDEELKRVLEKAADLKARLKDAHRKLLEDLERIIRELKKKLDE
VVEENKRSVDELKR
>STREPII-3plus1_LOCK_3-relooped
(SEQ ID NO: 90)
[MKDAVIKVIEELIRANAAV(WSHFQFEK)GDLVRVNKTVWKELLRVNEKLARDLERAL]DERDVSAWETVKKAL
EDSSEKIERIVEEDERVAKESSDRIRRLVEEDKRVADEILDLIEKGGDTDTLAKLVEEWSRTSKKLLDDVLKLHK
DWSDDSRRLLEEILRVHEELIRAVKEILDRGGAPEEVVRELEKVLKESLDTLEEIIRRLDEANARTVKRVADVIR
ELEDANAKVLEEIERK
>STREPII-2plus1_LOCK_3-relooped
(SEQ ID NO: 91)
[MEEAASKAIKDVRDIVKKVKDSLKE(WSHFQFEK)VDRLSEELKEWLKDVERVLKELT]DREEASEEELKRVID
ELRDRDRKVRRRVERVIEESAKRDDESRKRLTRAVEKLRADLKKLSVEGASDEVLKEIEEIIRRLEAEVRRVNAE
VNASTEDLAREVEEVLRATNELIESLERR
>BimLOCKR_a_short_Nterm
(SEQ ID NO: 27094)
[MDEARKAIARVKRESKRI(EIWIAQELRRIGDEFNAYYA)EAEKLATDEL]WHRLLEASTKLQRLNIRLAEALL
EAIARLQELNLELYYLAVELTDPKRIRDEIKEVKDKSKEIIRRAEKEIDDAAKESEKILEEAREAISGSGSELAK
LLLKAIAETQDLNLRAAKAFLFAAAKLQELNIRAVELLVKLTDPATIREALEHAKRRSKEIIDEAERAIRAAKRE
SERIIEEARRLIEKGSGSGSELARELLRAHAQLQPLNLELLRELLRALAQLQELNLDLLRLASE
>BimLOCKR_g
(SEQ ID NO: 27095)
(M3LVDEL(EIWIAQELRRIGDEFNAYYA)ALKRWVDVVRKVVEDLIRLNEDVVRVVEKV]GVDESAIERVRRII
EELNRALDAVLKKNEDLVRRLTELLDKLLEENRRLVEELDEDLKRRGGTEEVIDTILELIERSIERLKRLLDELL
RIVREALKDNARVADENLKALKEILDELRKDGVSDEELKRVLEKAADLHARLKDAHRKLLEDLERIIRELKKKLD
EWEENKRSVDELKR
>reloop_strepLOCKRh
(SEQIDNO: 27096)
[MKDAVIKVIEELIRANAAV(WSHPQFEK)GDLVRVNKTVWKELLRVNEKLARDLERALDER)DVSAWETVKKAL
EDSSEKIERIVEEDERVAKESSDRIRRLVEEDKRVADEILDLIEKGGDTDTLAKLVEEWSRTSKKLLDDVLKLHK
DWSDDSRRLLEEILRVHEELIRAVKEILDRGGAPEEVVRELEKVLKESLDTLEEIIRRLDEANARTVKRVADVIR
ELEDANAKVLEEIERK
>reloop_strepLOCKRi
(SEQ ID NO: 27097)
[MEEAASKAIKDVRDIVKKVKDSLKE(WSHPQFEK)VDRLSEELKEWLKDVERVLKELTDREEA]SEEELKRVID
ELRDRDRKVRRRVERVIEESAKRDDESRKRLTRAVEKLRADLKKLSVEGASDEVLKEIEEIIRRLEAEVRRVNAE
VNASTEDLAREVEEVLRATNELIEELEPP
>spyLOCKRa_2
(SEQ ID NO: 27098)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEAR(AHIVMVDAYK)KRIVEDAERLIREAAAASEKISREAERLIR]
>spyLOCKRa_8
(SEQ ID NO: 27099)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDPDEARKAIARVKRESK(AHIVMVDAYK)REAAAASEKISREAERLIR]
>tevLOCKRa_1
(SEQ ID NO: 27100)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEAR(ENLYFQGS)ESKRIVEDAERLIREAAAASEKISREAERLIR]
>tevLOCKRa_6
(SEQ ID NO: 27101)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAIARVKRESKRIV(ENLYFQGS)EAAAASEKISREAEPLIR]
>lucLOCKRa_l
(SEQ ID NO: 27102)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEAR(VSGWRLFKKIS)RIVEDAERLIREAAAASEKISREAERLIR)
>lucLOCKRa_3
(SEQ ID NO: 27103)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DSARKAIARYKRESK(VSGWRLFKKIS)EAAAASEKISREAERLIR)
>rhimLOCKRa_8
(SEQ ID NO: 27104)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAI(IQIG)RESKRIVEDAERLIREAAAASEKIS(VQLG)RLIR]
>rhimLOCKRa_19
(SEQ ID NO: 27105)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAIARVKRESKRIV(IQIG)RLI(VQLG)AASEKISREAERLR]
>rhimLOCKRa_22
(SEQ ID NO: 27106)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP(DEARKAIARVKRESKRIV(IQIG)RLIREAAAASEKIS(VQLG)RLIR]
>gcn4LOCKRa_4
(SEQ ID NO: 27107)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DESVKE(LEDKVEELLSKNYHLENEVAPLKKLVGER)SREAERLIR]
>cc-DiLOCKRa_6
(SEQ ID NO: 27108)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAIA(GEIAALKQEIAALKEENAALEWEIAALKQGG)ERLIR]
>cc-aLOCKRa_6
(SEQ ID NO: 27109)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DFARKATARVKR(GLEQEIAALEKENAAIEWEIAALEQGG)ERLIR]
>cc-bLOCKRa_6
(SEQ ID NO: 27110)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAIARVKR(GLKQKIAALKYKNAALKKKIAALKQGG)ERLIR]
>tev-spyLOCKRa_short_40
(SEQ ID NO: 27111)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAI(ENLYFQGS)RIVEDAE(AHIVMVDAYK)EKISREAERLIR]
>tev-spyLOCKRa_short_57
(SEQ ID NO: 27112)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAIARV(ENLYFQGS)EDAERLIREA(AHIVMVDAYK)AERLIR]
>tev-spyLOCKRa_short_63
(SEQ ID NO: 27113)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAIARVK(ENLYFQGS)DAERLIREA(AHIVMVDAYK)AERLIR]
>tev-spyLOCKRa_29
(SEQ ID NO: 27114)
SKEAAKKLQDLNIELARKLLEASTKLQRLNIRLAEALLEAIARLQELNLELVYLAVELTDPKRIRDEIKEVKDKS
KEIIRRAEKEIDDAAKESKKILEEARKAIRDAAEESRKILEEGSGSGSDALDELQKLNLELAKLLLKAIAETQDL
NLRAAKAFLEAAAKLQELNIRAVELLVKLTDPATIRRALEHAKRRSKEIIDEAERAIRAAKRESERIIEEARRLI
EKAKEESERIIREGSGSGDP[DIKKLQDLNIELARELLRAHAQLQRLNLELLRELLRALAQLQELNLDLLRLASE
LTDPDEARKAIARVK(ENLYFQGS)DAERLIREAAAASE(AHIVMVDAYK)REAAAASEKISRE]
>tev-spyLOCKRa_32
(SEQ ID NO: 27115)
SKEAAKKLQDLNIELARKLLEASTKLQRLNIRLAEALLEAIARLQELNLELVYLAVELTDPKRIRDEIKEVKDKS
KEIIRRAEKEIDDAAKESKKILEEARKAIRDAAEESRKILEEGSGSGSDALDELQKLNLELAKLLLKAIAETQDL
NLRAAKAFLEAAAKLQELNIRAVELLVKLTDPATIRRALEHAKRRSKEIIDEAERAIRAAKRESERIIEEARRLI
EKAKEESERIIREGSGSGDP[DIKKLQDLNIELARELLRAHAQLQRLNLELLRELLRALAQLQELNLDLLRLASE
LTDPDEARKAIARVK(ENLYFQGS)DAERLIREAAAASEKISREAE(AHIVMVDAYK)EKISRE]
>Bim-fretLOCKRa_short
(SEQ ID NO: 27116)
(VSKGEELFTGVVPILVELDGDVNGHKFSVSGEGEGDATYGKLTLKFICTTGKLPVPWPTLVTTLSWGVQCFARY
PDHMKQHDFFKSAMPEGYVQERTIFFKDDGNYKTRAEVKFEGDTLVNRIELKGIDFKEDGNILGHKLEYNYFSDN
VYITADKQKNGIKANFKIRHNIEDGGVQLADHYQQNTPIGDGPVLLPDNHYLSTQSKLSKDFNEKRDHMVLLEFV
TAAGITLE)LARKLLEASTKLQRLNIRLAEALLEAIARLQELNLELVYLAVELTDPKRIRDEIKEVKDKSKEIIR
RAEKEIDDAAKESEKILEEAREAISGSGSELAKLLLKAIAETQD1NLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLR
ELLRALAQLQELNLDLLRLASELT[D(EIWIAQELRRIGDEFNAYYA)DAERLIREAAAASEKISREAERLIR](
VSKGEELFTGVVPILVELDGDVNGHKFSVSGEGEGDATYGKLTLKLICTTGKLPVPWPTLVTTLGYGVQCFARYP
DHMKQHDFFKSAMPEGYVQERTIFFKDDGNYKTRAEVKFEGDTLVNRIELKGIDFKEDGNILGHKLEYNYNSHNV
YITADKQKNGIKANFKIRHNIEDGGVQIADHYQQNTPIGDGPVLLPDNHYLSYQSKLSKDPNEKRDHMVLLEFVT
AAGITLGMDELYKGSGC)
>fretLOCKRa_short
(SEQ ID NO: 27117)
(VSKGEELFTGVVPILVELDGDVNGHKFSVSGEGEGDATYGKLTLKFICTTGKLPVPWPTLVTTLSWGVQCFARY
PDHMKQHDFFKSAMPEGYVQERTIFFKDDGNYKTRAEVKFEGDTLVNRIELKGIDFKEDGNILGHKLEYNYFSDN
VYITADKQKNGIKANFKIRHNIEDGGVQLADHYQQNTPIGDGPVLLPDNHYLSTQSKLSKDFNEKRDHMVLLEFV
TAAGITL)ELARKLLEASTKLQRLNIRLAEALLEAIARLQELNLELVYLAVELTDPKRIRDEIKEVKDKSKEIIR
RAEKEIDDAAKESEKIEEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLR
ELLRALAQLQELNLDLLRLASELTDP[DEARKAIARVKRESNAYYADAERLIREAAAASEK](VSKGEELFTGVV
PILVELDGDVNGHKFSVSGEGEGDATYGKLTLKLICTTGKLPVPWPTLVTTLGYGVQCFARYPDHMKQHDFFKSA
MPEGYVQERTIFFKDDGNYKTRAEVKFEGDTLVNRIELKGIDFKEDGNILGHKLEYNYNSHNVYITADKQKNGIK
ANFKIRHNIEDGGVQLADHYQQNTPIGDGPVLLPDNHYLSYQSKLSKDPNEKRDHMVLLEFVTAAGITLGMDELY
KGSGC)
E18_KRAB_full
(SEQ ID NO: 27120)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LTGS[(RTLVTFKDVFVDFTREEWKLLDTAQQIVYRNVMLENYKNLVSLGYG)SDEARKAIARVKRESKRIVEDA
ERLIREAAAASEKISREAERLIREAAAASEKISRE]
E18_KRAB_N13t
(SEQ ID NO: 27121)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LTGS[(RTLVTFKDVFVDFTREEWKLLDTAQQIVYRNVMLENYKNLVSLGY)GSSKRIVEDAERLIREAAAASEK
ISREAERLIREAAAASEKISRE]
E18_KRAB_C9t
(SEQ ID NO: 27122)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LTGS[(RTLVTFKDVFVDFTREEWKLLDTAQQIVYRNVMLENYKNLVSLGY)GSDEARKAIARVKRESKRIVEDA
ERLIREAAAASEKISREAERLIREAA]
E18_KRAB_Cterm1
(SEQ ID NO: 27123)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LT[DEARKAIARVKRESKRIVEDAE(RTLVTFKDVFVDFTREEWKLLDTAQQIVYRNVMLENYKNLVSLGY)]
E18_KRAB_Cterm2
(SEQ ID NO: 27124)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LT[DEARKAIAPVKRESKRIVEDAERLI(RTLVTFKDVFVDFTREEWKLLDTAQQIVYRNVMLENYKNLVSLGY)
E18_KRAB_Cterm3
(SEQ ID NO: 27125)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LT[DEARKAIARVKRESKPIVEDAERLIREAAAASEKISRTLVTFKDVFVDFTREEWKLLDTAQQIVYRNVMLEN
YKNLVSLGY)]
>3plus1_Cage_Nterm_GFP11_668
(SEQ ID NO: 27278)
DEAKELLDEIRKAVKESEDRLEKLLRDYEKELRRDHMVLHEYVNAAGITLEELRRGSLDAKELLKTLEDLLREVL
EVARRVVETLKELNRRVLEVVREDIERNERLLRRVLDTLRRGGVDERRIKDLERLIRESLKKAEEVLREAAEKSR
EIVDEIREVLKRADEALKRIIKKIRETRGADALSRLLEELLRVVDDLIRVLKELIDKSRKVIEELLELLKRINEE
NLKVLAEIIK
>3plus1_Cage_Cterm_GFP11_668
(SEQ ID NO: 27279)
DEAKELLDEIRKAVKESEDRLEKLLRDYEKELRRLEKELRDLKRRIEEKLEELRRGSLDAKELLKTLEDLLREVL
EVARRVVETLKELNRRVLEVVREDIERNERLLRRVLDTLRRGGVDERRIKDLERLIRESLKKAEEVLREAAEKSR
EIVDEIREVLKRADEALKRIIKKIRETRGADADHMVLHEYVNAAGITIRVLKELIDKSRKVIEELLELLKRINEE
NLKVLAEIIK
>3plus1_Cage_Cterm_GFP11_668
(SEQ ID NO: 27280)
DEAKELLDEIRKAVKESEDRLEKLLRDYEKELRRLEKELRDLKRRIEEKLEELRRGSLDAKELLKTLEDLLREVL
EVARRVVETLKELNRRVLEVVREDIERNERLLRRVLDTLRRGGVDERRIKDLERLIRESLKKAEEVLREAAEKSR
EIVDEIREVLKRADEALKRIIKKIRETRGADARDHMVLHEYVNAAGITRVLKELIDKSRKVIEELLELLKRINEE
NLKVLAEIIK
>3plus1_Cage_Cterm_GFP11_668
(SEQ ID NO: 27281)
DEAKELLDEIRKAVKESEDRLEKLLRDYEKELRRLEKELRDLKRRIEEKLEELRRGSLDAKELLKTLEDLLREVL
EVARRVVETLKELNRRVLEVVREDIERNERLLRRVLDTLRRGGVDERRIKDLERLIRESLKKAEEVLREAAEKSR
EIVDEIREVLKRADEALKRIIKKIRETRGADALSRDHMVLHEYVNAAGITLKELIDKSRKVIEELLELLKRINEE
NLKVLAEIIK
>3plus1_Cage_Nterm_GFP11_668
(SEQ ID NO: 27282)
DEAKELLDEIRKAVKESEDRLEKLLRDYEKELRRLEKELRDLKRRIEEKLEELRRGSLDAKELLKTLEDLLREVL
EVARRVVETLKELNRRVLEVVREDIERNERLLRRVLDTLRRGGVDERRIKDLERLIRESLKKAEEVLREAAEKSR
EIVDEIREVDKRADEALKRIIKKIRDHMVLKEYVNAAGITLRVVDDLIRVLKELIDKSRKVIEELLELLKRINEE
NLKVLAEIIK
>3plus1_Cage_Nterm_GFP11_669
(SEQ ID NO: 27283)
SEKEKLLKESEEEVRRLRRTLEELLRKYREVLERLRDHMVLHEYVNAAGITRLKEVLDRSGLDIDTIIKEVEDLL
KTVLDRLRELLDKIARLTKEAIEVVREIIERIVRHAERVKDELRKGGADKRKLDRVDRLIKENTRHLKEILDRIE
DLVRRSEKKLRDIIREVRRLIEELRKKAEEIKKDPDERLVKTLIEDVERVIKRILELITRVAEDNERVLERIIRE
LTDNLEFHLKIVREIVK
>3plus1_Cage_Nterm_GFP11_670
(SEQ ID NO: 27284)
SEKEDLARKLRKLVEELTREYEELVKKLERLIEEIERDHMVLHEYVNAAGITISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPTGDEALRKLVELLVEVLRRLIRVNRELVKLLREVLERLLRILRESVKKLKR
LIEKVIKDAT
>3plus1_Cage_Cterm_GFP11_670
(SEQ ID NO: 27285)
SEKEDLARKLRKLVEELTREYEELVKKLERLIEEIEKVSEESVRKLEKLLREISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPTGDEARDHMVLHEYVNAAGITRVNRELVKLLREVLERLLRILRESVKKLKR
LIEKVIKDAT
>3plus1_Cage_Cterm_GFP11_670
(SEQ ID NO: 27286)
SEKEDLARKLRKLVEELTREYEELVKKLERLIEEIEKVSEESVRKLEKLLREISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPTGDERDHMVLHEYVNAAGITIRVNRELVKLLREVLERLLRILRESVKKLKR
LIEKVIKDAT
>3plus1_Cage_Cterm_GFP11_670
( SEQ ID NO: 27287)
SEKEDLARKLRKLVEELTREYEELVKKLERLIEEIEKVSEESVRKLEKLLREISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPTGRDHMVLHEYVNAAGITRLIRVNRELVKLLREVLERLLRILRESVKKLKR
LIEKVIKDAT
>3plus1_Cage_Nterm_GFP11_670
(SEQ ID NO: 27288)
SEKEDLARKLRKLVEELTREYEELVKKLERLIEEIRDHMVLHEYVNAAGITEISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPTGDEALRKLVELLVEVLRRLIRVNRELVKLLREVLERLLRILRESVKKLKR
LIEKVIKDAT
>3plus1_Cage_Cterm_GFP11_670
(SEQ ID NO: 27289)
SEKEDLARKLRKLVEELTREYEELVKKLERLIEEIEKVSEESVRKLEKLLREISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPTRDHMVLKEYVNAAGITRRLIRVNRELVKLLREVLERLLRILRESVKKLKR
LIEKVIKDAT
>3plus1_Cage_Nterm_GFP11_670
(SEQ ID NO: 27290)
SEKEDLARKLRKLVEELTREYEELVKKLERLIERDHMVLHEYVNAAGITLREISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPTGDEALRKLVELLVEVLRRLIRVNRELVKLLREVLERLLRILRESVKKLKR
LIEKVIKDAT
>3plus1_Cage_Nterm_GFP11_670
(SEQ ID NO: 27291)
SEKEDLARKLRKLVEELTREYEELVKKLERLIEEIEKRDHMVLHEYVNAAGITSEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPTGDEALRKLVELLVEVLRRLIRVNRELVKLLREVLERLLRILRESVKKLKR
LIEKVIKDAT
>3plus1_Cage_Nterm_GFP11_670
(SEQ ID NO: 27292)
SEKEDLARKLRKLVEELTREYEELVKKLERLIEEIEKVSEESRDHMVLHEYVNAAGITKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPTGDEALRKLVELLVEVLRRLIRVNRELVKLLREVLERLLRILRESVKKLKR
LIEKVIKDAT
>3plus1_Cage_Nterm_GFP11_670
(SEQ ID NO: 27293)
SEKEDLARKLRKLVEELTREYEELVKKLERLIEEIEKVSEESVRDHMVLHEYVNAAGITLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPTGDEALRKLVELLVEVLRRLIRVNRELVKLLREVLERLLRILRESVKKLKR
LIEKVIKDAT
>3plus1_Cage_Cterm_GFP11_671
(SEQ ID NO: 27294)
SEEEDLLERVKRVLDELIEIVDRNHELNRRVVETSAALVERLLEEVERALETLEREIPGSSLLDKAIKDLRDVLR
RVKEKVKRSIEELKEVLEESRRVLEEVVRKLREVIDRVRRLVEKGVDLRDLIRELKRVLEEAVKLIERLVRLNTR
AAEKDNESLRELVRAIKEALKRAVDAVRKGGLDSRAVKKLDRDHMVLHEYVNAAGITNEELWRALVELNKESVRR
LREIVERVARDLEETAR
>3plus1_Cage_Cterm_GFP11_671
(SEQ ID NO: 27295)
SEEEDLLERVKRVLDELIEIVDRNHELNRRVVETSAALVERLLEEVERALETLEREIPGSSLLDKAIKDLRDVLR
RVKEKVKRSIEELKEVLEESRRVLEEVVRKLREVIDRVRRLVEKGVDLRDLIRELKRVLEEAVKLIERLVRLNTR
AAEKDNESLRELVRAIKEALKRAVDAVRKGGLDSRARDHMVLHEYVNAAGITDVVRRNEELWRALVELNKESVRR
LREIVERVARDLEETAR
>3plus1_Cage_Cterm_GFP11_671
(SEQ ID NO: 27296)
SEEEDLLERVKRVLDELIEIVDRNHELNRRVVETSAALVERLLEEVERALETLEREIPGSSLLDKAIKDLRDVLR
RVKEKVKRSIEELKEVLEESRRVLEEVVRKLREVIDRVRRLVEKGVDLRDLIRELKRVLEEAVKLIERLVRLNTR
AAEKDNESLRELVRAIKEALKRAVDAVRKGGLDSRRDHMVLHEYVNAAGITEDVVRRNEELWRALVELNKESVRR
LREIVERVARDLEETAR
>3plus1_Cage_Cterm_GFP11__671
(SEQ ID NO: 27297)
SEEEDLLERVKRVLDELIEIVDRNHELNRRVVETSAALVERLLEEVERALETLEREIPGSSLLDKAIKDLRDVLR
RVKEKVKRSIEELKEVLEESRRVLEEVVRKLREVIDRVRRLVEKGVDLRDLIRELKRVLEEAVKLIERLVRLNTR
AAEKDNESLRELVRAIKEALKRAVDAVRKGGLRDHMVLHEYVNAAGITKKLEDVVRRNEELWRALVELNKESVRR
LREIVERVARDLEETAR
>3plus1_Cage_Cterm_GFP11_672
(SEQ ID NO: 27298)
DETEEVIKKIAKLIDELIETLKKIVEDSAKLIKELIDLLEEIIKRSADLIRELEKVSKRGDRDRLKKLIDRMRRE
TERALEELRRVIEELKEATEPALKTIEDLIREIEEAIRRELGDEDLAREILEVIRRILELLREDMELNLRALEEI
LRLLEEVIKLNKDAIDEILELLKRDHMVLHEYVNAAGITESKKLVRDVVEEAKRLLKENRDSTRKIIEDIRRLLR
KIEDSTR
>3plus1_Cage_Cterm_GFP11_672
(SEQ ID NO: 27299)
DETEEVIKKIAKLIDELIETLKKIVEDSAKLIKELIDLLEEIIKRSADLIRELEKVSKRGDRDRLKKLIDRMRRE
TERALEELRRVIEELKSATERALKTIEDLIREIEEAIRRSLGDEDLAREILEVIRRILELLREDMELNLRALEEI
LRLLEEVIKLNKDAIDEILELLKGRDHMVLHEYVNAAGITSKKLVRDVVEEAKRLLKENRDSTRKIIEDIRRLLR
KIEDSTR
>3plus1_Cage_Nterm_GFP11_67>3
(SEQ ID N0:27300)
SKEETLRKEAEDLLRRLEELTRRLEKKARELLERAKKLSRDLARDHMVLHEYVNAAGITEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVTDLSRKNKRILEELLKAIETLSDENKKILTEILDAL
RRLVEKIEK
>3plus1_Cage_Nterm_GFP11_67>3
(SEQ IB NO: 27301)
SKEETLRKEAEDLLRRLEELTRRLEKKARELLERAKKLSRRDHMVLHEYVNAAGITGVPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVTDLSRKNKRILEELLKAIETLSDENKKILTEILDAL
RRLVEKIEK
>3plus1_Cage_Cterm_GFP11_67>3
(SEQ ID NO: 27302)
SKEETLRKEAEDLLRRLEELTRRLEKKARELLERAKKLSRDLAEELKRLLKELREKGVPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEARDHMVLHEYVNAAGITRKNKRILEELLKAIETLSDENKKILTEILDAL
RRLVEKIEK
>3plus1_Cage_Nterm_GFP11_67>3
(SEQ ID NO: 27303)
SKEETLRKEAEDLLRRLEELTRRLEKKARELLERARDHMVLHEYVNAAGITELREKGVPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVTDLSRKNKRILEELLKAIETLSDENKKILTEILDAL
RRLVEKIEK
>3plus1_Cage_Nterm_GFP11_67>3
(SEQ ID NO: 27304)
SKEETLRKEAEDLLRRLEELTRRLEKKARELLERAKKLSRDHMVLHEYVNAAGITKGVPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVTDLSRKNKRILEELLKAIETLSDENKKILTEILDAL
RRLVEKIEK
>3plus1_Cage_Nterm_GFP11_67>3
(SEQ ID NO: 27305)
SKEETLRKSAEDLLRRLEELTRRLEKKARELLERAKKLRDHMVLHEYVNAAGITEKGVPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVTDLSRKNKRILEELLKAIETLSDENKKILTEILDAL
RRLVEKIEK
>3plus1_Cage_Nterm_GFP11_67>3
(SEQ ID NO: 27306)
SKEETLRKEAEDLLRRLEELTRRLEKKARELLERAKKLSRDLPDHMVLHEYVNAAGITPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVTDLSRKNKRILEELLKAIETLSDENKKILTEILDAL
RRLVEKIEK
>3plus1_Cage_Nterm_GFP11_674
(SEQ ID NO: 27307)
SEREEVKEKLDRLLEEVEKTVRELKREHDELLKEVEKLVRDLKRDHMVLHEYVNAAGITKEILDVIREKVRTNKE
ILDRVLEVVEEHLRRNKEILDKLLDDIRKVVEDAKRILGIGDDETLRRAVRRILEELRKLVEDIRKKVKDSLETL
ERALEEAEERIRRSLEDLKRVLKEAKDKTKDKDRLDKVEELVKKLLEDTKRTVDRVRELVRKILKKSRETLEELE
RLIEKILRSLEKDAR
>3plus1_Cage_Cterm_GFP11_674
(SEQ ID NO: 27308)
SEREEVKEKLDRLLEEVEKTVRELKREHDELLKEVEKLVRDLKKEHDELLKKVKDDGVPKEILDVIREHVRTNKE
ILDRVLEVVEEHLRRNKEILDKLLDDIRKVVEDAKRILGIGDDETLRRAVRRILEELRKLVEDIRKKVKDSLETL
ERALEEAEERIRRSLEDLKRVLKEAKDKTKDKDRDHMVLHEYVNAAGITKRTVDRVRELVRKILKKSRETLEELE
RLIEKILRSLEKDAR
>3plus1_Cage_Nterm_GFP11_673
(SEQ ID NO: 27309)
SERETVKRRLEELLKEVKRTLDKLKEEHDRLLEDVRRVVSELRDHMVLHEYVNAAGITPEELLRVIAKVLETNKR
ILDDLLRVVKKHVDLNKEILDRILEMIKEIVERVKRVLGDGDEKTLRDKIRDIIRRLEDAAREAEERVRRSLEEL
KKAVEKIRKKIEDSLRELEEALKRVRDKEEDDKRLEDISRLVKRLLDESRRVLRELEETIRKRAEESKRVLEEVK
RLVEKLIRSLPKEAE
>3plus1_Cage_Nterm_GFP11_676
(SEQ ID NO: 27310)
SEDEIIKKIIEDLRRVLKEVEEIHKEVEERLDKRDHMVLHEYVNAAGITDRVLDEVKRIGDVETVLRIAIEAVRR
ALEIVRKVTEDLIRVVRKLLEIVDAVNKRVKKAVEDVLKLLRDTLKGSKDVERVLDEARRLLEKLHRDSRRILKR
IAELLDELKELSERILERIKRLLEEVERRGSDDERLLEELIEELKDLIRRHIELLRRLIERLKELLKKHIEDLRR
LLEEIEKLLEEARK
>3plus1_Cage_Nterm_GFP11_677
(SEQ ID NO: 27311)
SREEVLRELEEVIEDNRRLLEELIEKSKKVLDESLKLIDELLRRDHMVLHEYNNAAGITSKGDDEETARRIVTKI
RKELEDVLDEVRRIVKEILDRDRRVAEDLKRVVDENLEKLKKVLDELDKKLGDRRLVEILRDIVRLLIEINERAL
RELLKLHEEHTRIVAELLRELERVVEELRELVELIARGEISEDDLKRVVDEVEKKLRELKRRYKEALERIKEKIK
ELKDRYERAVREWAELRKTTK
>3plus1_Cage_Cterm_GFP11_677
(SEQ ID NO: 27312)
SREEVLRELEEVIEDNRRLLEELIEKSKKVLDESLKLIDELLRRLEEVLERVLRLLEELSKGDDEETARRIVTKI
RKELEDVLDEVRRIVKEILDRDRRVAEDLKRVVDENLEKLKKVLDELDKKLGDRRLVEILRDIVRLIIEINERAL
RELLKLHEEHTRIVAELLRELERVVEELRRLVELIARGEISEDDRDHMVLHEYVNAAGITRRYKEALERIKEKIK
ELKDRYERAVREWAELRKTTK
>3plus1_Cage_Cterm_GFP11_677
(SEQ ID NO: 27313)
SREEVLRELEEVIEDNRRLLEELIEKSKKVLDESLKLIDELLRRLEEVLERVLRLLEELSKGDDEETARRIVTKI
RKELEDVLDEVRRIVKEILDRDRRVAEDLKRVVDENLEKLKKVLDELDKKLGDRRLVEILRDIVRLLIEINERAL
RELLKLHEEHTRIVAELLRELERVVEELRRLVELIAPGERDHMVLHEYVNAAGITLRELKRRYKEALERIKEKIK
ELKDRYERAVREWAELRKTTK
>3plus1_Cage_Nterm_GFP11_678
(SEQ ID NO: 27314)
SKAEEIAEKLDRLLEENRRALEEITTRLDDLLRRDHMVLHEYVNAAGITLDDLRRGGIREELLKRIADVIRDIMR
LLKELHDHTAEVIKTIKKLLKELHDINKEIIERLKRLKDGNVPKEELLKPVEELVRTSARLTTEVLKTVEKLIRD
DKRLSEEILKRVKELISELKRGVDSERVKEILERILRVVEEAVRLNEESLRRILDVVRKAVKLDRESLKKILDVV
EEAVR
>3plus1_Cage_Cterm_GFP11_678
(SEQ ID NO: 27315)
SKAEEIAEKLDRLLEENRRALEEITTRLDDLLRRNKDALRKVMEKLKRLLDDLRRGGIREELLKRIADVIRDIMR
LLKELHDHTAEVIKTIKKLLKELHDINKEIIERLKRLKDGNVPKEELLKRVEELVRTSARLTTEVLKTVEKLIRD
DKRLSEEILKRVKELIEELRDHMVLHEYVNAAGITLRVVEEAVRLNEESLRRILDVVRKAVKLDRESLKKILDVV
EEAVR
>3plus1_Cage_Nterm_GFP11_678
(SEQ ID NO: 27316)
SKAEEIALKLDRLLEENRRALEEITTRLDDLLRRNKDRDHMVLHEYVNAAGITRRGGIREELLKRIADVIRDIMR
LLKELHDHTAEVIKTIKKLLKELHDINKEIIERLKRLKDGNVPKEELLKRVEELVRTSARLTTEVLKTVEKLIRD
DKRLSEEILKRVKELISELKRGVDSERVKEILERILRVVEEAVRLNEESLRRILDVVRKAVKLDRESLKKILDVV
EEAVR
>3plus1_Cage_Cterm_GFP11_678
(SEQ ID NO: 27317)
SKAEEIAEKLDRLLEENRRALEEITTRLDDLLRIWKDALRKVMEKLKRLLDDLRRGGIREELLKRIADVIRDIMR
LLKELHDHTAEVIKTIKKLLKELHDINKEIIERLKRLKDGNVPKEELLKRVEELVRTSARLTTEVLKTVEKLIRD
DKRLSEEILKRVKELIEELKFGRDHMVLHEYVNAAGITVEEAVRLNEESLRRILDVVRKAVKLDRESLKKILDVV
EEAVR
>3plus1_Cage_Nterm_GFP11_678
(SEQ ID NO: 27318)
SKAEEIAEKLDRLLEENRRALEEITTRLDDLLRRNKRDHMVLHEYVNAAGITLRRGGIREELLKRIADVIRDIMR
LLKELHDHTAEVIKTIKKLLKELHDINKEIIERLKRLKDGNVPKEELLKRVEELVRTSARLTTEVLKTVEKLIRD
DKRLSEEILKRVKELIEELKRGVDSERVKEILERILRVVEEAVRLNEESLRRILDVVRKAVKLDRESLKKILDVV
EEAVR
>3plus1_Cage_Nterm_GFP11_678
(SEQ ID NO: 27319)
SKAEEIAEKLDRLLEENRRALEEITTRLDDLLRRNKDALRDHMVLHEYVNAAGITGGIREELLKRIADVIRDIMR
LLKELHDHTAEVIKTIKKLLKELHDINKEIIERLKRLKDGNVPKEELLKRVEELVRTSARLTTEVLKTVEKLIRD
DKRLSEEILKRVKELIEELKRGVDSERVKEILERILRVVEEAVRLNEESLRRILDVVRKAVKLDRESLKKILDVV
EEAVR
>3plus1_Cage_Cterm_GFP11_679
(SEQ ID NO: 27320)
SPVEELKKLIEDILRISREVVERIKRVAEDIHRINRRVLDDLRKLIEDILRTVEEILARKVGDTEIAERLRDTIA
RVVDEIAKLLEEHEKRSRELLEEIRKLLEDILRRSERAVEEIRELLKKGVSTKDVLRILEEILREHLELLERVVR
RIEEILRELLKTIEEIVKRIKEILEELKEVLKRGRVKDDEVERDHMVLHEYVNAAGITYRRLLEEIKRKLEEILR
RVEELHRRLRRKLEEIDR
>3plus1_Cage_Nterm_GFP11_679
(SEQ ID NO: 27321)
SRVEELKKLIEDILRISREVVERIKRVAEDIKRINRRVRDHMVLHEYVNAAGITEILARKVGDTEIAERLRDTIA
RVVDEIAKLLEEHEKRSRELLEEIRKLLEDILRRSERAVEEIRELLKKGVSTKDVLRI1EEILREHLELLERVVR
RIEEILPELLKTIEEIVKRIKEILEELKEVLKRGRVKDDEVEREIRRVKEDLDRILEEYRRLLEEIKRKLEEILR
RVEELHRRLPRKLEEIDR

In each embodiment, the N-terminal and/or C-terminal 60 amino acids of each cage polypeptides may be optional, as the terminal 60 amino acid residues may comprise a latch region that can be modified, such as by replacing all or a portion of a latch with a bioactive peptide. In one embodiment, the N-terminal 60 amino acid residues are optional; in another embodiment, the C-terminal 60 amino acid residues are optional: in a further embodiment, each of the N-terminal 60 amino acid residues and the C-terminal 60 amino acid residues are optional. In one embodiment, these optional N-terminal and/or C-terminal 60 residues are not included in determining the percent sequence identity. In another embodiment, the optional residues may be included in determining percent sequence identity.

As disclosed herein, bioactive peptides to be sequestered by the polypeptides of the disclosure are located within the latch region. The latch region is denoted by brackets in the sequence of each cage polypeptide. The bioactive peptide may be added to the latch region without removing any residues of the latch region, or may replace one or more (1, 2, 3, 4, 5, 6, 7, 8, 9, 10, or more) amino acid residues in the cage scaffold latch region to produce the final polypeptide. Thus, the latch region may be significantly modified upon inclusion of the bioactive peptide. In one embodiment, the optional residues are not included in determining percent sequence identity. In another embodiment, the latch region residues may be included in determining percent sequence identity. In a further embodiment, each of the optional residues and the latch residues are not included in determining percent sequence identity.

The cage polypeptide including the degron may be a cage scaffold polypeptide (i.e.: without a bioactive peptide) For example, see SEQ ID NOS:1-17, 2034-14317, 27359 ,-28465 and certain cage polypeptides listed in Table 2, Table 3, Table 4, and/or Table 5 or may further include a sequestered bioactive peptide (present as a fusion with the cage scaffold polypeptide) in the latch region of the cage scaffold polypeptide, as described in more detail herein (for example, see SEQ ID NOS:18-49, 51-52, 54-59, 61. 65, 67-2033, 27094-27117, 27120-27125, and certain cage polypeptides listed in Table 2, 3, 4, and/or 5). In a specific embodiment, the cage polypeptides share 40%, 45%, 50% , 55%, 60%, 65%, 70%, 75%, 80%, 85%, 90%, 91%, 92%, 93%, 94%, 95%, 96%, 97%, 98%, 99%, or 100% sequence identity along their length to the amino acid sequence of a cage polypeptide in Table 2, Table 3, Table 4, and/or Table 5, and also comprise one or more degrons.

In another specific embodiment, the cage polypeptides comprise a polypeptide having at least 40%, 45%, 50%, 55%, 60%, 65%. 70%, 75%, 80%, 85%, 90%. 91%, 92%, 93%, 94%, 95%, 96%, 97%, 98%. 99%, or 100% sequence identity along their length to the amino acid sequence of a cage polypeptide in Table 3, and include one or more degrons. In another specific embodiment, the cage polypeptides share 40%, 45%, 50%, 55%, 60%, 65%, 70%, 75%, 80%, 85%, 90%. 91%, 92%, 93%, 94%, 95%, 96%, 97%, 98%, 99%, or 100% sequence identity along their length to the amino acid sequence of a cage polypeptide in. Table 4, and include one or more degrons. In one embodiment of each of these embodiments, the optional N-terminal and/or C-terminal 60 residues are not included in determining the percent sequence identity. In another embodiment, the optional residues may he included in determining percent sequence identity.

In another specific embodiment, the cage polypeptides comprise a polypeptide having at least 40%, 45%. 50%, 55%, 60%, 65%, 70%, 75%, 80%, 85%, 90%, 91%, 92%, 93%, 94%, 95%, 96%, 97%, 98%, 99%, or 100% sequence identity along their length to the amino acid sequence of a cage polypeptide selected from the group consisting of SEQ ID NOS: 27359 -28465 or a cage polypeptide listed in Table 7.

In some aspects, both a latch region and a key polypeptide can bind to or interact with a structural region in the corresponding cage polypeptide. The interaction between a latch region and a structural region in a cage polypeptide can be intramolecular interaction, and the interaction between a key polypeptide and a structural region of the corresponding cage polypeptide can be intermolecular interaction. However, in some aspects, the affinity of the latch region to the structural region of the cage polypeptide is higher than the affinity of the key polypeptide to the structural region of the cage polypeptide in the absence of an effector polypeptide.

In some aspects, the affinity of the latch region to the structural region of the cage polypeptide is at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 16 fold, at least about 17 fold, at least about 18 fold, at least about 19 fold, at least about 20 fold, at least about 21 fold, at least about 22 fold, at least about 23 fold, at least about 24 fold, at least about 25 fold, at least about 26 fold, at least about 27 fold, at least about 28 fold, at least about 29 fold, or at least about 30 fold higher than the affinity of the key polypeptide to the structural region of the cage polypeptide in the absence of an effector polypeptide. In some aspects, the affinity of the latch region to the structural region of the cage polypeptide is at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2.0 fold, at least about 2.1 fold, at least about 2.2 fold, at least about 2.3 fold, at least about 2.4 fold, at least about 2.5 fold, at least about 2.6 fold, at least about 2.7 fold, at least about 2.8 fold, at least about 2.9 fold, or at least about 3.0 fold higher than the affinity of the key polypeptide to the structural region of the cage polypeptide in the absence of an effector polypeptide. In some aspects, the affinity of the latch region to the structural region of the cage polypeptide is at least about 30 fold, at least about 40 fold, at least about 50 fold, at least about 60 fold, at least about 70 fold, at least about 80 fold, at least about 90 fold. at least about 100 fold, at least about 110 fold, at least about 120 fold, at least about 130 fold, at least about 140 fold, at least about 150 fold, at least about 160 fold, at least about 170 fold, at least about 180 fold, at least about 190 fold, at least about 200 fold, at least about 210 fold, at least about 220 fold, at least about 230 fold, at least about 240 fold, at least about 250 fold, at least about 260 fold, at least about 270 fold, at least about 280 fold, at least about 290 fold, at least about 300 fold, e.g., about 30 fold to about 300 fold, e.g., about 100 fold to about 300 fold, about 50 fold to about 100 fold, higher than the affinity of the key polypeptide to the structural region of the cage polypeptide in the absence of an effector polypeptide.

In other embodiments, the intramolecular Latch-Cage affinity is higher than the intermolecular Key-Cage affinity, and in the presence of the Effector protein, the intermolecular Key-Cage affinity is higher than the intramolecular Latch-Cage affinity. As a result, the function of the bioactive peptide is dependent on the presence of Cage, Key, and Effector protein.

In certain embodiments, the intermolecular Key-Cage interaction may outcompete the Latch-Cage interaction in the absence of Effector protein. In the absence of Key, the Latch-Cage affinity is higher than the Latch-Effector protein affinity (via binding of the Bioactive peptide to the Effector protein), and in the presence of Key, the Latch-Effector protein affinity (via binding of the Bioactive peptide to the Effector protein) is higher than the Latch-Cage affinity. As a result, the function of the bioactive peptide is dependent on the presence of Cage, Key, and Effector protein.

As disclosed in the examples that follow, exemplary cage (and key) polypeptides of the disclosure have been identified and subjected to mutational analysis. Furthermore, different designs starting from the same exemplary cage and key polypeptides yield different amino acid sequences while maintaining the same intended function. In various embodiments, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg, Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that the desired activity is retained. Amino acids can be grouped according to similarities in the properties of their side chains (in A. L, Lehninger, in Biochemistry, second ed., pp. 73-75, Worth Publishers, New York (1975)): (1) non-polar: Ala (A), Val (V). Leu (L), Ile (I), Pro (P), Phe (F), Trp (W), Met (M); (2) uncharged polar: Gly (G), Ser (S), Thr (T), Cys (C), Tyr (Y), Asn (N), Gin (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Mn, Gin; (3) acidic: Asp, Glu, (4) basic: His, l.ys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gin or into H is; Asp into Glu; Cys into Ser; Gin into Asn; Glu into Asp; Gly into Ala. or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In a second aspect, the disclosure provides kits comprising:
(a) the cage polypeptide of any embodiment or combination of embodiments of the first aspect; and
(b) a key polypeptide capable of binding to the cage polypeptide structural region, thereby displacing the latch region and activating the one or more degron.

In a third aspect, the disclosure provides degron LOCKR switches comprising:
(a) the cage polypeptide of any embodiment or combination of embodiments of the eleventh aspect; and
(b) a key polypeptide capable of binding to the cage polypeptide structural region, thereby displacing the latch region and activating the one or more degron.

As disclosed in detail herein, when a key is expressed and activates the cage polypeptide by interacting with the structural region and displacing the latch region from its interaction with the structural region (i.e.: via higher affinity binding than the latch region), the degron targets the cage polypeptide, and any functional polypeptide domains fused to the cage polypeptide, for degradation. In this way, a functional polypeptide domain of interest fused to the cage polypeptide having a degron can be conditionally degraded in a titratable manner via expression of the key.

In one embodiment of the kits and degron LOCKR switches of the second and third aspects, the key polypeptide comprises an amino acid sequence having at least 45%, 50%, 55%, 60%, 65%, 70%, 75%, 80%, 85%, 90%, 91%, 92%, 93%, 94%, 95%, 96%, 97%, 98%, 99%, or 100% sequence identity along the length of the amino acid sequence of a key protein selected from (not including optional amino acid residues) SEQ II) NOS: 14318-26601, 26602-27015, 27016-27050, 27322 to 27358, and key polypeptides in Table 2 or 5 (polypeptides with an odd-numbered SEQ ID NO between SEQ ID NOS: 27127 and 27277), Table 3, and/or Table 4, not including optional amino acid residues. As noted, key polypeptides may include residues that are optional; these residues are provided in parentheses and in one embodiment are not included in determining the percent sequence identity. In another embodiment, the optional residues may be included in determining percent sequence identity.

In another embodiment, the key polypeptides comprise a polypeptide having at least 40%, 45%, 50%, 55%, 60%, 65%, 70%, 75%, 80%, 85%, 90%, 91%, 92%, 93%. 94%, 95%, 96%, 97%, 98%. 99%, or 100% sequence identity along its length to the amino acid sequence of a key polypeptide selected from the group consisting of SEQ_{.} ID NOS: 26602-27050, and 27322-27358. and 28477-28486 as detailed below.

Amino Acid Sequences of Additional
Keys for degronLOCKRs
(SEQ ID NOs: 28477-28486)
>key_a
(SEQ ID NO: 28177)
EARKAIARVKRESKRIVEDAERLIREAAA
ASEKISREAERLIREAAAASEKISRE
>key_a_m4
(SEQ ID 240: 28478)
DEARKAIARVKRESKRIVEDAERLIREAA
AASEKISREAERLIREAAAASEK
>key_a_m9
(SEQ ID NO: 23479)
DEARKAIARVKRESKRIVEDAERLIREAA
AASEKISREAERLIREAAK
>key_a_m12
(SEQ ID NO: 28480)
DEARKAIARVKRESKRIVEDAERLIREAA
AASEKISREAERLIR
>key_a_m15
(SEQ ID NO: 28481)
DEARKAIARVKRESKRIVEDAERLIREAA
AASEKISREAER
>key_b
(SEQ ID NO: 28432)
NKEEIEKLAKEAREKLKKAEKEHKEIHDK
LRKKNKKAREDLKKKADELRETNKRVN
>key c
(SEQ ID NO: 28483)
SSEKVRRELKESLKENHKQNQKLLKDHKR
AQEKLNRELEELKKKHKKTLDDIRRES
>key_d
(SEQ ID NO: 28484)
DTVKRILEELRRRFEKLAKDLDDIARKLL
EDHKKHNKELKDKQRKIKKEADDAARS
>key_d_m4
(SEQ ID NO: 23485)
RILEELRRRFEKLAKDLDDIARKLLEDHK
KHNKELKDKQRKIKKEADDAARS
>key_d_m7
(SEQ ID NO: 28486)
EELRRRFEKLAKDLDDIARKLLEDHKKHN
KELKDKQRKIKKEADDAARS

Ley sequences are normal text
6His-MBP-TEV. 6His-TEV, and flexible linker sequences are underlined text
sequence in bold, italics, are optional residues necessary for biotinylation of MBP_key
all sequences in parentheses are optional
Any number of consecutive amino acids from the N or C terminus in the non-optional key sequence may be removed to tune responsiveness

>SB76 C-helix
(SEQ ID NO: 27016)
DEARKAIAPNERESKRIVEDAERLIREAAAASEKIS
>SB73_C-helix-biotin
(SEQ ID NO: 27017)
DEARKAIARVKPESKRIVEDAERLIREAAAASEKIS
GSGK-Biotin
>pb_MBP
(SEQ ID NO: 27018)
(MDP)DEARKAIARVKRESKRIVEDAERLIREAAA
ASEKISREA(SSGLVPRGSHMKIEEGKLVIWINGD
KGYNGLAEVGKKFEKDTGIKVTVEHPDKLEEKFPQ
VAATGDGPDIIFWAHDREGGYAQSGELAEITEDKA
FQDKLYPFTWDAVRYNGKLIAYPIAVEALSLIYNK
DLLPNPPKTWEEIPALDKEDKAKGKSALMENLQEP
YFTWPLIAADGGYAFKYENGKYDIKDVGVDNAGAK
AGLTFLVDLIKNKHMNADTDYSIAEAAFNKGETAM
TINGPWAWSNIDTSKVNYGVTVLPTFKGQPSKPFV
GVLSAGINAASPNKELAKEFLENYLLTDEGLEAVN
KDKPLGAVADKSYEEELVKDPRIAATMENAQKGEI
MPNIPQMSAFWYAVRTAVINAASGRQTVDEALKDA
QTNSSGSGLNDIFEAQ [symbol] IEWHEDEHHHHHH)
> p9_1BP
(SEQ ID NO: 27019)
(MDP)DEARKAIARVKRESKRIVEDAERLIREAAA
ASEKISREAERLIREAA(SSGLVPRGSHMKIEEGK
LVIWINGDKGYNGLAEVGKKFEKDTGTKVTVEHPD
KLEEKFPQVAATGDGPDIIFWAHDRFGGYAQSGLL
AEITPDKAFQDKLYPFTWDAVPYNGKLIAYPIAVE
ALSLIYNKDLLPNPPKTWEEIPALDKELKAKGKSA
LMENLQEPYFTWPLIAADGGYAFKYENGKYDIKDV
GVDNAGAKAGLTFLVDLIKNKHMNADTDYSIAEAA
FNKGETAMTINGPWAWSNIDTSKVNYGVTVLPTFK
GQPSKPFVGVLSAGINAASPNKELAKEFDENYLLT
DEGLEAVNKDKPLGAVALKSYEEELVKDPRIAATM
ENAQKGEIMPNIPQMSAFWYAVRTAVINAASGRQT
VDEALKDAQTNSSGSGLNDIFEAQ [symbol] IEWHE
LEHHHHHH)
>p16MBP
(SEQ ID NO: 27020)
(MDP)DEARKAIARVKRESKRIVEDAERLIREAAA
ASEKISREAERLIREAAAASEKISRE(SSGLVPRG
SHMKIEEGKLVIWINGDKGYNGLAEVGKKFEKDTG
IKVTVEHPDKLEEKFPQVAATGDGPDIIFWAHDRF
GGYAQSGLLAEITPDKAFQDKLYPFTWDAVRYNGK
LIAYPIAVEALSLIYNKDLLPNPPKTWEEIPALDK
ELKAKGKSALMENLQEPYFTWPLIAADGGYAFKYE
NGKYDIKDVGVDNAGAKAGLTFLVDLIKNKHMNAD
TDYSIAEAAFNKGETAMTINGPWAWSNIDTSKVNY
GVTVLPTFKGQPSKPTVGVLSAGINAASPNKELAK
EFLENYLLTDEGLEAVNKDKPLGAVALKSYEEELV
KDPRIAATMENAQKGEIMPNIPQMSAFWYAVRTAV
INAASGRQTVDEALKDAQTNSSGSGLNDIFEA
Q [symbol] IEWHELEHHHHHH)
>MBP_p18 (aka. p76)
(SEQ ID 140: 27021)
(MGSSHHHHHASSGLVPRGSHMKIEEGKLVIWING
DKGYNGLAEVGKKFEKDTGIKVTVEHPDKLEEKFP
QVAATGDGETIIFWAHDREGGYAQSGLLAEITPDK
NFODKLYPFTWDAVRYNGKLIAYPIAVEALSLIYN
KDLLPNPPKTWEEIPALDKELKAKGKSALMENLQE
PYFTWPLIAADGGYAFKYENGKYDIKDVGVDNAGA
KAGLTFINDLIKNKHMNADTDYSTAEAAFNKGETA
MTINGPWAWSNIDTSKVNYGVTVLPTEKGQPSKPF
VGVLSAGINAASPNKELAKEFLENYLLTDEGLEAV
NKDKPLGAVALKSYEEELVKDPRIAATMENAQKGE
IMPNIPQMSAFWYAVRTAVINAASGRQTVDEALKD
AQTNSGSGSGENLYFQ)DEARKAIARVKRESKRIV
EDAERLIREAAAASEKISRE(AERLIREAAAASEK
ISRE)
>key_b
(SEQ ID NO: 27022)
(M)NKEEIEKLAKEAREKEKLKAEKEHKEIHDKLR
KKNKKAREDLKKKADELPETNKRVN(GSENLYFGS
GSGKIEEGKLVIWINGDKGYNGLAEVGKKFEKDTG
IKVTVEHPDKLEEKFPQVAATGDGPDIIFWAHDRF
GGYAQSGLLAEITPDKAFQDKLYPFTWDAVRYNGK
LIAYPIAVEALSLIYNKDLLPNPPKTWEEIPALDK
ELKAKGKSALMENLQEPYFTWPLIAADGGYAFKYE
NGKYDIKDVGVDNAGAKAGLTFLVDLIKNKHMNAD
TDYSIAEAAFNKGETAMTINGPWAWSNIDTSKVNY
GVTVLPTFKGQPSKPTVGVLSAGINAASPNKELAK
EFLENYLLTDEGLEAVNKDKPLGAVALKSYEEELV
KDPRIAATMENAQKGEIMPNIPQMSAFWYAVRTAV
INAASGRQTVDEALKDAQTNLEHHHHHH)
>key_c
(SEQ ID NO: 27023)
(M)SSEKVRRELKESLKENHKQNQKLLKDHKRAQE
KLNRELEELKKKHKKTLDDIRRES(GSENLYFQGS
GSGKIEEGKLVIWINGDKGYNGLAEVGKKFEKDTG
IKVTVEHPDKLEEKFPQVAATGDGPDIIFWAHDRF
GGYAQSGLLAEITPDKAFQDKLYPFTWDAVRYNGK
LIAYPIAVEALSLIYNKDLLPNPPKTWEEIPALDK
ELKAKGKSALMENLQEPYFTWPLIAADGGYAFKYE
NGKYDIKDVGVDNAGAKAGLTFLVDLIKNKHMNAD
TDYSIAEAAFNKGETAMTINGPWAWSNIDTSKVNY
GVTVLPTFKGQPSKPTVGVLSAGINAASPNKELAK
EFLENYLLTDEGLEAVNKDKPLGAVALKSYEEELV
KDPRIAATMENAQKGEIMPNIPQMSAFWYAVRTAV
INAASGRQTVDEALKDAQTNLEHHHHHH)
>key_d
(SEQ ID NO: 27024)
(M)DTVKRILEELRRRFEKLAKDLDDIARKLLEDH
KKHNKELKDKQRKIKKEADDAARS(GSENLYFQGS
GSGKIEEGKLVIWINGDKGYNGLAEVGKKFEKDTG
IKVTVEHPDKLEEKFPQVAATGDGPDIIFWAHDRF
GGYAQSGLLAEITPDKAFQDKLYPFTWDAVRYNGK
LIAYPIAVEALSLIYNKDLLPNPPKTWEEIPALDK
ELKAKGKSALMENLQEPYFTWPLIAADGGYAFKYE
NGKYDIKDVGVDNAGAKAGLTFLVDLIKNKHMNAD
TDYSIAEAAFNKGETAMTINGPWAWSNIDTSKVNY
GVTVLPTFKGQPSKPTVGVLSAGINAASPNKELAK
EFLENYLLTDEGLEAVNKDKPLGAVALKSYEEELV
KDPRIAATMENAQKGEIMPNIPQMSAFWYAVRTAV
INAASGRQTVDEALKDAQTNLEHHHHHH)
>key_e
(SEQ ID NO: 27025)
(M)DDVERRLEKANKESKKEAEELTEEAKKANEKTK
EDSKELTKENRKTNKTIKDEARS(GSENLYFQGSG
SGKIEEGKLVIWINGDKGYNGLAEVGKKFEKDTGI
KVTVEHPDKLEEKFPQVAATGDGPDIIFWAHDREG
GYAQSGLLAEITPDKAFQDKLYPFTWDAVRYNGKL
IAYPIAVEALSLIYNKDLLPNPPKTWEEIPALDKE
LKAKGKSALMENLQEPYFTWPLIAADGGYAFKYEN
GKYDIKDVGVDNAGAKAGLTFLVDLIKNKHMNADT
DYSIAEAAFNKGETAMTINGPWAWSNIDTSKVNYG
VTVLPTFKGQPSKPTVGVLSAGINAASPNKELAKE
FLENYLLTDEGLEAVNKDKPLGAVALKSYEEELVK
DPRIAATMENAQKGEIMPNIPQMSAFWYAVRTAVI
NAASGRQTVDEALKDAQTNLEHHHHHH)
>key_f
(SEQ ID NO: 27026)
(M)DDEERRSEKTVQDAKREIKKVEDDLQRLNEEQ
KKKVKKQEDENQKTLKKHKDDARS(GSENLYEQGS
GSGKIEEGKLVIWINGDKGYNGLAEVGKKFEKDTG
IKVTVEHPDKLEEKFPQVAATGDGPDIIFWAHDRF
GGYAQSGLLAEITPDKAFQDKLYPFTWDAVRYNGK
LIAYPIAVEALSLIYNKDLLPNPPKTWEEIPALDK
ELKAKGKSALMENLQEPYFTWPLIAADGGYAFKYE
NGKYDIKDVGVDNAGAKAGLTFLVDLIKNKHMNAD
TDYSIAEAAFNKGETAMTINGPWAWSNIDTSKVNY
GVTVLPTFKGQPSKPTVGVLSAGINAASPNKELAK
EFLENYLLTDEGLEAVNKDKPLGAVALKSYEEELV
KDPRIAATMENAQKGEIMPNIPQMSAFWYAVRTAV
INAASGRQTVDEALKDAQTNLEHHHHHH)

### Additional Keys:

Key Sequences are Normal Text
(6His-MBP-TEV, 6His-TEV, and flexible linker sequences are underlined text)
(Co-localization domain is bolded text)
(Positions that can be mutated to any amino acid to tune responsiveness are underlined bolded text. These are exemplary but not exhaustive.)
Any number of consecutive amino acids from the N or C terminus in the non-optional key sequence may be removed to tune responsiveness)

(all sequences in parentheses are optional)

>p76-long
(SEQ ID NO: 27027)
(MGSSHHHHHHSSGLVPRGSHMKIEEGKLVIWINGDKGYNGLAEVGKKFEKDTGIKVTVEHPDKLEEKFPQVAAT
GDGPDIIFWAHDRFGGYAQSGLLAEITPDKAFQDKLYPFTWDAVRYNGKLIAYPIAVEALSLIYNKDLLPNPPKT
WEEIPALDKELKAKGKSALMFNLQEPYFTWPLIAADGGYAFKYENGKYDIKDVGVDNAGAKAGLTFLVDLIKNKH
MNADTDYSIAEAAFNKGETAMTINGPWAWSNIDTSKVNYGVTVLPTFKGQPSKPFVGVLSAGINAASPNKELAKE
FLENYLLTDEGLEAVNKDKPLGAVALKSYEEELVKDPRIAATMENAQKGEIMPNIPQMSAFWYAVRTAVINAASG
RQTVDEALKDAQTNSGSGSGENLYFQ)DEARKAIARVKRESKRIVEDAERLIREAAAASEKISREAERLIREAAA
ASEKISPE
>p76-short
(SEQ ID NO: 27028)
(MGSSHHHHHHSSGLVPRGSHMKIEEGKLVIWINGDKGYNGLAEVGKKFEKDTGIKVTVEHPDKLEEKFPQVAAT
GDGPDIIFWAHDRFGGYAQSGLLAEITPDKAFQDKLYPFTWDAVRYNGKLIAYPIAVEALSLIYNKDLLPNPPKT
WEEIPALDKELKAKGKSALMFNLQEPYFTWPLIAADGGYAFKYENGKYDIKDVGVDNAGAKAGLTFLVDLIKNKH
MNADTDYSIAEAAFNKGETAMTINGPWAWSNIDTSKVNYGVTVLPTFKGQPSKPFVGVLSAGINAASPNKELAKE
FLENYLLTDEGLEAVNKDKPLGAVALKSYEEELVKDPRIAATMENAQKGEIMPNIPQMSAFWYAVRTAVINAASG
RQTVDEALKDAQTNSGSGSGENLYFQ)DEARKAIARVKRESKRIVEDAERLIREWASEKISREAERLIR
>k76-long
(SEQ ID NO: 27029)
(MGSSHHHHHHSSGLVPRGSHMKIEEGKLVIWINGDKGYNGLAEVGKKFEKDTGIKVTVEHPDKLEEKFPQVAAT
GDGPDIIFWAHDRFGGYAQSGLLAEITPDKAFQDKLYPFTWDAVRYNGKLIAYPIAVEALSLIYNKDLLPNPPKT
WEEIPALDKELKAKGKSALMFNLQEPYFTWPLIAADGGYAFKYENGKYDIKDVGVDNAGAKAGLTFLVDLIKNKH
MNADTDYSIAEAAFNKGETAMTINGPWAWSNIDTSKVNYGVTVLPTFKGQPSKPFVGVLSAGINAASPNKELAKE
FLENYLLTDEGLEAVNKDKPLGAVALKSYEEELVKDPRIAATMENAQKGEIMPNIPQMSAFWYAVRTAVINAASG
RQTVDEALKDAQTNSGSGSGENLYFQ)DEARKAIARVKRESKRIVEDAERLIREAAQASEKISREARELIERAAQ
ASEKISRE
>k76-short
(SEQ ID NO: 27030)
(MGSSHHHHHHSSGLVPRGSHMKIEEGKLVIWINGDKGYNGLAEVGKKFEKDTGIKVTVEHPDKLEEKFPQVAAT
GDGPDIIFWAHDRFGGYAQSGLLAEITPDKAFQDKLYPFTWDAVRYNGKLIAYPIAVEALSLIYNKDLLPNPPKT
WEEIPALDKELKAKGKSALMFNLQEPYFTWPLIAADGGYAFKYENGKYDIKDVGVDNAGAKAGLTFLVDLIKNKH
MNADTDYSIAEAAFNKGETAMTINGPWAWSNIDTSKVNYGVTVLPTFKGQPSKPFVGVLSAGINAASPNKELAKE
FLENYLLTDEGLEAVNKDKPLGAVALKSYEEELVKDPRIAATMENAQKGEIMPNIPQMSAFWYAVRTAVINAASG
RQTVDEALKDAQTNSGSGSGENLYFQ)DEARKAIARVKRESKRIVEDAERLIREAAQASEKISREAERLIR
>p76_GLISE
(SEQ ID NO: 27031)
(MGSHHHHHHGSGSENLYFQGSGGS)DEARKAIARVKRSSKRIVEDAEGLISEAAAASEKISREAERLIREAAAA
SEKISRE
>p76_GSSEKIS
(SEQ ID NO: 27032)
(MGSHHHHHHGSGSENLYFQGSGGS)DEARKAIARVKRSSRRIVEDAERLIREAAGSSEKISREAERLIREAAAA
SEKISRE
p76_R26G
(SEQ ID NO: 27033)
(MGSHHHHHHGSGSENLYFQGSGGS)DEARKAIARVKRESKRIVEDAERLIGEAAAASEKISREAERLIREAAAA
SEKISRE
>p76-short_E19G
(SEQ ID NO: 27034)
(MGSHHHHHHGSGSENLYFQGSGGS)DEARKAIARVKRESKRIVGDAERLIREANANSEKISREAERLIR
>p76-short_GLISE_E01_EGFR
(SEQ ID NO: 27035)
(MGSHHHHHHGSGSENLYFQGSGGS)DEARKAIAPVKRESKRIVEDAEGLISEPAAASEKISREAERLIR
>p76-short_AE_EGFP
(SEQ ID NO: 27036)
(MGSHHHHHHGSGSENLYFQGSGGS)DEARKNIARVAEESKRIVEDAERLIRENANASEKISREAERLIR
>p76-short_AAE_EGFR
(SEQ ID NO: 27037)
(MGSHHHHHHGSGSENLYFQGSGGS)DEAAKAIARVAEESKRIVEDAERLIREAAAASEKISREAERLIR
>p76-short_EE_EGFR
(SEQ ID NO: 27038)
(MGSHHHHHHGSGSENLYFQGSGGS)DEARKAIARVKRESKRIVEDAERLIREAAEASEEISREAERLIR
>p76-spytag
(SEQ ID NO: 27039)
(MGSHHHHHHGSGSENLYFQGSGGSMKIEEGKLVIWINGDKGYNGLAEVGKKFEKDTGIKVTVEHPDKLEEKFPQ
VAATGDGPDIIFWAHDRFGGYAQSGLLAEITPDKAFQDKLYPFTWDAVRYNGKLIAYPIAVEALSLIYNKDLLPN
PPKTWEEIPALDKELKAKGKSALMFNLQEPYFTWPLIAADGGYAFKYENGKYDIKDVGVDNAGAKAGLTFLVDLI
KNKHMNADTDYSIAEAAFNKGETAMTINGPWAWSNIDTSKVNYGVTVLPTFKGQPSKPFVGVLSAGINAASPNKE
LAKEFLENYLLTDEGLEAVNKDKPLGAVALKSYEEELVKDPRIAATMENAQKGEIMPNIPQMSAFWYAVRTAVIN
AASGRQTVDEALKDAQTNSGSGSGENLYFQ)DEARKAIARVKRESKRIVEDAERLIREAAAASEKISREAERLIR
EAAAASEKISRE(GGGSGSGSGSGKPGQASGS)AHIVMVDAYKPTK
>p76-short-3pytag
(SEQ ID NO: 27040)
(MGSHHHHHHGSGSENLYFQGSGGSMKIEEGKLVIWINGDKGYNGLAEVGKKFEKDTGIKVTVEHPDKLEEKFPQ
VAATGDGPDIIFWAHDRFGGYAQSGLLAEITPDKAFQDKLYPFTWDAVRYNGKLIAYPIAVEALSLIYNKDLLPN
PPKTWEEIPALDKELKAKGKSALMFNLQEPYFTWPLIAADGGYAFKYENGKYDIKDVGVDNAGAKAGLTFLVDLI
KNKHMNADTDYSIAEAAFNKGETAMTINGPWAWSNIDTSKVNYGVTVLPTFKGQPSKPFVGVLSAGINAASPNKE
LAKEFLENYLLTDEGLEAVNKDKPLGAVALKSYEEELVKDPRIAATMENAQKGEIMPNIPQMSAFWYAVRTAVIN
AASGRQTVDEALKDAQTNSGSGSGENLYFQ)DEARKAIARVKRESKRIVEDAERLIREAAAASEKISREAERLIR
(GGGSGSGSGSGKPGQASGS)AHIVMVDAYKPTK
>sfGFP_VMAn_p18
(SEQ ID NO: 27041)
(MGSSHHHHHHSSGLVPRGSHMSKGEELFTGVVPILVELDGDVNGHKFSVRGEGEGDATNGKLTLKFICTTGKLP
VPWPTLVTTLTYGVQCFARYPDHMKQHDFFKSAMPEGYVQERTISFKDDGTYKTRAEVKFEGDTLVNRIELKGID
FKEDGNILGHKLEYNFNSHNVYITADKQKNGIKANFKIRHNVEDGSVQLADHYQQNTPIGDGPVLLPDNHYLSTQ
SVLSKDPNEKRDHMVLLEFVTAAGITHGMDELYKSGSGSGCFAKGTNVLMADGSIECIENIEVGNKVMGKDGRPR
EVIKLPRGRETMYSVVQKSQHRAHKSDSSREVPELLKFTCNATHELVVRTPRSVRRLSRTIKGVEYFEVITFEMG
QKKAPDGRIVELVKEVSKSYPISEGPERANELVESYRKASNKAYFEWTIEARDLSLLGSHVRKATYQTYAPILYG
GSGGS)DEARKAIARVKRESKRIVEDAERLTREAAAASEKISREAERLIREAAAASEKISRE
>p18_VMAc_mCherry
(SEQ ID NO: 27042)
(MGSHHHHHHGSGSENLYFQGSG)DEARKAIARVKRESKRIVEDAERLIREAAAASEKISREAERLIREAAAASE
KISRE(GSGGSGSGGGVLLNVLSKCAGSKKFRPAPAAAFARECRGFYFELQELKEDDYYGITLSDDSDHQFLLAN
QVVVHNCGSGGSVSKGEEDNMAIIKEEKRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGP
VMQKKTMGWEASSERHYPEDGALKGEIKQRLKIKDGGHYDAEVKTTYKAKKPVQLPGAYNVNIELDITSHNEDYT
IVEQYERAEGRHSTGGMDELYK)
(Cognate Keys for 2plus1 and 3plus1 STREPII-LOCKR functional Cage designs):
>2plus1_KEY_100000.fasta alt STREP_2plus1_1
(SEQ ID NO: 27043)
DKVRKVAEVAEKVLRDIDKLDRESKEAFRRTNGEISKLDEDTRRVAERVKKAIEDLAK
>2p1us1_KEY_2
(SEQ ID NO: 27044)
SEVDEIIADNERALDEVRREVEEIDKENAERLKEWVEEAREILDRLAKALEEIR
>2p1us1_KEY_3
(SEQ ID NO: 27045)
PEEALSKTIKDRDIVKKVKDELKEWRDRNKELVDRLSEELKEWLKDVERVLKELTDKDR
>2pius1_KEY_4
(SEQ ID NO: 27046)
DERVREELKKLLTRVEEEHRKVLETDKKILKEARKESKEVNDPDRELLERLEESVR
>3p1us1_KEY_1
(SEQ ID NO: 27047)
SRLVKKIDEIV-KEVAKKLEDVVRANEELWRKLVELNKESVARLREAVERVARDLEETAR
>3p1us1_KEY_2
(SEQ ID NO: 27048)
SDEEPLEKVVKEVIEKVRRILEKWKKDIDKVVKELRRILEEWEKIIREVLDKVR
>3pEus1_KEY_3
(SEQ ID NO: 27049)
DKDAVIKVIEKLIPANAAVWDAIIKINEDLVIWNKTVWKELLRVNEKLARDLERVVK
>3plus1_KEY_4
(SEQ ID NO: 27050)
SLVDELRKSLERNVRVSEEVARRLKEALKRWVDVVRKVVEDLIRLNEDVVRVVEK
SEQ ID NOs: 26,602-27,015:
>3plus1_GFP11_Key_Cterm_1
(SEQ ID NO: 26602)
SGSKEVDDILERAVEVVRRVIKALKEVLERHVDATREVIERVKRVNKRLLEAVREVVT
>3plus1_GFP11_Key_Cterm_2
(SEQ ID NO: 26603)
GVPEEIDRELKPVVEELRRLHEEIKERLDDVARRSEEELRRIIKKLKEVVKEIRKKLK
>3plus1_GFP11_Key_Cterm_3
(SEQ ID NO: 26604)
DLLRKLEEELRRIKEKLRKALEELEREHRELEKELDKLHDESRKEHERIEEELRR
>3plus1_GFP11_Key_Cterm_4
(SEQ ID NO: 26605)
DEDLLEKIKRVIREHIKALEKLARDLKEILRRHIEALKELARDLAEVIRKLLEDVKR
>3plus1_GFP11_Key_Cterm_5
(SEQ ID NO: 26606)
DLERLRRKVEELEDRLRRDLEKLARDSAELMRELERILDRYARESEELDRRDAE
>3plus1_GFP11_Key_Cterm_6
(SEQ ID NO: 26607)
DLEDILPKNLDRLRKLLERLREILRENLEALKKTLKRLEDVVRREIEDLKRERK
>3plus1_GFP11_Key_Cterm_7
(SEQ ID NO: 26608)
DLERLRRKVEELEDRLRRLLEKLARDSAELMRELERILDRYARESEELDRRLAE
>3plus1_GFP11_Key_Cterm_8
(SEQ ID NO: 26609)
SGSKEVLDILERAVEVVRRVIKALKEVLERHVDATREVIERVKRVNKRLLEAVREVVT
>3plus1_GFP11_Key_Cterm_9
(SEQ ID NO: 26610)
DIERLRRKVEELEDRLRRLLEKLARDSAELMRELERILDRYARESEELDRRLAE
>3plus1_GFP11_Key_Cterm_10
(SEQ ID NO: 26611)
RLIEEVVRLLRENLDVVRRILEALAKLIKELLEALEEVLRRNKELIRELLRVLDEALK
>3plus1_GFP11_Key_Cterm_11
(SEQ ID NO: 26612)
DIVRAMEEVIRRLIEILRRDVELNLDVAKKLLELLKEDSKLNLDVARELLELDDR
>3plus1_GFP11_Key_Cterm_12
(SEQ ID NO: 26613)
DIVRAMEEVIRRLIElLRRDVELNLDVAKKLLELLKEDSKLN1DVARELLELLDR
>3plus1_GFP11_Key_Cterm_13
(SEQ ID NO: 26614)
RLIEEVVRLLRENLDVVRRILEALAKLIKELLEALEEVLRRNKELIRELLRVLDEALK
>3plus1_GFP11_Key_Cterm_14
(SEQ ID NO: 26615)
RLIEEVVRLLRENLDVVRRILEALAKLIKELLEALEEVLRRNKELIRELLRVLDEALK
>3plus1_GFP11_Key_Cterm_15
(SEQ ID NO: 26616)
DLLRKLEEELRRIKEKLRKALEELEREHRELEKELDKLHDESRKEHERIEEELRR
>3plus1_GFP11_Key_Cterm_16
(SEQ ID NO: 26617)
DLLRKLEEELRRIKEKLRKALEELEREHRELEKELDKLHDESRKEHERIEEELRR
>3plus1_GFP11_Key_Cterm_17
(SEQ ID NO: 26618)
ELAREVERVIKELLDKSKEILERIERAIDELLKVSEEILKLSEDASEELLKILREFAK
>3plus1_GFP11_Key_Cterm_18
(SEQ ID NO: 26619)
DVKDIIRTILEVARDLLRLLEEDSRTNSEVVKRLLDLLREDSKANSEVVKRILDVLRE
>3plus1_GFP11_Key_Cterm_19
(SEQ ID NO: 26620)
DLERLRRKVEELEDRLRRLLEKLARDSAELMRELERILDRYARESEELDRRLAE
>3plus1_GFP11_Key_Cterm_20
(SEQ ID NO: 26621)
DLERLRRKVEELEDRLRRLLEKLARDSAELMRELERILDRYARESEELDRRLAE
>3plus1_GFP11_Key_Cterm_21
(SEQ ID NO: 26622)
RLIEEVVRLLRENLDVVRRILEALAKLIKELLEALEEVLRRNKELIRELLRVLDEALK
>3plus1_GFP11_Key_Cterm_22
(SEQ ID NO: 26623)
DLEDILRKNLDRLRKLLERLREILRENLEALKKTLKRLEDVVREILEDLKRERK
>3plus1_GFP11_Key_Cterm_23
(SEQ ID NO: 26624)
DLLRKLEESLPPIKEKLRKALEELEREHRELEKELDKLHDESPKEKERIEEELRR
>3plus1_GFP11_Key_Cterm_24
(SEQ ID NO: 26625)
DEDLLEKIKRVIREHIKALEKLARDLKEILRRHIEALKELARDLAEVIRKLLEDVKR
>3plus1_GFP11_Key_Cterm_25
(SEQ ID NO: 26626)
ELVRIAIEVLKPLLEIIEELVRLNNEILERLLKIVRELHKDNIKILEDLLRIIEEVLR
>3plus1_GFP11_Key_Cterm_26
(SEQ ID NO: 26627)
ELVRIAIEVLKRLLEIIEELVRLNNEILERLLKIVRELHKDNIKILEDLLRIIEEVLR
>3plus1_GFP11_Key_Cterm_27
(SEQ ID NO: 26628)
RLARLLKALADKILIRVLEEILKINEELNRKIIKFARENLERNRRVNKKVIEVLREAAR
>3plus1_GFP11_Key_Cterm__28
(SEQ ID NO: 26629)
DLERLRRKVEELEDRLRRLLEKLARDSAELMRELERILDRYARESEELDRRLAE
>3plus1_GFP11_Key_Cterm_29
(SEQ ID NO: 26630)
ELVRIAIEVLKRLLEIIEELVRLNNEILERLLKIVRELHKDNIKILEDLLRIIEEVLR
>3plus1_GFP11_Key_Cterm_30
(SEQ ID NO: 26631)
ELVRIAIEVLKRLLEIIEELVRLNNEILERLLKIVRELHKDNIKILEDLLRIIEEVLR
>3plus1_GFP11_Key_Cterm_31
(SEQ ID NO: 26632)
DIVRAMEEVIRRLIEILRRDVELNLDVAKKLLELLKEDSKLMLDVARELLELLDR
>3plus1_GFP11_Key_Cterm_32
(SEQ ID NO: 26633)
RKIAK1TEE1KRLLEDLARDTRRVIEEAKRLLKEWRDRNKEVADTLKKLLEDLIRKIR
>3plus1_GFP11_Key_Cterm_33
(SEQ ID NO: 26634)
DLLRKLEEELRRIKEKLRKALEELEREHRELEKELDKLHDESRKEKERIEEELRR
>3plus1_GFP11_Key_Cterm_34
(SEQ ID NO: 26635)
DLLRKLEEELRRIKEKLRKALEELEREHRELEKELDKLHDESRKEKERIEEELRR
>3plus1_GFP11_Key_Cterm_35
(SEQ ID NO: 26636)
RKIAKIIEELKRLLEDLARDTRRVIEEAKRLLKEWRDRNKEVADTLKKLLEDLIRKIR
>3plus1_GFP11_Key_Cterm_36
(SEQ ID NO: 26637)
ELVRIAIEVLKRLLEIIEELVRLNNEILERLLKIVRELHKDNIKILEDLLRIIEEVLR
>3plus1_GFP11_Key_Cterm_37
(SEQ ID NO: 26638)
TVRRLREALKKLEDDLRKIERDAEREYKKLKDELEELTERYRREIRKLKEELKADRK
>3plus1_GFP11_Key_Cterm_38
(SEQ ID NO: 26639)
DEAERRRRELKDKLDRLREEHEEVKRRLEEELTPLRETHKKIEKELREALKRVRDRST
>3plus1_GFP11_Key_Cterm_39
(SEQ ID NO: 26640)
DEAERRRRSLKDKLDRLREEHEEVKRRLEEELTRLRETKKKIEKELREALKPVRDRST
>3plus1_GFP11_Key_Cterm_40
(SEQ ID NO: 26641)
DLEDILRKNLDRLRKLLERLREILRENLEALKKTLKRLEDWREILEDLKRERK
>3plus1_GFP11_Key_Cterm_41
(SEQ ID NO: 26642)
DIERLRRKVEELEDRLRRLLEKLARDSAELMRELERIIDRYARESESLDRRIAE
>3p1us1_GFP11_Key_Cterm_42
(SEQ ID NO: 26643)
DEAERRRRELKDKLDRLREEHEEVKPRLEEELTRLRETHKKIEKELREALKRVRDRST
>3p1us1_GFP11_Key_Cterm_43
(SEQ ID NO: 26644)
DEAERRRRELKDKLDRLREEHEEVKPRLEEELTRLRETHKKIEKELREALKRVRDRST
>3p1us1_GFP11_Key_Cterm_44
(SEQ ID NO: 26645)
DEAERRRRELKDKLDRLREEHEEVKRPIEEELTRLRETHKKIEKELREALKRVRDRST
>3p1us1_GFP11_Key_Cterm_45
(SEQ ID NO: 26646)
DKAVEELEKALEEIKRRLKEVIDRYEDELRKLRKEYKEKIDKYERKLEEIERRERT
>3p1us1_GFP11_Key_Cterm_46
(SEQ ID NO: 26647)
DVKRALEELVSRLRKLLEDVKKASEDIVREVERIVRELAKRSDEILKKLEDIVEKLRE
>3p1us1_GFP11_Key_Cterm_47
(SEQ ID NO: 26648)
DVKRALEELVSRLRKLLEDVKKASEDIVREVERIVRELAKRSDEILKKLEDIVEKLRE
>3p1us1_GFP11_Key_Cterm_48
(SEQ ID NO: 26649)
DEAERRRRELKDKLDRLREEHEEVKRRLEEELTRLRETHKKIEKELREALKRVRDRST
>3p1us1_GFP11_Key_Cterm_49
(SEQ ID NO: 26650)
DVKRkLEELVSRLRKLLEDVKKk8EDIVREVERIVRELAKRSDEILKKLEDIVEKLRE
>3p1us1_GFP11_Key_Cterm_50
(SEQ ID NO: 26651)
EVKRRLEEKERRIRTRYEELRRRLRKRVKDYEDKLREIEKKVRRDAERIEEELERAKK
>3p1us1_GFP11_Key_Cterm_51
(SEQ ID NO: 26652)
DFAERRRRELKDKLDRLREEHEEVKRRLEEELTRLRETHKKIEKELREALKRVRDRST
>3p1us1_GFP11_Key_Cterm_52
(SEQ ID NO: 26653)
KIAEEIERELEELPRMIKRLHEDLERKIKESEDELREIEAPIEEKIPPLEEKLERKRR
>3p1us1_GFP11_Key_Cterm_53
(SEQ ID NO: 26654)
KIAEEIERELEELRRMIKRLHEDLERKLKESEDELREIEARIEEKIRRLEEKLERKRR
>3p1us1_GFP11_Key_Cterm_54
(SEQ ID NO: 26655)
DKAVEELEKNLEEIKRRLKEVIDRYEDELRKLRKEYKEKIDKYEPKLEEIERRERT
>3p1us1_GFP11_Key_Cterm_55
(SEQ ID NO: 26656)
KIAEEIERELEELRRMIKRLHEDLERKLKESEDELREIEARLEEKIRRLEEKIERKRP
>3p1us1_GFP11_Key_Cterm_56
(SEQ ID NO: 26657)
ELVRIAIEVLKRLLEIIEELVPLNNEILERLLKIVRELHKDNIKILEDLLRIIEEVLR
>3p1us1_GFP11_Key_Cterm_57
(SEQ ID NO: 26658)
DEVEREIRRVKEDLDRILEEYRRLLEEIKRKLEEILRRVEELHRRLRRKLEEIDR
>3p1us1_GFP11_Key_Cterm_58
(SEQ ID NO: 266591
DVKPALEELVSRLRKLLEDVKKASEDIVREVERIVRELAKRSDEILKKLEDIVEKLRE
>3p1us1_GFP11_Key_Cterm_59
(SEQ ID NO: 26660)
DEAERRRRELKDKLDRLREEHEEVKRRLEEELTRLRETHKKIEKELREALKRVRDRST
>3p1us1_GFP11_Key_Cterm_60
(SEQ ID NO: 26661)
DEAERRRRELKDKLDRLREEHEEVKRRLEEELTRLRETKKKIEKELREALKRVRDRST
>3p1us1_GFP11_Key_Cterm_61
(SEQ ID NO: 26662)
TLREVVRKVLEEAKRLLDELEEVHKRVKKELEDIIEENRRVVKRVRDELREIKRELDE
>3p1us1_GFP11_Key_Cterm_62
(SEQ ID NO: 26663)
DVKRALEELVSRLRKLLEDVKKASEDIVREVERIVRELAKRSDEILKKLEDIVEKLRE
>3p1us1_GFP11_Key_Cterm_63
(SEQ ID NO: 26664)
DVKRALEELVSPLRKLLEUVKKASEDIVREVERIVRELAKRSDEILKKLEDIVEKLRE
>3p1us1_GFP11_Key_Cterm_64
(SEQ ID NO: 26665)
DEAERRRRELKDKLDRLREEHEEVKRRLEEELTRLRETHKKIEKELREALKRVRDRST
>3p1us1_GFP11_Key_Cterm_65
(SEQ ID NO: 26666)
DEAEPPRRELKDKLDRLREEHEEVKRRLEEELTRLRETHKKIEKELREALKRVPDRST
>3p1us1_GFP11_Key_Cterm_66
(SEQ ID NO: 26667)
KIAEEIERELEELRRMIKRLHEDLERKLKESEDELREIEARLEEKIRRLEEKLERKRR
>3p1us1_GFP11_Key_Cterm_67
(SEQ ID NO: 26668)
DVKRALEELVSRLAKLLEDVKKASEDIVREVERIVREIAKRSDEILKKLEDIVEKLRE
>3p1us1_GFP11_Key_Nterm_68
(SEQ ID NO: 26669)
SEAERLADEVREAVKKSEEDNETLVREVEYAVRELKKNNKTWVDEVRKLMKRIVDLLR
>3p1us1_GFP11_Key_Nterm_69
(SEQ ID NO: 26670)
SEAERLADEVRKAVKKSEEDNETLVREVEKAVRELKKNNKTWVDEVRKLMKRIVDLLR
>3p1us1_GFP11_Key_Nterm_70
(SEQ ID NO: 26671)
DKDKRLEELLKRLKELNDKTFEELERILEELKRANEASLREAERILEELRARIEGGNL
>3p1us1_GFP11_Key_Nterm_71
(SEQ ID NO: 26672)
SEAEEDLEELIKELAELLKDVIRKEEKINRRINKTLEDIIRRLKEISKEAEELRKGTV
>3plus1_GFP11_Key_Nterm__72
(SEQ ID NO: 26673)
SDKEEIKRRVEKTARDLETEHDKIKKRLEDTVRDIKRELDELLEKYERVLRKIEKTLR
>3plus1_GFP11_Key_Nterm_73
(SEQ ID NO: 26674)
SEAEKIREALETNLRLLEELIKRLKEILDTHNELLRRVIETLERLLKELLELLEEGGL
>3plus1_GFP11_Key_Nterm_74
(SEQ ID NO: 26675)
SEAEKIREALETNLRLLEELIKRLKEILDTHNELLRRVIETLERLLKELLELLEEGGL
>3plus1_GFP11_Key_Nterm_75
(SEQ ID NO: 26676)
SKEERLREVAEKKKKDLEDIVKRVDEAAKETARRLEEILKRLEEVLKKILDDLEKGPD
>3plus1_GFP11_Key_Nterm_76
(SEQ ID NO: 26677)
SLEEITKRLLELVEENLARHEEILRELLELAKRLAKEDRDILEEVLKLIEELLKLLED
>3plus1_GFP11_Key_Nterm_77
(SEQ ID NO: 26678)
SKEETLKRLLDELEKRNRETVERLERLLKELEDRNRASLEELEAVLEELERKIEESGL
>3plus1_GFP11_Key_Nterm_78
(SEQ ID NO: 26679)
SKEETLKRLLDELEKRNRETVERLERLLKELEDRNRASLSELEAVLEELERKIEESGL
>3plus1_GFP11_Key_Nterm_79
(SEQ ID NO: 26680)
SKEETLKRLLDELEKRNRETVERLERLLKELEDRNRASLEELEAVLEELERKIEESGL
>3plus1_GFP11_Key_Nterm_80
(SEQ ID NO: 26681)
STREKAKKVLDTLRADNEDMKRVVEKILRALKRTNERAEKLAREITEEIKRILKEVGV
>3plus1_GFP11_Key_Nterm_81
(SEQ ID NO: 26602)
DAEEVVKRLADVLRENDETIRKVVEDLVRIAEENDRLKKKLVEDIAEILRRIVELLRR
>3plus1_GFP11_Key_Nterm_82
(SEQ ID NO: 26603)
SKEETLKRLLDELEKRNRETVERLERLLKELEDRNRASLEELEAVLEELERKIEESGL
>3plus1_GFP11_Key_Nterm_83
(SEQ ID NO: 26604)
STREKAKKVLDTLRADNEDMKRVVEKILRALKRTNERAEKLAPEITEEIKRILKEVGV
>3plus1_GFP11_Key_Nterm_84
(SEQ ID NO: 26685)
SKEEEVEKVLPKWEEILRRLIEENKRANDKIRREYEELVKEIRRVLEEIKEVAERLGV
>3plus1_GFP11_Key_Nterm_05
(SEQ ID NO: 26686)
DREKSVRDIEEDLKRVLDKLRRRVETSKEELKKVLKADKENADELEKTLRDVVRELDR
>3plus1_GFP11_Key_Nterm_86
(SEQ ID NO: 26687)
SDKEEIKRRVEKTARDLETEHDKIKKRLEDTVRDIKRELDELLEKYERVLRKIEKTLR
>3plus1_GFP11_Key_Nterm_87
(SEQ ID NO: 26688)
STREKAKKVLDTLRADNEDMKRVVEKILRALKRTNERAEKLAREITEEIKRLLKEVGV
>3plus1_GFP11_Key_Nterm_88
(SEQ ID NO: 26689)
SKDEELARLIEELVERWRKIVEDLERDHRRLVKEIRELVERIRKKLEELVDRIRKNGI
>3plus1_GFP11_Key_Nterm_89
(SEQ ID NO: 26690)
SEAERLADEVRKAVKKSEEDNETLVREVEKAVRELKKNNKTWVDEVRKLMKRLVDLLR
>3plus1_GFP11_Key_Nterm_90
(SEQ ID NO: 26691)
SKDEELARLLEELVERWRKIVEDLERDHRRLVKEIRELVSRIRKKLEELVDRIRKNGI
>3p1us1_GFP11_Key_Nterm_91
(SEQ ID NO: 26692)
KEIEETLKELEDLNREMVETNRRVLEETRRLNKETVDRVKATLDELAKMLKKLVDDVR
>3plus1_GFP11_Key_Nterm_92
(SEQ ID NO: 26693)
SEAERLADEVRKAVKKSEEDNETLVREVEKAVRELKKNNKTWVDEVRKLMKRLVDLLR
>3plus1_GFP11_Key_Nterm_93
(SEQ ID NO: 26694)
SKEETLKRLLDELEKRNRETVERLERLLKELEDRNRA3LSELEAVLEELERKIEESGL
>3plus1_GFP11_Key_Nterm_94
(SEQ ID NO: 26695)
DKAEVLREALKLLKDLLEELIKIHEESLKRILDLIDTLVKVHEDALRALKELLERSGL
>3plus1_GFP11_Key_Nterm_95
(SEQ ID NO: 26696)
SKEEEVEKVLRKWEEILRRLIEENKRANDKIRREYEELVKEIRRVLEEIKEVAERLGV
>3plus1_GFP11_Key_Nterm_96
(SEQ ID NO: 26697)
SKEETLKRLLDELEKRNRETVERLERLLKELEDRNRASLEELEAVLEELERKIEESGL
>3plus1_GFP11_Key_Cterm_97
(SEQ ID NO: 26698)
SERVKEILERILRVVEEAVRLNEESLRRILDVVRKAVKLDRESLKKILDVVEEAVR
>3plus1_GFP11_Key_Cterm_98
(SEQ ID NO: 26699)
SERVKEILERILRVVEEAVRLNEESLRRILDVVRKAVKLDRESLKKILDVVEEAVR
>3plus1_GFP11_Key_Cterm_99
(SEQ ID NO: 26700)
DERRIAERIRELLRESKKLVRDVVSEAKRLLKENRDSTRKIIEDIRRLLRKIEDSTR
>3plus1_GFP11_Key_Cterm_100
(SEQ ID NO: 26701)
DALSRLLEELLRVVDDLIRVLKELIDKSRKVIEELLELLKRINEENLKVLAEIIK
>3plus1_GFP11_Key_Cterm_101
(SEQ ID NO: 26702)
DERRIAERIRELLPESKKLVRDVVEEAKRLLKENRDSTRKIIEDIRRLLRKIEDSTR
>3plus1_GFP11_Key_Cterm_102
(SEQ ID NO: 26703)
EALRKLVELLVEVLRRLIRVNRELVKLLREVLERILRILRESVKKLKPLIEKVTKDAT
>3plus1_GFP11_Key_Cterm_103
(SEQ ID NO: 26704)
EALRKLVELLVEVLRRLIRVNRELVKLLREVLERLLRILRESVKKLKRLIEKVIKDAT
>3plus1_GFP11_Key_Cterm_104
(SEQ ID NO: 26706)
AAKRLVEELLKAVTDLSRKNKRILEELLKAIETLSDENKKIITEILDALRRLVEKIEK
>3plus1_GFP11_Key_Cterm_105
(SEQ ID NO: 26706)
EALRKLVELLVEVLRRLIRVNRELVKLLREVLERLLRILRESVKKLKRLIEKVIKDAT
>3plus1_GFP11_Key_Cterm_l06
(SEQ ID NO: 26707)
EALRKLVELLVEVLRRLIRVNRELVKLLREVLERLLRILRESVKKLKRLIEKVIKDAT
>3plus1_GFP11_Key_Cterm_107
(SEQ ID NO: 26708)
DALSRLLEELLRVVDDLIRVLKELIDKSRKVIEELLELLKRINEENLKVLAEIIK
>3plus1_GFP11_Key_Cterm_108
(SEQ ID NO: 26709)
DALSPLLESLLRVVDDLIRVLKELIDKSRKVIEELLELLKRINEENLKVLAEIIK
>3plus1_GFP11_Key_Cterm_109
(SEQ ID NO: 26710)
RAVKKLDEIVKEVAKKLEDVVRANEELWRALVELNKESVRRLREIVERVARDLEETAR
>3plus1_GFP11_Key_Cterm_110
(SEQ ID NO: 26711)
DRLDKVEELVKKLLEDTKRTVDRVRELVRKILKKSRETLEELERLISKILRELEKDAR
>3plus1_GFP11_Key_Cterm_111
(SEQ ID NO: 26712)
DALSRLLEELLRVVDDLIRVLKELIDKSRKVIEELLELLKRINEENLKVLAEIIK
>3plus1_GFP11_Key_Cterm_112
(SEQ ID NO: 26713)
RAVKKLDEIVKEVAKKLEDVVRANEELWRALVELNKESVRRLREIVERVARDLEETAR
>3plus1_GFP11_Key_Cterm_113
(SEQ ID NO: 26714)
RAVKKLDEIVKEVAKKLEDVVRANEELWRALVELNKESVRRLREIVERVARDLEETAR
>3plus1_GFP11_Key_Cterm_114
(SEQ ID NO: 26715)
SEDDLKRVVDEVEKKLRELKRRYAEALERIKEKIKELKDRYERAVREVVAELRKTTK
>3plus1_GFP11_Key_Cterm_115
(SEQ ID NO: 26716)
RAVKKLDEIVKEVAKKLEDVVRANEELWRALVELNKESVRPIREIVEPVARDLEETAR
>3plus1_GFP11_Key_Cterm_116
(SEQ ID NO: 26717)
DEVEREIRRVKEDLDRILEEYRRLLEEIKRKLEEILRRVSELHRRLRRKLEEIDR
>3plus1_GFP11_Key_Cterm_117
(SEQ ID NO: 26718)
SEDDLKRVVDEVEKKLRELKFRYAEALERIKEKIKELKDRYERAVREVVAELRKTTK
>3plus1_GFP11_Key_Nterm_118
(SEQ ID NO: 26719)
DEAKELLDEIRKAVKE5EDRLEKLLRDYEKELRPLEKELRDLKRRIEEKLEELRRGSL
>3plus1_GFP11_Key_Nterm_119
(SEQ ID NO: 26720)
SEKEDAARKLRKLVEELTREYEELVKKLERLIEEIEKVSEESVRKLEKLLAEISEEVR
>3plus1_GFP11_Key_Nterm_120
(SEQ ID NO: 26721)
SEDEIIKKIIEDLRRVLKEVEEIHKEVEERLDKVLKEAEEMHKEVLKELDRVLDEVKR
>3plus1_GFP11_Key_Nterm_121
(SEQ ID NO: 26722)
SKAEEIAEKLDRLLEENRRALEEITTRLDDLLRRNKDALRKVMEKLKRLLDDLRRGGI
>3plus1_GFP11_Key_Nterm_22
(SEQ ID NO: 26723)
SEKEKLLKESEEEVRRLRRTLEELLRKYREVLERLRKELRET.EEFVRDWRRLKEVLD
>3plus1_GFP11_Key_Nterm_123
(SEQ ID NO: 26724)
SEKEDAARKLRKLVEELTREYE2LVKKLERLIEEIEKVSEESVRKLEKLLAEISEEVR
>3plus1_GFP11_Key_Nterm_124
(SEQ ID NO: 26725)
SKEETLRKEAEDLLRRLEELTRRLEKKARELLERAKKLSRDLAEELKRLLKELREKGV
>3plus1_GFP11_Key_Nterm_125
(SEQ ID NO: 26726)
SEKEDAARKLRKLVEELTREYEELVKKLERLIEEIEKVSEESVRKLEKLIAEISEEVR
>3plus1_GFP11_Key_Nterm_126
(SEQ ID NO: 26727)
SKAEEIAEKLDRLLEENRRALEEITTRLDDLLRRNKDALRKVMEKLKRLLDDLRRGGI
>3plus1_GFP11_Key_Nterm_127
(SEQ ID NO: 26728)
SKAEEIAEKLDRLLEENRRALEEITTRLDDLLRRNKDALRKVMEKLKRLLDDLRRGGI
>3plus1_GFP11_Key_Nterm_128
(SEQ ID NO: 26729)
SKAEEIAEKLDRLLEENRPALEEITTRLDDLLRRNKDALRKVMEKLKRLLDDLRRGGI
>3plus1_GFP11_Key_Nterm_129
(SEQ ID NO: 26730)
SKEETLRKEAEDLLRRLEELTRRLEKKARELLERAKKLSRDLAEELKRLLKELREKGV
>3plus1_GFP11_Key_Nterm_130
(SEQ ID NO: 26731)
SRVEELKKLIEDILRISREVVERIKRVAEDIHRINRRVLDDLRKLIEDILRTVEEILA
>3plus1_GFP11_Key_Nterm_131
(SEQ ID NO: 26732)
SKEETLRKEAEDLLRRLEELTRRLEKKARELLERAKKLSRDLAEELKRLLKELREKGV
>3plus1_GFP11_Key_Nterm_132
(SEQ ID NO: 26733)
SKAEEIAEKLDRLLEENRRALEEITTRLDDLLRRNKDALRKVMEKLKRLLDDLRRGGI
>3plus1_GFP11_Key_Nterm_133
(SEQ ID NO: 26734)
SKEETLRKEAEDLLRRLEELTRRLEKKARELLERAKKLSRDLAEELKRLLKELREKGV
>3plus1_GFP11_Key_Nterm_134
(SEQ ID NO: 26735)
SEKEDAARKLRKLVEELTREYEELVKKLERLIEEIEKVSEESVRKLEKLLAEISEEVR
>3plus1_GFP11_Key_Nterm_l35
(SEQ ID NO: 26736)
SKEETLRKEAEDLLRRLEELTRRLEKKARELLERAKKLSRDLAEELKRLLKELREKGV
>3plus1_GFP11_Key_Nterm_136
(SEQ ID NO: 26737)
SERETVKRRLEELLKEVKRTLDKLKEEHDRLLEDVRRVVEELKREHDKLLKEVKDSGV
>3plus1_GFP11_Key_Nterm_137
(SEQ ID NO: 26738)
SEKEDAARKLRKLVEELTREYEELVKKLERLXEEIEKVSEESVRKLEKLLAEISEEVR
>3plus1_GFP11_Key_Nterm_138
(SEQ ID NO: 26739)
SKEETLRKEAEDLLRRLEELTRRLEKKARELLERAKKLSRDLAEELKRLLKELREKGV
>3plus1_GFP11_Key_Nterm_139
(SEQ ID NO: 26740)
KEREEVKEKLDRLLEEVEKTVRELKREHDELLKEVEKLVRDLKKEHDELLKKVKDDGV
>3plus1_GFP11_Key_Nterm_140
(SEQ ID NO: 26741)
SREEVLRELEEVIEDNRRLLEELIEKSKKVLDESLKLIDELLRRLEEVLERVLRLLEE
>2plus1_GFP11_Key_Cterm_1
(SEQ ID NO: 26742)
DEVVKRVRDLLDTVRRRNEKVNEDVKRMNDKLRRDNEDVIRRVEKLLRELEEKRRT
>2plus1_GFP11_Key_Cterm_2
(SEQ ID NO: 26743)
SEDSVERIARELERNLDDLARVLKESEDDLAEILRRLKEVLEESERDLERVEREVRK
>2plus1_GFP11_Key_Cterm_3
(SEQ ID NO: 26744)
SKELLEKAKAVVDEIKRIAEESLKRLEDLSRDHKRRAKELNDEIAKVVDELAKPAT
>2plus1_GFP11_Key_Cterm_4
(SEQ ID NO: 26745)
SKEKIDRIIRELERILEEAKKKHEDVLRRLEDSLRRVAELLKAALDRLREIVDRLRR
>2plus1_GFP11_Key_Cterm_5
(SEQ ID NO: 26746)
DEVVKRVRDLLDTVRRRNEKVNEDVKRMNDKLRRDNEDVIRRVEKLLRELEEKRRT
>2plus1_GFP11_Key_Cterm_6
(SEQ ID NO: 26747)
DIKTLLDRVRKLAEEDAERLDRLRRESEELNERVRRVDKKLLEEIRRKAKKVEDDTR
>2plus1_GFP11_Key_Cterm_7
(SEQ ID NO: 26748)
DAETLLRELEKLSRDNKELLKKIEKEIRDLIKEDKERNIELSERLPKLVEELKKKAT
>2plus1_GFP11_Key_Cterm_8
(SEQ ID NO: 26749)
DEWKRVRDLLDTVRRRNEKVNEDVKRMNDKLRRDNEDVIRRVEKLLRELEEKRRT
>2plus1_GFP11_Key_Cterm_9
(SEQ ID NO: 26750)
DIKTLLDRVRKLAEEDAERLDRLRRESEELNERVRRVDKXLIEEIRRKAKKVEDDTR
>2plus1_GFP11_Key_Cterm_10
(SEQ ID NO: 26751)
SEELSAEVKKLLDEVRKALARHKDENDKLLKEIEDSLRRHKEENDRLLEKLKESTR
>2plus1_GFP11_Key_Cterm_11
(SEQ ID NO: 26752)
DADDVLARVEELAKRAHDENERLIREVEELVRAHNKRNKELVDEVKRLVEKVIEEER
>2plus1_GFP11_Key_Cterm_12
(SEQ ID NO: 26753)
SKEKIDRIIRELERILEEAKKKHEDVLRRLEDSLRRVAELLKAALDRLREIVDRLRR
>2plus1_GFP11_Key_Cterm_13
(SEQ ID NO: 26754)
DEVVKRVRDLLDTVRRRNEKVNEDVKRMNDKLRPDNEDVIRRVEKLLRELEEKRRT
>2plus1_GFP11_Key_Cterm_14
(SEQ ID NO: 26755)
SEELSAEVKKLLDEVRKALARHKDENDKLLKEIEDSLRRHKEENDRLLEKLKESTR
>2plus1_GFP11_Key_Cterm_15
(SEQ ID NO: 26756)
DAETVLRSAEDIVAKNRKLAEEVLRRVKKIVEENRKIASEVLDDVRKLVEDVLARAS
>2plus1_GFP11_Key_Cterm_16
(SEQ ID NO: 26757)
DADDVLARVEELAKRAHDENERLIREVEELVRAHNKRNKELVDEVKRLVEKVIEEER
>2plus1_GFP11_Key_Cterm_17
(SEQ ID NO: 26768)
SKEKIDRIIPELERILEEAKKKHEDVLRRLEDSLRRVAELLKAALDRLREIVDRLRR
>2plus1_GFP11_Key_Cterm_18
(SEQ ID NO: 26759)
DEEKLKDLIPKLRDILRRAAEAHXKLIDDARESLERAKREHEKLIDRLKKIIEELER
>2plus1_GFP11_Key_Cterm_l9
(SEQ ID NO: 26760)
DIKTLLDRVRKLAEEDAERLDRLRRESEELNERVRRVDKKLLEEIRRKAKKVEDDTR
>2plus1_GFP11_Key_Cterm_20
(SEQ ID NO: 26761)
DATRVIEEAKRILDEARKLNEETIRRSEELVRRIERVIESIIKRSEKLLEDVARESK
>2plus1_GFP11_Key_Cterm_21
(SEQ ID NO: 26762)
SEELSAEVKKLLDEVRKALARHKDENDKLLKEIEDSLRRHKEENDRLLEKLKESTR
>2plus1_GFP11_Key_Cterm_22
(SEQ ID NO: 26763)
DAETVLPSAEDIVAKNRKLAEEVLRRVKKIVEENRKIASEVLDDVRKLVEDVLARAS
>2plus1_GFP11_Key_Cterm_23
(SEQ ID NO: 26764)
DADDVLARVEELAKRAHDENERlTrEVEELVRAHNKRNKELVDEVKRLVEKVIEEER
>2plus1_GFP11_Key_Cterm_24
(SEQ ID NO: 26766)
SKELLEKAKAVVDEIKRLAEESLKRLEDLSRDHKRRAKELNDEIAKVVDELAKRAT
>2plus1_GFP11_Key_Cterm_25
(SEQ ID NO: 26766)
DEEVLKKLAEIVRRVKEENRKVNEEVEKRLRELEEENKKVIEDLKSTVEELVERLR
>2plus1_GFP11_Key_Cterm_26
(SEQ ID NO: 26767)
DEEVLKKLAEIVRRVKEENRKVNESVEKRLRELEEENKKVIEDLKSTVEELVERLR
>2plus1_GFP11_Key_Cterm_27
(SEQ ID NO: 26768)
DKLLKEARDLIPEIEKRLEELLKRVEKLTEDAKRDLERSNREHKELADRIKETAR
>2plus1_GFP11_Key_Cterm_28
(SEQ ID NO: 26769)
DKDSARELERIVKENAELAERVFREVEKIVRENTKLAEDSVRELKRLVEELKKRAK
>2plus1_GFP11_Key_Cterm_29
(SEQ ID NO: 26770)
SKEKIDRIIRELERILEEAKKKHEDVLRRLEDSLRRVAELLKAALDRLREIVDRLRR
>2plus1_GFP11_Key_Cterm_30
(SEQ ID NO: 26771)
DEEKLKDLTRKLRDILRRAAEAHKKLIDDARESLERAKRSHEKLTDRLKKILEELER
>2plus1_GFP11_Key_Cterm_31
(SEQ ID NO:26772)
DEWKRVRDIiLDTVRRRNEKVNEDVKRMNDKLRRDNEDVIRRVEKLLRELEEKRRT
>2plus1_GFP11_Key_Cterm_32
(SEQ ID NO: 26773)
DEEVLRTLEEIIRRLTKELEDVLRSYERELRRLEEENKRVIDKTEEEIRRLADRLRR
>2plus1_GFP11_Key_Cterm_33
(SEQ ID NO: 26774)
DERILRELSERVKELEKEAREILKRSEDETDKLREKAERILEDLERANRRTMDEARR
>2plus1_GFP11_Key_Cterm_34
(SEQ ID NO: 26775)
SEELSAEVKKLLDEVRKALARHKDENDKLLKEIEDSLRRHKEENDRLLEKLKESTR
>2plus1_GFP11_Key_Cterm_35
(SEQ ID NO: 26776)
LPEEVLRELEELLKESEERIKRIEEEIKKIIDKSREDIKRVLEEIERLNAKAADDLRK
>2plus1_GFP11_Key_Cterm_36
(SEQ ID NO: 26777)
DADDVLARVEELAKRAHDENERLIREVEELVRAHNKRNKELVDEVKRLVEKVIEEER
>2plus1_GFP11_Key_Cterm_37
(SEQ ID NO: 26778)
DEEVLKKLAEIVRRVKEENRKVNEEVEKRLRELEEENKKVIEDLKSTVEEDVERLR
>2plus1_GFP11_Key_Cterm_38
(SEQ ID NO: 26779)
DKLLKEARDLIREIEKRLEELLKRVEKLTEDAKRDLERSNREHKELADRIKETAR
>2plus1_GFP11_Key_Cterm_39
(SEQ ID NO: 26780)
DEEVLRTLEEIIRRLTKELEDVLREYERELRRLEEENKRVIDKTEEEIRRLADRLRR
>2plus1_GFP11_Key_Cterm_40
(SEQ ID NO: 26781)
DRRIEKVLKEIEEKIREVIKEWERVHREVEELLKRLIDENRKVLDEIRKLLEEKSK
>2plus1_GFP11_Key_Cterm_41
(SEQ ID NO: 26782)
DERILRELEERVKELFIKEARElLKRSEDETDKLREKAERILEDLERANRRTMDEARR
>2plus1_GFP11_Key_Cterm_42
(SEQ ID NO: 26783)
SEELSAEVKKLLDEVRKALAPHKDENDKLLKEIEDSLERHKEENDRLLEKLKESTR
>2plus1_GFP11_Key_Cterm_43
(SEQ ID NO: 26784)
DEEVLKKLAEIVRRVKEENRKVNEEVEKRLRELEEENKKVIEDLKSTVEELVERLR
>2plus1_GFP11_Key_Cterm_44
(SEQ ID NO: 26785)
SKEKIDRIIRELERILSEAKKKHEDVLRRLEDSLRRVAELLKAALDRLREIVDRLRR
>2plus1_GFP11_Key_Cterm_45
(SEQ ID NO: 26786)
DRRIEKVLKEIEEKI.REVIKEWERVHREVESLLKRLIDENRKVLDEIRKLLEEKSK
>2plus1_GFP11_Key_Cterm_46
(SEQ ID NO: 26787)
TLRELARSIRKLSAENKERLKELLRELKKLSDENKERIKKLLSDAEKIIEDVARRAK
>2plus1_GFP11_Key_Cterm_47
(SEQ ID NO: 26788)
DERILRELEEPVKELEKEAREILKRSEDETDKLREKAERILEDLERANRRTMDEARR
>2plus1_GFP11_Key_Cterm_48
(SEQ ID NO: 26789)
EKLKELRDVIAEVAKRIDELDEYTRESIRPAKKEIERLNPETKKVIEEVVKRIEEERK
>2plus1_GFP11_Key_Cterm_49
(SEQ ID NO: 26790)
DERVREELKKLLTRVEEEHRKVLETDKKILKEAHKESKEVNDRDRELLERLEESVR
>2plus1_GFP11_Key_Cterm_50
(SEQ ID NO: 26791)
DADDVLARVEELAKRAHDENEPLIREVEELVRAHNKRNKELVDEVKRLVEKVIEEER
>2plus1_GFP11_Key_Cterm_51
(SEQ ID NO: 26792)
TVKRLLDELRELLERLKRTIEELLKRNRDLLADAEEKARRLLEENRKLLKAARDTAT
>2plus1_GFP11_Key_Cterm_52
(SEQ ID NO: 26793)
DEEVLKKLAEIVRRVKEENRKVNEEVEKRLRELEEENKKVIEDLKSTVEELVERLR
>2plus1_GFP11_Key_Cterm_53
(SEQ ID NO: 26794)
SKEKIDRIIRELER1LEEAKKKHEDVLRRLEDSLRRVAELLKAALDRLREIVDRLRR
>2plus1_GFP11_Key_Cterm_54
(SEQ ID NO: 26795)
DERILRELEERVKELEKEAREILKRSEDETDKLREKAERILEDLERANRRTMDEARR
>2plus1_GFP11_Key_Cterm_55
(SEQ ID NO: 26796)
DATRVIEEAKRILDEARKLNEETIRRSEELVRRIERVIEEIIKRSEKLLEDVARESK
>2plus1_GFP11_Key_Cterm_56
(SEQ ID NO: 26797)
EAAREIIKRLREVNKRTKEKLDELIKHSEEVLERVKRLIDELRKHSEEVLEDLRRRAK
>2plus1_GFP11_Key_Cterm_57
(SEQ ID NO: 26798)
EKLKELRDVIAEVAKRIDELDEYTRESIFRAKKEIERLNRETKKVIEEVVKRIEEERK
>2plus1_GFP11_Key_Cterm_58
(SEQ ID NO: 26799)
ELLRRIKKLLDEIKKAIEDSSREIKRLLEESERVMKRSSEDIKPTLDDTRRVVEEVRR
>2plus1_GFP11_Key_Cterm_59
(SEQ ID NO: 26800)
SKAIKDVRDIVKKVKDELKEWRDRNKELVDRLSEELKEWLKDVERVLKELTDKDR
>2plus1_GFP11_Key_Cterm_60
(SEQ ID NO: 26801)
DERVREELKKLLTRVEEEHRKVLETDKKILKEAHKESKEVNDRDRELLERLEESVR
>2plus1_GFP11_Key_Cterm_61
(SEQ ID NO: 26802)
DIDKLLKELRDLVEKIKKDLKELLERYEEIVRRIKELLKDLNREAEEVVRRLKEELR
>2plus1_GFP11_Key_Cterm_62
(SEQ ID NO: 26803)
DADDVLARVEELAKRAHDENERLIREVEELVRAHNKRNKELVDEVKRLVEKVIEEER
>2plus1_GFP11_Key_Cterm_63
(SEQ ID NO: 26804)
DEEVLKKLAEIVRRVKEENRKVNEEVEKRLRELEEENKKVIEDLKSTVEELVERLR
>2plus1_GFP11_Key_Cterm_64
(SEQ ID NO: 26805)
EREEELKEVADRVKEKLDRLNRENEKSSEELKRELDKINDENRETSERLKREIDETTR
>2plus1_GFP11_Key_Cterm_65
(SEQ ID NO: 26806)
SKEKIDRIIRELERILEEAKKKHEDVLRRLEDSLRRVAELLKAALDRLREIVDRLRR
>2plus1_GFP11_Key_Cterm_66
(SEQ ID NO: 26807)
SKEKIDRIIRELERILEEAKKKHEDVLRRLEDSLRRVAELLKAALDRLREIVDRLRR
>2plus1_GFP11_Key_Cterm_67
(SEQ ID NO: 26808)
TKDLLDENSKRSNEISREVKKDLERTVRENKKIVDEVAKALEDTVDKNRRIVEEVTT
>2plus1_GFP11_Key_Cterm_68
(SEQ ID NO: 26809)
DEWKRVRDLLDTVRRRNEKVNEDVKPMNDKLRRDNEDVIRRVEKLLRELEEKRRT
>2plus1_GFP11_Key_Cterm_69
(SEQ ID NO: 26810)
DRRIEKVLKEIEEKIREVIKEWERVHREVEELLKRLIDENRKVLDEIRKLLEEKSK
>2plus1_GFP11_Key_Cterm_70
(SEQ ID NO: 26811)
ELLRRIKKLLDETKKAIEDSSREIKRLLEESERVMKRSSEDIKRTLDDTRRWEEVRR
>2plus1_GFP11_Key_Cterm_71
(SEQ ID NO: 26812)
SEELSAEVKKLLDEVRKALARHKDENDKLLKEIEDSLRRHKEENDRLLEKLKESTR
>2plus1_GFP11_Key_Cterm_72
(SEQ ID NO: 26813)
DIDKLLKELRDLVEKIKKDLKELLERYEEIVRRIKELLKDLNREAEEWRRLKEELR
>2plus1_GFP11_Key_Cterm_73
(SEQ ID NO: 26814)
DADDVLARVEELAKRAHDENERLIREVEELVRAHNKRNKELVDEVKRLVEKVIEEER
>2plus1_GFP11_Key_Cterm_74
(SEQ ID NO: 26815)
DEEVLKKLAEIVRRVKSENRKVNEEVEKRLRELEEENKKVIEDLKSTVEELVERLR
>2plus1_GFP11_Key_Cterm_75
(SEQ ID NO: 26816)
DEEVLKKLAEIVRPVKSENRKVNEEVEKRLRELEEENKKVIEDLKSTVEELVERLR
>2plus1_GFP11_Key_Cterm_76
(SEQ ID NO: 26817)
SKEKIDRIIRELERILEEAKKKHEDVLRRLEDSLRRVAELLKAALDRLREIVDRLRR
>2plus1_GFP11_Key_Cterm_77
(SEQ ID NO: 26818)
SEELREELKKLERKIEKVAKEIHDHDKEVTERLEDLLRRITEHARKSDREIEETAR
>2plus1_GFP11_Key_Cterm_78
(SEQ ID NO: 26819)
DRRIEKVLKEIEEKIREVIKEWERVHREVEELLKRLIDENRKVLDEIRKLLEEKSK
>2plus1_GFP11_Key_Cterm_79
(SEQ ID NO: 26820)
DATRVIEEAKRILDEARKLNEETIRRSEELVRRIERVIEEIIKRSEKLLEDVARESK
>2plus1_GFP11_Key_Cterm_80
(SEQ ID NO: 26821)
EkLkELEDVIAEVAKRIDELDEYTRESIRRAKKEIERLNRETKKVIEEWKRIEEERK
>2plus1_GFP11_Key_Cterm_81
(SEQ ID NO: 26822)
ELLRRIKKLLDEIKKAIEDSSREIKRLLEESERVMKRSSEDIKRTLDDTRRVVEEVRR
>2plus1_GFP11_Key_Cterm_82
(SEQ ID NO: 26823)
DIDKLLKELRDLVEKIKKDLKELLERYEEIVRRIKELLKDLNREAEEVVRRLKEELR
>2plus1_GFP11_Key_Cterm_83
(SEQ ID NO: 26824)
EAKKKLDEVLERAKRTIDRLLETSDRSLEKVEADLRRLNEELDRSLERAERTIRELAK
>2plus1_GFP11_Key_Cterm_84
(SEQ ID NO: 26825)
DEEVLKKLAEIVRRVKEENRKVNEEVEKRLRELEEENKKVIEDLKSTVEELVERLR
>2plus1_GFP11_Key_Cterm_85
(SEQ ID NO: 26826)
DEEVLKKLAEIVRRVKEENRKVNEEVEKRLRELEEENKKVIEDLKSTVEELVERLR
>2plus1_GFP11_Key_Cterm_86
(SEQ ID NO: 26827)
DRRIEKVLKEIEEKIREVIKEWERVHREVEELLKRLIDENRKVLDEIRKLLEEKSK
>2plus1_GFP11_Key_Cterm_87
(SEQ ID NO: 26828)
DERILRELEERVKELEKEAREILKRSEDETDKLREKAERILEDLERANRRTMDEARR
>2plus1_GFP11_Key_Cterm_38
(SEQ ID NO: 26829)
DLKRVEEPAREVSRRNEESMRRVKEDADRVSEANKEVLDRVREEVKRLIEEVRETLR
>2plus1_GFP11_Key_Cterm_89
(SEQ ID NO: 26830)
EKLKELRDVIAEVAKRIDELDEYTRESIRRAKKEIERLNRETKKVIEEVVKRIEEERK
>2plus1_GFP11_Key_Cterm_90
(SEQ ID NO: 26831)
ELLRRIKKLLDEIKKAIEDSSREIKRLLEESERVMKRSSEDIKRTLDDTRRVVEEVRR
>2plus1_GFP11_Key_Cterm_91
(SEQ ID NO: 26832)
ELLRRIKKLLDEIKKAIEDSSREIKRLLEESERVMKRSSEDIKRTLDDTRRVVEEVRR
>2plus1_GFP11_Key_Cterm_92
(SEQ ID NO: 26833)
LPEEVLRELEELLKESEERIKRIEEEIKKIIDKSREDIKRVLEEIERLNAKAADDLRK
>2plus1_GFP11_Key_Cterm_93
(SEQ ID NO: 26834)
EAKKKLDEVLERAKRTIDRLLETSDRSLEKVEADLRRLNEELDRSLERAERTIRELAK
>2plus1_GFP11_Key_Cterm_94
(SEQ ID NO: 26835)
DEEVLKKLAEIVRRVKEENRKVNEEVEKRLRELEEENKKVIEDLKSTVEELVERLR
>2plus1_GFP11_Key_Cterm_95
(SEQ ID NO: 26836)
DKLLKEARDLIRETEKRLEELLKRVEKLTEDAKRDLERSNREHKELADRIKETAR
>2plus1_GFP11_Key_Cterm_96
(SEQ ID NO: 26637)
DKLLKEARDLIPEIEKRLEELLKRVEKLTEDAKRDLERSNREHKELADRIKETAR
>2plus1_GFP11_Key_Cterm_97
(SEQ ID NO: 26838)
DIVRKIERIVETIEREVRESVKKVEEIARDIRRKVDESVKNVEKLLRDVDKKARDRKK
>2plus1_GFP11_Key_Cterm_98
(SEQ ID NO: 26839)
DEIKRIVDEVPERLKRIVDENAKIVEDARRALEKIVKENSEILRRLKKELRELRK
>2plus1_GFP11_Key_Cterm_99
(SEQ ID NO: 26840)
DRRIEKVLKEIEEKIREVIKEWERVHREVEELLKRLIDENRKVLDEIRKLLEEKSK
>2plus1_GFP11_Key_Cterm_100
(SEQ ID NO: 26841)
DLKRVEEPAREVSRRNEESMRPVKEDADRVSEANKEVLDRVREEVKRLIEEVPETLR
>2plus1_GFP11_Key_Cterm_101
(SEQ ID NO: 26842)
DATRVIEEAKRILDEARKLNEETIRRSEELVRRIERVIEEIIKRSEKLLEDVAPESK
>2plus1_GFP11_Key_Cterm_102
(SEQ ID NO: 26843)
DAETIERVVRELLEENKEVLRKTEEAVKRSTETNKRLLEASKEVADRLRERIKEAAK
>2plus1_GFP11_Key_Cterm_103
(SEQ ID NO: 26844)
EKLKELRDVIAEVAKRIDELDEYTRESIRRAKKEIERLNRETKKVIEEVVKRIEEERK
>2plus1_GFP11_Key_Cterm_104
(SEQ ID NO: 26845)
ELLRRIKKLLDEIKKAIEDSSREIKRLLEESERVMKRSSEDIKRTLDDTRRVVEEVRR
>2plus1_GFP11_Key_Cterm_105
(SEQ ID NO: 26846)
DEVVERAERISEENKRRVEDVARKSKELVEDVRRHSEEVVRRVEELVKEVEERVR
>2plus1_GFP11_Key_Cterm_106
(SEQ ID NO: 26847)
EAKKKLDEVLERAKRTIDRLLETSDRSLEKVEADLKRLNEELDRSLERAERTIRELAK
>2plus1_GFP11_Key_Cterm_107
(SEQ ID NO: 26848)
TAERARETLKRLLDENRDRSKKVKEEIRRILEDLTRTTSRVKPEIAKLLKELEDTAR
>2plus1_GFP11_Key_Cterm_108
(SEQ ID NO: 26649)
EAVRRLKEILERLKEEVRRSLEELRKEVERLKKEVEDSLRELKKSLEEWVKSLEEATR
>2plus1_GFP11_Key_Cterm_109
(SEQ ID NO: 26850)
DERILRELEERVKELEKEAREILKRSEDETDKLREKAERILEDLERANRRTMDEARR
>2plus1_GFP11_Key_Cterm_110
(SEQ ID NO: 26851)
DATRVIEEAKRILDEARKLNEETIRRSEELVRRIERVIEEIIKRSEKLLEDVAPESK
>2plus1_GFP11_Key_Cterm_111
(SEQ ID NO: 26852)
DKARKVAEVAEKVLRDIDKLDRESKEAFRATNEEIAKLDEDTARVAERVKKAIEDLAK
>2plus1_GFP11_Key_Cterm_112
(SEQ ID NO: 26853)
EKLKELRDVIAEVAKRIDELDEYTRESIRRAKKEIERLNRETKKVIEEVVKRIEEERK
>2plus1_GFP11_Key_Cterm_113
(SEQ ID NO: 26854)
ELLRRIKKLLDEIKKAIEDSSREIKRLLEESERVMKRSSEDIKRTLDDTRRVVEEVRR
>2plus1_GFP11_Key_Cterm_114
(SEQ ID NO: 26855)
DKVERWREVEKLHEEDRKRLEESTRSVRKLLEELKRELEKSTRSVKALVDELRERVR
>2plus1_GFP11_Key_Cterm_l15
(SEQ ID NO: 26856)
DEWERAERISEENKRRVEDVARKSKELVEDVRRHSEEVVRRVEELVKEVEERVR
>2plus1_GFP11_Key_Cterm_116
(SEQ ID NO: 26857)
EAKKKLDEVLERAKRTIDRLLETSDRSLEKVFADLRRLNEELDRSLERAERTIRELAK
>2plus1_GFP11_Key_Cterm_117
(SEQ ID NO: 26858)
TAERARETLKRLLDENRDRSKKVKEEIRRILEDLTRTTERVKREIAKLLKELEDTAR
>2plus1_GFP11_Key_Cterm_118
(SEQ ID NO: 26859)
DERILRELSEPVKELEKEAREILKRSEDETDKLREKAERILEDLERANRRTMDEARR
>2plus1_GFP11_Key_Cterm_119
(SEQ ID NO: 26860)
DERILRELEERVKELEKEAREILKRSEDETDKLREKAERILEDLERANRRTMDEARR
>2plus1_GFP11_Key_Cterm_120
(SEQ ID NO: 26861)
DLKRVEERAREVSRRNEESMRRVKEDADRVSEANKEVLDRVREEVKRLIEEVRETLR
>2plus1_GFP11_Key_Cterm_21
(SEQ ID NO: 26862)
DKARKVAEVAEKVLRDIDKLDRESKEAFRATNEEIAKLDEDTARVAERVKKAIEDLAK
>2plus1_GFP11_Key_Cterm_122
(SEQ ID NO: 26863)
ELLRRIKKLLDEIKKAIEDSSREIKRLLEESERVMKRSSSDIKRTLDDTRRVVEEVRR
>2plus1_GFP11_Key_Cterm_123
(SEQ ID NO: 26864)
SKAIKDVRDIVKKVKDELKEWRDRNKELVDRLSEELKEWLKDVERVLKELTDKDR
>2plus1_GFP11_Key_Cterm_124
(SEQ ID NO: 26865)
DKVERVYREVEKLHEEDRKRLEESTRSVRKLLEELKRELEKSTRSVKALVDELRERVR
>2plus1_GFP11_Key_Cterm_125
(SEQ ID NO: 26866)
DIDKLLKELRDLVEKIKKDLKELLERYEEIVRRIKELLKDLNREAEEVVRRLKEELR
>2plus1_GFP11_Key_Cterm_126
(SEQ ID NO: 26867)
EAKKKLDEVLERAKRTIDRLLETSDRSLEKVEADLRRLNEELDRSLERAERTIRELAK
>2plus1_GFP11_Key_Cterm_127
(SEQ ID NO: 26868)
DKLLKEARDLIFEIEKRLEELLKRVEKLTEDAKRDLERSNREHKELADRIKETAR
>2plus1_GFP11_Key_Cterm_128
(SEQ ID NO: 26869)
TAERARETLKRLLDENRDRSKKVKEEIRRILEDLTRTTERVKREIAKLLKELEDTAR
>2plus1_GFP11_Key_Cterm_129
(SEQ ID NO: 26870)
DEIKRIVDEVRERLKRIVDENAKIVEDARRALEKIVKENEEILRRLKKELRELRK
>2plus1_GFP11_Key_Cterm_130
(SEQ ID NO: 26871)
DRIEEELKRLIDTIREKNREVEKRARDSNRDLKRTNDEIAKEVRELIKKLREDLK
>2plus1_GFP11_Key_Cterm_l31
(SEQ ID NO: 26872)
DERILRELEERVKELEKEAREILKRSEDETDKLREKAERILEDLERANRRTMDEARR
>2plus1_GFP11_Key_Cterm_132
(SEQ ID NO: 26873)
DAETIERVVRERELLENKEVLFKTEEAVKRSTETNKRLLEASKEVADRLRERIKEAAK
>2plus1_GFP11_Key_Cterm_133
(SEQ ID NO: 26871)
DKARKVAEVAEKVLRDIDKLDRESKEAFRATNEEIAKLDEDTARVAERVKKAIEDLAK
>2plus1_GFP11_Key_Cterm_l34
(SEQ ID NO: 26875)
DKVERVVRSVEKLHEEDRKRLEESTRSVRKLLEELKRELEKSTRSVKALVDELRERVR
>2plus1_GFP11_Key_Cterm_135
(SEQ ID NO: 26876)
DIERILRELEAVLKKLTDESERLNREVERVSRDTKKKSKELNEELKAVLDEVKRKAD
>2plus1_GFP11_Key_Cterm_136
(SEQ ID NO: 26877)
DEWERAERISEENKRRVEDVARKSKELVEDVRRHSEEVVRRVEELVKEVEERVR
>2plus1_GFP11_Key_Cterm_137
(SEQ ID NO: 26878)
EAKKKLDEVLERAKRTIDRLLETSDRSLEKVEADLRRLNEELDRSLERAERTIRELAK
>2plus1_GFP11_Key_Cterm_138
(SEQ ID NO: 26879)
TAERARETLKPLLDENRDRSKKVKSEIRRILEDLTRTTERVKREIAKLLKELEDTAR
>2plus1_GFP11_Key_Cterm_139
(SEQ ID NO: 26880)
DEIKRIVDEVRERLKRIVDENAKIVEDARRALEKIVKENEEILRRLKKELRELRK
>2plus1_GFP11_Key_Cterm_l40
(SEQ ID NO: 26881)
DPRIEKVLKEIEEKIREVIKEWERVHREVEELLKRLIDENRKVLDEIRKLLEEKSK
>2plus1_GFP11_Key_Cterm_l41
(SEQ ID NO: 26882)
DLKRVEERAREVSRRNEESMRRVKEDADRVSEANKEVLDRVREEVKRLIEEVRETLR
>2plus1_GFP11_Key_Cterm_142
(SEQ ID NO: 26883)
DAETIERVVRELLEENKEVLRKTEEAVKRSTETNKRLLSASKEVADRLRERIKEAAK
>2plus1_GFP11_Key_Cterm_143
(SEQ ID NO: 26884)
DKARKVAEVAEKVLRDIDKLDRESKEAFRATNEEIAKLDEDTARVAERVKKAIEDLAK
>2plus1_GFP11_Key_Cterm_144
(SEQ ID NO: 26885)
ELLRRIKKLLDEIKKAIEDSSREIKRLLEESERVMKRSSEDIKRTLDDTRRVVEEVRR
>2plus1_GFP11_Key_Cterm_115
(SEQ ID NO: 26886)
DKVERVVREVEKLHEEDRKRLEESTRSVRKLLEELKRELSKSTRSVKALVDELRERVR
>2plus1_GFP11_Key_Cterm_146
(SEQ ID NO: 26887)
EAKKKLDEVLERAKRTIDRLLETSDRSLEKVEADLRRLNEELDRSLERAERTIRELAK
>2plus1_GFP11_Key_Cterm_147
(SEQ ID NO: 26088)
TAERARETLKRLLDENRDRSKKVKEEIRRILEDLTRTTERVKREIAKLLKELEDTAR
>2plus1_GFP11_Key_Cterm_148
(SEQ ID NO: 26889)
DEIKRIVDEVRERLKRIVDENAKIVEDARRALEKIVKENEEILRRLKKELRELRK
>2plus1_GFP11_Key_Cterm_149
(SEQ ID NO: 26890)
DKARKVAEVAEKVLRDIDKLDRESKEAFRATNEEIAKLDEDTARVAEKVKKAIEDLAK
>2plus1_GFP11_Key_Cterm_150
(SEQ ID NO: 26891)
DEVTKVKKVADDVIAEIKKLDDETRRVIEDTNKKIADLDKATRDVVRKVLEEVKKLEK
>2plus1_GFP11_Key_Cterm_151
(SEQ ID NO: 26892)
DIDKLLKELRDLVEKIKKDLKELLERYEEIVRRIKELLKDLNREAEEVVRRLKEELR
>2plus1_GFP11_Key_Cterm_152
(SEQ ID NO: 26893)
TAERARETLKRLLDENRDRSKKVKEEIRRILEDLTRTTERVKREIAKLLKELEDTAR
>2plus1_GFP11_Key_Cterm_153
(SEQ ID NO: 26894)
DEIKRIVDEVRERLKRIVDENAKIVEDARRALEKIVKENEEILRRLKKELRELRK
>2plus1_GFP11_Key_Cterm_154
(SEQ ID NO: 26895)
RLVREVEDLVRRLVRRSEKSNEEVKRTVEELVRRMEESNDRVRDLVRRLVEELKRAVD
>2plus1_GFP11_Key_Cterm_155
(SEQ ID NO: 26896)
PKARKVAEVAEKVLRDIDKLDRESKEAFRATNEEIAKLDEDTARVAERVKKAIEDLAK
>2plus1_GFP11_Key_Cterm_156
(SEQ ID NO: 26897)
EAKKKLDEVLERAKETIDRLLETSDRSLEKVEADLRRLNEELDRSLERAERTIRELAK
>2plus1_GFP11_Key_Cterm_157
(SEQ ID NO: 26898)
RLVREVEDLVRRLVRRSEKSNEEVKRTVEELVRRMEESNDRVRDLVRRLVEELKRAVD
>2plus1_GFP11_Key_Cterm_158
(SEQ ID NO: 26899)
DKARKVAEVAEKVLRDIDKLDRESKEAFRATNEEIAKLDEDTARVAERVKKAIEDLAK
>2plus1_GFP11_Key_Cterm_159
(SEQ ID NO: 26900)
DEVTK7KKVADDVLAEIKKLDDETRRVIEDTNKKIADLDKATRDVVRKVLEEVKKLEK
>2plus1_GFP11_Key_Cterm_160
(SEQ ID NO: 26901)
TAERARETLKRLLDENRDRSKKVKEEIRRILEDLTRTTERVKREIAKLLKELEDTAR
>2plus1_GFP11_Key_Cterm_161
(SEQ ID NO: 26502)
DLKPVEERAREVSRRNEESMRRVKEDADRVSEANKEVLDRVREEVKRLIEEVRETLR
>2plus1_GFP11_Key_Cterm_162
(SEQ ID NO: 26903)
DKARKVAEVAEKVLRDIDKLDRESKEAFRATNEEIAKLDSDTARVAERVKKAIEDLAK
>2plus1_GFP11_Key_Cterm_163
(SEQ ID NO: 26904)
DEVTKVKKVADDVLAEIKKLDDETRRVIEDTNKKIADLDKATRDVVRKVLEEVKKLEK
>2plus1_GFP11_Key_Cterm_l64
(SEQ ID NO: 26905)
DKVERVVREVEKLHEEDRKRLEESTRSVRKLLEELKRELEKSTRSVKALVDELRERVR
>2plus1_GFP11_Key_Cterm_165
(SEQ ID NO: 26906)
EAKKKLDEVLERAKRTIDRLLETSDRSLEKVEADLRRLNEELDRSLERAERTIRELAK
>2plus1_GFP11_Key_Cterm_166
(SEQ ID NO: 26907)
TAERARETLKRLLDENRDRSKKVKEEIPRILEDLTRTTERVKREIAKLLKELEDTAR
>2plus1_GFP11_Key_Cterm_167
(SEQ ID NO: 26908)
TAERARETLKRLLDENRDRSKKVKEEIRRILEDLTRTTERVKREIAKLLKELEDTAR
>2plus1_GFP11_Key_Cterm_168
(SEQ ID NO: 26909)
DKARKVAEVAEKVLRDIDKLDRESKEAFRATNEEIAKLDEDTARVAERVKKAIEDLAK
>2plus1_GFP11_Key_Cterm_169
(SEQ ID NO: 26910)
DEVTKVKKVADDVLAEIKKLDDETRRVIEDTNKKIADLDKATRDVVRKVLEEVKKLEK
>2plus1_GFP11_Key_Cterm_170
(SEQ ID NO: 26911)
DKVERVVREVEKLHEEDRKRLEESTRSVRKLLEELKFELEK5TRSVKALVDELRERVR
>2plus1_GFP11_Key_Cterm_171
(SEQ ID NO: 26912)
TAERARETLKRLLDENRDRSKKVKEEIRRILEDLTRTTERVKREIAKLLKELEDTAR
>2plus1_GFP11_Key_Cterm_172
(SEQ ID NO: 26913)
DKARKVAEVAEKVLRDIDKLDRESKEAFRATNEEIAKLDEDTARVAERVKKAIEDLAK
>2plus1_GFP11_Key_Cterm_173
(SEQ ID NO: 26914)
RLVREVEDLVRRLVRRSEKSNEEVKRTVEELVRRMEESNDRVRDLVRRLVELLKRAVD
>2plus1_GFP11_Key_Nterm_174
(SEQ ID NO: 26915)
SESDEVIRDLARLLDELARHVDDSVRRMDEVVKRSTREADELAKRLDELVKEVEKKPG
>2plus1_GFP11_Key_Nterm_175
(SEQ ID NO: 26916)
SRAETVLKEVTDKIKKLADSSDELLRRNKENIDELKKSSEELLRRLTKAIEEIEKGSV
>2plus1_GFP11_Key_Nterm_176
(SEQ ID NO: 26917)
SVDEVLKEIEDALRRLKEEVERVLKENEDELRRLEEEVRRVLKEDEELLESLKRGVGE
>2plus1_GFP11_Key_Nterm_177
(SEQ ID NO: 26918)
SEVDEIIKELERLLAEIARENERIIRESRKLADEVRKRNEDAIRKLEELVARLADAVR
>2plus1_GFP11_Key_Nterm_178
(SEQ ID NO: 26919)
SEVDDVLPRLEELIKTLEDINAKSLEDIKKLIDDLAKILEDALRKHEKLIRELREAKK
>2plus1_GFP11_Key_Nterm_179
(SEQ ID NO: 26920)
SEVDDVLRRLEELIKTLEDTNAKSLEDIKKLIDDLAKILEDALRKHEKLIRELREAKK
>2plus1_GFP11_Key_Nterm_l80
(SEQ ID NO: 26921)
SRAETVLKEVTDKIKKLAD8SDELLRRNKENIDELKKSSEELLRRLTKAIEEIEKGSV
>2plus1_GFP11_Key_Nterm_181
(SEQ ID NO: 26922)
KEVEDAVKELEDLLRANEDKTRSIVEDMRASNKDLEDHSRASEEEVRKLLDDLRRAGV
>2plus1_GFP11_Key_Nterm_182
(SEQ ID NO: 26923)
SEVDDVLRRLEELIKTLEDINAKSLEDIKKLIDDLAKILEDALRKHEKLIRELREAKK
>2plus1_GFP11_Key_Nterm_183
(SEQ ID NO: 26924)
SESDEVIRDLARLLDELARHVDDSVRRMDEVVKRSTREADELAKRLDELVKEVEKKFG
>2plus1_GFP11_Key_Nterm_184
(SEQ ID NO: 26925)
SESDDVIRKLRELLEELRTHVEKSTRDLRKILEDSTRHAKRSIEELERLLEEVRKKPG
>2plus1_GFP11_Key_Nterm_185
(SEQ ID NO: 26926)
SRAETVLKEVTDKIKKLADSSDELLRRNKENIDELKKSSEELLRRLTKAIEEIEKGSV
>2plus1_GFP11_Key_Nterm_186
(SEQ ID NO: 26927)
SEAEKAKET1DRLADRVRKLLEEIKRSLDDSRRKSKETVEENEKTLDRMRKEVDAAKR
>2plus1_GFP11_Key_Nterm_187
(SEQ ID NO: 26928)
SEAEKAKETIDRIADRVRKLLEEIKRSLDDSRRKSKETVEENEKTLDRMRKEVDAAKR
>2plus1_GFP11_Key_Nterm_188
(SEQ ID NO: 26929)
SEVEELIKRLAKVLKELVDKVRKVIEDTKELLERLKRRSEDHIRKLREVLKEAKDQPI
>2plus1_GFP11_Key_Nterm_189
(SEQ ID NO: 26930)
SELEEIEKKVRELTKRHRELVERVRKTVKELIETNRRLLETLTERIKRVLEEVRDLER
>2plus1_GFP11_Key_Nterm_190
(SEQ ID NO: 26931)
SSEERLRAVIEDLKRLAEESRKRHKELIDELAKAVERISRRHKKLLDEIKAVVDDIRR
>2plus1_GFP11_Key_Nterm_191
(SEQ ID NO: 26932)
DEIRKVVKEITDLLKASNDKNRKVVEEIRDLLRKSKKLADELVERLRALVEDLRRRID
>2plus1_GFP11_Key_Nterm_192
(SEQ ID NO: 26933)
STAETVAESVERVLKHSDDLIKEVEDVNRRVEEEIKRVIRELEEENERLVAEVRKGVK
>2plus1_GFP11_Key_Nterm_193
(SEQ ID NO: 26931)
SEVDEIIKELERLLAEIAPENERIIRESRKLADEVRKRN2DAIRKLEELVARLADAVR
>2plus1_GFP11_Key_Nterm_194
(SEQ ID NO: 26935)
SEIDEVLTRLRKISKDLNETSDRVNERARKIIDDIKKESKRVNDEAREIVERLKREID
>2plus1_GFP11_Key_Nterm_195
(SEQ ID NO: 26936)
5EDEDLDRVAEKLAREHKKSVEEIKRVLKSADEE5KKLVRDTERVTEEIKREVEEARR
>2plus1_GFP11_Key_Nterm_196
(SEQ ID NO: 26937)
SSVEELLERLRRISEENKRRIEKLLREVEKVLRELKDRHRKLLKRVEEIIRKVKEEIK
>2plus1_GFP11_Key_Nterm_197
(SEQ ID NO: 26938)
SAADEVVERMKELVATVKRENDEVVKELKKLVKELEDDNRRVVEESKKSVEDLARRVG
>2plus1_GFP11_Key_Nterm_198
(SEQ ID NO: 26939)
SESDEVIRDLARLLDELARHVDDSVRRMDEVVKRSTREADELAKRLDELVKEVEKKPG
>2plus1_GFP11_Key_Nterm_199
(SEQ ID NO: 26940)
KEVEDAVKELEDLLRANEDKTRSIVEDMRASNKDLEDHSRASEEEVRKLLDDLRRAGV
>2plus1_GFP11_Key_Nterm_200
(SEQ ID NO: 26941)
DEIRKVVKEITDLLKASNDKNRKVVEEIRDLLRKSKKLADELVERLRALVEDLRRRID
>2plus1_GFP11_Key_Nterm_201
(SEQ ID NO: 26942)
SRVEEIIEDLRRLLEEIRKENEDSIRRSKELLDRVKEINDTIIAELERLLKDIEKEVR
>2plus1_GFP11_Key_Nterm_202
(SEQ ID NO: 26943)
SRVEEIIEDLRRLLEEIRKENEDSIRRSKELLDRVKEIKDTIIAELERLLKDIEKEVR
>2plus1_GFP11_Key_Nterm_203
(SEQ ID NO: 26944)
SEVDDVLRRLEELIKTLEDINAKSLEDIKKLIDDLAKILEDALRKHEKLIRELREAKK
>2plus1_GFP11_Key_Nterm_204
(SEQ ID NO: 26945)
SKLEEVEKAVRKVIEDSRRVNEEVNRRSEEVVRELEKVHREVNDASRRVVEKARRVLK
>2plus1_GFP11_Key_Nterm_205
(SEQ ID NO: 269746)
SESDEVTRDLARLLDELARHVDDSVRRMDEVVKRSTREADELAKRLDELVKEVEKKPG
>2plus1_GFP11_Key_Nterm_206
(SEQ ID NO: 26947)
SESDEVIRDLARLLDELARHVDDSVRRMDEVVKRSTREADELAKRLDELVKEVEKKPG
>2plus1_GFP11_Key_Nterm_207
(SEQ ID NO: 26948)
SESDDVIRKLRELLEELRTHVEKSIRDLRKILEDSTRHAKRSIEELERLLEEVRKKPG
>2plus1_GFP11_Key_Nterm_208
(SEQ ID NO: 26949)
DEVRELLERNPRLLEEIKKTVKDLIRANEELLKRIEDDAKRLIDRNEELLDELEKGLS
>2plus1_GFP11_Key_Nterm_209
(SEQ ID NO: 26950)
DEIRKVVKSITDLLKASNDKNRKVVEEIRDLLRKSKKLADELVERLRALVEDLRRRID
>2plus1_GFP11_Key_Nterm_210
(SEQ ID NO: 26951)
SRAETVLKEVTDKIKKLADSSDELLRRNKENTDELKKSSSELLRRLTKAIEEIEKGSV
>2plus1_GFP11_Key_Nterm_211
(SEQ ID NO: 26952)
DEEEDLERATKKLLDENRELLKRIAEELRRLLEELRRLTEESADRLRRLLKELKDRGV
>2plus1_GFP11_Key_Nterm_212
(SEQ ID NO: 26953)
DEIRKVVKEITDLLKASNDKNRKVVEEIRDLLRKSKKLADELVERLRALVEDLRRRID
>2plus1_GFP11_Key_Nterm_213
(SEQ ID NO: 26954)
SKEDRLREELKKLLARLAEEIERLKRALEESNKDLKRTIDASEKHLRDVNEDVKRGGV
>2plus1_GFP11_Key_Nterm_214
(SEQ ID NO: 26955)
SESDEVIRDLARLLDELARHVDDSVRRMDEVVKRSTREADELAKRLDELVKEVEKKPG
>2plus1_GFP11_Key_Nterm_215
(SEQ ID NO: 26956)
SESDEVIRDLARLLDELARHVDDSVRRMDEVVKRSTREADELAKRLDELVKEVEKKPG
>2plus1_GFP11_Key_Nterm_216
(SEQ ID NO: 26957)
SESDDVIRKLRELLEELRTHVEKSIRDLRKILEDSTRHAKRSIEELERLLEEVRKKPG
>2plus1_GFP11_Key_Nterm_217
(SEQ ID NO: 26958)
SRVEEIIEDLRRLLEEIRKENEDSIRRSKELLDRVKEIMDTIIAELERLLKDIEKEVR
>2plus1_GFP11_Key_Nterm_218
(SEQ ID NO: 26959)
SEVDEIIKELERLLAEIARENERIIRESRKLADEVRKRNSDAIRKLEELVARLADAVR
>2plus1_GFP11_Key_Nterm_219
(SEQ ID NO: 26960)
SSVEEL1ERLRRISEENKRRIEKLLREVEKVLRELKDRHRKLLKRVEEIIRKVKEEIK
>2plus1_GFP11_Key_Nterm_220
(SEQ ID NO: 26961)
SELEEVLRRIEALVRKAWKENEDVLREIERLVRTAHRLNKKVDDDSAKIAEDLKRGGR
>2plus1_GFP11_Key_Nterm_221
(SEQ ID NO: 26962)
SESDEVIRDLARLLDELARHVDDSVRRMDEVVKRSTREADELAKRLDELVKEVEKKPG
>2plus1_GFP11_Key_Nterm_222
(SEQ ID NO: 26963)
SESDDVIRKLRELLEELRTHVEKSIRDLRKILEDSTRHAKRSIEELERLLEEVRKKPG
>2plus1_GFP11_Key_Nterm_223
(SEQ ID NO: 26964)
SPAETVLKEVTDKIKKLADSSDELLRRNKENIDELKKS3EELLRRLTKAIEEIEKGSV
>2plus1_GFP11_Key_Nterm_224
(SEQ ID NO: 26965)
DEVRELLERNRRLLEEIKKTVKDLIRANEELLKRIEDDAKRLIDRNEELLDELEKGLS
>2plus1_GFP11_Key_Nterm_225
(SEQ ID NO: 26966)
5TEEVLDEIRKLHKTLTEDIKRVLREIEELHRRTIEENKEVLDKIAEDYKRVIDDVRT
>2plus1_GFP11_Key_Nterm_226
(SEQ ID NO: 26967)
SEIEKILKEIEDLARRDEEVSKKIVEDIRRLAKEVEDTSRDIVPKIEELAKRVLDRLR
>2plus1_GFP11_Key_Nterm_227
(SEQ ID NO: 26968)
SEAERLEARARELLRANEELMDDLRAKAEELLKRNDRLVKEIEKKVREVLAAIEELKR
>2plus1_GFP11_Key_Nterm_228
(SEQ ID NO: 26969)
DDLERAREEVADLIRKHEEKTRRILEESRRLNERHRELSARILDEIRKLAERIEELIK
>2plus1_GFP11_Key_Nterm_229
(SEQ ID NO: 26970)
DEEEDLEPAIKKLLDENRELLKRIAEELRRLLEELRRLTEESADRLRRLLKELKDRGV
>2plus1_GFP11_Key_Nterm_230
(SEQ ID NO: 26971)
DEIRKVVKEITDLLKASNDKNRKVVEEIRDLLRKSKKLADELVERLRALVEDLRRRID
>2plus1_GFP11_Key_Nterm_231
(SEQ ID NO: 26972)
DEIRKVVKEITDLLKASNDKNRKVVEEIRDLLRKSKKLADELVERLRALVEDLRRRID
>2plus1_GFP11_Key_Nterm_232
(SEQ ID NO: 26973)
STAETVEKKVEEVIRENEKSMRESEEKVDRSTKRIEDVLRRLEETIRKTSDDIAKGVK
>2plus1_GFP11_Key_Nterm_233
(SEQ ID NO: 26974)
SRVEEIIEDLRRLLEEIRKENEDSIRRSKELLDRVKEINDTIIAELERLLKDIEKEVR
>2plus1_GFP11_Key_Nterm_234
(SEQ ID NO: 26975)
SRVEE11EDLRRLLEEIRKENEDSIRRSKELLDRVKEINDTIIAELERLLKDIEKEVR
>2plus1_GFP11_Key_Nterm_235
(SEQ ID NO: 26976)
REVEEMIKELEELLKDLKEKNERASKRNRELVRRLEEENKRVIEELKKLVKELEDLVR
>2plus1_GFP11_Key_Nterm_236
(SEQ ID NO: 26977)
SEVDDVLRRLEELIKTLEDINAKSLEDIKKLIDDLAKILEDALRKHEKLIRELREAKK
>2plus1_GFP11_Key_Nterm_237
(SEQ ID NO: 26978)
SKLEEVEKAVRKVIEDSRRVNEEVNRRSEEVVRELEKVHREVNDASRRVVEKARRVLK
>2plus1_GFP11_Key_Nterm_238
(SEQ ID NO: 26979)
DEVEDVLRKIEKILDDHRKRIEKNSRDMARIIDEHRRKVEENSREMKKLVDDLKKAVD
>2plus1_GFP11_Key_Nterm_239
(SEQ ID NO: 26980)
SSVEELLERLRRISEENKRRIEKLLREVEKVLRELKDRHRKLLKRVEEIIRKVKEEIK
>2plus1_GFP11_Key_Nterm_240
(SEQ ID NO: 26981)
DEVEKVLEEIKRALDDLRKKVEESKREIKEALKAVEKHTRDSDTANKRTLAEIERGVK
>2plus1_GFP11_Key_Nterm_241
(SEQ ID NO: 26982)
SESDEVIRDLARLLDELARHVDDSVRRMDEVVKRSTREADELAKRLDELVKEVEKKPG
>2plus1_GFP11_Key_Nterm_242
(SEQ ID NO: 26983)
KEVEDAVKELEDLLRANEDKTRSIVEDMRASNKDLEDHSRASEEEVRKLLDDLRRAGV
>2plus1_GFP11_Key_Nterm_243
(SEQ ID NO: 26984)
DEIRKVVKEITDLLKASNDKNRKVVEEIRDLLRKSKKLADELVERLRALVEDLRRRID
>2plus1_GFP11_Key_Nterm_244
(SEQ ID NO: 26985)
STAETVAEEVERVLKHSDDLIKEVEDVNRRVEEEIKRVIRELEEENERLVAEVRKGVK
>2plus1_GFP11_Key_Nterm__245
(SEQ ID NO: 26986)
SRVEEIIEDLRRLLEEIRKENEDSIRRSKELLDRVKEINDTIIAELERLLKDIEKEVR
>2plus1_GFP11_Key_Nterm_246
(SEQ ID NO: 26987)
SEVDEIIKELEPLLAEIARENERIIRESFKLADEVRKRNEDAIRKLEELVARLADAVR
>2plus1_GFP11_Key_Nterm_247
(SEQ ID NO: 26988)
SEVDDVLRRLEELIKTLEDINAKSLEDIKKLIDDLAKILEDALRKHEKLIRELREAKK
>2plus1_GFP11_Key_Nterm_248
(SEQ ID NO: 26989)
SKLEEVEKAVRKViEDSRRVNEEVNRRSEEVVRELEKVHREVNDASRRVVEKARRVLK
>2plus1_GFP11_Key_Nterm_249
(SEQ ID NO: 26990)
SAEEVKEELKRIATKLKEEIKENIRRLEESVEKIAKELAENIKRLEDILRDVKRGLRD
>2plus1_GFP11_Key_Nterm_250
(SEQ ID NO: 26991)
SDVDRVLEEIRKLLEDLKRHSEKVSEENEDLLRANTELNKRVSEDNERLLEELKRLRE
>2plus1_GFP11_Key_Nterm_251
(SEQ ID NO: 26992)
DEVEDVLRKTEKILDDHRKRIEKNSRDMARIIDEHRPKVEENSREMKKLVDDLKKAVD
>2plus1_GFP11_Key_Nterm_252
(SEQ ID NO: 26993)
SSVEELLERLRRISEENKRRIEKLLREVEKVLRELKDRHRKLLKRVEEIIRKVKEEIK
>2plus1_GFP11_Key_Nterm_253
(SEQ ID NO: 26994)
DPEREVKKRLDEVRERIEPLLRRVEEESRRVAEEIRRLIEEVRRRNKKVTEEIRELLK
>2plus1_GFP11_Key_Nterm_254
(SEQ ID NO: 26995)
SRAETVLKEVTDKIKKLADSSDELLRRNKENIDELKKSSEELLRRLTKAIEEIEKGSV
>2plus1_GFP11_Key_Nterm_255
(SEQ ID NO: 26996)
SEIEKI1KEIEDLARRDEEVSKKIVEDIRRLAKEVEDTSRDIVRKIEELAKRVLDRLR
>2plus1_GFP11_Key_Nterm_256
(SEQ ID NO: 26997)
SEAEKAKETIDRIADRVRKLLEEIKRSLDDSRRKSKETVEENEKTLDRMRKEVDAAKR
>2plus1_GFP11_Key_Nterm_257
(SEQ ID NO: 26998)
SEVEELIXRLAKVLKELVDKVRKVIEDTKELLERLKRRSEDHIRKLREVLKEAKDQPI
>2plus1_GFP11_Key_Nterm_258
(SEQ ID NO: 26999)
DEIRKVVKEITDLLKASNDKNRKVVEEIRDLLRKSKKLADELVERLRALVEDLRRRID
>2plus1_GFP11_Key_Nterm_259
(SEQ ID NO: 27000)
DEIRKVVKEITDLLKASNDKNRKVVEEIRDLLRKSKKLADELVERLRALVEDLRRRID
>2plus1_GFP11_Key_Nterm_260
(SEQ ID NO: 27001)
STAETVEKKVEEVIRENEKSMRESEEKVDRSTKRIEDVLRRLEETIRKTSDDIAKGVK
>2plus1_GFP11_Key_Nterm_261
(SEQ ID NO: 27002)
SRVEEIIEDLRRLLEEIRKENEDSIRRSKELLDRVKEINDTIIAELERLLKDIEKEVR
>2plus1_GFP11_Key_Nterm_262
(SEQ ID NO: 27003)
REVEEMIKSLEELLKDLKEKNERASKRNRELVRRLEEENKRVTEELKKLVKELEDLVR
>2plus1_GFP11_Key_Nterm_263
(SEQ ID NO: 27004)
DAVEEAEKLIRKVIADSEKLLRDLADLNAKSIRRSEKLVEDDRRANEDVIRKLEELRR
>2plus1_GFP11_Key_Nterm_264
(SEQ ID NO: 27005)
SEVDDVLRRLEELIKTLEDINAKSLEDIKKLIDDLAKILEDALRKHEKLIRELREAKK
>2plus1_GFP11_Key_Nterm_265
(SEQ ID NO: 27006)
SEIERVKKRLEELLAEVEESTRRLEERLKRLLEEAKRSSEEVEKELRRLLEAVRRGLS
>2plus1_GFP11_Key_Nterm_266
(SEQ ID NO: 27007)
SDVDRVLEEIRKLLEDLKRHSEKVSEENEDLLRANTELNKRVSEDNERLLEELKRLRE
>2plus1_GFP11_Key_Nterm_267
(SEQ ID NO: 27008)
DEVEDVLRKIEKILDDHRKRIEKNSRDMARIIDEHRRKVEENSREMKKLVDDLKKAVD
>2plus1_GFP11_Key_Nterm_268
(SEQ ID NO: 27009)
SESDEVIRDLARLLDELARHVDDSVRRMDEVVKRSTREADELAKRLDELVKEVEKKPG
>2plus1_GFP11_Key_Nterm_269
(SEQ ID NO: 27010)
SRAETVLKEVTDKIKKLADSSDELLRRNKENIDELKKSSEELLRRLTKAIEEIEKGSV
>2p1us1_GFP11_Key_Nterm_270
(SEQ ID NO: 27011)
DEEEDDERAIKKLLDENRELLKRIAEELRRLLEELRRLTEESADRLRRLLKELKDRGV
>2plus1_GFP11_Key_Nterm_271
(SEQ ID NO: 27012)
DEIRKVVKEITDLLKASNDKNRKVVEEIRDLLRKSKKLADELVERLRALVEDLRRRID
>2plus1_GFP11_Key_Nterm_272
(SEQ ID NO: 27013)
DAVEEAEKLIRKVIADSEKLLRDLADLNAKSIRRSEKLVEDDRRANEDVIRKLEELRR
>2plus1_GFP11_Key_Nterm_m273
(SEQ ID NO: 27014)
SEDEDLDRVAEKLAREHKKSVEEIKRVLKSADEESKKLVRDTERVIEEIKREVEEARR
>2plus1_GFP11_Key_Nterm_274
(SEQ ID NO: 27015)
5KEDRLREELKKLLARLAEEIERLXRALEESNKDLKRTIDASEKHLRDVNEDVKRGGV
>3plus1_Key_668_Nterm
(SEQ ID NO: 27,322)
DEAKELLDEIRKAVKESEDRLEKLLRDYEKELRRLEKELRDLKRRIEEKLEELRRGSL
>3plus1_Key_668_Cterm
(SEQ ID NO: 27,323)
RGADALSRLLEELLRVVDDLIRVLKELIDKSRKVIEELLELLKRINEENLKVLAEIIK
>3plus1_Key_668_Cterm
(SEQ ID NO: 27,324)
SEKEKLLKESEEEVFRLRRTLEELLRKYREVLERLRKELREIEERVRDVVRRLKEVLD
>3plus1_Key_668_Cterm
(SEQ ID NO: 27,325)
SEKEDAARKLRKLVEELTREYEELVKKLERLIEEIEKVSEESVRKLEKLLAEISEEVR
>3plus1_Key_668_Cterm
(SEQ ID NO: 27,326)
EALRKLVELLVEVLRRLIRVNRELVKLLREVLERLLRILRESVKKLKRLIEKVIKDAT
>3plus1_Key_669_Nterm
(SEQ ID NO: 27,327)
RAVKKLDEIVKEVAKKLEDVVRANEELWRALVELNKESVRRLREIVERVARDLEETAR
>3plus1_Key_670_Nterm
(SEQ ID NO: 27,328)
SDERRIAERIRELLRESKKLVFDVVEEAKRLLKENRDSTRKIIEDIRRLLRKIEDSTR
3plus1_Key_670_Cterm
(SEQ ID NO: 27,329)
SKEETLRKEAEDLLRRLEELTRRLEKKARELLERAKKLSRDLAEELKRLLKELREKGV
3plus1_Key_670_Cterm
(SEQ ID NO: 27,330)
AAKRLVEELLKAVTDLSRKNKRILSELLKAIETLSDENKKILTEILDALRRLVEKIEK
3plus1_Key_670_Cterm
(SEQ ID NO: 27,331)
KEREEVKEKLDRLLEEVEKTVRELKREHDELLKEVEKLVRDLKKEHDELLKKVKDDGV
3plus1_Key_670_Nterm
(SEQ ID NO: 27,332)
DRLDKVEELVKKLLEDTKRTVDRVRELVRKILKKSRETLEELERLIEKILRELEKDAR
3plus1_Key_670_Cterm
(SEQ ID NO: 27,333)
SERETVKRRLEELLKEVKRTLDKLKEEHDRLLEDVRRVVEELKREHDKLLKEVKDSGV
3plus1_Key_670_Nterm
(SEQ ID NO: 27,334)
SEDEIIKKIIEDLRRVLKEVEEIHKEVEERLDKVLKEAEEMHKEVLKELDRVLDEVKR
3plus1_Key_670_Nterm
(SEQ ID NO: 27,335)
SREEVLRELEEVIEDNRRLLEELISKSKKVLDESLKLIDELLPRLEEVLERVLRLLEE
3plus1_Key_670_Nterm
(SEQ ID NO: 27,336)
ISEDDLKRVVDEVEKKLRELKRRYAEALERIKEKIKELKDRYEPAVREVVAELRKTTK
>3plus1_Key_670_Nterm
(SEQ ID NO: 27,337)
SKAEEIAEKLDRLLEENRRALEEITTRLDDLLRRNKDALRKVMEKLKRLLDDLRRGGI
>3plus1_Key_671_Cterm
(SEQ ID NO: 27,338)
VDSERVKEILERILRVVEEAVRLNEESLRRILDVVRKAVKLDRESLKKILDVVEEAVR
>3plus1_Key_671_Cterm
(SEQ ID NO: 27,339)
VKDDEVEREIRRVKEDLDRILEEYRRLLEEIKRKLEEILRRVEELKRRLRRKLEEIDR
>3plus1_Key_671_Cterm
(SEQ ID NO: 27,340)
SRVEELKKLIEDILRISREVVERIKRVAEDIHRINRRVLDDLRKLIEDILRTVEEILA
>3plus1_Key_671_Cterm
(SEQ ID NO: 27,341)
RGADALSRLLEELLRVVDDLIRVLKELIDKSRKVIEELLELLKRINEENLKVLAEIIK
>3plus1_Key_672_Cterm
(SEQ ID NO: 27,342)
RGADALSRLLEELLRVVDDLIRVLKELIDKSRKVIEELLSLLKRINEENLKVLAEIIK
>3plus1_Key_67>3_Nterm
(SEQ ID NO: 27,343)
EALRKLVELLVEVLRRLIRVNRELVKLLREVLERLLRILRESVKKLKRLIEKVIKDAT
>3plus1_Key_67>3_Nterm
(SEQ ID NO: 27,344)
SEKEDAARKLRKLVEELTREYEELVKKLEFLIEEIEKVSEESVRKLEKLLAEISEEVR
>3plus1_Key_67>3_Nterm
(SEQ ID NO: 27,345)
SEKEDAARKLPKLVEELTREYEELVKKLERLIEEIEKVSEESVRKLEKLLAEISEEVP
>3plus1Key_674_Nterm
(SEQ ID NO: 27,346)
SEKEDAARKLRKLVEELTREYEELVKKLERLIEEIEKVSEESVRKLEKLLAEISEEVR
>3plus1_Key_674_Cterm
(SEQ ID NO: 27,347)
RAVKKLDEIVKEVAKKLEDVVRANEELWRALVELNKESVRRLREIVERVARDLEETAR
>3plus1_Key_675_Nterm
(SEQ ID NO: 27,348)
RAVKKLDEIVKEVAKKLEDVVRANEELWRALVELNKESVRRLREIVERVARDLEETAR
>3plus1_Key_676_Nterm
(SEQ ID NO: 27,349)
RAVKKLDEIVKEVAKKLEDVVRANBELWRALVELNKESVRRLREIVERVARDLEETAR
>3plus1_Key_677_Nterm
(SEQ ID NO: 27,350)
SDERRIAERIRELLRESKKLVRDVVEEAKRLLKENRDSTRKIIEDIRRLLRKIEDSTR
>3plus1_Key_677_Cterm
(SEQ ID NO: 27,351)
SKEETLRKEAEDLLRRLEELTRRLSKKARELLERAKKLSRDLAEELKRLLKELREKGV
>3plus1_Key_678_Nterm
(SEQ ID NO: 27,352)
SKEETLRKEAEDLLRRLEELTRRLEKKARELLERAKKLSRDLAEELKRLLKELREKGV
>3plus1_Key_678_Cterm
(SEQ ID NO: 27,353)
SKEETLRKEAEDLLRRLEELTRRLEKKAPELLERAKKLSRDLAEELKRLLKELREKGV
>3plus1_Key_678_Cterm
(SEQ ID NO: 27,354)
ISEDDLKRVVDEVEKKLRELKRRYAEALERIKEKIKELKDRYERAVREVVAELRKTTK
>3plus1_Key_678_Nterm
(SEQ ID NO: 27,355)
SKAEEIAEKLDRLLEENRRALESITTPLDDLLRRNKDALRKVMEKLKRLLDDLRRGGI
>3plus1_Key_678_Nterm
(SEQ ID NO: 27,356)
VDSERVKEILERILRVVEEAVRLNEESLRRILDVVRKAVKLDRESLKKILDVVEEAVR
>3p1us1_Key_679_Cterm
(SEQ ID NO: 27,357)
SKAEEIAEKLDRLLEENRRALEEITTRLDDLLRRNKDALRKVMEKLKRLLDDLRRGGI
>3plus1_Key_679_Nterm
(SEQ ID NO: 27,358)
SKAEEIAEKLDRLLEENPRALEEITTRLDDLLRPNKDALRKVMEKLKRLLDDLRRGGI

In another specific embodiment, the key polypeptides comprise a polypeptide having at least 40%, 45%, 50%, 55%, 60%, 65%. 70%, 75%, 80%, 85%, 90%, 91%, 92%, 93%. 94%, 95%, 96%, 97%, 98%, 99%, or 100% sequence identity along their length to the amino acid sequence of a key polypeptide in Table 2 or 5 (polypeptides with an odd-numbered SEQ ID NO between SEQ ID NOS: 27127 and 27277), Table 3, and/or Table 4. in another specific embodiment, the key polypeptides comprise a polypeptide having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along their length to the amino acid sequence of a key polypeptide in Table 3. In another specific embodiment, the key polypeptides comprise a polypeptide having at least 40%, 45%, 50%, 55%, 60%, 65%, 70%. 75%, 80%, 85%, 90%. 91%, 92%, 93%, 94%, 95%, 96%, 97%, 98%, 99%, or 100% sequence identity along their length to the amino acid sequence of a key polypeptide in Table 4. In one embodiment of each of the above, the percent identify may be determined without the optional N- and C-terminal 60 amino acids; in another embodiment, the percent identify may be determined with the optional N- and C-terminal 60 amino acids.

In a specific embodiment, the polypeptides comprise a polypeptide having at least 40%. 45%, 50%, 55%. 60%, 65%, 70%, 75%, 80%, 85%, 90%, 91%, 92%, 93%, 94%, 95%, 96%, 97%, 98%, 99%, or 100% sequence identity along its length to the amino acid sequence of a key polypeptide selected from the group consisting of SEQ ID NOS: 28477-28486.

The polypeptides of the disclosure (i.e.: cage polypeptides and key polypeptides) may include additional residues at the N-terminus, C-terminus, internal to the polypeptide, or a combination thereof; these additional residues are not included in determining the percent identity of the polypeptides of the invention relative to the reference polypeptide. Such residues may be any residues suitable for an intended use, including but not limited to tags. As used herein, “tags” include general detectable moieties (i.e.: fluorescent proteins, antibody epitope tags, etc.), therapeutic agents, purification tags (His tags, etc.), linkers, ligands suitable-for purposes of purification, ligands to drive localization of the polypeptide, peptide domains that add functionality to the polypeptides. etc. Examples are provided herein.

In one embodiment, the polypeptides are fusion proteins that comprise a cage polypeptide disclosed herein fused to a key polypeptide disclosed herein. In one embodiment, the fusion protein comprises a cage polypeptide fused to a key polypeptide, wherein the cage polypeptide is not activated by the key polypeptide. As noted herein, orthogonal LOCKR designs (see FIG. 3) are denoted by lowercase letter subscripts: LOCKR, consists of Cageₐ and Keyₐ, and LOCKR_{b} consists of Cage_{b} and Key_{b}, etc. such that Cage₃ is only activated by Keyₐ, and Cage_{b} is only activated by Key_{b}, etc. Thus, for example, the fusion protein may comprise a cage, polypeptide fused to a key_{b} polypeptide. Such embodiments may be used, for example, in combinations to improve control of orthogonal LOCKR designs (ex: LOCKR 1 comprises a cageₐ-key_{b} fusion polypeptide, and LOCKR 2 comprises a cagey-key, fusion polypeptide, which can then be expressed in the same cell).

As used herein, “orthogonally” or “orthogonal” means that particular key polypeptides and cage polypeptides may function together, while others may not. Thus, two or more different orthogonal systems of key polypeptide and cage polypeptides may independently function in the same system, cell, or organism without interfering with each other. However, as clearly noted herein, multiple individual key polypeptides may function with a variety of different cage polypeptides, and, multiple individual cage polypeptides may function with a variety of different key polypeptides.

In one embodiment of the fusion proteins disclosed herein, the cage polypeptide and the key polypeptide components of the fusion protein comprise at least one cage polypeptide and at least one key polypeptide comprising an amino acid sequence having at least 45%, 50%, 55%, 60%, 65%, 70%, 75%, 80%, 85%, 90%. 91%, 92%, 93%, 94%, 95%, 96%, 97%, 98%, 99%. or 100% sequence identity along its length to a cage polypeptide and a key polypeptide, respectively, in different rows of Table 2, Table 3, Table 4, and/or Table 5 (i.e.: each cage polypeptide in row 1 column 1 of the table can be fused with any key polypeptide in row 1 column 2, and so on). In embodiments, relating to Tables 2, 3, 4, and 5, the degron is not included in the amino acid sequence of the cave polypeptide, arid would be added within the latch region, including but not limited to those degron amino acid sequences disclosed herein.

TABLE 2

| Row | | |
|---|---|---|
| number | Cage (column 1) | Key (column 2) |
| 1 | SB76L (SEQ ID NO: 1), | SB76_C-helix |
| | SB76L_17 (SEQ ID NO: 2), | (SEQ ID NO: 27016), |
| | SB76L_18 (SEQ ID NO: 3), | SB76_C-helix-biotin |
| | LOCKR_extend5 (SEQ ID NO: 4), | (SEQ ID NO: 27017), |
| | LOCKR_extend9 (SEQ ID NO: 5), | p5_MBP (SEQ ID NO: 27018), |
| | LOCKR_extend18 (SEQ ID NO: 6). | p9_MIBP (SEQ ID NO: 27019), |
| | miniLOCKRa_1 (SEQ ID NO: 12), | p18_MBP (SEQ ID NO: 27020), |
| | miniLOCKRa_2 (SEQ ID NO: 13), | p76-long (SEQ ID NO: 27027), |
| | aBcl2LOCKR (SEQ ID NO: 18), | p76-short (SEQ ID NO: 27028), |
| | pBimLOCKR (SEQ ID NO: 19), | k76-long (SEQ ID NO: 27029), |
| | BimLOCKR_extend5 (SEQ ID NO: 20), | k76-short (SEQ ID NO: 27030), |
| | BimLOCKR_extend9 (SEQ ID NO: 21), | p76_GLISE (SEQ ID NO: 27031), |
| | BimLOCKR_extend18 (SEQ ID NO: 22), | p76_GSSEKIS |
| | strepLOCKRa (all variants; SEQ ID NOs: 26-34), | (SEQ ID NO: 27032), |
| | SB13_LOCKR (SEQ ID NO: 35), | p76_R26G (SEQ ID NO: 27033), |
| | SB13_LOCKR_extend18 (SEQ ID NO: 37), | p76-short_E19G |
| | ZCX12_LOCKR (SEQ ID NO: 36), | (SEQ ID NO: 27034), |
| | ZCX12_LOCKR_extend18 (SEQ ID NO: 38) | p76-short_GLISE_E01_EGFR |
| | fretLOCKRa (SEQ ID NO: 39), | (SEQ ID NO: 27035), |
| | 1fix-latch_Mad1SID_t0_1 (SEQ ID NO: 61), | p76-short_AE_EGFR |
| | 1fix-latch_Mad1SID T0_2 (SEQ ID NO: 65), | (SEQ ID NO: 27036), |
| | 1fix-long-Bim-t0 (SEQ ID NO: 54), | p76-short_AAE_EGFR |
| | 1fix-long-GFP-t0 (SEQ ID NO: 55), | (SEQ ID NO: 27037), |
| | 1fix-short-BIM-t0 (SEQ ID NO: 56), | p76-short_EE_EGFR |
| | 1fix-short-GFP-t0 (SEQ ID NO: 57), | (SEQ ID NO: 27038) |
| | 1fix-short-noBim-t0 (SEQ ID NO: 16), | |
| | 1fix-short-noBim(AYYA)-t0 (SEQ ID NO: 17), | |
| | 1fix-short-Bim-t0-relooped (SEQ ID NO: 67), | |
| | 1fix-short-spytag-t0_2 (SEQ ID NO: 68), | |
| | 1fix-short-spytag-t0_8 (SEQ ID NO: 69), | |
| | 1fix-short-TEV-t0_1 (SEQ ID NO: 70), | |
| | 1fix-short-TEV-t0_6 (SEQ ID NO: 71). | |
| | 1fix-short-nanoBit-t0_1 (SEQ ID NO: 72), | |
| | 1fix-short-nanoBit-t0_3 (SEQ ID NO: 73), | |
| | 1fix-short-RHIM-t0_8 (SEQ ID NO: 74), | |
| | 1fix-short-RHIM-t0_19 (SEQ ID NO: 75). | |
| | 1fix-short-RHIM-t0_22 (SEQ ID NO: 76), | |
| | 1fix-short-gcn4-t0_4 (SEQ ID NO: 77), | |
| | 1fix-short-ccDi-t0_6 (SEQ ID NO: 78), | |
| | 1fix-short-cc-a-t0_6 (SEQ ID NO: 79), | |
| | 1fix-short-cc-b-t0_6 (SEQ ID NO: 80) | |
| 2 | LOCK-Rb (SEQ ID NO: 7), | key_b (SEQ ID NO: 27022) |
| | BimLOCKRb (SEQ ID NO: 23), | |
| | fretLOCKRb (SEQ ID NO: 40) | |
| 3 | LOCKRc (SEQ ID NO: 8), | key_c (SEQ ID NO: 27023) |
| | miniLOCKRc_1 (SEQ ID NO: 14). | |
| | miniLOCKRc_2 (SEQ ID NO: 15), | |
| | BimLOCKRc (SEQ ID NO: 24), | |
| | fretLOCKRc (SEQ ID NO: 41) | |
| 4 | LOCKRd (SEQ ID NO: 9), | key_d (SEQ ID NO: 27024) |
| | BimLOCKRd (SEQ ID NO: 25), | |
| | fretLOCKRd (SEQ ID NO: 42) | |
| 5 | LOCKRe (SEQ ID NO: 10) | key_e (SEQ ID NO: 27025) |
| 6 | LOCKRf (SEQ ID NO: 11) | key_f (SEQ ID NO: 27026) |
| 7 | 1fix_VMAc_C_BIMlatcht9 (SEQ ID 51) | sfGFP_VMAn_p18 |
| | sfGFP_VMAn_1fix_BIM_t0_latch | (SEQ ID NO: 27041) |
| | (SEQ ID NO: 52) | p18_VMAc_mCherry |
| | sfGFP_VMAn_1fix_BIM_t0_latch | (SEQ ID NO: 27042) |
| | (SEQ ID NO: 53) | |
| 8 | Spycatcher-1fix-long-GFP-t0 (SEQ ID NO: 58), | p76-spytag (SEQ ID NO: 27039), |
| | Spycatcher-1fix-short-GFP-t0 (SEQ ID NO: 59) | p76-short-spytag |
| | | (SEQ ID NO: 27040) |
| 9 | STREPII-2plus1_LOCK_1 (SEQ ID NO: 81) | 2plus1_Key_1 |
| | | (SEQ ID NO: 27043) |
| 10 | STREPII-2plus1_LOCK_2 (SEQ ID NO: 82) | 2plus1_Key_2 |
| | | (SEQ ID NO: 27044) |
| 11 | STREPII-2plus1_LOCK_3 (SEQ ID NO: 83) | 2plus1_Key_3 |
| | STREPII-2plus1_LOCK_3-relooped | (SEQ ID NO: 27045) |
| | (SEQ ID NO: 91) | |
| 12 | STREPII-2plus1_LOCK_4C (SEQ ID NO: 84) | 2plus1_Key_4C |
| | | (SEQ ID NO: 27046) |
| 13 | STREPII-3plus1_LOCK_1 (SEQ ID NO: 86) | 3plus1 _Key_1 |
| | | (SEQ ID NO: 27047) |
| 14 | STREPII-3plus1_LOCK_2 (SEQ ID NO: 87) | 3plus1_Key_2 |
| | | (SEQ ID NO: 27048) |
| 15 | STREPII-3plus1_LOCK_3 (SEQ ID NO: 88) | 3plus1_Key_3 |
| | STREPII-3plus1_LOCK_3-relooped | SEQ ID NO: 27049) |
| | (SEQ ID NO: 90) | |
| 16 | STREPII-3plus1_LOCK_4 (SEQ ID NO: 89) | 3plus1_Key_4 |
| | | (SEQ ID NO: 27050) |

In other embodiments of the kits and degron LOCKR switches of the second and third aspects, the one or more cage polypeptide and the one or more key polypeptide comprise at least one cage polypeptide and at least one key polypeptide comprising an amino acid sequence having at least 40%, 45%, 50%, 55%, 60%, 65%, 70%, 75%, 80%, 85%, 90%, 91%, 92%. 93%, 94%, 95%, 96%, 97%, 98%, 99%, or 100% sequence identity along the length of a cage polypeptide and a key poly⁻peptide, respectively, the in the same row of Table 6 or Table 7 (i.e.: all cage polypeptides listed in row #1 can be used together with all key polypeptides in row 1; etc.):

TABLE 3

| Cage Name | Cage Sequence | Key Name | Key Sequence |
|---|---|---|---|
| 2plus1_Cage_Cterm_2406 | SEVDEVVKEVEDLVRPNEELVEEVVRRVEKVVTDDRRLVEEVVREI | 2plus1_Key_Cterm_2406 | EKVLRKLEKVIREVRERSTRALRKVE |
| | RKIVKDVEDLARKLDKEELKPVLDEMRERIERLLEKLRRHSKKLDD | | EVIRRVREESERALRDLERVVKEVEK |
| | ELKRLLEELREHSRRVEKRLEDLLKELRERGVDEKVLRKLEKVIRE | | RMREAAR(SEQ ID NO: 27127) |
| | VRERSTRALRKVEEVIRRVREESERALRDLERVVKEVEKRMREAAR | | |
| | (SEQ ID NO: 27126) | | |
| 2plus1_Cage_Cterm_5398 | SVEELLRKLEEVLRKIREENERSLKELRDRAREIVKRNRETNRELE | 2Plus1_Key_Cterm_5398 | EDIVRKIERIVETIEREVRESVKKVE |
| | EVIKELEKRLSGADKEKVEELVRRIRRIVERVVEEDRRTVEEIEKI | | EIARDIRRKVDESVKNVEKLLRDVDK |
| | AREVVKRDRDSADPYRRTVEDVLRKATGSEDIVRKIERIVETIERE | | KARDRKK(SEQ ID NO: 27129) |
| | VRESVKKVEEIARDIRPKVDESVKNVEKLLRDVDKKARDRKK(SEQ | | |
| | ID NO: 27128) | | |
| 2plus1_Cage_Cterm_5405 | SESDDVIRKLRELLEELRTHVEKSIRDLRKILEDSTRHAKRSIEEL | 2plus1_Key_Cterm_5405 | EEKLKDLIRKLRDILRRAAEAHKKLI |
| | ERLLEEVRKKPGDEEVRKTVEEISRRVAENVKRLEDLYRRMEEEVK | | DDARESLERAKREHEKLIDRLKKILE |
| | KNLDRLRKRVEDIIREVEEARKKGVDEEKIKDLIRKLRDILRRAAE | | ELER(SEQ ID NO: 27131) |
| | AHKKLIDDARESDEPARPEHEKLIDRLKKILEELER(SEQ ID | | |
| | NO: 27130) | | |
| 2plus1_Cage_Cterm_5406 | DREREVKKRLDEVPERIERLLRRVEEESRRVAEEIRRLIEEVRRRN | 2plus1_Key_Cterm_5406 | EELREELKKLERKIEKVAKEIHDHDK |
| | KKVTEEIRELLKGLKDKEEVRRVLERLRKLNAESDELLERILERLR | | EVTERLEDLLRRITEHARKSDREIEE |
| | RLVEATNRLVKAIIEELRRLVEKIVREVPDSEELREELKKLERKIE | | TAR(SEQ ID NO: 27133) |
| | KVAKEIHDHDKEVTERLEDLLRRITEHARKSDREIEETAR(SEQ | | |
| | ID NO: 27132) | | |
| 2plus1_Cage_Cterm_5409 | SEAEELLKRLEDRAEEILRRLEEILRTSRKLAEDVLRELEKLLRES | 2plus1_Key_Cterm_5409 | KEVVDEIKRIVDEVRERLKRIVDENA |
| | ERRIREVLEELRGIKDKKELEDVIREVEKELDESLERSRELLKDVL | | KIVEDARRALEKIVKENEEILRRLKK |
| | KKLDDNLKESERLVEDIDRELAKILEDLKKAGVPKEVVDEIKRIVD | | ELRELRK(SEQ ID NO: 27135) |
| | EVRERLKRIVDENAKIVEDARRALEKIVKENEEILPRLKKELRELR | | |
| | K(SEQ ID NO: 27134) | | |
| 2plus1_Cage_Cterm_5410 | SRAETVLKEVTDKIKKLADSSDELLRRNKENIDELKKSSEELLRRL | 2plus1_Key_Cterm_5410 | DEVVKRVRDLLDTVRRRNEKVNEDVK |
| | TKAIEEIEKGSVDEETLEELIRRVEAELEAHHRELEKNSREDEKRN | | RMNDKLRRDNEDVIRRVEKLLRELEE |
| | RDHHAKLEEEMRRVEERLEREGIDDEVVKRVRDLLDTVRRRNEKVN | | KRRT(SEQ ID NO: 27137) |
| | EDVKRMNDKLRRDNEDVIRRVEKLLRELEEKRRT(SEQ ID | | |
| | NO: 27136) | | |
| 2plus1_Cage_Cterm_5413 | STEEVLDEIRKLHKTLTEDIKRVLREIEELHRRTIEENKEVLDKIA | 2plus1_Key_Cterm_5413 | AEELLRESKEAIKEVKRVLEELRKES |
| | EDYKRVIDDVRTKDTPNVEKLLKDLEKSAKENIEHNERTLREDDRV | | KRVVDETRKLSEENLEHSERVLRKVE |
| | LKEIRRRATELLKANEEMLRRIEEVARKGGVDAEELLRESKEAIKE | | EDLR(SEQ ID NO: 27139) |
| | VKRVLEELRKESKRVVDETRKLSEENLEHSERVLPKVEEDLR(SEQ | | |
| | ID NO: 27138) | | |
| 2plus1_Caqe_5414_GEP11_Cterm | SEIEKILKEIEDLARRDEEVSKKIVEDIRRLAKEVEDTSRDIVRKI | 2plus1_Key_Cterm_5414 | EDSERLVREVEDLVRRLVRRSEKSNE |
| | EELAKRVLDRLRKDGSKEELEKEVREVVKTLEELVKDNHRLIRRAV | | EVKRTVEELVRRMEESNDRVRDLVRR |
| | EEMKRLVEENHRHSREVVKELEDLVPELRKGSGSEDSERDHMVLHE | | LVEELKRAVD(SEQ ID |
| | YVNAAGITSEKSNEEVKRTVEELVRRMEESNDRVRDLVRRLVEELK | | NO: 27141) |
| | RAVD(SEQ ID NO: 27140) | | |
| 2plus1_Cage_5414_GFP11_Cterm | SEIEKILKEIEDLARRDEEVSKKIVEDIPPLAKEVEDTSRDIVRKI | 2plus1_Key_Cterm_5414 | EDSERLVREVEDIVRRLVRRSEKSNE |
| | EELAKRVLDRLRKDGSKEELEKEVREVVKTLEELVKDNHRLIRRAV | | EVKRTVEELVRRMEESNDRVRDLVRR |
| | EEMKRLVEENHPESREVVKELEDLVRELRKGSGSEDSERRDHMVLH | | IVEELKRAVD(SEQ ID |
| | EYVNAAGITEKSNEEVKRTVEELVRRMEESNDRVRDLVRRLVEELK | | NO: 27143) |
| | RAVD(SEQ ID NO: 27142) | | |
| 2plus1_Cage_5414_GFP11_Cterm | SEIEKILKEIEDLARRDEEVSKKIVEDIRRLAKEVEDTSRDIVRKI | 2plus1_Key_Cterm_5414 | EDSERLVREVEDLVRRLVRRSEKSNE |
| | EELAKRVLDRLRKDGSKEELEKEVREVVKTLEELVKDNHRLIRRAV | | EVKRTVEELVRRMEESNDRVRDLVRR |
| | EEMKRLVEENHRHSREVVKELEDLVRELRKGSGSEDSERLVREVRD | | LVEELKRAVD(SEQ ID |
| | HMVLHEYVNANGITEVKRTVEELVRRMEESNDRVRDLVRRLVEELK | | NO: 27145) |
| | RAVD(SEQ ID NO: 27144) | | |
| 2plus1_Cage_Cterm_5421 | SVDEVLKEIEDALRRIKEEVERVLKENEDELRRLEEEVRRVLKEDE | 2plus1_Key_Cterm_5421 | EKAIRDVAKEIRDRLKELEEEIEEVT |
| | ELLESLKRGVGESDEVDRVVDEIAKLSAEILEKVKKVVKEIRDSLE | | RRNLKLLADVEEEIRRVHEKTRRLLE |
| | TVKRRVDDVVRRLKELLDEIKRGSDEKAIRDVAKEIRDRLKELEEE | | TVLRRAT(SEQ ID NO: 27147 |
| | IEENTRRNLKLLADVEEEIRRVIIEKTRRLIZTVLRRAT (SEQ ID | | |
| | NO: 27146) | | |
| 2plus1_Cage_Cterm_5432 | DEIRKVVKEITDLLKASNDKNRKVVEEIRDLLRKSKKLADELVERL | 2plus1_Key_Cterm_5432 | SEDLKRVEERAREVSRRNEESMRRVK |
| | RALVEDLRRRIDKSGDKETAEDIVRRIIEELKPILKETEDLARRIN | | EDADRVSEANKEVLDRVREEVKRLIE |
| | REIERLVEEVERDNRDVNRAIEELLKDTARRGGSEDLKRVEEPARE | | EVRETLR(SEQ ID NO: 27149) |
| | VSRRNEESMRRVKEDADPVSEANKEVLDRVREEVKRLIEEVRETLR | | |
| | (SEQ ID NO: 27148) | | |
| 2plus1_Cage_Cterm_5435 | STAETVAEEVERVLKHSDDLIKEVEDVNRRVEEEIKRVIRELEEEN | 2plus1_Key_Cterm_5435 | EEAAREIIKRLREVNKRTKEKLDELI |
| | ERLVAEVRKGVKGEILAEIEKRLADNSEKVREVAERAKKLLEENTA | | KHSEEVLERVKRLIDELRKHSEEVLE |
| | RVKDILRESPKLVKDLLDEVRSTGSEEAAREIIKRLREVNKPTKEK | | DLRRRAK(SEQ ID NO: 27151) |
| | LDELIKHSEEVLERVKPLIDELRKHSEEVLEDLRRRAK(SEQ ID | | |
| | NO: 27150) | | |
| 2plus1_Caqe_Cterm_5437 | DEVREVAERLRRLVDESRKRNEEVIKESEALVDRVRKTNEEVMKRL | 2plus1_Key_Cterm_5437 | AETIERVVRELLEENKEVLRKTEEAV |
| | RELIDKLEKDIRRSGDKETVEKIIREVLSAIDELLKRVERTNAEIS | | KRSTETNKRLLEASKEVADRLRERIK |
| | KENARLLDEVRKTNEEISRRLAKLLEDIRROSODAETIERVVRELL | | EAAK(SEQ ID NO: 27153) |
| | EENKEVLRKTEEAVKRSTETNKRLLEASKEVADPLRERIKEAAK | | |
| | (SEQ ID NO: 27152) | | |
| 2plus1_Cage_Cterm_5439 | SRVEEIIEDLRRLLEEIRKENEDSIRRSKELLDRVKEINDTIIAEL | 2plus1_Key_Cterm_5439 | EDKARKVAEVAEKVLRDIDKLDRESK |
| | ERLLKDIEKEVREKGSESEEVKKALRAVLEELEKLLRRVAEINEEV | | EAFRATNEEIAKLDEDTARVAERVKK |
| | LRRNSKLVEEDERRNREVLKELARLVEELIREIGDEDKAPKVAEVA | | AIEDLAK(SEQ ID NO: 27155) |
| | EKVLRDIDKLDRESKEAFRATNEEIAKLDEDTARVAERVKKAIEDL | | |
| | AK(SEQ ID NO: 27154) | | |
| 2plus1_Cage_Cterm_5447 | SEADDVLKKLAETVKRIIERLKKLTDDSRRLVEEVHRRNDKLSKES | 2plus1_Key_Cterm_5447 | EELSAEVKKILDEVRKALARHKDEND |
| | AEAVRKAEERGlDEKDVRKLLEDLKKKSEEVAERNKRILDTLREIS | | KLLKEIEDSLRRHKEENDRLLEKLKE |
| | KRAEDEVRKVLKELEKTLKELEDRRPDSEELSAEVKKLLDEVRKAL | | STR(SEQ ID NO: 27157 |
| | ARHKDENDKLLKEIEDSLRRHKEENDRLLEKLKESTR(SEQ ID | | |
| | NO: 27156) | | |
| 2plus1_Cage_Cterm_5456 | SAEELLREVAELVKAVDEDLRRLLEEVRASNEEVIRRLEEILKRIE | 2plus1_Key_Cterm_5465 | EETVKRLLDELRELLERLKRTIEELL |
| | EENRKVVEELRRGGVSEDLVRESKRLVDESPRVIEKINKESADSVE | | KRNRDLLADAEEKARRLLEENRKLLK |
| | RTRETVDRLREELKRLVEEIAKMVKGGSSEETVKRLLDELRELLER | | AARDTAT(SEQ ID NO: 27159) |
| | LKRTIEELLKRNRDLLADAEEKARRLLEENRKLLKAARDTAT(SEQ | | |
| | ID NO: 27158) | | |
| 2plus1_Cage_Cterm_5470 | SKEDRLREELKKLLARLAEEIERLKRALEESNKULKPTIDASEKHL | 2plus1_Key_Cterm_5470 | EEELDKLLKEARDLIREIEKRLEELL |
| | RDVNEDVKRGGVSEELLRELERSTAENKERAKELLKRHEDLVRKVE | | KRVEKLTEDAKRDLERSNREHKELAD |
| | KELADLLRRLEEIVARVDEALKRGISEEELDKLLKEARDLIREIEK | | RIKETAR(SEQ ID NO: 27161) |
| | RLEELLKRVEKLTEDAKRDLERSNREHKELADRIKETAR(SEQ ID | | |
| | NO: 27160) | | |
| 2plus1_Cage_Nterm_2406 | SEVDEVVKEVEDLVRRNEELVEEVVRRVEKVVTDDRRLVEEVVREI | 2plus1_Key_Nterm_2406 | SEVDEVVKEVEDLVRRNEELVEEVVR |
| | RKIVKDVEDLARKLDKEELKRVLDEMREPIERLLEKLRRHSKKLDD | | RVEKVVTDDRRLVEEVVREIRKIVKD |
| | ELKRLLEELREHSRRVEKRLEDLLKELRERGVDEKVLRKLEKVIRE | | VEDLARK(SEQ ID NO: 27163) |
| | VRERSTRALRKVEEVIRRVREESERALRDIERVVKEVEKRMREAAR | | |
| | (SEQ ID NO: 27162) | | |
| 2plus1_Cage_Nterm_5406 | DREREVKKRLDEVRERIERLLPRVEEESRRVAEEIRRIIEEVRRRN | 2plus1_Key_Nterm_5406 | DREREVKKRLDEVRERIERLLRRVEE |
| | KKVTEEIRELLKGLKDKEEVRRVLERLRKINAESDELLERILERLR | | ESRRVAEEIRRLIEEVRRRNKKVTEE |
| | RLVEATNRLVKAIIEELRRLVEKIVREVPDSEELREELKKLERKIE | | IRELLKGL(SEQ ID NO: 27165) |
| | KVAKEINDHDKEVTERLEDLLRRITEHARKSDREIEETAR(SEQ | | |
| | ID NO: 27164) | | |
| 2plus1_Cage_Nterm_5409 | SEAEELLKRLEDRAEEILRRLEEILRTSRKLAEDVLRELEKLLRES | 2plus1_Key_Nterm_5409 | SEAEELLKRLEDRAEEILRRLEEILR |
| | ERRIREVLEELRGIKDKKELEDVIREVEKELDESLERSRELLKDVL | | TSRKLAEDVLRELEKLLRESERRIRE |
| | KKLDDNLKESERLVEDIDRELAKILEDLKKAGVPKEVVDEIKRIVD | | VLEELRGI(SEQ ID NO: 27167) |
| | EVRERLKRIVDENAKIVEDARRALEKIVKENEEILRRLKKELRELR | | |
| | K(SEQ ID NO: 27166) | | |
| 2plus1_Cage_Nterm_5410 | SRAETVLKEVTDKIKKLADSSDELLRRNKENIDELKKSSEELLRRL | 2plus1_Key_Nterm_5410 | SRAETVLKEVTDKIKKLADSSDELLR |
| | TKAIEEIEKGSVDEETIEELIRRVEAELEAHHRELEKNSREDEKRN | | RNKENIDELKKSSEELLRRLTKAIEE |
| | RDHHAKLEEEMRRVEERLEREGIDDEVVKRVRDLLDTVRRRNEKVN | | IEKGS(SEQ ID NO: 27169) |
| | EDVKRMNDKLRRDNEDVIRRVEKLLRELEEKRRT(SEQ ID | | |
| | NO: 27168) | | |
| 2plus1_Cage_Nterm_5413 | STEEVLDEIRKLNKTLTEDIKPVLREIEELHRRTIEENKEVLDKIA | 2plus1_Key_Nterm_5413 | STEEVLDEIRKLHKYLTEDIKRVLRE |
| | EDYKRVIDDVRTKDTPNVEKLLKDLEKSAKENIEHNERTLREDDRV | | IEELHRRTIEENKEVLDKIAEDYKRV |
| | LKEIRRRATELLKANEEMLPPIEEVARKGGVDNEELIRESKEAIKE | | IDDVRTKD(SEQ ID NO: 27171) |
| | VKRVLEELRKESKRVVDETRKLSEENLEHSERVLRKVEEDLR(SEQ | | |
| | ID NO: 27170) | | |
| 2plus1_Cage_5414_GFP11_Nterm | SEIEKILKEIEDLARRDEEVSKKIVEDIRRLAKEVEDTSPPDHMVL | 2plus1_Key_Nterm_5414 | SEIEKILKEIEDLARRDEEVSKKIVE |
| | HEYVNAAGITLRKDGSKEELEKEVREVVKTLEELVKDNHRLIRRAV | | DIRRLAKEVEDTSPLIVRKIEELAKR |
| | EEMKRLVEEKHRHSREVVKELEDLVRELRKGSGSEDSEPLVREVED | | VLDRLRKD(SEQ ID NO: 27173) |
| | LVRRLVRRSEKSNEEVKRTVEELVRRMEESNDRVRDLVRRLVEELK | | |
| | RAVD(SEQ ID NO: 27172) | | |
| 2plus1_Cage_5414_GFP11_Nterm | SEIEKILKEIEDLARRDEEVSKKIVEDIRRLAKEVEDTSRDIRDHM | 2plus1_Key_Nterm_5414 | SEIEKILKZIEDLARRDEEVSKKIVE |
| | VLHEYVNAAGITKDGSKEELEKEVREVVKTLEELVKDNHRLIRRAV | | DIRRLAKEVEDTSRDIVRKIEELAKR |
| | EEMKRLVEENHRHSREVVKELEDLVRELRKGSGSEDSERLVREVED | | VLDRLRKD(SEQ ID NO: 27175) |
| | LVRRLVRRSEKSNEEVKRTVEELVPRMEESNDRVRDLVRRLVEELK | | |
| | RAVD(SEQ ID NO: 27174) | | |
| 2plus1_Cage_Nterm_5439 | SRVEEIIEDLRRLLEEIRKENEDSIRRSKELLDRVKEINDTIIAEL | 2plus1_Key_Nterm_5439 | SRVEEIIEDLRRLLEEIRKENEDSIR |
| | ERLLKDIEKEVREKGSESEEVKKALRNVLEELFKLLRRVAEINEEV | | RSKELLDRVKEINDTIIAELERLLKD |
| | LRRNSKLVEEDERRNREVLKELARLVEELIREIGDZDKARKVAEVA | | IEKEVREKG(SEQ ID NO: 27177) |
| | EKVLRDIDKLDRESKENFRATNEEIAKLDEDTARVAERVKKAIEDL | | |
| | AK(SEQ ID NO: 27176) | | |
| 3plus_Cage_529_GFP11_Cterm | SEAEDLEELIKELAELLKDVIRKLEKINRRLVKILEDIIRRLKEIS | 3plus_Key_Cterm_529 | KDEAERRRRELKDKLDRLREEHEEVK |
| | KEAEEELRKGTVEDKDILRDLERRLREILEESDRLLEELKRRLEEI | | RRLEEELTRLRETHKKIEKELREALK |
| | LRKSKELLRRLEEVLREILKRNEEVKRSNLPKEELIKEIVKLLEEL | | RVRDRST(SEQ ID NO: 27179) |
| | LRVIEKTLEDNIRLLEELVEVIKEILEKHLTLLEELVRVIERILRE | | |
| | VGKDKDEAERRDHMVLHEYVNAAGITEEVKRRLEEELTRLRETHKK | | |
| | IEKELREALKRVRDRST(SEQ ID NO: 27178) | | |
| 3plus1_Cage_Cterm_263 | SLVDELRKSLERNVPVSEEVARRLKEALKRWVDVVRKVVEDLIRLN | 3plus1_Key_Cterm_263 | DEELKRVLEKNNDLHRRLKDRHRKLL |
| | EDVVRVVEKVTVDESAIERVRRIIEELNRKLDAVLKKNEDLVRRLT | | EDLERIIRELKKKLDEVVEENKRSVD |
| | ELLDKLLEENRRINEELDEDLKRRGGTEEVIDTILELIERSIERLK | | ELKR(SEQ ID NO: 27181) |
| | RLLDELLRIVREALKDNKRVADENLKKLKEILDELRKDGVEDEELK | | |
| | RVLEKAADLHRRLKDRHRKLLEDLERIIRELKKKLDEVVEENKRSV | | |
| | DELKR(SEQ ID NO: 27180) | | |
| 3plus1_Cage_Cterm_494 | SKEDKARELEKRLRDNLKKLEEVVRELNEVLNRNLEKLRRLAEELL | 3plus1_Key_Cterm_494 | EDLVRDIRRELKELEERARKILRDDE |
| | RALKRLLDKLRAGGLPKDELEDLRREVEDVLRRLEDLLRKLKKAND | | RDLRALEKRIRDIIREDREELERLKE |
| | ESLTRLEELLRRAEEENRRVLTTLRELLRGNGDDRDLARLVARLVE | | RARK(SEQ ID NO: 27183) |
| | ANNRALEELLRLVAKNVEDNNRVLEELLRLVKELNKRLLGRIRDED | | |
| | LVRDIRRELKELEEPARKILRDDERDLRALEKPIRDIIREDREELE | | |
| | RLKERARK(SEQ ID NO: 27182) | | |
| 3plus1_Cage_Cterm_500 | SEKEELKRILDKLLKELKRLSDELKATIDKILKILKEVSEEVKRTA | 3plus1_Key_Cterm_500 | EDELRKVEEDLKRLEDKLKKLLEDYE |
| | DELLDAIRRGGVDEEVLREIKREIEEIEKKLRKVNKEIEDEIREIK | | KKVRELEETLDDLIRKYEETLRRLEK |
| | KKLDEVDDKITKEVEKIKEALDKGGVDAKEVIKALKEILKEHADVF | | ELEEAEP(SEQ ID NO: 27185) |
| | EDVLRRIKEIIKRHRDVVKEVLEELRKILEKVAEVLKRQGRSEDEL | | |
| | RKVEEDLKRLEDKLKKILEDYEKKVRELEETLDDLLRKYEETLRRL | | |
| | EKELEEAER(SEQ ID NO: 27184) | | |
| 3plus1_Cage_506_GFP11_Cterm | SAEETLRRITEESKRVLEEITKKYEDLERESREVLRKLREDLDRIK | 3plus1_Key_Cterm_506 | RERLARLLKALADKLIRVLEEILKIN |
| | RELEDVLTKGGDDKDEVLTVLEKVLDELLKLHRENLRVIKELIRKV | | EELNPKIIKFARENLERNRRVNKKVI |
| | LEVDRENLEVLKRLLEKARDLLRRSGRDEKDLKKLEDELRKVKEEL | | EVLREAAR(SEQ ID NO: 27187) |
| | EKKHEASKRDIEDLEPELDKVTEEVERLIRKLKEELRRAAEGSDER | | |
| | DHMVLHEYVNAAGITIRVLEEILKINEELNRKIIKFARENLERNRR | | |
| | VNKKVIEVLREAAR(SEQ ID NO: 27186) | | |
| 3plus1_Cage_508_GFP11_Cterm | STEEILKRVKEILDELAKELRDIIRESLRTIEELLDELRRILEESE | 3plus1_Key_Cterm_508 | DEVKRRLEEKERRIRTRYEELRRRLR |
| | RTLEELVKTIKDGVKDSEELLRRLKRLLEDLRRAHEELLKRLKEAV | | KRVKDYEDKLREIEKKVRRDAERIEE |
| | DEHRDRLRKILEELERVLKELRKRIEELRRSGDRISEKDVLRKLEE | | ELERAKK(SEQ ID NO: 27189) |
| | VLRKALEILERLLKKIRESARELLKIIEEVLDANIKVMEKALKTIK | | |
| | ELLKDGRDHMVLHEYVNAAGITTRYEELRRRIPKPVKDYEDKLREI | | |
| | EKKVRRDAERIEEELERAKK(SEQ ID NO: 27188) | | |
| 3plus1_Cage_Cterm_510 | SEKEELLKLIKRVIELLKRVLEEHLRLVEDVIRRLKELLDSNEKIV | 3plus1_Key_Cterm_510 | EDLLRKAKKVITEVREKLKRNLEDVR |
| | REVIEDLKRLLDEVRGDKEELDRIKEKLEEVLERYKRRLEEIKRDL | | RVIEDVKRKSARILEEARRLIEEVER |
| | ERMLEDYKRELKRIEEDLRRVLEEVERTATRGEGPAEALTDKLRKI | | ELEKIRK(SEQ ID NO: 27191) |
| | LERALRELDKLSKKLDELLKKVLEELEKSNREIDKLLKDVLRRVEE | | |
| | GGASEDLLRKAKKVITEVREKLKPNLEDVRRV1EDVKRKSARILEE | | |
| | ARRLIEEVERELEKIRK(SEQ ID NO: 27190) | | |
| 3plus1_Cage_528_GFP211_Cterm | SEAEDLEELIKELAELLKDVIRKLEKINRRLVKILEDIIRRLKEIS | 3plus1_Key_Cterm_528 | KDEAERRRRELKDKLDPLREEHEEVK |
| | KEAEEELPKGTVEDKDILRDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST(SEQ ID NO: 27193) |
| | LRVIEKILEDNIRLLEELVEVIKEILEKHLRLLEELVPVIERILRE | | |
| | VGRDHMVLHEYVNAAGITLDRLREEHEEVKPRLEEELTRLRETHKK | | |
| | IEKELPFALKRVRDRST(SEQ ID NO: 21192) | | |
| 3plus1_Cage_528_GFP11_Cterm | SEAEDLEELIKELAELLKDVIRKLEKINRRLVKILEDIIRRLKEIS | 3plus1_Key_Cterm_528 | KDEAERRRRELKDKIDRLREEHEEVK |
| | KEAEEELRKGTVEDKDILRDLERRLREILEESDRLLEELKRRLEEI | | RRLEEELTRLRETNKKTEKELREALK |
| | LRKSKELLPRLEEVLREILKRAEEVKRSNL2KEELIKEIVKLLEEL | | RVRDPST(SEQ ID NO: 27195) |
| | LRVIEKILEDNIRLLEELVEVIKEILEKHLRLLEELVRVIERILRE | | |
| | VGKDKRDHMVLHEYVNAAGITLREEHEEVKRRLEEELTRLRETHKK | | |
| | IEKELREALKRVRDRST(SEQ ID NO: 27194) | | |
| 3plus1_Cage_528_GFP11_Cterm | SEAEDLEELIKELAELLKDVIRKLEKINRRLVKILEDIIRRLKEIS | 3plus1_Key_Cterm_528 | KDEAERRRRELKDKLDRLREEHEEVK |
| | KEAEEELPYGTVEDKDILRDLEPPIREILEESDRLLEELKPRLEEI | | RRLEEELTPLRETHKKIEKELREALK |
| | LRKSKELLRRLEEVLREILKRAEEVKRSNLPKEELIKEIVKLLEEL | | RVRDRST(SEQ ID NO: 27197) |
| | LRVIEKILEDNIRLLEELVEVIKEILEKHLRLLEELVRVIERILRE | | |
| | VGKDKDRAERDHMVLHEYVNAAGITHEEVKRRLEEEITRLPETHKK | | |
| | IEKELREALKRVRDRST(SEQ ID NO: 27196) | | |
| 3plus1_Cage_529_G5P11_Cterm | SEAEDLEELIKELAELLKDVIRKLEKINRRLVKILEDIIRRLKEIS | 3plus1_Key_Cterm_529 | KDEAERRRRELKDKLDRLREEHEEVK |
| | KRAEEELRKGTVEDKDILRDLERRLREILEESDRLLEELKRRLEEI | | RRLEEELTRLRETNKKIEKELREALK |
| | LRKSKELLRRLEEVLREILKRAEEVKRSNIPKEELIKEIVKLLEEL | | RVRDRSTSEQ ID NO: 27199) |
| | LIWIEKILEDNIRZLEELVEVIKEILEKH2RLLEELVPVIERILRE | | |
| | VGKRDHMVLHEYVNAAGITDRLREEHEEVKRRIEEELTRLRETHKK | | |
| | IEKELREALKRVRDRST(SEQ ID NO: 27198) | | |
| 3plus1_Cage_529_GFP11_Cterm | SEAEDLEELIKELAELLKDVIRKLEKINRRLVEILEDIIRRLKEIS | 3plus1_Key_Cterm_529 | KDEAEPPRRELKDKLDRLREEHEEVK |
| | KEAEEELRKGTVEDKDILRDLERRLREILEESDRLLEELKRRLEEI | | RRLEEELTRLRETHKKIEKELREALK |
| | LRKSKELLRRLEEVLREILKRAEEVKRSNIPKEELIKEIVKLLEEL | | RVRDRST(SEQ ID NO: 27201) |
| | LRVIEKILEDNIRLLEELVEVIKEILEKHLRLLEELVRVIERILRE | | |
| | VGKDRDHMVLHEYVNAAGITRLREEHEEVKRRLEEELTRLRETHKK | | |
| | IEKELRENLKRVRDRST(SEQ ID NO: 27200) | | |
| 3plus1_Cage_529_GFP11_Cterm | SEAEDLEELIKELAELLKDVIRKLEKINRRIVKILEDIIRRLKEIS | 3plus1_Key_Cterm_529 | KDEAERRRRELKDKLDRLREEHEEVK |
| | KEAEEELRKGTVEDKDILRDLERRLREILEESDRLLEELKRRLEEI | | RRLEEELTRLRETHKKIEKELREALK |
| | LRKSKELLRPLEEVLREILKRAEEVKRSNLPKEELIKEIVKLLEEL | | RVRDRST(SEQ ID NO: 27203) |
| | LPVIEKILEDNIRLLEELVEVIKEILEKHLRLLEELVRVIERILRE | | |
| | VGKDKDRDHMVLHEYVNAAGITREEHEEVKRRLEEELTRLRETHKK | | |
| | IEKELREALKRVRDRST(SEQ ID NO: 27202) | | |
| 3plus1_Cage_Cterm_530 | SDKEEIKRRVEKTARDLETEHDKIKKRLEDTVPDIKRELDELLEKY | 3plus1_Key_Cterm_530 | EKRLIEEVVRLLRENLDVVRRILEAL |
| | ERVLRKTEKTLREGDGDATDLVDKLLETVKRLNEEVLRTVKKIADE | | AKLIKELLEALEEVLRRNKELIRELL |
| | LPRLVEEVLRVLRESNETLARLLEEGNVGDEVREPLEEVLRRTEEI | | RVLDEALK(SEQ ID NO: 27205) |
| | IKTLEEIVKPLLDELRRLVEELRRISKKLKDELDKIKEKGKDEKRL | | |
| | IEEVVRLLRENLDVVPRILEALAKLIKELLEALEEVLRRNKELIRE | | |
| | LLRVLDEALK(SEQ ID NO: 27204) | | |
| 3plus1_Cage_534_GFP11_Cterm | DEDRIIEEIARLLEELLRELLELIKKLIETNRRLNEEHERAVRELA | 3plus1_Key_Cterm_534 | EKIAEEIERELEELRRMIKRLHEDLE |
| | RLLEELLDRLVKKGISDEKLKRIRERLKRALDDLERLHREINKRLE | | PKLKESEDELREIEARLEEKIRRLEE |
| | DLVRELEKLVREILKELKDALEELRRASARAGGEEVLRRLEEIVKK | | KLERKRR(SEQ ID NO: 27207) |
| | LLDLVRRILERLKEIHKDNVRLLRELNERLTRIVEDLVRLIREILR | | |
| | EAGVDEPDHMVLHEYVNAAGITIKRLHEDLERKLKESEDELREIEA | | |
| | RLEEKIRRLEEKLEPKRR(SEQ ID NO: 27206) | | |
| 3plus1_Cage_534_GFP11_Cterm | DEDRIIEEIARLLEELLRELLELIKKLIETNRRLNEEHERAVRELA | 3plus1_Key_Cterm_534 | EKIAEEIERELEELRRMIKPLHEDLE |
| | RLLEELLDRLVKKGISDEKLKRIRERLKRALDDLERLHREINKRLE | | RKLKESEDELREIENRLEEKIRRLEE |
| | DLVRELEKLVREIIKELKDALEELRRNSARNOGEEVLRRLEEIVKK | | KLERKRR(SEQ ID NO: 27209) |
| | LLDLVRRILERLKEIHKDNVRLLRELNERLTRIVEDLVRLIREILR | | |
| | EAGVDEKIRDHMVLHEYVNAAGITRLHEDIERKLKESEDELREIEA | | |
| | RLEEKIRRLEEKLERKRR(SEQ ID NO: 27208) | | |
| 3plus1_Cage_534_GFP11_Cterm | DEDRIIEEIARLLEELLRELLELIKKLIETNRPLNEENERAVRELA | 3plus1_Key_Cterm_534 | EKIAEEIERELEELRRMTKRLHEDLE |
| | RLLEELLDRLVKKGISDEKLKRIPERLKRALDDLERLHPEINKRLE | | RKLKESEDELREIEARLEEKIRRLEE |
| | DLVRELEKLVREILKELKDALEELRRASARAGGEEVLRRLEEIVKK | | KLERKRR(SEQ ID NO: 27211) |
| | LLDLVPPILERLKEINKDNVRLLRELNERLTRIVEDLVPLIREILR | | |
| | ENGVDEKIAEEIERDHMVLHEYVNAAGITLERKLKESEDELREIEA | | |
| | RLEEKIRRLEEKLERKRR(SEQ ID NO: 27210) | | |
| 3plus1_Cage_Cterm_539 | SEKEKLLKESEEEVRRLRRTLEELLRKYREVLERLRKELREIEERV | 3plus1_Key_Cterm_539 | ERLVKTLIEDVEAVIKRILELITRVA |
| | RDVVRRLKEVLDRKGLDIDTIIKEVEDLLKTVLDRLRELLDKIRRL | | EDNERVLERIIRELTDNLERNLKIVR |
| | TKEAIEVVREIIERIVRHAERVKDELRKEGGDKEKLDRVDRLIKEN | | EIVK(SEQ ID NO: 27213) |
| | TRHLKEILDRIEDLVRRSEKKLRDIIREVRRLIEELRKKAEEIKKG | | |
| | PDERLVKTLIEDVENVIKRILEIITRVAEDNERVLERIIRELTDNL | | |
| | ERHLKIVREIVK(SEQ ID NO: 27212) | | |
| 3plus1_Cage_Cterm_548 | DKAEVLREALKLLKDLLEELIKIHEESLKRILDLIDTLVKVHEDAL | 3plus1_Key_Cterm_548 | EEIDRELKRVVEELRRLHEEIKERLD |
| | RALKELLERSGLDERELRKVERMATESLRTIAKLKEELRDLARRSL | | DVAPPSEEELRRIIKKLKEVVEEIRK |
| | EKLREDLKRVDDTIRKVEEKVRRTGPSEELIEELIRTIEKLLKEIV | | KLK(SEQ ID NO: 27215) |
| | RINEEVLKAVRELLKTLLKLSEDVVRRIEEILRKGGVPEEIDRELK | | |
| | RVVEELRRLHEEIKERLDDVARRSEEELRRIIKKLKEVVKEIRKKL | | |
| | K(SEQ ID NO: 27214) | | |
| 3plus1_Cage_Cterm_556 | SERELIERWLELEKEILRLIRELVERLLKLHREILDTIKKLIRELL | 3plus1_Key_Cterm_556 | DDERRTLTELLKRMEDILEKVERTLK |
| | ELLEDIARKLGLDKEAKDELREIAKRVEDKLEKLERESRKVEEDLK | | KLLDDSARMAEEVKKTLKELLERSEK |
| | RKLKELTDESDTVEKRVRDVVRRGTQSREEIAEELLRLDRKLLKAV | | VAEDVRK(SEQ ID NO: 27217) |
| | EELLKEILDLNKKLLDDVRAILEETRRVIEKLLDRVRRGERTDDER | | |
| | RTLTELLKRMEDILEKVERTLKKLLDDSARMAEEVKKTLKELLERS | | |
| | EKVAEDVRK(SEQ ID NO: 27216) | | |
| 3plus1_Cage_Cterm_560 | SKKELLEEVVRRAIELLKRHLEKLKRILEEIVRLLEEHLEKVERVL | 3plus1_Key_Cterm_560 | EDKLKEIEDELRRLLEELRRLDKAIK |
| | EAILSLLDDLLRRGGDERAIRTLEDVKRRLREILERLADENAKAIK | | DRLRELKKDLDEANRRIKETLKKLLR |
| | RLNDLLDKLEKRNKEAIERLEEILEELKRVRRDEELLRVLETLLKI | | EVEK(SEQ ID NO: 27219) |
| | IEDILRENTKVLEDLLPLVEEILENYLRVVEELLRLAREILTEIVG | | |
| | DEDKLKEIEDELRRLLEELRRLDKAIKDRLRELKKDLDEANRRIKE | | |
| | TLKKLLREVEK(SEQ ID NO: 27218) | | |
| 3plus1_Cage_568_GEP11_Cterm | KEIEETLKELEDLNREMVETNRRVLEETRRLNKETVDRVKATLDEL | 3plus1_Key_Cterm_568 | KAVEELEKALEEIKRRLKEVIDRYED |
| | AKMLKKLVDDVRKOPTSEELKRLLAELEELLNRVVRRVEELLKKST | | ELRKLRKEYKEKIDKYERKLEEIERP |
| | DLLERAVKDSADALRRSHEVLKEVASRVKRAKDEGLPREEVLRLLR | | ERT(SEQ ID NO: 27221) |
| | ELLERENKVLKDIVRVSEKLLREHLKVLREIVEVLEELLERILKVI | | |
| | LDTTRDHMVLHEYVNAAGITKRRLKEVIDRYEDELRKLRKEYKEKI | | |
| | DKYERKLEEIERRERT(SEQ ID NO: 27220) | | |
| 3plus1_Cage_568_GFP11_Cterm | KEIEETLKELEDLNREMVETNRPVLEETRRINKETVDRVKATLDEL | 3plus1_Key_Cterm_568 | KAVEELEKALEEIKRRLKEVIDRYED |
| | AKMLKKLVDDVRKGPTSEELKRLLAELEELLARVVRRVEELLKKST | | ELRKLRKEYKEKIDKYERKLEEIERR |
| | DLLERAVKDSADALRRSHEVLKEVASRVKRAKDEGLPREEVLRLLR | | ERT(SEQ ID NO: 27223) |
| | ELLERHAKVLKDIVRVSEKLLREHLXVLREIVEVLEELLERILKVI | | |
| | LDTTGGDRDHMVLHEYVNAAGITLKEVIDRYEDELRKLRKEYKEKI | | |
| | DKYERKLEETERRERT(SEQ ID NO: 27222) | | |
| 3plus1_Cage_Cterm_572 | DEDELIRKLLEDLKDIVRKILELIERDLPLIERVVRRIVKVIRDDL | 3plus1_Key_Cterm_572 | SELADRIRKLIEDLERHTAKVLEDVK |
| | KKIKEVVDDIARGVPRTEELERVIKRIEELLRTSEEELDRILKEIE | | RAITELRKNSKDILEEVRKLIDELRK |
| | ELLRESRRRLEEVVSAVEELLRRVEEIVDKGRESKEDVIKLLREVV | | RIKEVED(SEQ ID NO: 27225) |
| | DDILRLVEEVVRTNLEIIKRILELIERVIRLNLSIIRDILRLLEGT | | |
| | VDSELADRIRKLIEDLERHTAKVLEDVKRAITELRKNSKDILEEVR | | |
| | KLIDELRKRIKEVED(SEQ ID NO: 27224) | | |
| 3plus1_Cage_Cterm_581 | SALETVKKLLEDSSEKIERIVEEDERVAKESSDRIRRLVEEDKRVA | 3plus1_Key_Cterm_581 | AEAVIKVIEKLIRANKRVWDALLKIN |
| | DEILDLIEKIGDTDTLLKLVEEWSRTSKKLLDDVLKLHKDWSDDSR | | EDLVRVNKTVWKELLRVNEKLARDLE |
| | RLLEEILRVEEELIPRVKEILDREGKPEEVVRELEKVLKESLDTLE | | RVVK(SEQ ID NO: 27227) |
| | EIIRRLDEANAATVKRVADVIRELEDINRKVLEEIKRGSDDAEAVI | | |
| | KVIEKLIRANKRVWDALLKINEDLVRVNKTVWKELLRVNEKLARDL | | |
| | ERVVK(SEQ ID NO: 27226) | | |
| 3plus1_Cage_Cterm_585 | SKEEKLKDDVRAVLEDLDRVLKELEKLSEDNLRELKRVLDRITDLH | 3plus1_Key_Cterm_585 | SKAAEDILRVLEKLVKVSREAIKLIL |
| | RRILDELRKGIGSEELLRRVEKVLKDNLDLLRKLVEEHKESSERDL | | ELSEHHVRVSTRIARLLLDVARKLAE |
| | KRVEDLVREIKEVLRKLLELEDPGTDIRKIEEEIERLLRKIRKAVE | | VIKEAER(SEQ ID NO: 27229) |
| | ESKDLNRRNSERIEEVARRSEELARRLIKEIPERODSKAAEDILRV | | |
| | LEKLVKVSPEAIKLILELSEHHVPVSTRIARLLLDVARKLAEVIKE | | |
| | AER(SEQ ID NO: 27228) | | |
| 3plus1_Cage_Cterm_587 | SEIEDVIRRLRKILEDLERVSEKLLREIKKILDEARRINEEVIKEI | 3plus1_Key_Cterm_587 | IEDLVREVERLIKRIEDSLRELEKTV |
| | KRVLEDAVRVFRDOSGSKEEIAKLVEELIRELAKLAKEVDEIHKRI | | RELLKRIKEASDKVREDVDRLIKELK |
| | VERLKALVEDAEPIHRKIVETLEEIVROVPSEELKRVVEAIVEVIK | | EAAD(SEQ ID NO: 27231) |
| | EHLKVLADVIRRIIKAIEENAETIKRVLEDIVRVLELVLRGEGSIE | | |
| | DLVREVERLIKRIEDSLRELEKTVRELLKRIKEASDKVREDVDRLI | | |
| | KELKEAAD(SEQ ID NO: 27230) | | |
| 3plus1_Cage_Cterm_605 | SREELDDRILEATAKILEDLKPLIDENLARLEEVVRELERIIDRNL | 3plus1_Key_Cterm_605 | DEIIRKLDELLKEVEKVHKEVKDRIR |
| | KLIRETLDELKKGSGSEETLEKIKKVDKELEDLTRRLLKKLEDLIR | | KLLEDHKRSLDEVKEKLERLLERAKE |
| | ETERRIREILKRIRDLLKEVKDPDKDLERLLEVLEEVLPVIAELAK | | VVEREKE(SEQ ID NO: 27233) |
| | ELLDSLRKVLKVVEEVLRLLNEVNKEVLDVIRELAKDOGSDEIIRK | | |
| | LDELLKEVEKVHKEVKDRIRKLLEDHKRSLDEVKKKLERLLEPAKE | | |
| | VVEREKK(SEQ ID NO: 27232) | | |
| 3plus1_Cage_Cterm_607 | SEREELLERIKEILKPVKDKLDEDLKRLKEILEKLKEKADRDIEEL | 3plus1_Key_Cterm_607 | SETAVRAIIRVLEKHLEAVRRVLEEL |
| | RRRIEEVREKLERTGRTDELVKEVLDTVRRNLENLKRLVEDILRKL | | LKVLkEHLETVRELJERLKRVLEEAI |
| | EENVKNLTDLVREILKLITELIKRLEDGGLPKEVLDALRRVLEKLE | | EVVERVAR(SEQ ID NO: 27235) |
| | ELLREILERLKRSLEAVKRKIEELLKELERSLDELRRALERIRKEI | | |
| | GDSETAVRAIIRVLEKHLEAVRRVLEELLKVLAEHLETVRELIERL | | |
| | KRVLEEAIEVVERVAR(SEQ ID NO: 27234) | | |
| 3plus1_Cage_Cterm_610 | SLEEITKRLLELVEENLARREEILRELLELAKRLAKEDRDILEEVL | 3plus1_Key_Cterm_610 | ERTLREVVRKVLEEAKRLLDELEEVE |
| | KLIEELLKLLEDNGSSEEDLKRLLKEVIEELRAVVKRVKDKWDEVV | | KRVKKELEDIIEENRRWKRVRDELR |
| | KRIEDLVKKLKELHDDTLRKLRELVRKIVTDISESGGEAEKVKRVV | | EIKRELDE(SEQ ID NO: 27231) |
| | EKILELVERLAKVVKESVEKLLEILRELAEVSKRVAEALLRLLEEL | | |
| | VRVIRIKDERTLREVVRKVLEEAHRLLDELEEVHKRVKKELEDIIE | | |
| | ENRRVVKRVRDELREIKPELDE(SEQ ID NO: 27236) | | |
| 3plus1_Cage_611_GFP11_Cterm | SLEEITKRLLELVEENLARHEEILRELLELAKRLAKEDRDILEEVL | 3plus1_Key_Cterm_611 | ERTLREVVRKVLEEAKRLLDELEEVH |
| | KLIEELLKLLEDNGSSEEDLKRLLKEVIEELRAVVKRVKDKVIDEVV | | KRVKKELEDIIEENRRVVKRVRDELR |
| | KRIEDLVKKLKELHDDTLRKLRELVRKIVTDISESGGEAEKVKRVV | | EIKRELDE(SEQ ID NO: 27239) |
| | EKILEDVERLAKVVKESVEKLLEILRELAEVSKRVAEALLRLLEEL | | |
| | VRVIRIKDERDHMVLHEYVNAAGITLLDELEEVRKRVKKELEDIIE | | |
| | ENRRVVKRVRDELREIKRELDE(SEQ ID NO: 27238) | | |
| 3plus1_Cage_Cterm_632 | SEKELVDDIRRTLEEILRLLRSLLEEVIRILEENEKLVRRHLKTVI | 3plus1_Key_Cterm_632 | DSLVREVEELIKRLEKKIDDLLKTSR |
| | DILRRVAKLLDENGIRTDETADRVLERLEKkHRELLEDYKRALEKIK | | DLVKRVLDLVDEVVKRVEDLVERVKE |
| | ETLERVLPEAEEVVKKIDDALRKLGGSKEVLKRLLEELLRLVEKIA | | KIDT(SEQ ID NO: 27241) |
| | EEIKRLLSELVRVTEELVRTNKELLEEAVRVIRKEVODDSLVREVE | | |
| | ELIKRZEKHIDDLLKTSRDLVKPVLDLVDEVVKRVEDLVERVKEKI | | |
| | DT(SEQ ID NO: 27240) | | |
| 3plus1_Cage_Cterm_641 | DEVEELLKRVRELLKASEELVRKILEDVKRDLERSIEEVEDLLRKV | 3plus1_Key_Cterm_641 | EDLIRDVDRVLKEIMDLLRDLVRRTK |
| | EELLKRLLDLVERGGSLDEILRELVRLLKEIVRKVLELNRKLVEDV | | ELLEELLRLLEELVRAHKELVRSILD |
| | IPIAKRLIELNAESVKEWDRILKLLRDGTGDKEELLKDAEFALEKY | | EIRK(SEQ ID NO: 27243) |
| | ERAVRYLLKELEEIIKEYVRRVEELLKELKDAVDKAKDEVRKGSGD | | |
| | EDLIRDVDRVLKEIMDLLRDLVRRTKELIEELLRLLEELVRAHKEL | | |
| | VRSILDEIRK(SEQ ID NO: 27242) | | |
| 3plus1_Cage_646_GFP11_Cterm | DAEEVVKRLADVLRENDETIRKVVEDLVRIAEENDRLWKKLVEDIA | 3plus1_Key_Cterm_646 | EDVKRALEELVSRLRKLLEDVKKASE |
| | EILRRIVEILRRGGVPEELLDRIAKVVKSIVEKAEKILERLNRVSK | | DIVREVERIVRELAKRSDEILKKLED |
| | AIAEKLKTIVDELNEVSKEIVKRAEDILRKGKDKETVLRAIRTLVK | | IVEKLRE(SEQ ID NO: 27245) |
| | EYADLSKEVLERVERIVREYVKLSDEVVKSLAEIVREELRIIEDLL | | |
| | RKGNRDHMVLHEYVNAAGITRKLLEDVKKASEDIVREVERIVRELA | | |
| | KRSDEILKKLEDIVEKLRE(SEQ ID NO: 27244) | | |
| 3plus1_Cage_646_GFP11_Cterm | DAEEVVKRLNDVLRENDETIRKVVEDLVRIAEENDRLWKKLVEDIA | 3plus1_Key_Cterm_646 | EDVKPALEELVSRLRKLLEDVKKASE |
| | EILRRIVELLRRGOVPEELLDRLAKVVKSIVEKAEKILERLNRVSK | | DIVREVERIVRELAKRSDEILKKLED |
| | AIAEKLKTIVDELNEVSKEIVKRAEDILRKGKDKETVLRALRTLVK | | IVEKLRE(SEQ ID NO: 27247) |
| | EYADLSKEVLERVERIVREYVKLSDEVVKSLAEIVEELIRIIEDLL | | |
| | RKGNLDEDRDHMVDHEYVNAAGITEDVKKASEDIVREVERIVRELA | | |
| | IKRSDEILKKLEDIVEKLRE(SEQ ID NO: 27246) | | |
| 3plus1_Cage_646_GFP11_Cterm | DAEEVVKRLADVLRENDETIRKVVEDLVRIAEENDRLWKKLVEDIA | 3plus1_Key_Cterm_646 | EDVKRALEEINSRLPKLLEDVKKASE |
| | EILRRIVELLRRGGVPEELLDRLAKVVKSIVEKAEKILERLNRVSK | | DIVREVEPIVRELAKRSDEILKKLED |
| | AIAEKLKTIVDELNEVSKEIVKRAEDILRKGKDKETVLRALRTLVK | | IVEKLRE(SEQ ID NO: 27249 |
| | EYADLSKEVLERVERIVREYVKLSDEVVKSLAEIVEELIPIIEDLL | | |
| | RKGNLDEDVRDHMVLHEYVNAAGITDVKKASEDIVREVERIVRELA | | |
| | KRSDEILKKLEDIVEKLRE(SEQ ID NO: 27248) | | |
| 3plus1_Cage_647_GFP11_Cterm | DAEEVVKRLADVLRENDETIRKVVEDLVRIAEENDRLWKKLVEDIA | 3plus1_Key_Cterm_647 | EDVKRALEELVSRLRKLLEDVKKASE |
| | EILRRIVELLRRGGVPEELLDRLAKVVKSIVEKAEKILERLNRVSK | | DIVREVERIVRELAKRSDEILKKLED |
| | AIAEKLKTIVDELNEVSKEIVKRAEDILKKGKDKETVLPALRTLVK | | IVEKLRE(SEQ ID NO: 27251) |
| | EYADLSKEVLERVERIVREYVKLSDEVVKSLAEIVEELIRIIEDLL | | |
| | RKGNLRDHMVLHEYVNAAGITKLLEDVKKASEDIVREVERIVRELA | | |
| | KRSDEILKKLEDIVEKLRE(SEQ ID NO: 27250) | | |
| 3plus1_Cage_647_GFP11_Cterm | DAEEVVKRLADVLRENDETIRKVVEDLVRIAEENDRLWKKLVEDIA | 3plus1_Key_Cterm_647 | EDVKRkLEELVSRIRKLLEDVKKASE |
| | EILRRIVELLRRGGVPEELLDRLAKVVKSIVEKAEKILERLNRVSK | | DIVREVERIVRELAKRSDEILKKLED |
| | AIAEKLKTIVDELNEVSKEIVKPAEDILRKGKDKETVLRAIRTLVK | | IVEKLRE(SEQ ID NO: 27253) |
| | EYADLSKEVLERVERIVREYVKLSDEVVKSLAEIVEELIRIIEDLL | | |
| | RKGNLDEDVKRALERDHMVLHEYVNAAGITSEDIVREVERIVRELA | | |
| | KRSDEILKKLEDIVEKLRE(SEQ ID NO: 27252) | | |
| 3plus1_Cage_Cterm_647 | DAEEVVKRLADVLRENDETIRKVVEDLVRIAEENDRLWKKLVEDIA | 3plus1_Key_Cterm_647 | EDVKRALEELVSRLRKLLEDVKKASE |
| | EILRRIVELLRRGGVPEELLDRLAKVVKSIVEKAEKILERLNRVSK | | DIVREVERIVRELAKRSDEILKKLED |
| | AIAEKLKTIVDELNEVSKEIVKRAEDILRKGKDKETVLRALRTLVK | | IVEKLRE(SEQ ID NO: 27255) |
| | EYADLSKEVLERVERIVREYVKLSDEVVKSLAEIVEELIRIIEDLL | | |
| | RKGNLDEDVKRALEELVSRLRKLLEDVKKASEDIVREVERIVRELA | | |
| | KRSDEILKKLEDIVEKLRE(SEQ ID NO: 27254) | | |
| 3plus1_Cage_Cterm_653 | DEEETLRRLLERKVELAKEYLDVSKEVIDRTTKLLDEYLKTSKRIV | 3plus1_Key_Cterm_653 | SREALEEARRRLEELLRELNEITKDL |
| | DATVELLERGDLGPDELIKRLAEELERSLRELEEEIKRLKRELEES | | EAKLEKLLRDLNELTKALEEELKRLL |
| | LKKLKEIIDRLAEEAEKLLAVLKPGEGSEEEALRALASLVRELIEV | | DELKKRTD(SEQ ID NO: 27257) |
| | LRENDERLRDVLRRLIEALRKNNEILERVLRKLVRAAEERGRDESS | | |
| | REALEEARRRLEELLRELNEITKDLEAKLEKLLRDLNELTKALEEE | | |
| | LKRLLDELKKRTD(SEQ ID NO: 27256) | | |
| 3plus1_Cage_Cterm_658 | DEERIIKTLEDINAKIVEDIKRILDKVAELNERLADAIRKILEETK | 3plus1_Key_Cterm_658 | KDTLRTVEKLVEDVKRRLDKLLEDYK |
| | RILEATTRKVRKDGEISEELLRPLEEKLRKLLEDLERVLAEHEDES | | RLIEEVKKELDKLLKEYEDALREIKK |
| | RRILEEVERLLKRHADASKELLDRARSVARGVKSDKELVDRLKKLI | | RIDE(SEQ ID NO: 27259) |
| | DDSLESVRELIERLKELLDRLVKSVEDLIRTIKELLDRLVEVLREG | | |
| | VSDKDTLRTVEKLVEDVKRRLDKLLEDYKRLIEEVKKELDKLIKEY | | |
| | EDALREIKKRIDE(SEQ ID NO: 27258) | | |
| 3plus1_Cage_Cterm_660 | TEEEVVEDVKRVLDESHDDLRRLIETLTRVIRESLKRIKEALEELE | 3plus1_Key_Cterm_660 | REEVKRVLEEARDELRRLLEEYKATI |
| | RVLKKLLDLLEGGPDAREVLDEIRKVLERLREVIEELLRINKEVLR | | EELERELERLLREHREVIRRIKEEID |
| | ELERVIRELLKKNEDLARRVRSGVKSRLLEVLERLARESLELNRAI | | KSSK(SEQ ID NO: 27261) |
| | LEELRKLVENSLPAVEKILKRLEEIVRKLLKLVEDGGPREEVKRVL | | |
| | EEARDELRRLLEEYKAITEELERELERLLREHREVIRRIKEEIDKS | | |
| | SK(SEQ ID NO: 27260) | | |
| 3plus1_Cage_Nterm_263 | SLVDELRKSLEPNVRVSEEVARRLKEALKRWVDVVRKVVEDLIRLN | 3plus1_Key_Nterm_263 | SLVDELRKSLEPNVRVSEEVARRLKE |
| | EDVVPVVEKVTVDESAIERVRRIIEELNRKLDAVLKKNEDLVRRLT | | ALKPNVDVVRKVVEDLIRLNEDVVRV |
| | ELLDKLLEENRRLVEELDEDLKRRGGTEEVIDTILELIERSIERLK | | VEKV(SEQ ID NO: 27263) |
| | RLLDELLRIVREALKDNKRVADENLKKLKEILDELRKDGVEDEELK | | |
| | RVLEKAADLHRRLKDRHRKLLEDLERIIRELKKKLDEVVEENKRSV | | |
| | DELKR(SEQ ID NO: 27262) | | |
| 3plus1_Cage_Nterm_500 | SEKEELKRLLDKLLKELKRLSDELKATIDKILKILKEVSEEVKRTA | 3plus1_Key_Nterm_500 | SEKEELKRLLDKLIKELKRLSDELKA |
| | DELLDAIPPGGVDEEVLREIKREIEEIEKKLRKVNKEIEDEIRETK | | TIDKILKILKEVSEEVXRTADELLDA |
| | KKLDEVDDKITKEVEKIKEALDKGGVDAKEVIKALKEILKEHADVF | | IRRG(SEQ ID NO: 27265) |
| | EDVLRRLKEIIKRHRDVVKEVLEELRKILEKVAEVLKRQGRSEDEL | | |
| | PKVEEDLKRLEDKLKKLLEDYEKKVRELEETLDDLLREYEETLRRL | | |
| | EKELEEAER(SEQ ID NO: 27264) | | |
| 3plus1_Cage_Nterm_510 | SEKEELLKLIKRVIELLKRVLEEHLRLVEDVIRRLKELLDSNEKIV | 3plus1_Key_Nterm_510 | SEKEELLKLIKRVIELLKRVLEEHLR |
| | REVIEDLKRLLDEVRGDKEELDRIKEKLEEVLERYKRRLEEIKRDL | | LVEDVIRRLKELLDSNEKIVREVIED |
| | ERMLEDYKRELKRIEEDLRRVLEEVERIATRGEGPAEALTDKLRKT | | LKRLLDEV(SEQ ID NO: 27267) |
| | LERALRELDKLSKKLDELLEKVLEELEKSNREIDKLLKDVLRRVEE | | |
| | GGASEDLLRKAKKVITEVREKLKPNLEDVRRVIEDVKRKSARILEE | | |
| | ARRLIEEVERELEKIRK(SEQ ID NO: 27266) | | |
| 3plus1_Cage_529_GFP11_Nterm | SEAEDLEELIKELAELLKDVIRKLEKINRRLVKILRDHMVLHEYVN | 3plus1_Key_Nterm_529 | SEAEDLEELIKELAELLKDVIRKLEK |
| | AAGITELPKGTVEDKDILRDLEPPLREILEESDRLLEELKRRLEEI | | INRRLVKILEDIIRRLKEISKEAEEE |
| | LRKSKELLRRLEEVLREILKRAEEVKRSNLPKEELIKEIVKLIEEL | | LRKG(SEQ ID NO: 27269) |
| | LRVIEKILEDNIRLLEELVEVIKEILEKHLRLLEELVRVIERILRE | | |
| | VGKDKDEAERRRRELKDKLDRLREEHEEVKPRLEEELTRLRETHKK | | |
| | IEKELPEALKRVRDRST(SEQ ID NO: 27268) | | |
| 3plus1_Cage_568_GFP11_Nterm | KEIEETLKELEDLNREMVETNRRVLEETRRLNKETVDRVKATRDHM | 3plus1_Key_Nterm_568 | KEIEETLKELEDLNREMVETNRRVLE |
| | VLHEY-VNAAGITKCPTSEELKRLLAELFELTARVVRRVEELLKKST | | ETRRLNKETVDRVKATLDELAKMLKK |
| | DLLEPAVKDSADALRRSHEVLKEVASRVKRAKDEGLPREEVLFLLR | | LVDDVPXG(SEQ ID NO: 27271) |
| | ELLERHAKVLKDIVRVSEKLLREHLKVLREIVEVLEELLERILKVI | | |
| | LDTTGGDKAVEELEKALEEIKRRLKEVIDRYEDELRKLREEYKEKI | | |
| | DKYERKLEEIERRERT(SEQ ID NO: 27270) | | |
| 3plus1_Cage_Nterm_581 | SALETVKKLLEDSSEKIERIVEEDERVAKESSDRIRRLVEEDKRVA | 3plus1_Key_Nterm_581 | SALETVKKLLEDSSEKIERIVEEDER |
| | DEILDLIEKIGDTDTLLKLVEEWSRTSKKLLDDVLKLHKDWSDDSR | | VAKESSDRIRRLVEEDKRVADEILDL |
| | RLLEEILRVHEELIRRVKEILDREGKPEEVVRELEKVLKESLDTLE | | IEKI(SEQ ID NO: 27273) |
| | EIIRRLDEANAATVKRVADVIRELEDINRKVLEEIKRGSDDAEAVI | | |
| | KVIEKLIPANKRVWDALLKINEDLVRVNKTVWKELLRVNEKLARDL | | |
| | ERVVK(SEQ ID NO: 27272) | | |
| 3plus1_Cage_610_GFP11_Nterm | SLEEITKRLLELVEENLARHEEILREELELAKRLAKRDHMVLHEYV | 3plus1_Key_Nterm_610 | SLEEITKRLLELVEENLARHEEILRE |
| | NAAGITLKLLEDNGSSEEDLKRLLKEVIEELRAVVKRVKDKWDEVV | | LLELAKRLAKEDRDILEEVLKLlEEL |
| | KRIEDLVEKLKELHDDTLRKLRELVRKIVTDISESGGEAEKVKRVV | | LKELEDN(SEQ ID NO: 27275) |
| | EKILELVERLAKVVKESVEKLLEILRELAEVSKRVAEAELRELEEL | | |
| | VRVIRIKDERTLREVVRKVLEEAKRLLDELEEVHKRVKKELEDIIE | | |
| | ENRRVVKRVRDELREIKRELDE(SEQ ID NO: 27274) | | |
| 3plus1_Cage_647_GFP11_Nterm | DAEEVVERLADVLRENDETIRKVVEDLVRIAEENDRLWRDHMVLHE | 3plus1_Key_Nterm_647 | DAEEVVKRLADVLRENDETIRKVVED |
| | YVNAAGITLLRRGGVPEELLDRLAKVVKSIVEKAEKILERENRVSK | | LVRIAEENDRLWKELVEDIAEILRRI |
| | AIAEKLETIVDELNEVSKEIVKPAEDIERKGKDKETVLRALRTLVK | | VELLRRG(SEQ ID NO: 27277) |
| | EYADLSKEVLERVERIVREYVKLGDEVVKSLAEIVEELIRIIEDLL | | |
| | PKGNLDEDVKRALEELVSRLRELLEDVKKASEDIVREVERIVRELA | | |
| | KRSDEILKKLEDIVEKLRE(SEQ ID NO: 27276) | | |

TABLE 4

| Row | | |
|---|---|---|
| number | Cage (column 1) | Key (column 2) |
| 1 | LOCKR_extend18 (SEQ ID NO: 6), | p18_MBP (SEQ ID NO: 27020), |
| | BimLOCKR_extend18 (SEQ ID NO: 22), | p76-long (SEQ ID NO: 27027), |
| | , | p76-short (SEQ ID NO: 27028), |
| | 1fix-long-Bim-t0 (SEQ ID NO: 54), | |
| | 1fix-long-GFP-t0 (SEQ ID NO: 55), | |
| | 1fix-short-BIM-t0 (SEQ ID NO: 56), | |
| | 1fix-short-GFP-t0 (SEQ ID NO: 57), | |
| 2 | LOCKRb (SEQ ID NO: 7), | key_b (SEQ ID NO: 27022) |
| 3 | LOCKRc (SEQ ID NO: 8), | key_c (SEQ ID NO: 27023) |

TABLE 5

| Cage Name | Cage Sequence | Key Name | Key Sequence |
|---|---|---|---|
| 2plus1_Cage_Cterm_2406 | SEVDEVVKEVEDLVRRNEELVEEVVRRVEKVVTDDRRLVEEVVREI | 2plus1_Key_Cterm_2406 | EKVLRKLEKVIREVRERSTRALRKVE |
| | RKIVKLVEDLARKLDKEELKRVLDEMRERIERLLEKLRRHSKKLDD | | EVIRRVREESERAIRDLERVVKEVEK |
| | ELKRLLEELREHSRRVEKRLEDLLKELRERGVDEKVLRKLEKVIRE | | RMREAAR(SEQ ID NO: 27127) |
| | VRERSTRALRKVEEVIRRVREESERALRDLERVVKEVEKRMREAAR | | |
| | (SEQ ID NO: 27126) | | |
| 2plus1_Cage_Cterm_5398 | SVEELLRKLEEVLRKIREENERSLKELRDRAREIVKRNRETNRELE | 2plus1_Key_Cterm_5398 | EDIVRKIERIVETIEREVRESVKKVE |
| | EVIKELEKRLSGADKEKVEELVRRIRRIVERVVEEDRRTVEEIEKI | | EIARDIRRKVDESVKNVEKLLRDVDK |
| | AREVVKRDRDSADRVRRTVEDVDRKATGSEDIVRKIERIVETIERE | | KARDRKK(SEQ ID NO: 27129) |
| | VRESVKKVEEIARDIRRKVDESVKNVEKLLRDVDKKARDRKK(SEQ | | |
| | ID NO: 27128) | | |
| 2plus1_Cage_Cterm_5405 | SESDDVIRKLRELLEELRTHVEKSIRDLRKILEDSTRNAKRSIEEL | 2plus1_Key_Cterm_5405 | EEKDKDLIRKLRDIIPPAAEANKKLI |
| | ERLLEEVPAAPGDEEVRKTVEEISRRVAENVKRLEDLYRPMEEEVK | | DDARESLERAKREHEKLIDRLKKILE |
| | KNLDRLRKRVEDIIREVEEARKKGVDEEKLKDLIRKLRDILRRAAE | | ELER(SEQ ID NO: 27131 |
| | AHKKLIDDARESLERAKREHEKLIDRLKKILEELER(SEQ ID | | |
| | NO: 27130) | | |
| 2plus1_Cage_Cterm_5406 | DREREVKKRLDEVREPIERLLRRVEEESRRVAEEIRRLIEEVRRRN | 2plus1_Key_Cterm_5406 | EELREELKKLERKIEEVAKEIHDHDK |
| | KKVTEEIRELLKGLKDKEEVRRVLERLRKLNAESDELLERILERLR | | EVTERLEDLLRRITEHARKSDREIEE |
| | RLVEATNRLVKAIIEELRRLVEKIVREVPDSEELREELKKLERKIE | | TAR(SEQ ID NO: 27133) |
| | KVAKEIHDHDKEVTERLEDLLRRITEHARKSDREIEETAR(SEQ | | |
| | ID NO: 27132) | | |
| 2plus1_Cage_Cterm_5409 | SEAEELLKRLEDRAEEILRRLEEILRTSPELAEDVLRELEKLLRES | 2plus1_Key_Cterm_5409 | KEVVDEIKRIVDEVRERLKRIVDENA |
| | ERRIREVLEELRGIKDKKELEDVIREVEKELDESLERSPELLKDVL | | KIVEDAPPALEKIVKENEEIDRRLKK |
| | KKLDDNLKESERLVEDIDRELAKILEDLKKAGVPKEVVDEIKRIVD | | ELRELRK(SEQ ID NO: 27135) |
| | EVRERLKRIVDENAKIVEDARRALEKIVKENEEILRRLKKELRELR | | |
| | K(SEQ ID NO: 27134) | | |
| 2plus1_Cage_5414_GFP11_Cterm | SEIEKILKEIEDLARRDEEVSKKIVEDIRRLAKEVEDTSRDIVRKI | 2plus1_Key_Cterm_5414 | EDSERLVREVEDLVRRLVRRSEKSNE |
| | EELAKPVLDRLRKDGSKEELEKEVREVVKTLEELVKDNHRLIRRAV | | EVKRTVEELVRRMEESNDRVRDLVRR |
| | EEMKRLVEENHPESREVVKELEDLVRELRKGSGSEDSERDHMVLHE | | LVEELKRAVD(SEQ ID |
| | YVNAAGITSEKSNEEVYRTVEELVRRMEESNDRVRDLVPRLVEELK | | NO: 27141) |
| | RAVD(SEQ ID NO: 27140) | | |
| 2plus1_Cage_Cterm_5421 | SVDEVLKEIEDALRRIKEEVEPVLKENEDELRRLEEEVPRVLKEDE | 2plus1_Key_Cterm_5421 | EKAIRDVAKEIRDRLKELEEEIEEVT |
| | ELLESLKRGVGESDEVDRVVDEIAKLSAEILEKVKKVVKEIRDSLE | | RRNLKLLADVEEEIPPVHEKTRRLLE |
| | TVKRRVDMIVRRLKELLDEIKRGSDEKAIRDVAKEIRDRLKELEEE | | TVLRRAT(SEQ ID NO: 27147) |
| | IEEVTTRNLKLLADVEEEIRRVHEKTRRLLETVLRRAT(SEQ ID | | |
| | NO: 27146) | | |
| 2plus1_Cage_Cterm_5432 | DEIRKVVKEITDLLKASNDKNRKVVEEIRDLLRKSKKLADELVERL | 2plus1_Key_Cterm_5432 | SEDLKPVEERAREVSRRNEESMRRVK |
| | RALVEDLRRRIDKSGDKETAEDIVRRIIEEIKRILKEIEDLARRIN | | EDADRVSEANKEVLDPVREEVKRLIE |
| | REIERLVEEVERDNRDVNRAIEELLKDIARRGGSEDLKRVEERARE | | EVRETLR(SEQ ID NO: 27149) |
| | VSRRNEESMRRVKEDADRVSEANKEVLDRVREEVKRLIEEVRETLR | | |
| | (SEQ ID NO: 27148) | | |
| 2plus1_Cage_Cterm_5435 | STAETVAEEVERVLKHSDDLIKEVEDVNRRVEEEIKRVIRELEEEN | 2plus1_Key_Cterm_5435 | EEAAREIIKRLREVNKRTKEKLDELI |
| | ERLVAEVRKGVKGEILAEIEKRLADNSEKVREVAERAKKLLEENTA | | KHSEEVLIRVKRLIDELRKHSEEVLE |
| | RVKDILRESPKLVKDLLDEVRGTGSEEAAREIIKRLREVNKRTKEK | | DLRRRAK(SEQ ID NO: 27151) |
| | LDELIKHSEEVIERVKRLIDELRKHSEEVLEDLRRRAK(SEQ ID | | |
| | NO: 27150) | | |
| 2plus1_Cage_Cterm_5439 | SRVEEIIEDLRRLLEEIRKENEDSIRRSKELLDRVKEINDTIIAEL | 2plus1_Key_Cterm_5439 | EDKARKVAEVAEKVLRDIDKLDRESK |
| | ERLLKDIEKEVREKGSESEEVKKALRAVLEELEKLLRRVAEINEEV | | EAFRATNEEIAKLDEDTARVAERVKK |
| | LRRNSKLVEEDERRNREVLKELARLVEELIREIGDEDKARKVAEVA | | AIEDLAK(SEQ ID NO: 27155) |
| | EKVLRDIDKLDRESKEAFRATNEEIAKLDEDTARVAERVKKAIEDL | | |
| | AK(SEQ ID NO: 27154) | | |
| 2plus1_Cage_Cterm_5447 | SEADDVLKKLAETVKRIIERLKKLTDDSRRLVEEVHPRNDKLSKES | 2plus1_Key_Cterm_5447 | EELSAEVKKLLDEVRKALARHKDEND |
| | AEAVRKAEERGIDEKDVRKLLEDLKKKSEEVAERNKRILDTLREIS | | KLLKEIEDSLRRHKEENDRLLEKLKE |
| | KRAEDEVRKVLKELEKTLKELEDRRPDSEELSAEVKKLLDEVRKAL | | STR(SEQ ID NO: 27157) |
| | ARHKDENDKLLKEIEDSLRRHKEENDRLLEKLKESTR(SEQ ID | | |
| | NO: 27156) | | |
| 2plus1_Cage_Cterm_5456 | SAEELLREVAELVKRVDEDLRRLLEEVRASNEEVIRRLEEILKRIE | 2plus1_Key_Cterm_5465 | EETVKRLLDELRELLERLKRTIEELL |
| | EENRKVVEELRRGGVSEDLVRESKRLVDESRRVIEKLVKESADSVE | | KRNRDLLADAEEKARRLLEENRKLLK |
| | RTRETVDRLREELKPLVEEIAKMVKGGSSEETVKRLLDELRELLER | | AARDTAT(SEQ ID NO: 27159) |
| | LKRTIEELKRNRDLLADAEEKARRLLEENRKLLKAARDTAT(SEQ | | |
| | ID NO: 27158) | | |
| 2plus1_Cage_Nterm_2406 | SEVDEVVKEVEDLVRRNEELVEEVVRRVEKVVTDDRRLVEEVVREI | 2plus1_Key_Nterm_2406 | SEVDEVVKEVEDLVRRNEELVEEVVR |
| | RKIVKDVEDLARKLDKEELKRVLDEMRERIERLLEKLRPHSKKLDD | | RVEKVVTDDRRLVEEVVREIRKIVKD |
| | ELKRLLEELREHSPRVEKRLEDLLKELRERGVDEKVLRKLEKVIRE | | VEDLARK(SEQ ID NO: 27163) |
| | VRERSTRALRKVEEVIRRVREESERALRDLERVVKEVEKRMPEAAR | | |
| | (SEQ ID NO: 27162) | | |
| 2plus1_Cage_Nterm_5406 | DREREVKKRLDEVRERIERLLRRVEEESRRVAEEIRRLIEEVRRRN | 2plus1_Key_Nterm_5406 | DREREVKKRLDEVRERIERLLRRVEE |
| | KKVTEEIRELLKGLKDKEEVRRVLERLRKLNAESDELLERILERLR | | ESRRVAEEIRRLIEEVRRRNKKVTEE |
| | PLVEATNRLVKAIIEELRRLVEKIVREVPDSEEIREELKKLERKIE | | IRELLKGL(SEQ ID NO: 27165) |
| | KVAKEIHDHDKEVTERLEDLLRRITEHARKSDREIEETAR(SEQ | | |
| | ID NO: 27164) | | |
| 2plus1_Cage_Nterm_5409 | SEAEELLKRLEDRAEEILRRLEEILRTSRKLAEDVLRELEKLLRES | 2plus1_Key_Nterm_5409 | SEAEELLKRLEDRAEEILRRIEEILR |
| | ERRIREVLEELRGIKDKKELEDVIREVEKELDESLERSRELIKDVL | | TSRKLAEDVLRELEKLLRESERRIRE |
| | KKLDDNLKESERLVEDIDRELAKILEDLKKAGVPKEVVDEIKRIVD | | VLEELRGI(SEQ ID NO: 27167) |
| | EVRERLKRIVDENAKIVEDARRALEKIVKENEEILRRLKKELRELR | | |
| | K(SEQ ID NO: 27166) | | |
| 3plus_Cage_529_GFP11_Cterm | SEAEDLEELIKELAELLKDVIRKLEKINRRLVKILEDIIRRLKEIS | 3plus_Key_Cterm_529 | KDEAERPRRELKDKIDRLREEHEEVK |
| | KEAEEELRKGTVEDKDILRDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST(SEQ ID NO: 27179) |
| | LRVIEKILEDNIRLLEELVEVIKEILEKHLRLLEELVRVIERILRE | | |
| | VGKDKDKAERRDHMVLHEYVNAAGITEEVKRPLEEELTRLRETHKK | | |
| | IEKELREAIKRVRDRST(SEQ ID NO: 27178) | | |
| 3plus1_Cage_Cterm_500 | SEKEELKRLLDKLLKELKRLSDELKATIDKILKILKEVSEEVKRTA | 3plus1_Key_Cterm_500 | EDELRKVEEDLKRLEDKLKKLLEDYE |
| | DELLDAIRRGGVDEEVLREIKREIEEIEKKLRKVNKEIEDEIREIK | | KKVRELEETLDDLLRKYEETLRRLEK |
| | KKLDEVDDKITKEVEKIKEALDKGGVDAKEVIKALKEILKEHADVF | | ELEEAER(SEQ ID NO: 27185) |
| | EDVLPPLKEIIKPHRDVVKEVLEELRKILEKVAEVLKRQGRSEDEL | | |
| | RKVEEDLKRLEDKLKKLLEDYEKKVRELEETLDDLLRKYEETLRRL | | |
| | EKELEEAER(SEQ ID NO: 27184) | | |
| 3plus1_Cage_Cterm_510 | SEKEELLKLIKRVIELLKRVLEEHLRLVEDVIRRLKELLDSNEKIV | 3plus1_Key_Cterm_510 | EDLLPKAKKVITEVREKLKPVLEDVR |
| | REVIEDLKRLDDEVRGDKEELDRIKEKLEEVLERYKPRLEEIKRDL | | RVIEDVKRKSARILEEAPRLIEEVER |
| | ERMLEDYKRELKRIEEDLRRVLEEVERIATRGEGPAEALIDKLPKI | | ELEKIRK(SEQ ID NO: 2/191) |
| | LERALRELDKLSKKLDELLKKVLEELEKSNREIDKLLKDVLRRVEE | | |
| | GGASEDLLPKAKKVITEVREKLKRNLEDVRRVIEDVKRKSARILEE | | |
| | ARRLIEEVERELEKIRK(SEQ ID NO: 27190) | | |
| 3plus1_Cage_528_GFP11_Cterm | SEAEDLEELIKELAELLKDVIRKLEKINPPLVKILEDIIRPLKEIS | 3plus1_Key_Cterm_528 | KDEAERPPRELKDKLDPLREEHEEVK |
| | KEAEEELPKGTVEDKDILRDLEPPLPETLEESDPLLEELYRRLEEI | | RRLEEELTRLRETHKKIEKELREALK |
| | LRKSKELLPPLEEVLREILKRAFEVKPSNLPKEELIKEIVKLLEEL | | RVRDRST(SEQ ID NO: 27193) |
| | LPVIEKILEDNIPILEELVEVIKEILEKHLPILEELVPVIERILRE | | |
| | VGRDHMVLHEYVNAAGITLDRLREEHEEVKRRLEEELTPIRETHKK | | |
| | IEKELPEALKRVPDRST(SEQ ID NO: 27192) | | |
| 3plus1_Cage_528_GFP11_Cterm | SEAEDLEELIKELAELLKDVIPXLEKINPRLVKILEDIIPPLKEIS | 3plus1_Key_Cterm_528 | KDEAERRRRELKDKLDRLREEHEEVK |
| | KEAEEELPKGTVEDKDILPDLEPPLREIDEESDPLLEELKPRLEEI | | RRLEEELTRLRETHKKIEKELREALK |
| | LRKSKELLPPLEEVLREILKPAEEVKPSNLPKEELIKEIVKLLEEL | | RVRDRST(SEQ ID NO: 27195) |
| | LRVIEKILEDNIRLLEELVEVIKEILEKHLRLLEELVRVIERILRE | | |
| | VGKDKPDHMVLHEYVNAAGITLREEHEEVKRRLEEELTPLRETHKK | | |
| | IEKELPEALKPVPDPST(SEQ ID NO: 27194) | | |
| 3plus1_Cage_528_GFP11_Cterm | SEAEDLEELIKELAELLKDVIRKLEKINRPLVKILEDIIPPLKEIS | 3plus1_Key_Cterm_528 | KDEAERPRRELKDKLDPEREEHEEVK |
| | KEAEEELRKGTVEDKDILPDLERPLREILEESDPLLEELKRRLEEI | | RRLEEELTRLRETHKKIEKELREALK |
| | LRKSKELLPPLEEVLREILKRAEEVKPSNLPKEELIKEIVKLLEEL | | RVRDRST(SEQ ID NO: 27197) |
| | LRVIEKILEDNIPLLEELVEVIKEILEKHLPILEELVPVIERILRE | | |
| | VGKDKDEAEPDHMVLHEYVNAAGITHEEVKRRLEEELTRIPETHKK | | |
| | IEKELREALKRVPDPST(SEQ ID NO: 27196) | | |
| 3plus1_Cage_529_GFP11_Cterm | SEAEDLEELIKELAELLKDVIRKLEKINRRLVKILEDIIRRLKEIS | 3plus1_Key_Cterm_529 | KDEAERRPRELKDKLDRLREEHEEVK |
| | KEAEEELPKGTVEDKDILPDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST(SEQ ID NO: 27299 |
| | LRVIEKILEDNIRLLEELVEVIKEILEKHLRLLEELVRVIERILRE | | |
| | VGKRDHMVLHEYVNAAGITDRLREEHEEVKRRLEEELTRLRETHKK | | |
| | IEKELREAIKRVRDRST(SEQ ID NO: 27198) | | |
| 3plus1_Cage_529_GF911_Cterm | SEAEDLEELIKELAELLKDVIRKLEKINRRLVKILEDIIRRLKEIS | 3plus1_Key_Cterm_529 | KDEAERRRRELKDKLDRLREEHEEVK |
| | KEAEEEIRKGTVEDKDILRDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST(SEQ ID NO: 27202) |
| | LRVIEKILEDNIRLLEELVEVIKEILEKHLPLLEELVRVIERILRE | | |
| | VGKDRDHMVLHEYVNAAGITRIREEHEEVKRRLEEELTPLPETHKK | | |
| | IEKELREALKRVRDRST(SEQ ID NO: 27200) | | |
| 3plus1_Cage_529_GFP11_Cterm | SEAEDLEELIKELAELLKDVIRKLEKTNRRLVKILEDIIRRLKEIS | 3plus1_Key_Cterm_529 | KDFAERRRRELKDKLDRLREEHEEVK |
| | KEAEEELRKGTVEDKDILRDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST(SEQ ID NO: 27203) |
| | LRVIEKILEDNIRLLEELVEVIKEILEKHLRLLEELVRVIERILRE | | |
| | VGKDKDRDHMVLHEYVNAAGITREEHEEVKRPLEEELTRLRETHKK | | |
| | IEKELREALKRVRDRST(SEQ ID NO: 27202) | | |
| 3plus1_Cage_534_GFP11_Cterm | DEDRIIEEIARLLEELLRELLELIKKLIETNPPLNEEHERAVRELA | 3plus1_Key_Cterm_534 | EKIAEEIERELEELPRMIKRLHEDLE |
| | RLLEELLDRLVKKGISDEKLKRIRERLKRALDDLERLHREINKRLE | | RKLKESEDELREIEARLEEKIRRLEE |
| | DLVRELEKLVREILKELKDALEELRRASARAGGEEVLRRLEEIVKK | | KLERKRR(SEQ ID NO: 27207) |
| | LLDLVRRILERLKEIHKDMVRLLRELNERLTPIVEDLVPLIREILR | | |
| | EAGVDERDHMVLHEYVNAAGITIKRLHEDLERKLKESEDELREIEA | | |
| | RLEEKIRRLEEKLERKRR(SEQ ID NO: 27206) | | |
| 3plus1_Cage_534_GFP11_Cterm | DEDRIIEEIARLLEELLRELLELIKKLIETNRRLNEEHERAVRELA | 3plus1_Key_Cterm_534 | EKIAEEIERELEELRRMIKRLHEDLE |
| | RLLEELLDRINKKGISDEKLKPIRERLKRALDDLERLHREINKRLE | | RKLKESEDELREIEARLEEKIRRLEE |
| | DLVRELEKLVREILKELKDALEELRRASARAGGEEVLRRLEEIVKK | | KLERKRR(SEQ ID NO: 27209) |
| | LLDLVRRILERLKEIHKDNVRLLRELNERLTRIVEDLVRLIREILR | | |
| | EAGVDEKIRDHMVLHEYVNAAGITRLHEDLERKLKESEDELREIEA | | |
| | RLEEKIRRLEEKLERKRR(SEQ ID NO: 27208) | | |
| 3plus1_Cage_534_GFP11_Cterm | DEDRIIEEIARLLEELLRELLELIKKLIETNRRLNEEHEPAVRELA | 3plus1_Key_Cterm_534 | EKIAEEIERELEELRRMIKRLHEDLE |
| | RLLEELLDRLVKKGISDEKLKRIRERLKRLDDLERLHREINKRLE | | RKLKESEDELRE1EARLEEKIRRLEE |
| | DLVRELEKLVREILKELKDALEELRRASARAGGEEVLPTLEEIVKK | | KLERKPP(SEQ ID NO: 27211) |
| | LLDLVRRILERLKEIHKDNVRLLRELNERLTRIVEDLVRLIREIL | | |
| | FIAGVDEKTAEETERDHMVLHEYVNAAGTTERKLKESEDELPETEA | | |
| | RLEEKIRRLEEKLERKRR(SEQ ID NO: 27210) | | |
| 3plus1_Cage_Cterm_539 | SEKEKLLKESEEEVRRLRRTLEELLRKYREVLERLRKELREIEERV | 3plus1_Key_Cterm_539 | ERLVKTLIEDVEAVIKRILELITRVA |
| | RDVVRRLKEVLDRKGIDIDTIIKEVEDLLKTVLDRLRELLDKIRRL | | EDNERVLERIIRELTDNLERHLKIVR |
| | TKEAIEVVREIIERIVRHAERVKDELRKEGGDKEKLDRVDRLIKEN | | EIVK(SEQ ID NO: 27213) |
| | TRHLKEILDRIEDLVRRSEKKLRDIIREVRRLIEELRKKAEEIKKG | | |
| | PDERLVKTLIEDVEAVIKRILELITRVAEDNERVLERIIRELTDNL | | |
| | ERHLKIVREIVK(SEQ ID NO: 27212) | | |
| 3plus1_Cage_Cterm_548 | DKAEVLPEALKLLKDLLEELIKIHEESIKRILDLIDTLVKVHEDAL | 3plus1_Key_Cterm_548 | EEIDRELKRVVEEIRRLHEEIKERLD |
| | RALKELLERSGLDERELRKVERMATESLPTIAKLKEELRDLARRSL | | DVARRSEEELRRIIKKLKEVVKEIRK |
| | EKLREDIKPVDDTLRKVEEKVPPTGPSEELIEELIRTIEKLLKEIV | | KLK(SEQ ID NO: 27215) |
| | RINEEVLKAVRELLKTLLKLSEDVVRRIEEILPKGGVPEEIDRELK | | |
| | RVVEELPRLHEETKERLDDVARPSEEELPRIIKKLKEVVKEIRKKL | | |
| | K(SEQ ID NO: 27214) | | |
| 3plus1_Cage_Cterm_556 | SERELIERWLELHKEILRLIRELVERLLKLHREILDTIKKLIRELL | 3plus1_Key_Cterm_556 | DDERRTLTELLKRMEDILEKVERTLK |
| | ELLEDIARKLGLDKEAKDELREIAKRVEDKLEKLERESPEVEEDLK | | KLLDDSARMAEEVKKTLKELLEPSEK |
| | RKLKELTDESDTVEKRVRDVVRRGTQSREEIAEELLRLDRKLLKAV | | VAEDVRK(SEQ ID NO: 27217) |
| | EELLKEILDLNKKLLDDVRAILEETRRVLEKLLDRVRRGERTDDER | | |
| | RTLTELLKRMEDILEKVERTLKKLLDDSARMAEEVKKTLKELLERS | | |
| | EKVAEDVRK(SEQ ID NO: 27216) | | |
| 3plus1_Cage_Cterm_560 | SKKELLEEVVRRAIELLKRHLEKLKRILEEIVRLLEENLEKVERVL | 3plus1_Key_Cterm_560 | EDKLKEIEDELRRLLEELRRLDKAIK |
| | EAILSLLDDLLRRGGDEPAIRTLEDVKRRLPEILERIADENAKAIK | | DRLRELKKDLDEANPPIKETLKKLLR |
| | RLADLLDKLEKRNKEAIERLEEILEELKRVRRDEELLRVLETLLKI | | EVEK(SEQ ID NO: 27219) |
| | IEDILRENTKVLEDLLPINEEILEAYLRVVEELLPLAREILTEIVG | | |
| | DEDKLKEIEDELPRLLEELRRLDKAIKDRLRELKKDLDEANRRIKE | | |
| | TLKKLLREVEK(SEQ ID NO: 27218) | | |
| 3plus1_Cage_568_GFP11_Cterm | KEIEETLKELEDLNREMVETNRRVLEETRPLNKETVDRVKATLDEL | 3p1us1_Key_Cterm_568 | KAVEELEKALEEIKERLKEVIDRYED |
| | AKMLKKLVDDVPKGPTSEELKPILAELEELLARVVRRVEELLKKST | | ELRKLPEETKEKIDKYERKLEEIERR |
| | DLLERAVKDSADALPPSHEVLKEVASRVKRAKDEGLPREEVLRLLR | | ERT(SEQ ID NO: 27221) |
| | ELLERHAKVLKDIVRVSEKLLREHLKVLREIVEVLEELLERILKVI | | |
| | LDTTPDHMVLBEYVNAAGITKPRLKEVIDRYEDELRKLPKEYKEKI | | |
| | DKYERKLEEIERRERT(SEQ ID NO: 27220) | | |
| 3plus1_Cage_568_GFP11_Cterm | KEIEETLKELEDLNREMVETNRRVLEETRRLNKETVDPVKATLDEL | 3plus1_Key_Cterm_568 | KAVEELEKALEEIKRRLKEVIDRYED |
| | AKMLKKLVDDVRKGPTSEELKPLLAELEELLARVVRRVEELLKKST | | ELRKLRKEYKEKIDKYERKLEEIERR |
| | DLLEPAVKDSADALRRSHEVLKEVASPVKRAKDEGLRREEVLRLLR | | ERT(SEQ ID NO: 27223) |
| | ELLERRAKVLKDIVRVSEKLLREHLKVLREIVEVLEELLERILKVI | | |
| | LDTTGGDRDHMVLHEYVNAAGITLKEVIDRYEDELRKIRKEYKEKI | | |
| | DKYERKLEEIERRERT(SEQ ID NO: 27222) | | |
| 3plus1_Cage_Cterm_581 | SALETVKKLLEDSSEKIERIVEEDERVAKESSDRIPPLVEEDKRVA | 3plus1_Key_Cterm_581 | AEAVIKVIEKLIRANKRVWDALLKIN |
| | DEILDDIEKIGDTDTLLKLVEEWSPTSKKLLDDVLKDNKDWSDDSR | | EDLVPVNKTVWKELLRVNEKLARDLE |
| | RLLEEILRVHEELIRRVKEILDREGKPEEVVRELEKVLKESLDTLE | | RVVK(SEQ ID NO: 27227) |
| | EIIRRLDEANAATVKRVADVIRELEDINPKVLEEIKRGSDDAEAVI | | |
| | KVIEKLIPANKRVWDALLKINEDLVRVNKTVWKELLPVNEKLARDL | | |
| | ERVVK(SEQ ID NO: 27226) | | |
| 3plus1_Cage_Cterm_585 | SKEEKLKDDVRAVLEDLDRVLKELEKLSEDNLRELKRVLDRITDLH | 3plus1_Key_Cterm_585 | SKAAEDILPVLEKLVKVSREAIKLIL |
| | RRILDELPKGIGSEELLPRVEKVIKDNLDLLRKLVEEHKESSERDL | | ELSEHHVRVSTRIAPLLLDVARKLAE |
| | KRVEDLVREIKEVLRKLLELEDPGTDIRKIEEEIERLDPKIPKAVE | | VIKEAER(SEQ ID NO: 27229) |
| | ESKDLNERNSERIEEVARRSEELAPRLLKEIRERGDSKAAEDILRV | | |
| | LEKLVKVSREAIKLIIELSEHNVRVSTRIAPILLDVARKLAEVIKE | | |
| | AER(SEQ ID NO: 27228) | | |
| 3plus1_Cage_Cterm_587 | SEIEDVINRLRKILEDLERVSEKLLREIKKILDEAPYLNEEVIKEI | 3plus1_Key_Cterm_587 | IEDLVREVERLIKRTEDSLRELEKTV |
| | KRVLEDAVRVFPDGSGSKEELAKLVEELIEELAKLAKEVDEIHKRI | | RELLKPIKEASDKVREDVDRLIKELK |
| | VERLKALVEDAERINPKIVETLEEIVRGVPSEELKRVVEAIVEVIK | | EAAD(SEQ ID NO: 27231) |
| | EHLKVLADVIRRIIKAIEENAETIKRVLEDIVRVLELVLRGEGSIE | | |
| | DLVREVERLIKRIEDSLRELEKTVRELLKRIKEASDKVREDVDRLI | | |
| | KELKEAAD(SEQ ID NO: 27230) | | |
| 3plus1_Cage_Cterm_605 | SREELLDRILEAIAKILEDLKRLIDENLARLEEVVRELERIIDRNL | 3plus1_Key_Cterm_605 | DEIIRKLDELLKEVENVHKEVKDRIR |
| | KLIREILDELKKGSGSEEILEKIKKVDKEIEDLIRRZLKKLEDLIR | | KLLEDHKRSLDEVKKKLERLDERAKE |
| | ETERRLREILKRIRDLLKEVKDRDKDLERLLEVLEEVLPVIAELAK | | VVEREKK(SEQ ID NO: 27233) |
| | ELLDSLRKVLKVVEEVLPLLNEVNKEVLDVIRELAKDGGSDEIIRK | | |
| | LDELLKEVEKVHKEVKDRIRKDLEDHKRSLDEVKKKLERLLERAKE | | |
| | VVEREKK(SEQ ID NO: 27232) | | |
| 3plus1_Cage_Cterm_607 | SEREELLERIKEILKRVKDKLDEDLKRLKEILEKLKEKADRDLEEL | 3plus1_Key_Cterm_607 | SETAVRAIIRVLEKHLEAVRRVLEEL |
| | RRRIEEVREKLERTGRTDELVKEVLDTVRRNLENLKRLVEDILRKL | | LKVLAEHLETVRELIERLKRVLEEAI |
| | EENVKNLTDLVREILKLITELIKRLEDGGLPKEVLDALRRVLEKLE | | EVVERVAR(SEQ ID NO: 27235) |
| | ELLREILERLKRSLEAVKRKIEELLKELERSLDELRRADERIRKEI | | |
| | GDSETAVRAIIRVLEKHLEAVRRVLEELLKVLAEHLETVRELIERL | | |
| | KRVLEEAIEVVEPVAR(SEQ ID NO: 27234) | | |
| 3plus1_Cage_611_GFP11_Cterm | SLEEITKRLLELVEENLARHEEILRELLELARRLAKEDRDILEEVL | 3plus1_Key_Cterm_611 | ERTLREVVRKVLEEAKRLLDELEEVH |
| | KLIEELLKLLEDNGSSEEDLKRLLKEVIEELRAVVKRVKDKWDEVV | | KRVKKELEDIIEENRRVVKRVRDELR |
| | KRIEDLVKKLKELHDDTLRKLRELVRKIVTDISESGGEAEKVKRVV | | EIKRELDE(SEQ ID NO: 27239) |
| | EKILELVERLAKVVKESVEKLLEILRELAEVSKRVAEALLRLLEEL | | |
| | VRVIRIKDERDHMVLHEYVNAAGITLLDELEEVHKRVKKELEDIIE | | |
| | ENRRVVKRVRDELREIKRELDE(SEQ ID NO: 27238) | | |
| 3plus1_Cage_Cterm_632 | SEKELVDDIRRILEEILRLDRSLLEEVIRLLEENEKLVRRHLKTVI | 3plus1_Key_Cterm_632 | DSLVREVEELIKRIEKKIDDLLKTSR |
| | DILRRVAKLLDENGIRTDEADPVLERLEKAHRELLEDYKPALEKIK | | DLVKPVLDLVDEVVKRVEDLVERVKE |
| | ETLERVLREAEEVVKKIDDALRKLGGSKEVLKRLLEELLRLVEKIA | | KIDT(SEQ ID NO: 27241) |
| | EEIKRLLSELVRVTEELVRINKELLEEAVRVIRKEVGDDSLVREVE | | |
| | ELIKRLEKHIDDLLKTSRDLVKRVLDLVDEVVKRVEDLVERVKEKI | | |
| | DT(SEQ ID NO: 27240) | | |
| 3plus1_Cage_646_GEP11_Cterm | DAEEVVKRLADVLRENDETIRKVVEDLVRIAEENDRLWKKLVEDIA | 3plus1_Key_Cterm_646 | EDVKRALEELVSRLRKLLEDVKKASE |
| | EILRRIVELLRRGGVPEELLDRIAKVVKSIVEKAEKILERLNRVSK | | DIVREVERIVRELAKPSDEILKKLED |
| | AIAEKLKTIVDELNEVSKEIVKRNEDILRKOKDKETVLRALRTLVK | | IVEKLRE(SEQ ID NO: 27245) |
| | EYADLSKEVLERVERIVREYVKLSDEVVKSLAEIVEELIRIIEDLL | | |
| | RKGNRDHMVLHEYVNAAGITRKLLEDVKKASEDIVREVERIVRELA | | |
| | KRSDEILKKLEDIVEKLRE(SEQ ID N: 27244) | | |
| 3plus1_Cage_546_GFP11_Cterm | DAEEVVKRLADVLPENDETIRKVVEDLVPIAFENDRINKKLVEDIA | 3plus1_Key_Cterm_646 | EDVKRALEELVSRLRKLLEDVKKASE |
| | EILPPIVELLRRGGVPEELLDRIAKVVKSIVEKAEKILEPLNRVSK | | DIVREVERIVRELAKRSDEILKKLED |
| | AIAEKLKTIVDELNEVSKEIVKRAEDILPKGKDKETVLRALRTLVK | | IVEKLRE(SEQ ID NO: 27247) |
| | EYADLSKEVLERVERIVREYNKLSDEVVKSLAEIVEELIRIIEDLL | | |
| | RKGNLDEDRDHMVLHEYVNAAGITEDVKKASEDIVREVERIVRELA | | |
| | KRSDEILKKLEDIVEKLPE(SEQ ID NO: 27246) | | |
| 3plus1_Cage_546_GFP11_Cterm | DAEEVVKRLADVLRENDETIPKVVEDLVRIAEENDRLWKKLVEDIA | 3plus1_Key_Cterm_646 | EDVKRALEELVSRLRKLLEDVKKASE |
| | EILRRIVELLRRGGVPEELLDRLAKVVKSIVEKAEKILERLNRVSK | | DIVREVERIVRELAKRSDEILKKLED |
| | AIAEKLKTIVDELNEVSKEIVKRAEDILRKGKDKETVLRALPTLVK | | IVEKLRE(SEQ ID NO: 27249) |
| | EYADLSKEVLERVERIVREYVKLSDEVVKSLAEIVEELIRIIEDLL | | |
| | RKGNLDEDVRDHMVLHETVNAAGITDVKKASEDIVREVERIVRELA | | |
| | KRSDEILKKLEDIVEKLRE(SEQ ID NO: 27248) | | |
| 3plus1_Cage_547_GF211_Cterm | DAEEVVKRLADVLRENDETIRKVVEDLVRIAEENDRLWKKLVEDIA | 3plus1_Key_Cterm_647 | EDVKRALEELVSRLRKLLEDVKKASE |
| | EILRRIVELLRRGGVPEELLDRLAKVVKSIVEKAEKILERLNRVSK | | DIVREVEPIVRELAKRSDEILKKLED |
| | AIAEKLKTIVDELNEVSKEIVKRNEDILRKGKDKETVLRALRTLVK | | IVEKLRE(SEQ ID NO: 27251) |
| | EYADLSKEVLEPVERIVREYVKLSDEVVKSLAEIVEELIRIIEDLL | | |
| | RKGNLRDHMVLHEYVNAAGITKLLEDVKKASEDIVREVERIVRELA | | |
| | KRSDEILKKLEDIVEKLRE(SEQ ID NO: 27250) | | |
| 3plus1_Cage_647_GFP11_Cterm | DAEEVVKPLADVLRENDETIRKVVEDINRIAEENDRLWKKLVEDIA | 3plus1_Key_Cterm_647 | EDVKRALEELVSRLRKLLEDVKKASE |
| | EILRRIVELLRRGGVPEELLDRLAKVVKSIVEKAEKILERLNRVSK | | DIVREVERIVRELAKRSDEILKKLED |
| | AIAEKLKTIVDELNEVSKEIVKRAEDILRKGKDKETVLRALRTLVK | | IVEKLRE(SEQ ID NO: 27253) |
| | EYADLSKEVLERVERIVREYVKLSDEVVKSLAEIVEELIRIIEDLL | | |
| | RKGNLDEDVKRALERDHMVLHEYVNAAGITSEDIVREVERIVRELA | | |
| | KRSDEILKKLEDIVEKLRE(SEQ ID NO: 27252) | | |
| p1us1_Cage_Cterm_547 | DAEEVVKRLADVLRENDETIRKVVEDLVRIAEENDRLWKKLVEDIA | 3plus1_Key_Cterm_647 | EDVKRALEELVSRKRKLLEDVKKASE |
| | EILRRIVELLPRGGVPEELLDRLAKVVKSIVEKAEKILERLNRVSK | | DIVREVERIVRELAKRSDEILKKLED |
| | AIAEKLKTIVDELNEVSKEIVKRAEDILRKGKDKETVLRALRTLVK | | IVEKLRE(SEQ ID NO: 27255) |
| | EYADLSKEVLERVERIVREYVKLSDEVYKSLAEIVEELIRIIEDLL | | |
| | RKGNLDEDVKRALEELVSRLRKLLEDVKKASEDIVREVERIVRELA | | |
| | KRSDEILKKLEDIVEKLRE(SEQ ID NO: 27254) | | |
| 3plus1_Cage_Cterm_653 | DEEETLRRLLERKVELAKEYLDVSKEVIDRTTKLLDEYLKTSKRIV | 3plus1_Key_Cterm_653 | SREALEEARRRLEELLRELNEITKDL |
| | DATVELLERGDLGRDELIKPLAEELERSLRELEEEIKRLKRELEES | | EAKLEKLLRDLNELTKALEEELKRLL |
| | LKKLKEIIDPLAEEAEKLLAVLKRGEGSEEEALRALASLVRELIEV | | DELKERTD(SEQ ID NO: 27257) |
| | LRENDERLRDVLRRLIEALRKNNEILERVLRKLVRAAEERGRDESS | | |
| | REALEEARRRLEELLRELNEITKDLEAKLEKLLRDLNELTKALEEE | | |
| | LKRLLDELKKRTD(SEQ ID NO: 27256) | | |
| 3plus1_Cage_Cterm_658 | DEERIIKTLEDINAELVEDIKRILDKVAELNEPLADAIRKILEETK | 3plus1_Key_Cterm_658 | KDTLRTVEKLVEDVKPRLDKLLEDYK |
| | RILEATTRKVRKDGEISEELLRRLEEKLRKLLEDLERVLAEHEDES | | RLIEEVKKELDKLLKEYEDALREIKK |
| | PRILEEVERLLKRHADASKELLDRARSVARGVKSDKELVDRLKKLI | | RIDE(SFQ ID NO: 27259) |
| | DDSLESVRELIERLKELLDRLVKSVEDLIRTIKELLDRLVEVLREG | | |
| | VSDKDTLRTVEKLVEDVKRRLDKLLEDYKRLIEEVKKELDKLLKEY | | |
| | EDALREIKKRIDE(SEQ ID NO: 27258) | | |
| 3plus1_Cage_Nterm_263 | SLVDELRKSLERNVRVSEEVARRLKEALKRWVDVVRKVVEDLIRLN | 3plus1_Key_Nterm_263 | SLVDELRKSLERNVRVSEEVARRLKE |
| | EDVVRVVEKVTVDESAIERVRRIIEELNRKLDAVLKKNEDLVRRLT | | ALKRWVDVVRKVVEDLIPLNEDVVRV |
| | ELLDKLLEENRRLVEELDEDLKRRGGTEEVIDTILELIERSIERLK | | VEKV(SEQ ID NO: 27263) |
| | RLLDELLRIVREALKDNKRVADENLKKLKEILDELRKDGVEDEELK | | |
| | RVLEKAADLHRRLKDPHRKLLEDLERIIRELKKKLDEVVEENKRSV | | |
| | DELKR(SEQ ID NO: 27262) | | |
| 3plus1_Cage_647_GFP11_Nterm | DAEEVVKRLADVLRENDETIRKVVEDLVRIAEENDPLWRDHMVLHE | 3plus1_Key_Nterm_647 | DAEEVVKRLNDVLRENDETIRKVVED |
| | YVNAAGITLLRRGGVPEELLDRLAKVVKSIVEKAEKILERLNRVSK | | LVRIAEENDRLWKKLVEDIAEILRRI |
| | AIAEKLKTIVDELNEVSKEIVKRAEDILRKGKDKETVLRALPTLVK | | VELLRRG(SEQ ID NO: 27277) |
| | EYADLSKEVLERVERIVREYVKLSDEVVKSIAEIVEELTRIIEDLL | | |
| | RKGNLDEDVKRALEELVSRLRKLLEDVKKASEDIVREVERIVRELA | | |
| | KPSDEILKKLEDIVEKLRE(SEQ ID NO: 27276) | | |

TABLE 6
Mapping Switch to Key that activates it

| Row | Cage polypeptide | Key polypeptide |
|---|---|---|
| 1 | 1fix-short_cODC_t11, | key_a |
| | (SEQ ID NO: 27, 373) | (SEQ ID NO: 27, 477), |
| | 1fix-short_cODC_t8 | key_a_m4 |
| | (SEQ ID NO: 27, 372), | (SEQ ID NO: 27, 478), |
| | 1fix-short_cODC_t5 | key_a_m9 |
| | (SEQ ID NO: 27, 371), | (SEQ ID NO: 27, 479), |
| | 1fix-short_cODC | key_a_m12 |
| | (SEQ ID NO: 27, 370), | (SEQ ID NO: 27, 480), |
| | 1fix-short_CODC_mut_t6 | key_a_m15 |
| | (SEQ ID NO: 27, 369), | (SEQ ID NO: 27, 481) |
| | 1fix-short_cODC_mut | |
| | (SEQ ID NO: 27, 368), | |
| | degron-miniLOCKRa_t12 | |
| | (SEQ ID NO: 27, 367), | |
| | degron-miniLOCKRa_2_t9 | |
| | (SEQ ID NO: 27, 366), | |
| | degron-miniLOCKRa_1_t12 | |
| | (SEQ ID NO: 27, 365), | |
| | degron-miniLOCKRa_1_t9 | |
| | (SEQ ID NO: 27, 364), | |
| | degonLOCKRa_320_t16 | |
| | (SEQ ID NO: 27, 363), | |
| | degonLOCKRa_324_t12 | |
| | (SEQ ID NO: 21, 362), | |
| | degonLOCKRa_CAonly | |
| | (SEQ ID NO: 27, 361), | |
| | degonLOCKRa_327_noPro | |
| | (SEQ ID NO: 27, 360), | |
| | degonLOCKRa_327 | |
| | (SEQ ID NO: 27, 359) | |
| 2 | degonLOCKRb, | key_b |
| | (SEQ ID NO: 27, 374) | (SEQ ID NO: 27, 482) |
| | degonLOCKRb_t13 | |
| | (SEQ ID NO: 27, 375) | |
| 3 | degonLOCKRc_t13 | key_c |
| | (SEQ ID NO: 27, 376), | (SEQ ID NO: 27, 483) |
| | degron-miniLOCKRc_1_t9 | |
| | (SEQ ID NO: 27, 378), | |
| | degron-miniLOCKRc_1_t13 | |
| | (SEQ ID NO: 27, 379), | |
| | degron-miniLOCKRc_2_t9 | |
| | (SEQ ID NO: 27, 380), | |
| | degron-miniLOCKRc_t13 | |
| | (SEQ ID NO: 27, 331), | |
| | degronLOCKRc_1fix_t13 | |
| | (SEQ ID NO: 27, 382) | |
| 4 | degonLOCKRd SEQ ID | key_d |
| | NO: 27, 383 | (SEQ ID NO: 27, 484), |
| | | key_d_m4 |
| | | (SEQ ID NO: 27, 4851, |
| | | key_d_m7 |
| | | (SEQ ID NO: 27, 477) |

TABLE 7

| | | | |
|---|---|---|---|
| degronSwitch_cODC_2plus1_5398_t15 | SVEELLRKLEEVLRKIREENERSLKELRDRAREIVKRN | 2plus1_Key_Cterm_5398 | EDIVRKIERIVETIEREVRESVKKVEEIARDIRR |
| | RETNRELEEVIKELEKRLSGADKEKVEELVRRIRRIVE | | KVDESVKKIVEKLLRDVDKKARDRKK (SEQ ID |
| | RVVEEDRRTVEEIEKIAREVVKRDRDSADPVRRTVEDV | | NO: 27129) |
| | LPKATGSEDIVRKIERIVETTEREVRELIMSCAQESRD | | |
| | IRRKVDESVYNVE SEQ ID NO: 27384) | | |
| degronSwitch_cODC_2plus1_5398_t12 | SVEELLRKLEEVLPEIREENERSLKELRDRAREIVKRN | 2plus1_Key_Cterm_5398 | EDIVRKIERIVETIEREVRESVKKVEEIARDIRR |
| | RETNRELEEVIKELEKRLSGADKEKVEELVRRIRRIVE | | KVDESVKNVEKLLRDVDKKARDRKK (SEQ ID |
| | RVVEEDRRTVEEIEKIAREVVKRDRDSADPYRRTVEDV | | NO: 27129) |
| | LRKATGSEDIVRKIERIVETIEREVRESVKLVMSCAQE | | |
| | SRRKVDESVYNVEKLL (SEQ ID NO: 27385) | | |
| degronSwitch_cODC_2plus1_5398_t8 | SVEELLRKLEEVLRKIREENERSIKELRDPAREIVKRN | 2plus1_Key_Cterm_5398 | EDIVRKIERIVETIEREVRESVKKVEEIARDIRR |
| | RETNRELEEVIKELEKRLSGADKEKVEELVRRIRRIVE | | KVDESVKNVEKLIRDVDKKARDRKK (SEQ ID |
| | RVVEEDRRTVEEIEKIAREVVKRDRDSADPVRRTVEDV | | NO: 27129) |
| | LPKATGSEDIVRKIERIVETIEREVRESVKKVEELAMS | | |
| | CAQESDESVYNVEKLLRDVD (SEQ ID NO: 21386) | | |
| degronSwitch_cODC_2plus1_5398_t4 | SVEELLRKLEEVLRKIREENERSIKELRDPAREIVKRN | 2plus1_Key_Cterm_5398 | EDIVRKIERIVETIEREVRESVKKVEEIARDIRR |
| | RETNRELEEVIKELEKRLSGADKEKVEELVRRIRRIVE | | KVDESVKNVEKLLRDVDKKARDRKK (SEQ ID |
| | RVVEEDRRTVEEIEKIAREVVKRDRDSADPVRRTVEDV | | NO: 27129) |
| | LRKATGSEDIVRKIERIVETIEREVRESVKKVEEIARD | | |
| | LRMSCAQESKNVEKLLRDVDKKAR (SEQ ID | | |
| | NO: 27387) | | |
| degronSwitch_cODC_2plus1_5398_t1 | SVEELLRKLEEVLRKIREENERSIKELRDRAREIVKRN | 2plus1_Key_Cterm_5398 | EDIVRKIERIVETIEREVRESVKKVEEIARDIRR |
| | RETNRELEEVIKELEKRLSGADKEKVEELVRRIRRIVE | | KVDESVKNVEKLLRDVDKKARDRKK (SEQ ID |
| | RVVEEDRRTVEEIEKIAREVVKRDRDSADRVRRTVEDV | | NO: 27129) |
| | LRKATGSEDIVRKIERIVETIEREVRESVKKVEEIARD | | |
| | IRRLGMSCAQESEKLLRDVDKKARDRK (SEQ ID | | |
| | NO: 27388) | | |
| degronSwitch_cODC_2plus1_5398_t0 | SVEELLRKLEEVLRKIREENERSLKELRDRAPEIVKRN | 2plus1_Key_Cterm_5398 | EDIVRKIERIVETIEREVRESVKKVEEIARDIRR |
| | RETNRELEEVIKELEKRLSGADKEKVEELVRRIRRIVE | | KVDESVKNIVEKLLRDVDKKARDRKK (SEQ ID |
| | RVVEEDRRTVEEIEKIAREVVKRDRDSADRVRRTVEDV | | NO: 27129) |
| | LRKATGSEDIVRKIERIVETIEREVPESVKKVEEIARD | | |
| | IRRKLDMSCAQESKLLPDVDKKARDRKK (SEQ ID | | |
| | NO: 27389) | | |
| degronSwitch_cODC_2plus1_5405_t15 | SESDDVIRKLRELLEELRTEVEKSIPDLRKILEDSTRH | 2p1us1_Key_Cterm_5405 | EEKLKDLIRKLRDILRPAAEAHKKIJDDARESLE |
| | AKRSIEELERLLEEVRKKPGDEEVRKTVEEISRRVAEN | | RAKREHEKLIDPLKKILEELER (SEQ ID |
| | VKRLEDLYRRMEEEVKKNLDRLRKRVEDIIREVEEARK | | NO: 27131) |
| | KGVDEEKLKDLIRKLRDILRRLAMSCAQESDDARESLE | | |
| | RAKREHE (SEQ ID NO: 27424) | | |
| degronSwitch_cODC2plus1_5405_t12 | SESDDVIRKLRELLEELRTHVEKSIRDLRKILEDSTRH | 2p1us1_Key_Cterm_5405 | EEKLKDLIRKLRDILPPAAEAHKKLIDDARESLE |
| | AKRSIEELERLLEEVPKKPGDEEVRKTVEEISRRVAEN | | RAKREHEKLIDPLKKILEELER (SEQ ID |
| | VKRLEDLYRRMEEENKKNLDRLRKPVEDIIREVEEARK | | NO: 27131) |
| | KGVDEEKLKDLIRKLRDILRRAAELHMSCAQESRESLE | | |
| | RAKREHEKLI (SEQ ID NO: 27425) | | |
| degronSwitch_cODC_2plus1_5405_t11 | SESDDVIRKLRELLEELRTHVEKSIRDLRKILEDSTRH | 2p1us1_Key_Cterm_5405 | EEKLKDLIRKLRDILRRAAEAHKKLIDDARESLE |
| | AKRSIEELERLLEEVRKKPGDEEVRKTVEEISPPVAEN | | RAKREHEKLIDREKKILEELER (SEQ ID |
| | VKRLEDLYRRMEEEVKKNLDRLRKPVEDIIREVEEARK | | NO: 27131) |
| | KGVDEEKLKDLIRKLPDILPRAAEALKMSCAQESESLE | | |
| | RAKREHEKLID (SEQ ID NO: 27426) | | |
| degron5witch_cODC_2plus1_5405_t8 | SESDDVIRKLPELLEELRTHVEKSIRDLRKILEDSTRH | 2plus1_Key_Cterm_5405 | EEKLKDLIRKLRDILPPAAEATIKKLIDDARESLE |
| | AKRSIEELERLLEEVRKKPGDEEVRKTVEEISRRVAEN | | RAKREHEKLIDPLKKILEELER (SEQ ID |
| | VKRLEDLYRRMEEEVKKNLDRLRKPVEDIIREVERARK | | NO: 27131) |
| | KGVDEEKLKDLIRKLRDILRRAAEAHKKLIMSCAQESE | | |
| | RAKREHEKLIDRLK (SEQ ID NO: 27427) | | |
| degronSwitch_cODC_2plus1_5405_t4 | SESDDVIRKLPELLEELRTEVEKSIRDLRKILEDSTRH | 2plus1_Key_Cterm_5405 | EEKLKDLIRKLRDILRRAAEAHKKLIDDARESLE |
| | AKPSIEELERLLEEVRKKPGDEEVRKTVEEISRRVAEN | | RAKREHEKLIDRLKKILEELER (SEQ ID |
| | VKRLEDLYRRMEEEVXKNLDRLRKPVEDIIREVERARK | | NO: 27131) |
| | KGVDEEKLKDLIRKLRDILRRAAEAHKKLIDDLRMSCA | | |
| | QESREHEKLIDRLKKILE (SEQ ID NO: 27428) | | |
| degronSwitch_cODC_2plus1_5406_t15 | DREREVKKRLDEVRERIERLLRRVEEESRRVAEEIRRL | 2plus1_Key_Cterm_5406 | EELREELKKLERKIEKVAKEIHDHDKEVTERLED |
| | IEEVRRRNKKVTEEIRELLKGLKDKEEVRRVLEPLRKL | | LLRRITEHARKSDREIEETAR (SEQ ID |
| | NAESDELLERILERLRRLVEATNPLVKAIIEELRRLVE | | NO: 27133) |
| | KIVREVPDSEELREELKKLERKIEKLAMSCAQESKEVT | | |
| | ERLEDLLRRIT (SEQ ID NO: 27430) | | |
| degronSwitch_cODC_2plus1_5406_t11 | DREREVKKRLDEVREPIERLLRRVEEESRRVAEEIRRL | 2plus1_Key_Cterm_5406 | EELREELKKLERKIEKVAKEIHDHDKEVTERLED |
| | IEEVRRRNKKVTEEIPELLKGLKDKEEVRRVLERLRKL | | LLRRITEHARKSDREIEETAR (SEQ ID |
| | NAESDELLERILERLRRLVEATNRINKAIIEELRRLVE | | NO: 27133) |
| | KIVREVPDSEELREELKELERKIEKVAKELKMSCAQES | | |
| | ERLEDLLRRITEHAR (SEQ ID NO: 27431) | | |
| degronSwitch_cODC_2plus1_5406_t4 | DREREVKKPLDEVRERIERLLRPVEEESRRVAEEIRRL | 2plus1_Key_Cterm_5406 | EELREELKKLERKIEKVAKEIHDHDKEVTERLED |
| | IEEVRRRNKKVTEEIRELLKGLKDKEEVRRVLEPLRKL | | LLRRITEHARKSDREIEETAR (SEQ ID |
| | NAESDELLERILERLRRLVEATNRLVKAIIEELRRLVE | | NO: 27133) |
| | KIVREVPDSEELREELKKLERKIEKVAKEIHDHDKELE | | |
| | MSCAQESPPITEHARKSDREIE (SEQ ID | | |
| | NO: 27432) | | |
| degronSwitch_cODC_2plus1_5409_t15 | SEAEELLKRLEDRAEEILRRLEEILRTSRKLAEDVLRE | 2plus1_Key_Cterm_5409 | KEVVDEIKRIVDEVRERLKRIVDENAKIVEDARR |
| | LEKLLRESEPRIREVLEELRGIKDKKELEDVIREVEKE | | ALEKIVKENEEILRRLKKELRELRK (SEQ ID |
| | LDESLERSRELLKDVLKKLDDNLKESERLVEDIDRELA | | NO: 27135) |
| | KILEDLKKAGVPKEVVDEIKRIVDEVRERLKRLVMSCA | | |
| | QESEDARRALEKIVKENE (SEQ ID NO: 27440) | | |
| degronSwitch_cODC_2plus1_5409_t12 | SEAEELLKRLEDRAEEILRRLEEILRTSRKLAEDVLRE | 2plus1_Key_Cterm_5409 | KEVVDEIKRIVDEVRERLKRIVDENAKIVEDARR |
| | LEKLLRESERRIREVLEELRGIKDKKELEDVIREVEKE | | ALEKIVKENEEILRRLKKELRELRK (SEQ ID |
| | LDESLERSRELLKDVLKKLDDNLKESERLVEDIDRELA | | NO: 27135) |
| | KILEDLKKAGVPKEVVDEIKRIVDEVRERLKRIVDLTM | | |
| | SCAQESRRALEKIVKENEEIL (SEQ ID | | |
| | NO: 27441) | | |
| degronSwitch_cODC_2plus1_5409_t11 | SEAEELLKRLEDRAEEILRRLEEILRTSRKLAEDVLRE | 2(p1us1_Key_Cterm_5409 | KEVVDEIKRIVDEVRERLKRIVDENAKIVEDARR |
| | LEKLLRESERRIREVLEELRGIKDKKELEDVIREVEKE | | ALEKIVKENEEILRRLKKELRELRK (SEQ ID |
| | LDESLERSRELLKDVLKKEDDNLKESERLVEDIDRELA | | NO: 27135) |
| | KILEDLKKAGVPKEVVDEIKRIVDEVRERLKRIVDELR | | |
| | MSCAQESRALEKIVKENEEILR (SEQ ID | | |
| | NO: 27442) | | |
| degronSwitch_cODC_2plus1_5409_t8 | SEAEELLKRLEDRAEEILRRLEEILRTSRKLAEDVLRE | 2plus1_Key_Cterm_5409 | KEVVDEIKRIVDEVRERLKRIVDENAKIVEDARR |
| | LEKLLRESERRIREVLEELRGIKDKKELEDVIREVEKE | | ALEKIVKENEEILRRLKKELRELRK (SEQ ID |
| | LDESLERSRELLKDVLKKLDDNLKESERLVEDIDRELA | | NO: 27135) |
| | KILEDLKKAGVRKEVVDEIKRIVDEVRERLKRIVDENA | | |
| | KLTMSCAQESEKIVKENEEILRRLK (SEQ ID | | |
| | NO: 27443) | | |
| degronSwitch_cODC_2plus1_5409_t0 | SEAEELLKRIEDRAEEILRRLEEILRTSRKLAEDVLRE | 2plus1_Key_Cterm_5409 | KEVVDEIKRIVDEVRERLKRIVDENAKIVEDARR |
| | LEKLLRESERRIREVLEELRGIKDKKELEDVIREVEKE | | ALEKIVKENEEILRRLKKELRELRK (SEQ ID |
| | LDESLERSRELLKDVLKKLDDNLKESERLVEDIDRELA | | NO: 27135) |
| | KILEDLKKAGVPKEVVDEIKRIVDEVRERLKRIVDENA | | |
| | KIVEDARRALEMSCAQESEILRRLKKELRELRK (SEQ | | |
| | ID NO: 27444) | | |
| degronSwitch_cODC_2plus1_5410_t12 | SRAETVLKEVTDKIKKLADSSDELLRRNKENIDELKKS | 2plus1_Key_Cterm_5410 | DEVVKRVRDLLDTVRRRNEKVNEDVKRMNDKLRR |
| | SEELLRRITKAIEEIEKGSVDEETLEELIRRVEAELEA | | DNEDVIRRVEKLLRELEEKRRT (SEQ ID |
| | HHRELEKNSREDEKRNRDHHAKLEEEMRRVEERLEREG | | NO: 27137) |
| | IDDEVVKPVRDLLDTVRRRNEKLMMSCAQESDKLRRDN | | |
| | EDVIRRVE (SEQ ID NO: 27445) | | |
| degronSwich_c0DC_2plus1_5410_t5 | SRAETVLKEVTDKIKKLADSSDELLRRNKENIDELKKS | 2plus1_Key_Cterm_5410 | DEVVKPVRDLLDTVRRRNEKVNEDVKRMNDKLRR |
| | SEELLPRLTKAIEEIEKGSVDEETLEELTPRVEAELEA | | DNEDVIRRVEKELEEKRRT (SEQ ID |
| | HHRELEKNSREDEKRNRDHHAKLEEEMRRVEERLEREG | | NO: 27137) |
| | IDDEVVERVRDLLDTVRRRNEKVNEDVKRLNMSCAQES | | |
| | EDVIRRVEKLLRELE (SEQ ID NO: 27446) | | |
| degronSwitch_cODC_2plus1_5410_t1 | SRAETVLKEVTDKIKKLADSSDELLRRNKENIDELKKS | 2plus1_Key_Cterm_5410 | DEVVKRVRDLLDTVRRRNEKVNEDVKRMNDKLRR |
| | SEELLRRLTKAIEEIEKGSVDEETLEELIRRVEAELEA | | DNEDVIRRVEKLLRELEEKRRT (SEQ ID |
| | HHRELEKNSREDEKRNRDHHAKLEEEMRRVEERLEREG | | NO: 27137) |
| | IDDEVVKRVPDLLDTVRRRNEKVNEDVKRMNDKLRMSC | | |
| | AQESRRVEKLLRELEEKRR (SEQ ID NO: 27447) | | |
| degronSwitch_cODC_2plus1_5413_t15 | STEEVLDEIRKLHKTLTEDIKRVLREIEELHRRTIEEN | 2plus1_Key_Cterm_5413 | AEELLRESKEAIKEVKRVLEELRKESKRVVDETR |
| | KEVLDKIAEDYKRVIDDVRTKDTPNVEKLLKDLEKSAK | | KLSEENLEHSERVLRKVEEDLR (SEQ ID |
| | ENIEHNERTLREDDRVLKEIRPRATELLKANEEMLRRI | | NO: 27139) |
| | EEVARKGGVDAEELLRESKEAIKEVKRLLMSCAQESKR | | |
| | VVDETRKLSEENL (SEQ ID NO: 27455) | | |
| degronSwitch_cODC_2plus1_5413_t11 | STEEVLDEIRKLHKTLTEDIKRVLREIEELHRRTIEEN | 2plus1_Key_Cterm_5413 | AEELLRESKEAIKEVKRVIEELRKESKRVVDETR |
| | KEVLDKIAEDYKRVIDDVRTKDTPNVEKLLKDLEKSAK | | KLSEENLEHSERVLRKVEEDLR (SEQ ID |
| | ENIEHNERTLREDDRVLKEIRRRATELLKANEEMLRRI | | NO: 27139) |
| | EEVARKGGVDAEELLRESKEAIKEVKRVLEELKMSCAQ | | |
| | ESDETRKLSEENLEHSE (SEQ ID NO: 27456) | | |
| degronSwitch_cOIDC_2plus1_5413_t7 | STEEVLDEIRKLHKTLTEDIKRVLREIEELHRRTIEEN | 2plus1_Key_Cterm_5413 | AEELLRESKEAIKEVKPVLEELRKESKRVVDETR |
| | KEVLDKIAEDYKRVIDDVRTKDTPNVEKLLKDLEKSAK | | KLSEENLEHSERVLRKVEEDLR (SEQ ID |
| | ENIEHNERTLREDDRVLKEIRRRATELLKANEEMLRRI | | NO: 27139) |
| | EEVARKGGVDAEELLRESKEAIKEVKRVLEELRKELEM | | |
| | SCAQESKLSEENLEHSERVLR (SEQ ID | | |
| | NO: 27457) | | |
| degronSwitch_cODC_2plus1_5413_t4 | STEEVLDEIRKIHKTLTEDIKRVLREIEELHRRTIEEN | 2plus1_Key_Cterm_5413 | AEELLRESKRAIKEVKRVLEELRKESKRVVDETR |
| | KEVLDKIAEDYKRVIDDVRTKDTPNVEKLLKDLEKSAK | | KLSEENLEHSERVLRKVEEDLR (SEQ ID |
| | ENIEHNEPTLPEDDRVLKEIRRPATELLKANEEMLRRI | | NO: 27139) |
| | EEVARKGGVDAEELLPESKEAIKEVKRVLEELPKESKR | | |
| | LTMSCAQESEENLEHSERVLRKVE (SEQ ID | | |
| | NO: 27458) | | |
| degronSwitch_cODC_2plus1_5413_t0 | STEEVLDEIRKLHKTLTEDIKRVLREIEELHRRTIEEN | 2plus1_Key_Cterm_5413 | AEELLRESKEAIKEVKRVLEELRKESKRVVDETR |
| | KEVLDK1AEDYKRVIDDVRTKDTPNVEKLLKDLEKSAK | | KLSEENLEHSERVLRKVEEDLR (SEQ ID |
| | ENIEHNERTLREDDRVLKEIRRRATELLKANEEMLRRI | | NO: 27139) |
| | EEVARKGGVDAEELLRESKEAIKEVKRVLEELRKESKR | | |
| | VVDELRMSCAQESEHSERVLRKVEEDLR (SEQ ID | | |
| | NO: 27459) | | |
| degronSwitch_cODC_2plus1_5414_t15 | SEIEKILKEIEDLARRDEEVSKKIVEDIRRIAKEVEDT | 2plus1_Key_Cterm_5414 | EDSERLVREVEDLVRPLVPRSEKSNEEVKRTVEE |
| | SRDIVRKIEELAKRVIDRLRKDGSKEELEKEVREVVKT | | LVRRMEESNDPVRDLVRRLVEELKRAVD (SEQ |
| | LEELVKDNHRLIRRLVEEMKRLVEENHRNSREVVKELE | | ID NO: 27141) |
| | DLVRELPRKGSGSEDSERLREVEDLVRRINRRSEKLDM | | |
| | SCAQESEELVRRMEESNDRVR (SEQ ID | | |
| | NO: 27460) | | |
| degronSwitch_cODC_2plus1_5414_t11 | SEIEKILKEIEDLARRDEEVSKKIVEDIRRLAKEVEDT | 2plus1_Key_Cterm_5414 | EDSERLVREVEDLVRRLVRRSEKSNEEVKRTVEE |
| | SRDIVRKIEELAKRVLDRLRKDGSKEELEKEVPEVVKT | | LVRRMEESNDRVRDLVRRLVEELKRAVD (SEQ |
| | LEELVKDNHRLIRRAVEEMKRLVEENHRHSREVVKELE | | ID NO: 27141) |
| | DLVRELRKGSGSEDSERLVREVEDLNRRLVPRSEKSNE | | |
| | ELKMSCAQESRRMEESNDRVRDLVR (SEQ ID | | |
| | NO: 27461) | | |
| degronSwitch_cODC_2plus1_5414_t7 | SEIEKILKEIEDLAPPDEEVSKKIVEDIRRLAKEVEDT | 2plus1_Key_Cterm_5414 | EDSERLVREVEDLVRPIVRRSEKSNEEVKRTVEE |
| | SRDIVRKIEELAKRVLDRLRKDGSKEELEKEVREVVKT | | LVRRMEESNDRVRDLVRRLMEELKRAVD (SEQ |
| | LEELVKDNHRLIRRAVEHMKRLVEENHRHSREVVKELE | | ID NO: 27141) |
| | DLVRELRKGSGSEDSERLVREVEDLVRRLVRRSEKSNE | | |
| | EVKRTLEMSCAQESESNDRVRDLVRRLVE (SEQ ID | | |
| | NO: 27462) | | |
| degronSwitch_cODC_2plus1_5414_t4 | SEIEKILKEIEDLARRDEEVSKKIVEDIRRLAKEVEDT | 2plus1_Key_Cterm_5414 | EDSERLVREVEDLVRALVRRSEKSNEEVKRTVEE |
| | SRDIVRKIEELAKPVLDPLRKDGSKEELEKEVREVVKT | | LVRRMEESNDRDLVRRLVEELKRAVD (SEQ |
| | LEELVKDNHRLIRRIVEEMKRLVEENHRHSREVVKELE | | ID NO: 27141) |
| | DLVRELRKGSGSEDSERLVREVEDLVRRINRRSEKSNE | | |
| | EVKRTVEELVMSCAQESDRVRDLVRRLVEELK (SEQ | | |
| | ID NO: 27463) | | |
| degronSwitch_cODC_2plus1_5414_t0 | SEIEKILKEIEDLARRDEEVSKKIVEDIRRLAKEVEDT | 2plus1_Key_Cterm_5414 | EDSERLVREVEDLVRRIVRRSEKSNEEVKRTVEE |
| | SRDIVRKIEELAKRVLDRLRKDGSKEELEKEVREVVKT | | LVRRMEESNDRVRDLVRRLVEELKRAVD (SEQ |
| | LEELVKDNHRLIRRAVEEMKRLVEENHRHSREVVKELE | | ID NO: 27141) |
| | DLVRELREGSGSEDSERLVREVEDLVRRINRRSEKSNE | | |
| | EVKRTVEELVRPLLMSCAQESDLVRRLVEELKRAVD | | |
| | (SEQ ID NO: 27464) | | |
| degronSwitch_cODC_2plus1_5421_t11 | SVDEVLKEIEDALRRLKEEVERVLKENEDELRRLEEEV | 2plus1_Key_Cterm_5421 | EKAIRDVAKEIRDPIKELEEEIEEVTRRNLKLLA |
| | RRVLKEDEELLESLKRGVGESDEVDRVVDEIAKLSAEI | | DVEEEIRRVHEKTRRLLETVLRRAT (SEQ ID |
| | LEKVKKVVKEIRDSLETVKRPMDDVVRRLKELIDEIKR | | NO: 27147) |
| | GSDEKTURDVAKEIRDRLKELEEEIEELIMSCAQESAD | | |
| | VEEEIRRVHEKTR (SEQ ID NO: 27492) | | |
| degronSwitch_cODC_2plus1_5421_t7 | SVDEVLKEIEDALRRLKEEVERVLKENEDELRRLEEEV | 2plus1_Key_Cterm_5421 | EKAIRDVAKEIRDRLKELEEEIEEVTRRNLKLLA |
| | RRVLKEDEELLESLKRGVGESDEVDPVVDEIAKLSAEI | | DVEEEIRRVHEKTRRLLETVLRRAT (SEQ ID |
| | LEKVKKVVKEIRDSLETVKPRVDDVVRRLKELLDEIKR | | NO: 27147) |
| | GSDEKAIRDVAKEIRDRLKEIEEEIEEVTRRLLMSCAQ | | |
| | ESEEIRPVHEKTRRLLE (SEQ ID NO: 27493) | | |
| degronSwitch_cODC_2plus1_5421_t4 | SVDEVLKEIEDALRPLKEEVERVLKENEDELRRLEEEV | 2plus1_Key_Cterm_5421 | EKAIRDVAKEIPDRLKEIEEEIREVTRRNLKLLA |
| | RRVLKEDEELLESLKRGVGESDEVDRVVDEIAKLSAEI | | DVEEEIRRVHEKTRRLLETVLRRAT (SEQ ID |
| | LEKYKKVVKEIRDSLETVKPRVDDVVRRLKELLDEIKR | | NO: 27147) |
| | GSDEKAIRDVAKEIRDRLKELEEEIEEVTRRNLKLLMS | | |
| | CAQESRRVHEKTRRLLETVL (SEQ ID NO: 27494) | | |
| degronSwitch_cODC_2plus1_5421_t0 | SVDEVLKEIEDALRRLKEEVERVLKENEDELRRLEEEV | 2plus1_Key_Cterm_5421 | EKAIRDVAKEIRDRLKELEEEIEEVTRRNLKLLA |
| | RRVLKEDEELLESLKRGVGESDEVDRVVDEIAKLSAEI | | DVEEEIRRVEEKTRRLLETVLRRAT (SEQ ID |
| | LEKVKKVVKEIRDSLETVKPRVDDVVRRLKELIDEIKR | | NO: 27147) |
| | GSDEKAIRDVAKEIRDRLKELEEEIEEVTRRNLKLLAD | | |
| | LEMSCAQESEKTRRLLETVLRRAT (SEQ ID | | |
| | NO: 27495) | | |
| degronSwitch_cODC_2plus1_5432_t15 | DEIRKVVKEITDLLKASNDKNRKVVEEIRDLLRKSKKL | 2plus1_Key_Cterm_5432 | SEDLKRVEERAREVSPRNEESMRRVKEDADRVSE |
| | ADELVERLRALVEDLPPRIDKSGDKETAEDIVPRIIEE | | ANKEVLDPVREEVKRLIEEVRETLR (SEQ ID |
| | LKRILKEIEDLARRINREIERLVEEVERDNRDVNRAIE | | NO: 27149) |
| | ELLKDIARRGGSEDIKRVEEPAREVSRRNEELMMSCAQ | | |
| | ESDRVSEANKEVLDRVR (SEQ ID NO: 27543) | | |
| degronSwitch_cODC_2plus1_5432_t11 | DEIRKVVKEITDLLKASNDKNRKVVEEIRDLLRKSKKL | 2plus1_Key_Cterm_5432 | SEDLKRVEEPAREVSRRNEESMRPVKEDADRVSE |
| | ADELVERLRALVEDLRRRIDKSGDKETAEDIVRRIIEE | | ANKEVLDRVREEVKRLIEEVRETLR (SEQ ID |
| | LKRILKEIEDLARRINREIERLVEEVERDNRDVNRAIE | | NO: 27149) |
| | ELLKDIARRGGSEDLKRVEERAREVSRRNEESMRRLKM | | |
| | SCAQESEANKEVLDRVREEVK (SEQ ID | | |
| | NO: 27544) | | |
| degronSwitch_cODC_2plus1_5432_t8 | DEIRKVVKEITDLLKASNDKNRKVVEEIRDLLRKSKKL | 2plus1_Key_Cterm_5432 | SEDLKRVEERAREVSPRNEESMRRVKEDADRVSE |
| | ADELVERLRALVEDLRRRIDKSGDKETAEDIVRRIIEE | | ANKEVLDRVREEVKRLIEEVRETLR (SEQ ID |
| | LKRILKEIEDLNRRINREIERLVEEVERDNRDVNRAIE | | NO: 27149) |
| | ELLKDIAPRGGSEDLKRVEERAREVSRRNEESMRRVKE | | |
| | LAMSCAQESKEVLDRVREEVKRLI (SEQ ID | | |
| | NO: 27545) | | |
| degronSwitch_cODC_2plus1_5432_t4 | DEIRKVVKEITDLLKASNDKNRKVVEEIRDLLRKSKKL | 2plus1_Key_Cterm_5432 | SEDLKRVEERAREVSRRNEESMRPYKEDADRVSE |
| | ADELVERLRALVEDIRRRIDKSGDKETAEDIVRRIIEE | | ANKEVLDRVREEVKRLIEEVRETLR (SEQ ID |
| | LKRILKEIEDLARRINREIERLVEEVERDNRDVNRAIE | | NO: 27149) |
| | ELLKDIARRGGSEDLKRVEERNREVSRRNEESMRRVKE | | |
| | DADRLKMSCAQESDRVREEVKRLIEEVR (SEQ ID | | |
| | NO: 27546) | | |
| degronSwitch_cODC_2plus1_5432_t1 | DEIRKVVKEITDLLKASNDKNRKVVEEIRDLLRKSKKL | 2plus1_Key_Cterm_5432 | SEDLKRVEERAREVSRRNEESMRPVKEDADRVSE |
| | ADELVERLRALVEDLPPPIDKSGDKETAEDIVPRIIEE | | ANKEVLDRVREEVKPLIEEVRETLR (SEQ ID |
| | LKRILKEIEDLARRINREIEPLVEEVERDNRDVNRAIE | | NO: 27149) |
| | ELLKDIARRGGSEDLKRVEERAREVSRRNEESMPRVKE | | |
| | DADRVSELTMSCAQESREEVKPIIEEVRETL (SEQ | | |
| | ID NO: 27547) | | |
| degronSwitch_cODC_2plus1_5435_t15 | STAETVAEEVERVLKHSDDLIKEVEDVNRRVEEEIKRV | 2plus1_Key_Cterm_5435 | EEAAREIIKRLREVNKRTKEKLDELIKHSEEVLE |
| | IRELEEENERLVAEVRKGVKGEILAEIEKRLADNSEKV | | RVKRLIDELRKHSEEVLEDLRRRAK (SEQ ID |
| | REVAERAKKLLEENTARVKDILRESRKLVKDILDEVRG | | NO: 27151) |
| | TGSEEAAREIIKRLREVNKRTKEILMSCAQESEEVLER | | |
| | VKRLIDELR (SEQ ID NO: 27562) | | |
| degronSwitch_cODC_2plus1_5435_t14 | STAETVAEEVERVLKHSDDLIKEVEDVNRRVEEEIKRV | 2plus1_Key_Cterm_5435 | EEAAREIIKRLREVNKRTKEKLDELIKESEEVLE |
| | IRELEEENERLVAEVRKGVKGEILAEIEKRLADNSEKV | | RVKRLIDELRKHSEEVLEDLRRRAK (SEQ ID |
| | REVAERAKKLLEENTARVKDILRESRKLVKDLLDEVRG | | NO: 27151) |
| | TGSEEAAREIIKRLREVNKRTKEKLDMSCAQESEVLER | | |
| | VKRLIDELRK (SEQ ID NO: 27563) | | |
| degronSwitch_cODC_2plus1_5435_t11 | STAETVAEEVERVLKESDDLIKEVEDVNRRVEEEIKRV | 2plus1_Key_Cterm_5435 | EEAAREIIKRLREVNKRTKEKLDELIKHSEEVLE |
| | IRELEEENERLVAEVRKCNKGEILAEIEKRLADNSEKV | | RVKRLIDELRKHSEEVLEDLRRRAK (SEQ ID |
| | REVAERAKKLLEENTARVKDILRESRKLVKDLLDEVRG | | NO: 27151) |
| | TGSEEAAREIIKRLREVNKRTKEKLDELIMSCAQESER | | |
| | VKRLIDELRKHSE (SEQ ID NO: 27564) | | |
| degronSwitch_cODC_2plus1_5435_t8 | STAETVAEEVERVLKHSDDLIKEVEDVNRRVEEEIKRV | 2plus1_Key_Cterm_5435 | EEAAREIIKBLREVNKRTKEKLDELIKESEEVLE |
| | IRELEEENERLVAEVRKGVKGEILAEIEKRLADNSEKV | | RVKRLIDELRKHSEEVLEDLRRRAK (SEQ ID 1 |
| | REVAERAKKLLEENTARVKDILRESRKLVKDLLDEVRG | | NO: 27151) |
| | TGSEEAAREIIKRLREVNKRTKEKIDELIKLAMSCAQE | | |
| | SKRLIDELRKHSEEVL (SEQ ID NO: 27565) | | |
| degronSwitch_cODC_2plus1_5435_t4 | STAETVAEEVERVLKHSDDLIKEVEDVNRRVEEEIKRV | 2plus1_Key_Cterm_5435 | EEAAREIIKRLREVNKRTKEKLDELIKHSEEVLE |
| | IRELEEENERLVAEVRKGVKGEILAEIEKRLADNSEKV | | RVKRLIDELRKHSEEVLEDLRRRAK (SEQ ID |
| | REVAERAKKLLEENTARVKDILRESRKLVKDILDEVRG | | NO: 27151) |
| | TGSEEAAPEIIKRLREVNKRTKEKLDELIKHSEELLMS | | |
| | CAQESDELRKHSEEVLEDLR (SEQ ID NO: 27566) | | |
| degronSwitch_cODC_2plus1_5435_t0 | STAETVAEEVERVLKHSDDLIKEVEDVNRRVEEEIKRV | 2plus1_Key_Cterm_5435 | EKAAREIIKRLREVNKRTKEKLDELIKHSEEVLE |
| | IRELEEENERLVAEVRKGVKGEILAEIEKRLADNSEKV | | RVKRLIDELRKHSEEVLEDLRRRAK (SEQ ID |
| | REVAERAKKLLEENTARVKDILRESRKLVKDLLDEVRG | | NO: 27151) |
| | TGSEEAAREIIKRLREVNKRTKEKLDELIKHSEEVLEP | | |
| | LKMSCAQESKHSEEVLEDLRRRAK (SEQ ID | | |
| | NO: 27567) | | |
| degronSwitch_cODC_2plus1_5439_t15 | SRVEEIIEDLRRLLEEIRKENEDSIRRSKELIDRVKEI | 2plus1_Key_Cterm_5439 | EDKARKVAEVAEKVLRDIDKLDRESKEAFRATNE |
| | NDTIIAELEPLLKDIEKEVREKGSESEEVKKALRAVLE | | EIAKLDEDTARVAERVKKAIEDLAK (SEQ ID |
| | ELEKLLRRVAEINEEVLRRNSKLVEEDERRNPEVLKEL | | NO: 27155) |
| | ARLVEELIREIGDEDKARKVAEVAEKVLRDIDKLLMSC | | |
| | AQESRATNEEIAKLDEDTA (SEQ ID NO: 27587) | | |
| degronSwitch_cODC_2plus1_5439_t11 | SPVEEIIEDLRRELEEIRKENEDSIRRSKELLDRVKEI | 2plus1_Key_Cterm_5439 | EDKARKVAEVAEKVLPDIDKLDRESKEAFRATNE |
| | NDTIIAELERLLKDIEKEVREKGSESEEVKKALRAVLE | | EIAKLDEDTARVAERVKKAIEDLAK (SEQ ID |
| | ELEKDLRRVAEINEEVLRRNSKLVEEDERRNREVLKEL | | NO: 27155) |
| | ARLVEELIREIGDEDKARKVAEVAEKVLRDIDKLDREL | | |
| | KMSCAQESEEIAKLDEDTARVAE (SEQ ID | | |
| | NO: 27588) | | |
| degronSwitch_cODC_2plus1_5439_t8 | SRVEEIIEDLRRLLEEIRKENEDSIRRSKELLDRVKEI | 2plus1_Key_Cterm_5439 | EDKARKVAEVAEKVLRDIDKLDRESKEAFRATNE |
| | NDTIIAELERLLKDIEKEVREKGSESEEVKKAIRAVLE | | EIAKLDEDTARVAERVKKAIEDLAK (SEQ ID |
| | ELEKLLPDVAEINEEVLRRNSKLVEEDERRNREVLKEL | | NO: 27155) |
| | ARLVEELIREIGDEDKAPKVAEVAEKVLRDIDKLDRES | | |
| | KELFMSCAQESAKLDEDTARVAERVK (SEQ ID | | |
| | NO: 27589) | | |
| degronSwitch_cODC_2plus1_5439_t7 | SRNEEIIEDLPRELEEIRKENEDSIRRSKELLDRVKEI | 2plus1_Key_Cterm_5439 | EDKARKVAEVAEKVLPDIDKLDRESKEAFRATNE |
| | NDTIIAELERLLKDIEKEVREKGSESEEVKKALRAVLE | | EIAKLDEDTARVAERVKKAIEDLAK (SEQ ID |
| | ELEKLLRRVAEINEEVLRRNSKLVEEDERRNREVLKEL | | NO: 27155) |
| | ARLVEELIREIGDEDKARKVAEVAEKVLRDIDKLDRES | | |
| | KEALRMSCAQESKLDEDTARVAERVKK (SEQ ID | | |
| | NO: 27390) | | |
| degronSwitch_cODC_2plus1_5439_t4 | SRVEEIIEDLRRLLEEIRKENEDSIRRSKELLDRVKEI | 2plus1_Key_Cterm_5439 | EDKARKVAEVAEKVLRDIDKLDRESKRAFRATNE |
| | NDTIIAELERILKDIEKEVREKGSESEEVKKALRAVLE | | EIAKLDEDTARVAERVKKAIEDLAK (SEQ ID |
| | ELEKLLRRVAEINEEVERRNSKLVEEDERRNREVLKEL | | NO: 27155) |
| | ARLVEELIREIGDEDKARKVAEVAEKVLPDIDKLDRES | | |
| | KEAFRALAMSCAQESEDTARVAERVKKAIE (SEQ ID | | |
| | NO: 27591) | | |
| degronSwitch_cODC_2plus1_5439_t1 | SRVEEIIEDLRPLLEEIRKENEDSIRRSKELIDRVKEI | 2plus1_Key_Cterm_5439 | EDKARKVAEVAEKVLRDIDKLDRESKEAFRATNE |
| | NDTIIAELERLLKDIEKEVREKGSESEEVKKALRAVLE | | EIAKLDEDTARVAERVKKAIEDLAK (SEQ ID |
| | ELEKLLRRVAEINEEVLRRNSKLVEEDERRNREVLKEL | | NO: 27155) |
| | ARLVEELIREIGDEDKARKVAEVAEKVERDIDKLDRES | | |
| | KEAFRATNELIMSCAQESARVAERVKKAIEDLA (SEQ | | |
| | ID NO: 27592) | | |
| degronSwitch_cODC_2plus1_5447_t15 | SEADDVLKKLAETVKRIIERLKKLTDDSRRLVEEVHRR | 2plus1_Key_Cterm_5447 | EELSAEVKKLLDEVRKALAREKDENDKLLKEIED |
| | NDKLSKESAEAVRKAEERGIDEKDVRKLLEDIKKKSEE | | SLRRHKEENDRLLEKLKESTR (SEQ ID |
| | VAERNKRILDTLREISKRAEDEVRKVLKELEKTLKELE | | NO: 27157) |
| | DRRPDSEELSAEVKKELDEVRKLEMSCAQESDKLLKEI | | |
| | EDSLRRHK (SEQ ID NO: 27634) | | |
| degronSwitch_cODC_2plus1_5447_t11 | SEADDVLKKLAETVKRIIERLKKLTDDSRRLVEEVHRR | 2plus1_Key_Cterm_5447 | EELSAEVKKLLDEVRKALARHKDENDKLLKEIED |
| | NDKLSKESAEAVRKAEERGIDEKDVRKLLEDLKKKSEE | | SLRRHKEENDRLLEKLKESTR (SEQ ID |
| | VAERNKRILDTLREISKRAEDEVRKVLKELEKTLKELE | | NO: 27157) |
| | DRRPDSEELSAEVKKLLDEVRKALARLLMSCAQESKEI | | |
| | EDSLRRHKEEND (SEQ ID NO: 27635) | | |
| degronSwitch_cODC_2plus1_5447_t8 | SEADDVLKKLAETVKRIIERLKKLTDDSRRLVEEVHRR | 2plus1_Key_Cterm_5447 | EELSAEVKKLLDEVRKALARHKDENDKLLKEIED |
| | NDKLSKESAEAVRKAEERGIDEKDVRKLLEDLKKKSEE | | SLRRHKEENDRLLEKLKESTR (SEQ ID |
| | VAERNKRILDTLREISKRAEDEVRKVLKELEKTLKELE | | NO: 27157) |
| | DRRPDSEELSAEVKKLLDEVRKALAREKDLAMSCAQES | | |
| | EDSLRRHKEENDRLL(SEQ ID NO: 27636) | | |
| degronSwitch_cODC_2plus1_5447_t7 | SEADDVLKKLAETVKRIIERLKKLTDDSRRLVEEVHRR | 2plus1_Key_Cterm_5447 | EELSAEVKKLLDEVRKALARHKDENDKLLKEIED |
| | NDKLSKESAEAVRKAEERGIDEKDVRKLLEDLKKKSEE | | SLRRHKEENDRLLEKLKESTR (SEQ ID |
| | VAERNKRILDTLREISKRAEDEVRKVLKELEKTLKELE | | NO: 27157) |
| | DRRPDSEELSAEVKKLLDEVRKALARHKDELLMSCAQE | | |
| | SDSLRRHKEENDRLLE (SEQ ID NO: 27637) | | |
| degronSwitch_cODC_2plus1_5447_t4 | SEADDVLKKLAETVKRIIERLKKLTDDSRRLVEEVHRR | 2plus1_Key_Cterm_5447 | EELSAEVKKLLDEVRKALARHKDENDKLLKEIED |
| | NDKLSKESAEAVRKAEERGIDEKDVRKLLEDLKKKSEE | | SLRRHKEENDRLLEKLKESTR (SEQ ID |
| | VAERNKRILDTLREISKRAEDEVRKVLKELEKTLKELE | | NO: 27137) |
| | DRRPDSEELSAEVKKLLDEVRKALARHKDENDKLMMSC | | |
| | AQESRRHKEENDRLLEKLK (SEQ ID NO: 27638) | | |
| degronSwitch_cODC_2plus1_5447_t1 | SEADDVLKKLAETVKRIIERLKKLIDDSRRLVEEVHRR | 2plus1_Key_Cterm_5447 | EELSAEVKKLLDEVRKALARHKDENDKLLKEIED |
| | NDKLSKESAEAVRKAEERGIDEKDVRKLLEDLKKKSEE | | SLRRHKEENDRLLEKLKESTR (SEQ ID |
| | VAERNKRILDTLREISKRAEDEVRKVLKELEKTLKELE | | NO: 27157) |
| | DRRPDSEELSAEVKKLLDEVRKALARHKDENDKLLKLI | | |
| | MSCAQESKEENDRLLEKLKEST (SEQ ID | | |
| | NO: 27639) | | |
| degronSwitch_cODC_2plus1_5465_t15 | SAEELLREVAELVKRVDEDLRRLIEEVRASNEEVIRRL | 2plus1_Key_Cterm_5465 | EETVKRLLDELRELLERLKRTIEELLKRNRDLLA |
| | EEILKRIEEENRKVVEELRRGGVSEDLVRESKRLVDES | | DAEEKARRLLEENRKLLKAARDTAT (SEQ ID |
| | RRVIEKLVKESADSVERTRETVDRLREELKRLVEEIAK | | NO: 57159) |
| | MVKGGSSEETVKRLIDELRELLERLKRLIMSCAQESRD | | |
| | LLADAEEKARRLL (SEQ ID NO: 27726) | | |
| degronSwitch_cODC_2plus1_5465_t14 | SAEELLREVAELVKRVDEDLRRLLEEVRASNEEVIRRL | 2plus1_Key_Cterm_5465 | EETVKRILLDELRELLERLKRTIEELLKRNRDLIA |
| | EEILKRIEEENRKVVEELRRGGVSEDLVRESKRLVDES | | DAEEKARRLLEENRKLLKAARDTAT (SEQ ID |
| | RRVIEKINKESADSVERTRETVDRLREELKRLVEEIAK | | NO: 57159) |
| | MVKGGSSEETVKRLLDELRELLERLKRTLEMSCAQESD | | |
| | LLADAEEKARRLLE (SEQ ID NO: 27727) | | |
| degronSwitch_cODC_2plus1_5465_t11 | SAEELIREVAELVKRVDEDLRRLIEEVRASNEEVIRRL | 2plus1_Key_Cterm_5465 | EETVKPLIDELRELLERLKRTIEELIKRNRDLLA |
| | EEILKRIEEENRKVVEELPPGGVSEDLVRESKRLVDES | | DAEEKARRILEENRKLLKAARDTAT (SEQ ID |
| | RRVIEKLVKESADSVERTRETVDRLREELKRLVEEIAK | | NO: 57159) |
| | MVKGGSSEETVKRILDELRELLERLKRTIEELLMSCAQ | | |
| | ESADAEEKARRLLEENR (SEQ ID NO: 27728) | | |
| degronSwitch_cODC_2plus1_5465_t4 | SAEELLREVAELVKRVDEDLRRLLEEVRASNEEVIRRL | 2plus1_Key_Cterm_5465 | EETVKRLLDELRELLERLKRTIEELLKRNRDLLA |
| | EEILKRIEEENRKVVEELPRCCVSEDLVRESKPLVDES | | DAEEKARRLLEENRKLLKAARDTAT (SEQ ID |
| | RRVIEKLVKESADSVERTRETVDRLREELKPLVEEIAK | | NO: 57159) |
| | MVKGGSSEETVKRLLDELRELLERLKRTIEEILKRNRD | | |
| | LCMSCAQESRRLLEENRKLLKAAR (SEQ ID | | |
| | NO: 27729) | | |
| degronSwitch_cODC_2plus1_5465_t0 | SAEELLREVAELVKRVDEDLRRLLEEVRLSNEEVIRRL | 2plus1_Key_Cterm_5465 | EETVKRLLDELRELLERLKRTIEELLKRNRDLLA |
| | EEILKRIEEENRKVVEELRRGGVSEDLVRESKRLVDES | | DAEEKARRLLEENRKLLKAARDTAT (SEQ ID |
| | RRVIEKLVKESADSVERTRETVDRLREELKRLVEEIAK | | NO: 57159) |
| | MVKGGSSEETVKRLLDELRELLERLKRTIEELIKRNRD | | |
| | LLADLLMSCAQESEENRKLLKAARDTAT(SEQ ID | | |
| | NO: 27730) | | |
| degronSwitch_cODC_3plus1_494_t8 | SKEDKARELEKRLRDNLKKLEEVVRELAEVLKRNLEKL | 3plus1_Key_Cterm_494 | EDLVRDIRRELKELEERARKILRDDERDLRALEK |
| | RRLAEELLRALKRLLDKLRAGGLPKDELEDLPREVEDV | | RIPDIIREDREELERLKEPARK (SEQ ID |
| | LRRLEDLLRKLKKANDESLTRLEELLRRAEEENRRVLT | | NO: 27183) |
| | TLRELLRGNGDDRDLARLVARLVEANNRALEELLRLVA | | |
| | KNVEDNNRVLEELLRLVKELAKRLLGRIRDEDLVRDIR | | |
| | RELKELEEPAPKILRDLKMSCAQESKRIRDIIREDREE | | |
| | LE (SEQ ID NO: 27805) | | |
| degronSwitch_cODC_3plus1_494_t1 | SKEDKARELEKRLRDNLKKLEEVVRELAEVLKRNLEKL | 3plus1_Key_Cterm_494 | EDLVRDIRRELKELEERARKILPDDERDLRALEK |
| | RRLAEELLRALKRLLDKLRAGGLPKDELEDLRREVEDV | | PIRDIIREDREELERLKERARK (SEQ ID |
| | LRRLEDLLRKLKKANDESLTRLEELLRRAEEENRRVLT | | NO: 27183) |
| | TLRELLRGNGDDRDLARLVARLVEANNRALEELLRLVA | | |
| | KNVEDNNRVLEELLRLVKELAKRLLGRIRDEDLVRDIR | | |
| | RELKELEEPARKILRDDERDLRAIEMSCAQESREDREE | | |
| | LERLKERAR (SEQ ID NO: 27806) | | |
| degronSwitch_cODC_3plus1_500_t15 | SEKEELKPLLDKLLKELKRLSDEIKATIDKILKILKEV | 3plus1_Key_Cterm_500 | EDELRKVEEDLKRLEDKLKKLLEDYEKKVRELEE |
| | SEEVKRTADELLDAIRRGGVDEEVLPEIKREIEEIEKK | | TLDDLLRKYEETLRRLEKELEEAER (SEQ ID |
| | LRKVNKEIEDEIREIKKKIDEVDDKITKEVEKIKEALD | | NO: 27185) |
| | KGGVDAKEVIKALKEILKEHADVFEDVLRPLKEIIKRH | | |
| | PDVVKEVIEELRKILEKVAEVLKRQGRSEDELRKVEED | | |
| | LKRLEDKIKKLLMSCAQESRELEETLDDLLRKYE | | |
| | (SEQ ID NO: 27819) | | |
| degronSwitch_cODC_3plus1_506_t15 | SAEETLPRITEESKRVLEEITKKYEDLERESREVLRKL | 3plus1_Key_Cterm_506 | RERLARLLKALADKLIRVLEEILKINEELNRKII |
| | REDLDRIKRELEDVLTKGGDDKDEVLTVLEKVLDELLK | | KFARENLERNRRVNKKVIEVLREAAR (SEQ ID |
| | LHRENLRVIKELLRKVLEVDRENLEVLKRLLEKARDLL | | NO: 27187) |
| | RRSGRDEKDLKKLEDELRKVKEELEKKHEASKRDIEDL | | |
| | ERELDKVTEEVERLLRKLKEELRRAAEGSDEDRERLAR | | |
| | LLKALADKLIRVLEELLMSCAQESRKIIKFARENLERN | | |
| | R (SEQ ID NO: 27844) | | |
| degronSwitch_cODC_3plus1_506_t8 | SAEETLRRITEESKRVLEEITKKYEDLERESREVLRKL | 3plus1_Key_Cterm_506 | RERLARLLKALADKLIRVLEEILKINEELNRKII |
| | REDLDRIKRELEDVLTKGGDDKDEVLTVLEKVLDELLK | | KFARENLERNRRVNKKVIEVLREAAR (SEQ ID |
| | LHRENLRVIKELLRKVLEVDRENIEVLKRLLEKARDLL | | NO: 27187) |
| | RRSGRDEKDLKKLEDELRKVKEELEKKHEASKRDIEDL | | |
| | ERELDKVTEEVERLLRKLKEELRRAAEGSDEDRERLAR | | |
| | LLKALADKLIRVLEEILKINEELNMSCAQESRENLERN | | |
| | RRVNKKVI (SEQ ID NO: 27845) | | |
| degronSwitch_cODC_3plus1_506_t7 | SAEETLRRITEESKRVLEEITKKYEDLERESREVLRKL | 3plus1_Key_Cterm_506 | RERLARLLKALADKLIRVLEEILKINEELNRKII |
| | REDLDRIKRELEDVLTKGGDDKDEVLTVLEKVLDELLK | | KFARENLERNRRVNKKVIEVLREAAR (SEQ ID |
| | LHRENLRVIKELLRKVLEVDRENIEVLKRLLEKARDLL | | NO: 27187) |
| | RRSGRDEKDLAALEDEIRKVKEELEKKHEASKRDIEDL | | |
| | ERELDKVTEEVERLLRKLKEELRPAAEGSDEDRERLAR | | |
| | LLKALADKLIRVLEEILKINEELLKMSCAQESENLERN | | |
| | RRVNKKVIE (SEQ ID NO: 27846) | | |
| degronSwitch_cODC_3plus1_506_t4 | SAEETLRRITEESKRVLEEITKKYEDLERESREVLRKL | 3plus1_Key_Cterm_506 | RERLARLLKALADKLIRVIEEILKINEELNRKII |
| | REDLDRIKRELEDVLTKGGDDKDEVLTVLEKVLDELLK | | KFARENLERNRRVNKKVIEVLREAAR (SEQ ID |
| | LHRENLRVIKELIRKVLEVDRENLEVIKRLLEKARDLL | | NO: 21187) |
| | RRSGRDEKDLKKLEDEIRKVKEELEKKHEASKRDIEDL | | |
| | ERELDKVTEEVERLLRKLKEELRRAAEGSDEDRERLAR | | |
| | LLKALADKLIRVLEEILKINEELNPKLIMSCAQESERN | | |
| | RRVNKKVIEVLR (SEQ ID NO: 27847) | | |
| degronSwitch_cODC_3plus1_506_t1 | SAEETLRRITEESKRVLEEITKKYEDLERESREVLRKL | 3plus1_Key_Cterm_506 | RERLARLLKALADKLIRVIEEILKINEELNRKII |
| | REDLDRIKRELEDVLTKGGDDKDEVLTVLEKVLDELLK | | KFARENLERNRRVNKKVIEVLREAAR (SEQ ID |
| | LHRENLRVIKELLRKVLEVDRENLEVLKRLLEKARDLL | | NO: 27187) |
| | RRSSRDEKDLKKLEDEIRKVKEELEKKHEASKRDIEDL | | |
| | ERELDKVTEEVERLLRKLKEELRRAAEGSDEDRERLAR | | |
| | LLKALADKLIRVLEEILKINEELNRKIIKLAMSCAQES | | |
| | RRVNKKVIEVLREAA (SEQ ID NO: 27848) | | |
| degronSwitch_cODC_3plus1_508_t15 | STEEILKRVKEILDELAKELRDIIRESLRTIEELLDEL | 3plus1_Key_Cterm_508 | DEVKRRLEEKERRIRTRYEELRRRLRKRVKDYED |
| | RRILEESERTLEELVKTIKDGVKDSEELLRRLKRLLED | | KLREIEKKVRRDAERIEEELERAKK (SEQ ID |
| | LRRAHEELLKRLKEAVDEHRDRLRKILEELERVLKELR | | NO: 27189 |
| | KRIEELPRSGDRISEKDVIRKLEEVLPKALEILERLLK | | |
| | KIRESARELLKIIEFVLDANIKVMEEALKTIKELLKDG | | |
| | PDEVKRPLEEKERRIRTRYEELKMSCAQESKDYEDKLR | | |
| | EIEKKVR (SEQ ID NO: 27852) | | |
| degronSwitch_cODC_3plus1_510_t15 | SEKEELLKLIKRVIELLKRVLEEHLRLVEDVIRRLKEL | 3plus1_Key_ Cterm_510 | EDLLRKAKKVITEVREKLKRNLEDVRRVIEDVKR |
| | LDSNEKIVREVIEDLKRLLDFVRGDKEELDRIKEKLEE | | KSARILEEARRLIEEVERELEKIRK (SEQ ID |
| | VLERYKRRLEEIKRDLERMLEDYKRELKRIFEDLRRVL | | NO: 57191) |
| | EEVERIATRGEGRAEALIDKLRKIIERkLRELDKLSKK | | |
| | LDELLKKVLEELEKSNREIDKLLKDVLRRVEEGGASED | | |
| | LLRKAKKVITEVREKLKRLLMSCAQESEDVKRKSARIL | | |
| | EEAR (SEQ ID NO: 27855) | | |
| degronSwitch_cODC_3plus1_510_t8 | SEKEELLKLIKRVIELLKRVLEEHLRLVEDVIRRLKEL | 3plus1_Key_Cterm_510 | EDLLRKAKKVITEVREKLKRNLEDVRRVIEDVKR |
| | LDSNEKIVREVIEDLKRLLDEVRGDKEELDRIKEKLEE | | KSARILEEARRLIEEVERELEKIRK (SEQ ID |
| | VLERYKRRLEEIKRDLERMLEDYKRELKRIEEDLRRVL | | NO: 57191) |
| | EEVERIATRGEGPAEALIDKLRKILERALRELDKLSKK | | |
| | LDELLKKVLEELEKSNREIDKLLKDVLRRVEEGGASED | | |
| | LLRKAKKVITEVREKLKRNLEDVRRLIMSCAQESARIL | | |
| | EEARRLIEEVE (SEQ ID NO: 27856) | | |
| degronSwitch_cODC_3plus1_510_t4 | SEKEELLKLIKRVIELLKRVLFEHLRLVEDVIRRLKEL | 3plus1_Key_Cterm_510 | EDLLRKAKKVITEVREKLKRNLEDVRRVIEDVKR |
| | LDSNEKIVREVIEDLKRLLDEVRGDKEELDRIKEKLEE | | KSARILEEARRLIEEVERELEKIRK (SEQ ID 1 |
| | VLERYKRRLEEIKRDLERMLEDYKRELKRIEEDLRRVL | | NO: 57191) |
| | EEVERIATRGEGPAEALIDKLRKILERALRELDKLSKK | | |
| | LDELLKKVLEELEKSNREIDKLLKDVLRRVEEGGASED | | |
| | LLRKAKKVITEVREKLKRNLEDVRRVIEDLKMSCAQES | | |
| | EEARRLIEEVERELE (SEQ ID NO: 27857) | | |
| degronSwitch_cODC_3plus1_510_t1 | SEKEELLKLIKRVIELLKRVLEENLRLVEDVIRRLKEL | 3plus1_Key_Cterm_510 | EDLLRKAKKVITEVRFKLKRNLEDVRRVIEDVKR |
| | LDSNEKIVREVIEDLKRLLDEVRGDKEELDRIKEKLEE | | KSARILEEARRLIEEVERELEKIRK (SEQ ID |
| | VLERYKRRLEEIKRDLERMLEDYKRELKRIEEDLRRVL | | NO: 57191) |
| | EEVERIATRGEGPAEALIDKLRKILERNLRELDKLSKK | | |
| | LDELLKKVLEFLEKSNREIDKLLKDVLPRVEEGGASED | | |
| | LLRKAKKVITEVREKIKPNLEDVRRVIEDVKRLAMSCA | | |
| | QESRRLIEEVERELEKIR (SEQ ID NO: 27858) | | |
| degronSwitch_cODC_3plus1_528_t14 | SEAEDLEELIKELAELLKDVIRKLEKINRRLVKILEDI | 3plus1_Key_Cterm_528 | KDEAERRRRELKDKLDRLREEHEFVKPRLEEELT |
| | IRRLKEISKEAEEELRKGTVEDKDILRDLERRLREILE | | RLRETHKKIEKELREALKPVRDRST (SEQ ID |
| | ESDRLLEELKRRLEEILRKSKELLRRLEEVLREILKRA | | NO: 57193) |
| | EEVKRSNLPKEELIKEIVKLLEELLRVIEKILEDNIRL | | |
| | LEELVEVIKEILEKELRLLEELVRVIERILREVGKDKD | | |
| | EAERRRRELKDKLDRIREELLMSCAQESEELTRLRETH | | |
| | KKIEK (SEQ ID NO: 27912) | | |
| degronSwitch_cODC_3plus1_528_t11 | SEAEDLEELIKELAELLKDVIRKLEKINRRLVKILEDI | 3plus1_Key_Cterm_528 | KDEAERRPRELKDKLDRLREEHEEVKRRLEEELT |
| | IRRLKEISKEAEEELRKGTVEDKDILPDLERRLREILE | | RLRETHKKIEKELREALKRVRDRST (SEQ ID |
| | ESDRLLEELKRRLEEILRKSKELLRRLEEVLREILKRA | | NO: 57193) |
| | EEVKRSNLPKEELIKEIVKLLEELLRVIEKILEDNIRL | | |
| | LEELVEVIKEILEKHLRLLEELVRVIEPILREVGKDKD | | |
| | EAERRRRELKDKLDRLREEHEELKMSCAQESTRLRETH | | |
| | KKIEKELR (SEQ ID NO: 27913) | | |
| degronSwitch_cODC_3plus1_528_t4 | SEAEDLEELIKELAELLKDVIRKLEKINRRIVKILEDI | 3plus1_Key_Cterm_528 | KDRAERRRRELKDKLDRLREEHEEVKRRLEEELT |
| | IRRLKEISKEAEEELRKGTVEDKDILRDLERRLREILE | | RLRETHKKIEKELREALKRVRDRST (SEQ ID |
| | ESDRLLEELKRRLEEILRKSKELLPRLEEVLREILKRA | | NO: 57193) |
| | EEVKPSNLPKEELIKEIVKLLEELLRVIEKILEDNIRL | | |
| | LEELVEVIKEILEKHLRLLEELVRVIEPILREVGKDKD | | |
| | EAERPPREIKDKLDRIPEEHEEVKRRLEELLMSCAQES | | |
| | KKIEKELREALKRVR (SEQ ID NO: 27914) | | |
| degronSwitch_cODC_3plus1_528_t0 | SEAEDLEELIKELAELLKDVIRKLEKINRPIVKILEDI | 3plus1_Key_Cterm_528 | KDEAERRRRELKDKLDRLREEHEEVKRRLEEELT |
| | IRPLKEISKENEEELRKGTVEDKDILRDLERRLREILE | | RLRETHKKIEKELREALKRVRDRST (SEQ ID |
| | ESDRLLEELYPRLEEILRKSKELLRPLEEVLREILKRA | | NO: 57193) |
| | EEVKRSNLPKEELIKEIVKLLEELLRVIEKILEDNIRL | | |
| | LEELVEVIKEILEKHLRLLEELVRVIERILREVGKDKD | | |
| | EAERRRRELKDKLDRLREEHEEVKRRLEEELTRLRMSC | | |
| | AQESKELREALKRVRDRST (SEQ ID NO: 27915) | | |
| degronSwitch_cODC_3plus1_529_t15 | SEAEDLEELIKEIAELLKDVIRKLEKINRRLVKILEDI | 3plus1_Key_Cterm_529 | KDEAERRRRELKDKLDRLREEHEEVKRRLEEELT |
| | IRRLKEISKEAEEEDRKGTVEDKDILRDLERRLREILE | | RLRETHKKIEKELREALKRVRDRST (SEQ ID |
| | ESDRLLEELKRRLEEILRKSKELLRPLEEVLREILKRA | | NO: 27199) |
| | EEVKRSNLPKEELIKEIVKLLEELLRVIEKILEDNIRL | | |
| | LEELVEVIKEILEKHLRLLEELVRVIERILREVGKDKD | | |
| | EAERRRRELKDKLDRLRELHMSCAQESEEELTRLRETH | | |
| | KKIE (SEQ ID NO: 27916) | | |
| degronSwitch_cODC_3plus1_529_t14 | SEAEDLEELIKEIAELLKDVIRKLEKINRRLVKILEDI | 3plus1_Key_Cterm_529 | KDEAERRRRELKDKLDRLREEHEEVKRRLEEELT |
| | IRRLKEISKEAEEELPKGTVEDKDILRDLERRIREILE | | RLRETHKKIEKELREALKRVRDRST (SEQ ID |
| | ESDRLLEELKRRLEEILRKSKELLRRLEEVLREILKRA | | NO: 27199) |
| | EEVKRSNLPKEELIKEIVKLLEELLRVIEKILEDNIRL | | |
| | LEELVEVIKEILEKHLRLLEELVRVIERILREVGKDKD | | |
| | EAERRRRELKDKLDRLREELKMSCAQESEELTRLRETH | | |
| | KKIEK (SEQ ID NO: 27917) | | |
| degronSwitch_cODC_3plus1_529_t4 | SEAEDLEELIKELAELLKDVIRKLEKINRRLVKILEDI | 3plus1_Key_Cterm_529 | KDEAERRRRELKDKLDRLREEHEEVKRRLEEELT |
| | IRRLKEISKEAEEELRKGTVEDKCILRDLERRIREILE | | RLRETHKKIEKELREALKRVRDRST (SEQ ID |
| | ESDRLLEELKPRLEEILRKSKELLRRLEEVLREILKRA | | NO: 27199) |
| | EEVKRSNLPKEELIKEIVKLLEELLRVIEKILEDNIRL | | |
| | LEELVEVIKEILEKHLRLLEELVPVIERILREVGKDKD | | |
| | EAERRRRELKDKLDPIPEEHEEVERRLEELLMSCAQES | | |
| | KKIEKELREALKRVR (SEQ ID NO: 27918) | | |
| degronSwitch_cODC_3plus1_529_t0 | SEAEDLEELIKELAELLKDVIRKLEKINRRLVKILEDI | 3plus1_Key_Cterm_529 | KDEAERRRRELKDKLDRLREEHEEVKRRLEEELT |
| | IRRLKEISKEAEEEDRKGTVEDKDILRDLERKLREILE | | RLRETHKKIEKELREALKRVRDRST (SEQ ID |
| | ESDRLLEELKRRLEEILRKSKELLRRLEEVLREILKRA | | NO: 27199) |
| | EEVKRSNLPKEELIKEIVKLLEELLRVIEKILEDNIRL | | |
| | LEELVEVIKEILEKHLRLLEELVRVIERILREVGKDKD | | |
| | EAERRRRELKDKLDRLREEHEEVKRRLEEELTRLRMSC | | |
| | AQESKELREALKRVRDRST (SEQ ID NO: 27919) | | |
| degronSwitch_cODC_3plus1_530_t12 | SDKEEIKPRVEKTARDLETEHDKIKKRLEDTVRDIKRE | 3plus1_Key_Cterm_530 | EKRLIEEVVRLLRENLDVVRRILEALAKLIKELL |
| | LDELLEKYERVLRKIEKTLREGDGDATDLVDKLLETVK | | EALEEVLRRNKELIRELLRVLDEALK (SEQ ID |
| | RLNEEVLRTVKKIADELRRLVEEVLRVLRESNETLARL | | NO: 27205) |
| | LEEGNVGDEVRERLEEVLRRTEEIIKTLEEIVERLLDE | | |
| | LRRLVEELRRISKKLEDELDKIKEKGKDEKRLIEEVVR | | |
| | LLRENLDVVRRILELLMSCAQESLEALEEVLRRNKELI | | |
| | (SEQ ID NO: 27920) | | |
| degronSwitch_cODC_3plus1_530_t11 | SDKEEIKRRVEKTARDLETEHDKIKKRLEDTVRDIKRE | 3plus1_Key_Cterm_530 | EKRLIEEVVRLLRENLDVVRRILEALAKLIKELL |
| | LDELLEKYERVLRKIEKTLREGDGDATDLVDKLLETVK | | EALEEVLRRNKELIRELLRVLDEALK (SEQ ID |
| | RLNEEVLRTVKKIADELRRLVEEVIRVLRESNETLARL | | NO: 27205) |
| | LEEGNVGDEVRERLEEVLRRTEEIIKTLEEIVKRLLDE | | |
| | LRRLVEELRRISKKLKDELDKIKEKGKDEKRLIEEVVR | | |
| | LLRENLDVVRRILEALKMSCAQESEALEEVLRRNKELI | | |
| | R (SEQ ID NO: 27921) | | |
| degronSwitch_cODC_3plus1_530_t8 | SDKEEIKRRVEKTARDLETEHDKIKKRLEDTVRDIKRE | 3plus1_Key_Cterm_530 | EKRLIEEVVPLLRENLDVVRRILEALAKLIKELL |
| | LDELLEKYERVLRKIEKTLREGDGDATDLVDKLLETVK | | EALEEVLRRNKELIRELLPVLDEALK (SEQ ID |
| | RLNEEVLRTVKKIADELRRLVEEVLRVLRESNETLARL | | NO: 27205) |
| | LEEGNVGDEVRERLEEVLRRTEEIIKTLEEIVKRLLDE | | |
| | LRRLVEELRRISKKLKDELDKIKEKGKDEKRLIEEVVR | | |
| | LLRENLDVVRRILEALAKLVMSCAQESEEVLRRNKELI | | |
| | RELL (SEQ ID NO: 27922) | | |
| degronSwitch_cODC_3plus1_530_t5 | SDKEEIKRRVEKTARDLETEHDKIKKRLEDTVRDIKRE | 3plus1_Key_Cterm_530 | EKRLIEEVVRILRENLDVVRRILEALAKLIKELL |
| | LDELLEKYERVLRKIEKTLREGDGDATDLVDKLLETVK | | EALEEVLRRNKELIRELLRVLDEALK (SEQ ID |
| | RLNEEVLRTVKKIADELPRLVEEVLRVLRESNETLARL | | NO: 27205) |
| | LEEGNVGDEVRERLEEVLRRTEEIIKTLEEIVKRLLDE | | |
| | LRRLVEELRRISKKLKDELDKIKEKGKDEKRLIEEVVR | | |
| | LLRENLDVVRRILEALAKLIKLLMSCAQESLRRNKELI | | |
| | RELLRVL (SEQ ID NO: 27923) | | |
| degronSwitch_cODC_3plus1_530_t4 | SDKEEIKRRVEKTARDLETEHDKIKKRLEDTVRDIKRE | 3plus1_Key_Cterm_530 | EKRLIEEVVRLLRENLDVVRRILEALAELIKELL |
| | LDELLEKYERVLRKIEKTLREGDGDATDLVDKLLETVK | | EALEEVLRRNKELIRELLRVLDEALK (SEQ ID |
| | RLNEEVLRTVKKIADELPRLVEEVLRVLRESNETLARL | | NO: 27205) |
| | LEEGNVGDEVRERLEEVLRRTEEIIKTLEETVKRLLDE | | |
| | LRRLVEELRRISKKLKDELDKIKEKGKDEKRLIEEVVR | | |
| | LLRENLDVVRRILEKLAKLIKELLMSCAQESRRNKELI | | |
| | RELLRVLD (SEQ ID NO: 27924) | | |
| degronSwitch_cODC_3plus1_530_t1 | SDKEEIKRRVEKTARDLETEHDKIKKRLEDTVPDIKRE | 3plus1_Key_Cterm_530 | EKRLIEEVVRLLRENLDVVRRILEALAKLIKELL |
| | LDELLEKYERVLRKIEKTLREGDGDATDLVDKLLETVK | | EALEEVLRRNKELIRELLPVLDEALK (SEQ ID |
| | RLNEEVLRTVKKIADELPRLVEEVLRVLRESNETLARL | | NO: 27205) |
| | LEEGNVGDEVRERLEEVLRRTEEIIKTLEEIVKRLLDE | | |
| | LRRLVEELRRISKKLKDELDKIKEKGKDEKRLIEEVVR | | |
| | LLRENLDVVRRILEALAKLIKELLELCMSCAQESKELI | | |
| | RELLRVLDEAL (SEQ ID NO: 27925) | | |
| degronSwitch_cODC_3plus1_530_t0 | SDKEEIKRRVEKTARDLETEHDKIKKRLEDTVPDIKRE | 3plus1_Key_Cterm_530 | EKRLIEEVVRLLRENLDVVRRILEALAKLIKELL |
| | LDELLEKYERVLRKIEKTLREGDGDATDLVDKLLETVK | | EALEEVLRRNKELIRELLRVLDEALK (SEQ ID |
| | RLNEEVLRTVYKIADELRRLVEEVLPVLRESNETLARL | | NO: 27205) |
| | LEEGNVGDEVRERLEEVLRRTEEIIKTLEEIVKRLLDE | | |
| | LRRLVEELRRISKKLKDELDKIKEKGKDEKRLIEEVVR | | |
| | LLRENLDVVRRILEALAKLIKELLEALKMSCAQESELI | | |
| | RELLRVLDEALK (SEQ ID NO: 27926) | | |
| degronSwitch_cODC_3plus1_534_t4 | DEDRIIEEIARLLEELLRELLELIKKLIETNRRLNEEH | 3plus1_Key_Cterm_534 | EKIAEEIERELEELRRMIKRLHEDLERKLKESED |
| | ERAVRELARLLEELLDRLNKKGISDEKLKRIRERLKPA | | ELREIEARLEEKIRRLEEKLERKRR (SEQ ID |
| | LDDLERLHREINKRLEDLVRELEKLVREILKELKDALE | | NO: 27207) |
| | ELRRASARAGGEEVLRRLEEIVKKLLDLVRRILERLKE | | |
| | IHKDNVRLLRELNERLTRIVEDLVRLIREILREAGVDE | | |
| | KIAEEIERELEELRRMIKPLHEDLERKLKELLMSCAQE | | |
| | SARLEEKIRRLEEKLE (SEQ ID NO: 27940) | | |
| degronSwitch_cODC_3plus1_539_t12 | SEKEKLLKESEEEVRRLRRTLEELLRKYREVLERLRKE | 3plus1_Key_Cterm_539 | ERLVKTLIEDVEAVIKRILELITRVAEDNERVLE |
| | LREIEERVRDVVRRLKEVLDRKGLDIDTIIKEVEDLLK | | RIIRELTDNLERHLKIVREIVK (SEQ ID |
| | TVLDRLRELLDKIRRLTKEAIEVVREIIERIVRHAERV | | NO: 27213) |
| | KDELRKEGGDKEKLDRVDRLIKENTRHLKEILDRIEDL | | |
| | VRRSEKKLRDIIREVRRLIEELRKKNEEIKKGPDERLV | | |
| | KTLIEDVEAVIKRILELAMSCAQESERVLERIIRELTD | | |
| | NL (SEQ ID NO: 27956) | | |
| degronSwitch_cODC_3plus1_539_t11 | SEKEKLLKESEEEVRRLRRTLEELLRKYREVLERLRKE | 3plus1_Key_Cterm_539 | ERLVKTLIEDVEAVIKRILELITRVAEDNERVLE |
| | LREIEEPVPDVVRRLKEVLDRKGLDIDTIIKEVEDLLK | | RIIRELTDNLERHLKIVREIVK (SEQ ID |
| | TVLDRLRELLDKIRRLTKEAIEVVREIIERIVPHAERV | | NO: 27213) |
| | KDELRKEGGDKEKLDRVDRLIKENTRHLKEILDRIEDL | | |
| | VRRSEKKLRDIIREVRRLIEEERKKAEEIKKGPDERLV | | |
| | KTLIEDVEAVIKRILELLEMSCAQESRVLERIIRELTD | | |
| | NLE (SEQ ID NO: 27957) | | |
| degronSwitch_cODC_3plus1_539_t8 | SEKEKLLKESEEEVRRLRRTLEELLRKYREVLEPLRKE | 3plus1_Key_Cterm_539 | ERLVKTLIEDVEAVIKRILELITRVAEDNERVLE |
| | LREIEERVRDVVRRLKEVLDRKGLDIDTIIKEVEDLLK | | RIIRELTDNLERHLKIVREIVK (SEQ ID |
| | TVLDRLRELLDKIRRLTKEAIEVVREIIERIVRHAERV | | NO: 27213) |
| | KDELRKEGGDKEKLDRVDRLIKENTRHLKEIEDRIEDL | | |
| | VRRSEKKLRDIIREVRRLIEELRKKAEEIKKGPDERLV | | |
| | KTLIEDVEAVIKRILELITRLAMSCAQESERIIRELTD | | |
| | NLERHL (SEQ ID NO: 27958) | | |
| degronSwitch_cODC_3plus1_539_t5 | SEKEKLLKESEEEVRRLRRTLEELLRKYREVLERLRKE | 3plus1_Key_Cterm_539 | ERLVKTLIEDVEAVIKRILELITRVAEDNERVLE |
| | LREIEERVRDVVRRLEEVLDRKGLDIDTIIKEVEDLLK | | RIIRELTDNLERHLKIVREIVK (SEQ ID |
| | TVLDRLRELLDKIRRLTKEAIEVVREIIERIVRHAERV | | NO: 27213) |
| | KDELRKEGGDKEKLDRVDRLIKENTRELKEILDRIEDL | | |
| | VRRSEKKLRDIIREVRRLIEELRKKNEEIKKGPDERLV | | |
| | KTLIEDVEAVIKRILELITRVAELVMSCAQESIRELTD | | |
| | NLERHLKIV (SEQ ID NO: 27959) | | |
| degronSwitch_cODC_3plus1_539_t4 | SEKEKLLKESEEEVRRLRRTLEELLRKYREVLERLRKE | 3plus1_Key_Cterm_539 | ERLVKTLIEDVENVIKRILELITRVAEDNERVLE |
| | LREIEERVRDVVRRLKEVLDRKGLDIDTIIKEVEDLLK | | RIIRELTDNLERHLKIVREIVK (SEQ ID |
| | TVLDRLRELLDKIRRLTKEAIEVVREITERIVRHAERV | | NO: 27213) |
| | KDELRKEGGDKEKLDPVDRLIKENTRHLKEILDRIEDL | | |
| | VRRSEKKLRDIIREVRRLIEELRKKAEEIKKGRDERLV | | |
| | KTLIEDVEAVIKRILELITRVAEDLYMSCAQESPELTD | | |
| | NLERHLKIVR (SEQ ID NO: 27960) | | |
| degronSwitch_cODC_3plus1_539_t0 | SEKEKLLKESEEEVRRLRRTLEELIPKYREVLERLRKE | 3plus1_Key_Cterm_539 | EPLVKTLIEDVEAVIKRILELITRVAEDNERVLE |
| | LREIEERVRDVVRRLKEVLDRKGLDIDTIIKEVEDLLK | | RIIRELTDNLERHLKIVREIVK (SEQ ID |
| | TVLDRLRELLDKIRRLTKEAIEVVPEIIERIVRHNERV | | NO: 27213) |
| | KDELRKEGGDKEKLDRVDRLIKENTRHLKEILDRIEDL | | |
| | VRRSEKKLPDIIREVRRLIEELRKYAEEIKKGRDERLV | | |
| | KTLIEDVEAVIKRILELITRVAEDNERVEKMSCAQESD | | |
| | NLERHLKIVREIVK (SEQ ID NO: 27961) | | |
| degronSwitch_cODC_3plus1_548_t15 | DKAEVEREALKLLKDLLEELIKIHEESLKRILDLIDTL | 3plus1_Key_Cterm_548 | EEIDRELKPVVEELRRLHEEIKERLDDVARRSEE |
| | VKVHEDALRALKELLERSGLDERELRKVERMATESLRT | | ELPRIIKKIKEVVKEIRKKLK (SEQ ID |
| | IAKLKEELRDLARRSLEKLREDLKRVDDTLRKVEEKVR | | NO: 27215) |
| | RTGPSEELIEELIRTIEKLLKEIVRINEEVLKAVRELL | | |
| | KTLLKLSEDVVRRIEEILRKGGVPEEIDRELKRVVEEL | | |
| | RRLHMSCAQESDDVAPPSEEELRRII (SEQ ID | | |
| | NO: 27985) | | |
| degronSwitch_cODC_3plus1_548_t11 | DKAEVLREALKLLKDLLEELIKIHEESLKRILDLIDTL | 3plus1_Key_Cterm_548 | EEIDRELERNNEELPPLHEEIKERLDDVARRSEE |
| | VKVHEDALRALKELLEPSGLDERELRKVERMATESLRT | | ELRRIIKKLKEVVIKEIRKKLK (SEQ ID |
| | IAKLKEELRDLARRSLEKLREDLKRVDDTLRKVEEKVR | | NO: 27215) |
| | RTGPSEELIEELIRTIEKELKEIVRINEEVLKAVRELL | | |
| | KTLLKLSEDVVERIEEILRKGGVPEEIDRELKRVVEEL | | |
| | PRLHEELNMSCAQESRPSEEELRRIIKKLK (SEQ ID | | |
| | NO: 27986) | | |
| degronSwitch_cODC_3plus1_548_t8 | DKAEVEREADKELKDLLEELIKIHEESLKRILDLIDTL | 3plus1_Key_Cterm_548 | EEIDRELERVVEELRRLHEEIKERLDDVARRSEE |
| | VKVHEDALRALKELLERSGLDERELRKVERMATESLRT | | ELRRIIKKLKEVVIKEIRKKLK (SEQ ID |
| | IAKLKEELRDLARRSLEKLREDLKEVDDTLREVEEKVR | | NO: 27215) |
| | RTGPSEELIEELIRTIEKELKEIVRINEEVLKAVRELL | | |
| | KTLLKESEDVVRRIEEILRKGGVPEEIDRELKIVVEEL | | |
| | RRLHEEIKELLMSCAQESEEELRRIIKKLKEVV (SEQ | | |
| | ID NO: 27987) | | |
| degronSwitch_cODC_3plus1_548_t4 | DKAEVEREALKLLEDLLEELIKIHEESEKRILDLIDTL | 3plus1_Key_Cterm_548 | EEIDRELKRVVEELRPLHEEIKERLDDVARRSEE |
| | VKVHEDALRALKELLERSGLDERELRKVERMATESLRT | | ELRRIIKKLKEVVIKEIRKELK (SEQ ID |
| | IAKLKEELRDLARRSLEKLREDLKPVDDTLRKVEEKVR | | NO: 27215) |
| | RTGPSEELIEELIRTIEKLLKEIVRINEEVLKAVRELL | | |
| | KTLLKESEDVVRRIEEILREGGVPEEIDRELKRVVEEL | | |
| | RRLHEEIKERLDDLGMSCAQESRRIIKKLKEVVKEIR | | |
| | (SEQ ID NO: 27988) | | |
| degronSwitch_cODC_3plus1_548_t0 | DKAEVEREALKELKDLLEELIKIHEESEKRILDLIDTL | 3plus1_Key_Cterm_548 | EEIDRELKRVVEELRRLHEEIKERLDDVARRSEE |
| | VKVHEDALRALKELLERSGLDERELRKVERMATESLRT | | ELRRIIKKIKEVVKEIRKKLK (SEQ ID |
| | IAKLKEELRDLARRSLEKLREDLKRVDDTLRKVEEKVR | | NO: 27215) |
| | RTGPSEELIEELIRTIEKLLKEIVRINEEVEKAVRELL | | |
| | KTLLKLSEDVVRRIEEILPKGCNPEEIDRELKPVVEEL | | |
| | RRLHEEIKERLDDVARRLLMSCAQESKKLKEVVKEIRK | | |
| | KLK (SEQ ID NO: 27989) | | |
| degronSwitch_cODC_3plus1_556_t5 | SERELIERWLELHKEILRLIRELVERLLKLHREILDTI | 3plus1_Key_Cterm_556 | DDERRTLTELLKRMEDILEKVERTLKKILDDSAR |
| | KKLIRELLELLEDIARKLGLDKEAKDELREIAKRVEDK | | MAEEVKKTLKELLERSEKVAEDVRK (SEQ ID |
| | LEKLERESRKVEEDLKRKLKELTDESDTVEKRVRDVVR | | NO: 27217) |
| | RGTQSREEIAEELLRLDRKLLKAVEELLKEILDLNKKL | | |
| | LDDVRAILEETRRVLEKLLDRVRRGERTDDERRTLTEL | | |
| | LKRMEDILEKVERTEKKLLDLAMSCAQESKKTLKELLE | | |
| | RSEKVA (SEQ ID NO: 28016) | | |
| degronSwitch_cODC_3plus1_556_t4 | SERELIERWLELHKEILRLIRELVEPLLKLHREILDTI | 3plus1_Key_Cterm_556 | DDERRTLTELLKRMEDILEKVERTIKKLLDDSAR |
| | KKLIRELLELLEDIARKLGLDKEAKDELREIAKRVEDK | | MAEEVKKTLKELLERSEKVAEDVRK (SEQ ID |
| | LEKLERESRKVEEDLRRKLKELTDESDTVEKRVRDVVR | | NO: 27217) |
| | RGTQSREEIAEELLRLDRKLLKAVEELLKEILDLNKKL | | |
| | LDDVRAILEETRRVLEKLLDRVRRGERTDDERRTLTEL | | |
| | LKRMEDILEKVERTLKKLLDDLLMSCAQESKTLKELLE | | |
| | RSEKVAE (SEQ ID NO: 28017) | | |
| degronSwitch_cODC_3plus1_556_t1 | SERELIERWLELHKEILRLIRELVERLLKLHREILDTI | 3plus1_Key_Cterm_556 | DDERRTLTELIKRMEDILEKVERTLKKLLDDSAR |
| | KKLIRELLELLEDIARKLGLDKEAKDELREIAKRVEDK | | MAEEVKKTLKELLERSEKVAEDVRK (SEQ ID |
| | LEKLERESRKVEEDLKRKLKELTDESDTVEKRVRDVVR | | NO: 27217) |
| | RGTQSREEIAEELLRLDRKLLKAVEELLKEILDLNKKL | | |
| | LDDVRAILEETRRVLEKLLDRVPRGERTDDERRTLTEL | | |
| | LKRMEDILENVERTLKKLLDDSARLAMSCAQESKELLE | | |
| | RSEKVAEDVR (SEQ ID NO: 28018) | | |
| degronSwitch_cODC_3plus1_560_t15 | SKKELLEEVVRRAIELLKRHLEKLKRILEEIVRLLEEH | 3plus1_Key_Cterm_560 | EDKLKEIEDELPRLLEELRRLDKAIKDRLRELKK |
| | LEKVERVLEAILSLLDDLLRRGGDERAIRTLEDVKRRL | | DLDEANPRIKETLKKILREVEK (SEQ ID |
| | REILERLADENAKAIKPLADLLDKLEKRNKFAIERLEE | | NO: 27219) |
| | ILEELKRVRRDEELLRVLETLLKIIEDILRENTKVLED | | |
| | LLPLVEEILEANLRVVEELLRIAREILTEIVGDEDKLK | | |
| | EIEDELPRLLEELQMSCAQESDRDRELKKDLDEANR | | |
| | (SEQ ID NO: 28034) | | |
| degronSwitch_cODC_3plus1_560_t12 | SKKELLEEVVRRAIELLKRHLEKIKRILEEIVRLLEEH | 3plus1_Key_Cterm_560 | EDKLKEIEDELPRLLEELRRLDKAIKDPLRELKK |
| | LEKVERVLEAILSLLDDLLRRGGDERAIRTLEDVKRRL | | DLDEANRRIKETLIKKILREVEK (SEQ ID |
| | REILERLADENAKAIKPLADLLDKIEKRNKEAIERLEE | | NO: 27219) |
| | ILEELKRVRRDEELLRVLETLLKIIEDILRENTKVLED | | |
| | LLPLVEEILEANLRVVEELLRLAREILTEIVGDEDKLK | | |
| | EIEDELRRILEELRRLVMSCAQESPELKKDLDEANRRI | | |
| | K (SEQ ID NO: 28035) | | |
| degronSwitch_cODC_3plus1_560_t8 | SKKELLEEVVRRAIELLKRHLEKLKRILEEIVRLLEEH | 3plus1_Key_Cterm_560 | EDKLKEIEDELPRLLEELRRLDKAIKDRLRELKK |
| | LEKVERVLEAILSLLDDLLRRGGDERAIRTLEDVKRRL | | DLDEANRRIKETLKKLLREVEK (SEQ ID |
| | REILERLADENAKAIKPLADLLDKLEKRNKEAIERLEE | | NO: 27219) |
| | ILEELKRVRRDEELLRVLETLLKIIEDILRENTKVLED | | |
| | LLRLVEEILEANLRVVEELLRLAREILTEIVGDEDKLK | | |
| | EIEDELRRLLEELRRLDKALKMSCAQESKDLDEANRRI | | |
| | KETLK (SEQ ID NO: 28036) | | |
| degronSwitch_cODC_3plus1_560_t1 | SKKELLEEVVRRAIELLKRHLEKLKRILEEIVRLLEEH | 3plus1_Key_Cterm_560 | EDKLKEIEDELPRLLEELRRLDKAIKDRLRELKK |
| | LEKVERVLEAILSLLDDLLRRGGDERAIRTLEDVKRRL | | DLDEANPRIKETLKKLLREVEK (SEQ ID |
| | REILERLADENAKAIKRLADLLDKLEKRNKEAIERLEE | | NO: 27219) |
| | ILEELKRVRRDEELLRVLETLLKIIEDILRENTKVLED | | |
| | LLELVEEILEANLRVVEELLELAREILTKIVGDEDKLK | | |
| | EIEDELRRLLEELERLDKAIKDRLPELYMSCAQESRRI | | |
| | KETLKKLLREVE (SEQ ID NO: 28037) | | |
| degronSwitch_cODC_3plus1_568_t15 | KEIEETLKELEDLNREMVETNRRVLEETRRLNKETVDR | 3plus1_Key_Cterm_568 | KAVEELEKALEEIKRRLKEVIDRYEDELRKLRKE |
| | VKATLDELAKMLKKLVDDVRKGPTSEELKRLLAELEEL | | YKEKIDKYERKLEEIERRERT (SEQ ID |
| | LARVVRRVEELLKKSTDLLERAVKDSADALRRSHEVLK | | NO: 27221) |
| | EVASRVKPAKDEGLEREEVLRLLRELLERHAKVLKDIV | | |
| | RVSEKLLREHLKVLREIVEVLEELLERILKVILDTTGG | | |
| | DKAVEELEKALEEIKRRLTMSCAQESDELRKLRKEYKE | | |
| | KID (SEQ ID NO: 28046) | | |
| degronSwitch_cODC_3plus1_572_t15 | DEDELIRKLLEDLKDIVRKILELIERDLRDIERVVRRI | 3plus1_Key_Cterm_572 | SELADRIRKLIEDLERHTAKVLEDVKRITELRK |
| | VKVIRDDLKKIKEVVDDIARGVPRTEELERVIKRIEEL | | NSKDILEEVRKLIDELRKRIKEVED (SEQ ID |
| | LRTSEEELDRILKEIEELIRESRRRLEEVVSAVEELLR | | NO: 27225) |
| | RVEEIVDKGRESKEDVIKLLREVVDDILRLVEEVVRTN | | |
| | LEIIKRILELIEPVIRLNLSIIRDILRLLEGTVDSELA | | |
| | DRIRKLIEDLERHTAKLLMSCAQESTELRKNSKDILEE | | |
| | VR (SEQ ID NO: 28059) | | |
| degronSwitch_cODC_3plus1_572_t8 | DEDELIRKLLEDLKDIVRKILELIERDLRDIERVVRRI | 3plus1_Key_Cterm_572 | SELADRIRKLIEDLERHTAKVLEDVKRAITELRK |
| | VKVIRDDLKKIKEVVDDIARGVPRTEELERVIKRIEEL | | NSKDILEEVRKLIDELEKRIKEVED (SEQ ID |
| | LRTSEEELDRILKEIEELLRESRRRLEEVVSAVEELLR | | NO: 27225) |
| | RVEEIVDKGRESKEDVIKLLREVVDDILRLVEEVVRTN | | |
| | LEIIKRILELIERVIRLNLSIIRDILRLLEGTVDSELA | | |
| | DRIRKLIEDLERHTAKVLEDVKRLMMSCAQESKDILEE | | |
| | VRKLIDELR (SEQ ID NO: 28060) | | |
| degronSwitch_cODC_3plus1_572_t7 | DEDELIRKLIEDLKDIVRKILELIERDLEDIERVVRRI | 3plus1_Key_Cterm_572 | SELADRIRKLIEDLERHTAKVLEDVKRAITELRK |
| | VKVIRDDLKKIKEVVDDIARGVERTEELERVIKRIEEL | | NSKDILEEVRKLIDELRKRIKEVED (SEQ ID |
| | LRTSEEELDPILKEIEELLRESRRELEEVVSAVEELLR | | NO: 27225) |
| | RVEEIVDKGRESKEDVIKLLREVVDDILRINEEVVRTN | | |
| | LEIIKRILELIERVIRLNLSIIRDILPLLEGTVDSELA | | |
| | DRIRKLIEDLERHTAKVLEDVKRALSMSCAQESDILEE | | |
| | VRKLIDELPK (SEQ ID NO: 28061) | | |
| degronSwitch_cODC_3plus1_572_t4 | DEDELIRKLLEDLKDIVRKILELIERDLEDIERVVRRI | 3plus1_Key_Cterm_572 | SELADRIRKLIEDLERHTAKVLEDVKRAITELRK |
| | VKVIRDDLKKIKEVVDDIARGVPRTEELERVIKRIEEL | | NSKDILEEVRKLIDEIRKPIKEVED (SEQ ID |
| | LRTSEEELDPILKEIEELLRESRREIEEVVSAVEELLR | | NO: 27225) |
| | RVEEIVDKGRESKEDVIKLLREVVDDILRLVEEVVRTN | | |
| | LEIIKRILELIERVIRLNLSIIRDILRLLEGTVDSELA | | |
| | DRIRKLIEDLERHTAKVLEDVKRAITELAMSCAQESEE | | |
| | VRKLIDELRKRIK (SEQ ID NO: 28062) | | |
| degronSwitch_cODC_3plus1_572_t1 | DEDELIRKLLEDLKDIVRKILELIERDLRDIERVVRRI | 3plus1_Key_Cterm_572 | SELADRIPKLIEDLERHTAKVLEDVKRAITELRK |
| | VKVIRDDLKKIKEVVDDIARGVPRTEELERVIKRIEEL | | NSKDILEEVRKLIDELRKRIKEVED (SEQ ID |
| | LRTSEEELDRILKEIEELLRESPRRLEEVVSAVEELLR | | NO: 27225) |
| | RVEEIVDKGRESKEDVIKLLRETVDDILPLVEEVVRTN | | |
| | LEIIKRILELIERVIRLNLSIIRDILRLLEGIVDSELA | | |
| | DRIRKLIEDLERHTAKVLEDVKRAITELRKLAMSCAQE | | |
| | SRKLIDELRKRIKEVE (SEQ ID NO: 28063) | | |
| degronSwitch_cODC_3plus1_581_t15 | SALETVKKLLEDSSEKIERIVEEDERVAKESSDRIRRL | 3plus1_Key_Cterm_581 | AEAVIKVIEKLIRNNKRVWDALLKINEDLVRVNK |
| | VEEDKRVADEILDLIEKIGDTDTILKLVEEWSRTSKKL | | TVWKELLRVNEKLARDLERVVK (SEQ ID |
| | LDDVLKLHKDWSDDSRRLLEEILRVHEELIRPVKEILD | | MC: 27227) |
| | REGKPEEVVRELEKVLKESLDTLEEIIRRLDEANAATV | | |
| | KRVADVIRELEDINRKVLEEIKRGSDDAEAVIKVIEKL | | |
| | IRANKRLWMSCAQESEDLVRVNKTVWKELL (SEQ ID | | |
| | NO: 28095) | | |
| degronSwitch_cODC_3plus1_581_t14 | SALETVKKLLEDSSEKIERIVEEDERVAKESSDRIRRL | 3plus1_Key_Cterm_581 | AEAVIKVIEKLIRANKRVWDALLKINEDLVRVNK |
| | VEEDKRVADEILDLIEKIGDTDTLLKLVEEWSRTSKKL | | TVWKELLPVNEKLARDLERVVK (SEQ ID |
| | LDDVLKLHKDWSDDSRRLLEEILRVHEELIRRVKEILD | | NO: 27227) |
| | REGKPEEVVRELEKVLKESLDTLEEIIRRLDENNAATV | | |
| | KRVADVIRELEDINRKVLEEIKROSDDAEAVIKVIEKL | | |
| | IRANKRVLDMSCAQESDLVRVNKTVWKELLR (SEQ | | |
| | ID NO: 28096) | | |
| degronSwitch_cODC_3plus1_581_t12 | SALETVKKLLEDSSEKIERIVEEDERVAKESSDRIRRL | 3plus1_Key_Cterm_581 | AEAVIKVIEKLIRANKRVWDALLKINEDLVRVNK |
| | VEEDKRVADEILDLIEKIGDTDTLLKLVEEWSRTSKKL | | TVWKELLRVNEKLARDLERVVK (SEQ ID |
| | LDDVLKLHKDWSDDSRRLLEEILRVHEELIRRVKEILD | | NO: 27227) |
| | REGKPEEVVRELEKVLKESLDTLEEIIRRLDEANAATV | | |
| | KRVADVIPELEDINPKVLEEIKRGSDDAEAVIKVIEKL | | |
| | IRANKRVWDLLMSCAQESVRVNKTVWKELLRVN (SEQ | | |
| | ID NO: 28097) | | |
| degronSwitch_cODC_3plus1_581_t11 | SALETVKKLLEDSSEKIERIVEEDERVAKESSDRIRRL | 3plus1_Key_Cterm_581 | AEAVIKVIEKLIRANKRVWDALLKINEDLVRVNK |
| | VEEDKRVADEILDLIEKIGDTDTLLKLVEEWSRTSKKL | | TVWKELLPVNEKLARDLERVVK (SEQ ID |
| | LDDVLKLHKDWSDDSRRLLEEILRVHEELIRRVKEILD | | NO: 27227) |
| | REGKPEEVVRELEKVLKESLDTLEEIIRRLDEANAATV | | |
| | KRVADVIRELEDINRKVLEEIKRGSDDAEAVIKVIEKL | | |
| | IRANKRVWDALLMSCAQESRVNKTVWKELLRVNE | | |
| | (SEQ ID NO: 28098) | | |
| degronSwitch_cODC_3plus1_585_t15 | SKEEKLKDDVRAVLEDLDRVLKELEKLSEDNLRELKRV | 3plus1_Key_Cterm_585 | SKAAEDILRVLEKLVKVSRaNIKLILELSEHHVR |
| | LDRITDLHRRILDELPKGIGSEELLRRVEKVLKDNLDL | | VSTRIARLLLDVARKLNEVIKEJNEP (SEQ ID |
| | LRKLVEEHKESSERDLKRVEDLVREIKEVLRKLLELED | | NO: 27229) |
| | RGTDIRKIEEEIERLLRKIRKAVEESKDLNRRNSERIE | | |
| | EVARRSEELARRLLKEIRERGDSKAAEDILRVLEKLVK | | |
| | VSRELCMSCAQESEHHVRVSTRIARLLL (SEQ ID | | |
| | NO: 28118) | | |
| degronSwitch_cODC_3plus1_585_t12 | SKEEKLKDDVRAVLEDLDRVLKELEKLSEDNLRELKRV | 3plus1_Key_Cterm_585 | SKAAEDILRVLEKINKVSREAIKLILELSEHHVR |
| | LDRITDLHRRILDELPKGIGSEELLRRVEKVLKDNLDL | | VSTRIARLILDVARKLAEVIKEAER (SEQ ID |
| | LRKLVEEHKESSERDLKRVEDLVREIKEVLRKLLELED | | NO: 27229) |
| | RGTDIRKIEEEIERLIRKIRKAVEESKDLNRRNSERIE | | |
| | EVARRSEELARRLLKEIRERGDSKAAEDILRVLEKLVK | | |
| | VSREAIKLIMSCAQESVPIVSTRIARLLLDVA (SEQ | | |
| | ID NO: 28119) | | |
| degronSwitch_cODC_3plus1_585_t11 | SKEEKLKDDVRAVLEDLDRVLKEIEKLSEDNLRELKRV | 3plus1_Key_Cterm_585 | SKAAEDILRVLEKLNKVSREAIKLILELSEHHVR |
| | LDRITDLHRRILDELRKGIGSEELLRRVEKVLKDNLDL | | VSTRIARLLLDVARKLAEVIKEAER (SEQ ID |
| | LRKLVEEHKESSERDLKRVEDLVREIKEVLRKLLELED | | NO: 27229) |
| | RGTDIRKIEEEIERLLRKIRKAVEESKDLNRRNSERIE | | |
| | EVARRSEELkRRLLKEIRERGDSKAAEDILRVLEKLVK | | |
| | VSREAIKLLIMSCAQESRVSTRIARLLLDVAR (SEQ | | |
| | ID NO: 28120) | | |
| degronSwitch_cODC_3plus1_585_t8 | SKEEKLKDDVRAVLEDLDRVLKELEKLSEDNLRELKRV | 3plus1_Key_Cterm_585 | SKAAEDILRVLEKLVKVSREAIKLILELSEHHVR |
| | LDRITDLHRRILDELRKGIGSEELLRRVEKVLKDNLDL | | VSTRIARLLLDVARKLAEVIKEAER (SEQ ID |
| | LRKLVEEHKESSERDLKRVEDLVREIKEVLRKLLELED | | NO: 27229) |
| | RGTDIRKIEEEIERLLRKIRKNVEESKDLNRRNSERIE | | |
| | EVARRSEELARRLLKEIRERGDSKAAEDILRVLEKLVK | | |
| | VSREAIKLILELAMSCAQESTRIARLLLDVARKLA | | |
| | (SEQ ID NO: 28121) | | |
| degronSwitch_cODC_3plus1_585_t4 | SKEEKLKDDVRAVLEDLDRVLKELEKLSEDNLRELKRV | 3plus1_Key_Cterm_585 | SKAAEDILRVLEKLVKVSREAIKLILELSEHHVR |
| | LDRITDLHRRILDELRKGIGSEELLRRVEKVIKDNLDL | | VSTRIARLLLDVARKLAEVIKEAER (SEQ ID |
| | LRKLVEEHKESSERDLKRVEDLVREIKEVLRKLLELED | | NO: 27229) |
| | RGTDIRKIEEEIERLLRKIRKAVEESKDLNRRNSERIE | | |
| | EVARRSEELARRLLKEIPERGDSKAAEDILRVLEKLVK | | |
| | VSREAIKLILELSEHLIMSCAQESPLLLDVARKLAEVI | | |
| | K (SEQ ID NO: 28122) | 3plus1_Key_Cterm_585 | SKAAEDILRVLEKLVKVSREAIKLILELSEHHVR |
| degronSwitch_cODC_3plus1_585_t1 | SKEEKLKDDVRAVLEDLDRVLKELEKLSEDNLRELKRV | | VSTRIARLLLDVARKLAEVIKEAER (SEQ ID |
| | LDRITDLHRRILDELRKGIGSEELLRRVEKVLKDNLDL | | NO: 27229) |
| | LRKLVEEHKESSERDLKPVEDLVREIKEMLRKLLELED | | |
| | RGTDIRKIEEEIERLLRKIRKAVEESKDLNRRNSERIE | | |
| | EVARRSEELARRLIKEIRERGDSKAAEDILRVLEKLVK | | |
| | VSREAIKLILELSEHHVRLSMSCAQESLDVARKLAEVI | | |
| | KEAE (SEQ ID NO: 28123) | | |
| degronSwitch_cODC_3plus1_585_t0 | SKEEKLKDDVRAVLEDLDRVLKELEKLSEDNLRELKRV | 3plus1_Key_Cterm_585 | SKAAEDILPVLEKLVKVSRRAIKLILELSEHHVR |
| | LDRITDLHRRILDELRKGIGSEELLRRVEKVLKDNLDL | | VSTRIARLLIDVARKLAEVIKEAER (SEQ ID |
| | LRKLVEEHKESSERDLKPVEDLVREIKEVLRKLLELED | | NO: 27229) |
| | RGTDIRKIEEEIERLLRKIRKAVEESKDLNRRNSERIE | | |
| | EVARPSEELARRLLKEIRERGDSKAAEDILRVLEKLVK | | |
| | VSREAIKLILELSEHHVRVLTMSCAQESDVARKLAEVI | | |
| | KEAER (SEQ ID NO: 28124) | | |
| degronSwitch_cODC_3plus1_587_t15 | SEIEDVIRRLRKILEDLEPVSEKLLREIKKILDEARRL | 3plus1_Key_Cterm_587 | IEDLVREVERLIKRIEDSLRELEKTVRELLKRIK |
| | NEEVIKEIKRVLEDAVRVFRDGSGSKEELAKLVEELIR | | EASDKVREDVDRLIKELKEAAD (SEQ ID |
| | ELAKLAKEVDEIHKRIVEPIKALVEDAERIHRKIVETL | | NO: 27231) |
| | EEIVRGVPSEELKRVVEAIVEVIKEHLKVLADVIRRII | | |
| | KAIEENAETIKRVLEDIVRVLEIVIRGEGSIEDLVREV | | |
| | ERLIKRIEDLVMSCAQESRELLKRIKEASDKVR (SEQ | | |
| | ID NO: 28131) | | |
| degronSwitch_cODC_3plus1_587_t8 | SEIEDVIRRLRKILEDLERVSEKLIREIKKILDEARRL | 3plus1_Key_Cterm_587 | IEDLVREVERLIKRIEDSLRELEKTVRELLKRIK |
| | NEEVIKEIKRVLEDAVRVFRDGSGSKEELAKLVEELIR | | EASDKVREDVDRLIKELKEAAD (SEQ ID |
| | ELAKLAKEVDEIHKRIVERLKALVEDAERIHREIVETL | | NO: 27231) |
| | EEIVRGVPSEELKRVVEAIVEVIKEHLKVLADVIRRII | | |
| | KAIEENAETIKRVLEDIVRVLELVLRGEGSIEDLVREV | | |
| | ERLIKRIEDSIRELEKLVMSCAQESKEASDKVREDVDR | | |
| | LI (SEQ ID NO: 28132) | | |
| degronSwitch_cODC_3plus1_587_t4 | SEIEDVIRRLRKILEDLERVSEKLIREIKKILDEARRL | 3plus1_Key_Cterm_587 | IEDLVREVERLIKRIEDSLRELEKTVRELLKRIK |
| | NEEVIKEIKRVLEDAVRVFRDGSGSKEELAKLVEELIR | | EASDKVREDVDRLIKELKEAAD (SEQ ID |
| | ELAKLAKEVDEIHKRIVERLKALVEDAERIHRKIVETL | | NO: 27231) |
| | EEIVRGVPSEELKRVVEAIVEVIKEHLKVLADVIRRII | | |
| | KAIEENAETIKRVLEDIVRVLELVLRGEGSIEDLVREV | | |
| | ERLIKRIEDSLRELEKTVPELLMSCAQESDKVREDVDR | | |
| | LIKELK (SEQ ID NO: 28133) | | |
| degronSwitch_cODC_3plus1_587_t1 | SEIEDVIRRLRKILEDLERVSEKLIREIKKILDEARRL | 3plus1_Key_Cterm_587 | IEDLVREVERLIKRIEDSLRELEKTVRELLKRIK |
| | NEEVIKEIKRVLEDAVRVFRDGSGSKEELAKIVEELIR | | EASDKVREDVDPLIKEIKEAAD (SEQ ID |
| | ELAKLAKEVDEIHKRIVERLKALVEDAERIHRKIVETL | | NO: 27231) |
| | EEIVRGVPSEELKRVVEAIVEVIKEHLKVLADVIRRII | | |
| | KAIEENAETIKRVLEDIVRVLELVLRGEGSIEDLVREV | | |
| | ERLIKRIEDSLRELEKTVRELLKLAMSCAQESPEDVDR | | |
| | LIKELKEAN (SEQ ID NO: 28134) | | |
| degronSwitch_cODC_3plus1_587_t0 | SEIEDVIRRLRKILEDLERVSEKLLREIKKILDEARRL | 3plus1_Key_Cterm_587 | IEDLVREVERLIKRIEDSLRELEKTVRELLKRIK |
| | NEEVIKEIKPVLEDAVRVFRDGSGSKEELAKLVEELIR | | EASDKVREDVDRLIKELKEAAD (SEQ ID |
| | ELAKLAKEVDEIHKRIVERLKALVEDAERIHRKIVETL | | NO: 27231) |
| | EEIVRGVPSEELKRVVEAIVEVIKEHLKVLADVIRRII | | |
| | KAIEENAETIKRVLEDIVPVLELVLRGEGSIEDLVREV | | |
| | ERLIKRIEDSLRELEKTVRELLKPLAMSCAQESEDVDR | | |
| | LIKELKEAAD (SEQ ID NO: 28135) | | |
| degronSwitch_cODC_3plus1_605_t15 | SREELLDRILEAIAKILEDLKRLIDENLARLEEVVREL | 3plus1_Key_Cterm_605 | DEIIRKLDELLKEVEKVHKEVKDRIRKLLEDHKR |
| | ERIIDRNLKLIREILDELKKGSGSEEILEKIKKVDKEL | | SLDEVKKKLERLLERAKEVVEREKK (SEQ ID |
| | EDLIRRLLKKLEDLIRETERRLREILKRIRDILKEVKD | | NO: 27233) |
| | RDKDLERLLEVLEEVLRVIAELAKELLDSLRKVLKVVE | | |
| | EVLRLLNEVNKEVLDVIRELAKDGGSDEIIRKLDELLK | | |
| | EVEKVHKELKMSCAQESEDHKRSLDEVKKKLE (SEQ | | |
| | ID NO: 28213) | | |
| degronSwitch_cODC_3plus1_605_t8 | SREELLDRLLEALAKILEDLKRLIDENLARLEEVVREL | 3plus1_Key_Cterm_605 | DEIIRKLDELLKEVEKVHKEVKDRIRKLLEDHKR |
| | ERIIDRNLKLIREILDELKKGSGSEEILEKIKKVDKEL | | SLDEVKKKLERILERAKEVVEREKK (SEQ ID |
| | EDLIRRLLKKLEDLIPETERPLREILKPIRDLLKEVKD | | NO: 27233) |
| | RDKDLERLLEVLEEVLRVIAELAKELLDSLRKVLKVVE | | |
| | EVLPLLNEVNKEVLDVIRELAKDGGSDEIIRKLDELLK | | |
| | EVEKVHKEVKDRIRKILMSCAQESDEVKKKLEPLLERA | | |
| | K (SEQ ID NO: 28214) | | |
| degronSwitch_cODC_3plus1_605_t7 | SREELLDRILEALAKILEDLKPLIDENLARLEEVVREL | 3plus1_Key_Cterm_605 | DEIIRKLDELLKEVEKVHKEVKDRIRKLLEDHKR |
| | ERIIDRNLKLIREILDELKKGSGSEEILEKIKKVDKEL | | SLDEVKKKERLLERAKEVVEREKK (SEQ ID |
| | EDLIRRLLKKLEDLIRETERRLREILKRIRDLLKEVKD | | NO: 27233) |
| | PDKDLERLLEVLEEVLRVIAELAKELLDSLRKVLKVVE | | |
| | EVLRLLNEVNKEVLDVIRELAKDGGSDEIIRKLDELLK | | |
| | EVEKVHKEVKDRIRKLLEMSCAQESEVKKKLERLLERA | | |
| | KE (SEQ ID NO: 28215) | | |
| degronSwitch_cODC_3plus1_605_t4 | SREELLDRILEAIAKILEDLKRLIDENLARLEEVVREL | 3plus1_Key_Cterm_605 | DEIIRKLDELLKEVEKVHKEVKDRIRKLLEDHKR |
| | ERIIDPNIKLIREILDELKKGSGSEEILEKIKKVDKEL | | SLDEVKKKLERLLERAKEVVEREKK (SEQ ID |
| | EDLIRRLLKKLEDLIRETERRLREILKRIRDLLKEVKD | | NO: 27233) |
| | PDKDLERLLEVLEEVIRVIAELAKELLDSLPKVLKVVE | | |
| | EVLRLLNEVNKEVIDVIRELAKDCGSDETIRKIDELLK | | |
| | EVEKVHKEVKDRIPXLLEDLAMSCAQESKKLERLLERA | | |
| | KEVVE (SEQ ID NO: 28216) | | |
| degronSwitch_cODC_3plus1_605_t1 | SREEILDRILEAIAKILEDLKRLIDENLARLEEVVREL | 3plus1_Key_Cterm_605 | DEIIRKLDELLKEVEKVHKEVKDRIRKLLEDHKR |
| | ERIIDPNLKLIREILDELKKGSGSEEILEKIKKVDKEL | | SLDEVKKKLERLLERAKEVVEREKK (SEQ ID |
| | EDLIRRIIKKIEDLIRETERRLPEILKPIRDLIKEVKD | | NO: 27233) |
| | RDKDLERLIEVLEEVLRVIAELAKELLDSLPKVLKVVE | | |
| | EVLPLLNEVNKEVLDVIRELAKDGGSDETIRKLDELLK | | |
| | EVEKVHREVKDPIRKLLEDHKRLCMSCAQESERLLERA | | |
| | KEVVEREK (SEQ ID NO: 28217) | | |
| degronSwitch_cODC_3plus1_605_t0 | SREELLDPILEATAKILEDLKPLIDENLAPLEEVVREL | 3plus1_Key_Cterm_605 | DEIIRKLDELLKEVEKVHKEVKDRIRKLLEDHKR |
| | ERIIDPNLKLIREILDELKKGSGSEEILEKIKKVDKEL | | SLDEVKKKLERLLERAKEVVEREKK (SEQ ID |
| | EDLIRRLLKKLEDLIRETERRLREILKRIRDLLKEVKD | | NO: 27233) |
| | PDKDLERLLEVLEEVLPVIAELAKELLDSLRKVLKVVE | | |
| | EVLRLLNEVNKEVLDNIRELAKDGGSDEIIRKLDELLK | | |
| | EVEKVHEEVKDRIRKLLEDHKRSLDMSCAQESRLLERA | | |
| | KEVVEREKK (SEQ ID NO: 28218) | | |
| degronSwitch_cODC_3plus1_607_t11 | SEREELLERIKEILKRVKDKLDEDLKPLKEILEKLKEK | 3plus1_Key_Cterm_607 | SETAVRAIIRVLEKHLEAVRPVLEELLKVLAEHL |
| | ADRDLEELRRRIEEVREKLERTGRTDELVKEVLDTVRR | | ETVRELIEPLKRVLEEAIEVVERVAR (SEQ ID |
| | NLENLKRINEDILRKLEENVKNLTDLVREILKLITELI | | NO: 27235) |
| | KRLEDGGLPKEVLDALRRVLEKLEELLREILERLKRSL | | |
| | EAVKRKIEELLKELERSLDELRRALERIRKEIGDSETA | | |
| | VRAIIRVLEKHLEAVRRVLEELLMSCAQESETVRELIE | | |
| | RLKRVLE (SEQ ID NO: 28223) | | |
| degronSwitch_cODC_3plus1_607_t8 | SEREELLERIKEILKRVKDKLDEDIKRLKEILEKLKEK | 3plus1_Key_Cterm_607 | SETAVRAIIRVLEKHLEAVRRVLEELLKVLAEHL |
| | ADRDLEELRRRIEEVREKLERTGRTDELVKEVLDTVRR | | ETVRELIERLKRVLEEAIEVVERVAR (SEQ ID |
| | NLENLKRLVEDILRKLEENVKNLTDLVREILKLITELI | | NO: 27235) |
| | KRLEDGGLPKEVLDALRRVLEKLEELLREILERLKRSL | | |
| | EAVKRKIEELLKELERSLDELRRAIERIRKEIGDSETA | | |
| | VRAIIPVLEKHLEAVRRVLEELLKLQMSCAQESRELIE | | |
| | RLKRVLEEAI (SEQ ID NO: 28224) | | |
| degronSwitch_cODC_3plus1_607_t7 | SEREELLERIKEILKRVKDKLDEDIKRLKEILEKLKEK | 3plus1_Key_Cterm_607 | SETAVRAIIRVLEKHLEAVRRVLEELLKVLAEHL |
| | ADRDLEELRRRIEEVREKLERTGRTDELVKEVLDTVRR | | ETVRELIERLKRVLEEAIEVVERVAR (SEQ ID |
| | NLENLKPLVEDILRKLEENVKNLTDLVREILKLITELI | | NO: 27235) |
| | KRLEDGGLPKEVLDALPRVLEKLEELLREILERLKRSL | | |
| | EAVKRKIEELLKELEPSLDELRRALERIRKEIGDSETA | | |
| | VRAIIRVLEKBLEAVRRVLEELLKVLAMSCAQESELIE | | |
| | PLKRVLEEAIE (SEQ ID NO: 28225) | | |
| degronSwitch_cODC_3plus1_607_t4 | SEREELLERIKEILKRVKDKLDEDLKRLKEILEKLKEK | 3plus1_Key_Cterm_607 | SETAVRAIIPVLEKHLEAVRRVLEELLKVLAEHL |
| | ADRDLEELRRRIEEVREKLERTGRTDEINKEVLDTVRR | | ETVRELIERLKRVLEEAIEVVERVAR (SEQ ID |
| | NLENLKPLVEDILRKLEENVKNLTDLVREILKLITELI | | NO: 27235) |
| | KRLEDGGLPKEVLDALRRVLEKLEELLPEILERLKRSL | | |
| | EAVKRKIEELLKELEPSLDELPRALERIRKEIGDSETA | | |
| | VRAIIPVLEKHLEAVRRVLEELLKVLAELLMSCAQESE | | |
| | RLKRVLEEAIEVVE (SEQ ID NO: 28226) | | |
| degronSwitch_cODC_3plus1_607_t1 | SEREELLERIKETLKRVKDKLDEDLKPLKEILEKLKEK | 3plus1_Key_Cterm_607 | SETAVRAIIRVLEKHLEAVRRVLEELLKVLAEHL |
| | ADRDLEELRRRIEEVREKLERTGRTDELVKEVLDTVRR | | ETVRELIERLKRVLEEAIEVVERVAR (SEQ ID |
| | NLENLKPIVEDILRKLEENVKNLTDLVREILKLITELI | | NO: 27235) |
| | KRLEDGGLPKEVLDALPRVLEKLEELLREILEPLKKSL | | |
| | EAVKRKIEELLKELERSLDELPRALERIRKEIGDSETA | | |
| | VRAIIRVLEKHLEAVRRVLEELLKVLAEHLELVMSCAQ | | |
| | ESKRVLEEAIEVVERVA (SEQ ID NO: 28227) | | |
| degronSwitch_cODC_3plus1_607_t0 | SEREELLERIKEILKRVKDKLDEDLKKLKEILEKLKEK | 3plus1_Key_Cterm_607 | SETAVRAIIRVLEKHLEAVRRVLEELLKVLAEHL |
| | ADRDLEELRRRIEEVREKLERTGRTDELVKEVLDTVRR | | ETVRELIERLKRVLEEAIEVVERVAR (SEQ ID |
| | NLENLKRLVEDILRKLEENVKNLTDLVREILKLITELI | | NO: 27235) |
| | KRLEDGGLPKEVLDALRRVLEKLEELLREILERLKRSL | | |
| | EAVKRKIEELLKELERSLDELRRALERIRKEIGDSETA | | |
| | VRAIIRVLEKELEAVRRVLEELLKVLAEHLETLKMSCA | | |
| | QESRVLEEAIEVVERVAR (SEQ ID NO: 28228) | | |
| degronSwitch_cODC_3plus1_610_t1 | SLEEITKRLIELVEENLARKEEILRELLELAKRLAKED | 3plus1_Key_Cterm_610 | ERTLREVVRKVLEEAKRLLDELEEVHKRVKKELE |
| | RDILEEVLKLIEELLKLLEDNGSSEEDLKRLLKEVIEE | | DIIEENRPVVKPVRDELREIKRELDE (SEQ ID |
| | LRAVVKRVKDKWDEVVKRIEDLVKKLKELHDDTLRKLR | | NO: 27237) |
| | ELVRKIVTDISESGGEAEKVKRVVEKILELVERLAKVV | | |
| | KESVEKLLEILRELAEVSKRVARALLRLLEELVRVIRI | | |
| | KDERTZREVVRKVLEEAKRLLDELEEVHKRVKKELEDL | | |
| | IMSCAQESKRVRDELPEIKRELD (SEQ ID | | |
| | NO: 28237) | | |
| degronSwitch_cODC_3plus1_611_t1 | SLEEITKRLLELNEENLARHEEILRELLELAKRLAKED | 3plus1_Key_Cterm_611 | ERTLREVVRKVLEEAKRLILDELEEVHKRVKKELE |
| | RDILEEVLKIIEELLKLLEDNGSSEEDLKRLLKEVIEE | | DIIEENRRVVKRVRDELREIKRELDE (SEQ ID |
| | LRAVVKRVKDKWDEVVKRIEDLVKKLKELHDDTLRKLR | | NO: 27239) |
| | ELVRKIVTDISESGSEAEKVKRVVEKILELVERLAKVV | | |
| | KESVEKLLEILKELAEVSKRVAEALLRLLEELVRVIRI | | |
| | KDERTLREVVRKVLEEAKPLLDELEEVHKRVKKELEDL | | |
| | EMSCAQESKRVRDELREIKRELD (SEQ ID | | |
| | NO: 28238) | | |
| degronSwitch_cODC_3plus1_632_t15 | SEKELVDDIRRILEEILPLLRSLLEEVIRLLEENEKLV | 3plus1_Key_Cterm_632 | DSLVREVEELIKRLEKHKIDDLLKTSRDLVKRVLD |
| | RRHLKTVIDILRRVAKLLDENGIRTDEADRVLERLEKA | | LVDEVVKRVEDLVERVKEKIDT (SEQ ID |
| | HRELLEDYKRALEKIKETLERVLREAEEVVKKIDDALR | | NO: 27241) |
| | KLGGSKEVLKRLLEELLRLVEKIAEEIKRLLSELVRVT | | |
| | EELVRTNKELLEEAVRVIRKEVGDDSLVREVEELIKRL | | |
| | EKHLDMSCAQESDLATKRVLDLVDEVVK (SEQ ID | | |
| | NO: 28317) | | |
| degronSwitch_cODC_3plus1_632_t12 | SEKELVDDIRRILEEILRLLPSLLEEVIRLLEENEKLV | 3plus1 Key_Cterm_632 | DSLVREVEELIKPLEKKIDDLLKTSRDLVKRVLD |
| | RRHLYTVIDILRRVAKLLDENGIRTDEADRVLERLEKA | | LVDEVVKRVEDLVERVKEKIDT (SEQ ID |
| | HRELLEDYKRALEKIKETLERVLREAEEVVKKIDDALR | | NO: 27241) |
| | KLGGSKEVLKRLLEELLRLVEKIAEEIKRLLSELVRVT | | |
| | EELVRTNKELIEEAVRVIRKEVGDDSLVREVEELIKRL | | |
| | EKHIDDLLMSCAQESKRVLDLVDEVVKRVE (SEQ ID | | |
| | NO: 28318) | | |
| degronSwitch_cODC_3plus1_632_t8 | SEKELVDDIRRILEKLRPLLRSLLEEVIRLLEENEKLV | 3plus1_Key_Cterm_632 | DSLVREVEELIKRLEKHIDDLLKTSRDLVKRVLD |
| | RRHLKTVIDILRRVAKLLDENGIRTDEADRVLERLEKA | | LVDEVVKRVEDLVERVKEKIDT (SEQ ID |
| | HRELLEDYKRALEKIKETLERVLREAEEVVKKIDDALR | | NO: 27241) |
| | KLGGSKEVLKRLLEELLRLVEKIAEEIKRLLSELVRVT | | |
| | EELVRTNKELLEEAVRVIRKEVGDDSLVREVEELIKRL | | |
| | EKHIDDLLKTLRMSCAQESDLVDEVVKRVEDLVE | | |
| | (SEQ ID NO: 28319) | | |
| degronSwitch_cODC_3plus1_632_t5 | SEKELVDDIRRILEEILRLLRSLLEEVIRLLEENEKLV | 3plus1_Key_Cterm_632 | DSLVREVEELIKRLEKEIDDLLKTSRDLVKRVLD |
| | RRHLKTVIDILRRVAKLLDENGIRTDEADRVLERLEKA | | LVDEVVKRVEDLVERVKEKIDT (SEQ ID |
| | HRELLEDYKRALEKIKETLERVLREAEEVVKKIDDALR | | NO: 27241) |
| | KLGGSKEVLKRLLEELLRLVEKTAEEIKRLLSELVRVT | | |
| | EELVRTNKELLEKAVRVIRKEVGDDSLVREVEELIKRL | | |
| | EKHIDDLLKTSRDLVMSCAQESDEVVKRVEDLVERVK | | |
| | (SEQ ID NO: 28320) | | |
| degronSwitch_cODC_3plus1_632_t4 | SEKELVDDIRRILEEILRLLRSLLEEVIRLLEENEKLV | 3plus1_Key_Cterm_632 | DSLVREVEELIKRLEKHIDDLLKTSRDLVKRVLD |
| | RRHLKTVIDILRRVAKLLDENGIRTDEADRVLERLEKA | | LVDEVVKRVEDLVERVKEKIDT (SEQ ID |
| | HRELLEDYKPALEKIKETLERVLRENEEVVKKIDDALR | | NO: 27241) |
| | KLGGSKFVLKRLLEELLPLVEKTAEEIKRLLSELVRVT | | |
| | EELVRTNKELLEEAVRVIRKEVGDDSLVREVEELIKRL | | |
| | EKHIDDLLKTSRDLLKMSCAQESEVVYRVEDLVERVKE | | |
| | (SEQ ID NO: 28321) | | |
| degronSwitch_cODC_3plus1_632_t1 | SEKELVDDIRRILEEILRLLRSLLEEVIRLLEENEKLV | 3plus1_Key_Cterm_632 | DSLVREVEELIKRIEKHIDDLLKTSRDLVKRVLD |
| | RRHLKTVIDILRRVAKLLDENGIRTDEADRVLERLEKA | | LVDEVVKRVEDLVERVKEKIDT (SEQ ID |
| | HRELLEDYKRALEKIKETLERVDREAEZVVKKIDDALR | | NO: 27241) |
| | KLGGSKEVLKRLLEELLPLVEKIAEEIKRLDSELVRVT | | |
| | EELVRTNKELLEEAVRVIRKEVGDDSLVREVEELIKRL | | |
| | EKHIDDLLKTSRDLVKRLAMSCAQESKRVEDLVERVKE | | |
| | KID (SEQ ID NO: 28322) | | |
| degronSwitch_cODC_3plus1_641_t15 | DEVEELLKRVRELLKASEELVRKILEDVKRLLERSIEE | 3plus1_Key_Cterm_641 | EDLIRDVDRVLKEIMDLLRDLVRRTKELLEELLR |
| | VEDLLRKVEELLKRLLDLVERGGSLDEILRELVRLLKE | | LLEELVRAHKELVRSILDEIRK (SEQ ID |
| | IVRKVLELNPYLVEDVIRIAKRLLELNAESVKEWDRIL | | NO: 27243) |
| | KLLRDGTGDKEELLKDAEEALEKYERAVREILKELEEI | | |
| | IKEYVRPVEELLKELKDAVDKAKDEVRKGSGDEDLIRD | | |
| | VDRVLKEIMDLLMSCAQESELLEELLRLLEELVR | | |
| | (SEQ ID NO: 28358) | | |
| degronSwitch_cODC_3plus1_641_t4 | DEVEELLKRVRELLKASEELVRKILEDVKPLIERSIEE | 3plus1_Key_Cterm_641 | EDLIRDVDRVLKEIMDLLRDLVRRTKELLEELLR |
| | VEDLLRKVEELLKRLLDLVERGGSLDEILRELVRLLKE | | LLEELVRAHKELVRSILDEIRK (SEQ ID |
| | IVRKVLELNRKLVEDVIRIAKRLLELNAESVKEWDRIL | | NO: 27243) |
| | KLLRDGTGDKEELLKDAEEALEKYERAVREILKELEEI | | |
| | IKEYVRRVEELLKELKDAVDKAKDEVRKGSGDEDLIRD | | |
| | VDRVLKEIMDILRDLVRRTKELLEMSCAQESELVRAHK | | |
| | ELVRSILD (SEQ ID NO: 28359) | | |
| degronSwitch_cODC_3plus1_641_t1 | DEVEELLKPVRELLKASEELVRKILEDVKRLLERSIEE | 3plus1_Key_Cterm_641 | EDLIRDVDRVLKEIMDLLRDLVRRTKELLEELLR |
| | VEDLLRKVEELLKRLLDLVERGGSLDETDRELVRLLKE | | LLEEIVRAHKELVRSILDEIRK (SEQ ID |
| | IVRKVLELNRKLVEDVIRIAKRLLELNAESVKEWDRIL | | NO: 27243) |
| | KLLRDGTGDKEELLKDAEEALEKYERAVREILKELEEI | | |
| | IKEYVRFVEELLKELKDAVDKAKDEVRKGSGDEDLIRD | | |
| | VDRVLKEIMDLLRDLVRRTKELLEELLMSCAQESRAHK | | |
| | ELVRSILDEIR (SEQ ID NO: 28360) | | |
| degronSwitch_cODC_3plus1_646_t15 | DAEEVVKRLADVLRENDETIRKVVEDLVRIAEENDRLW | 3plus1_Key_Cterm_646 | EDVKRALEEINSRLRKLLEDVKKASEDIVREVER |
| | KKLVEDIAEILRRIVELLRRGGVPEELLDRLAKVVKSI | | IVRELAKRSDEILKKLEDIVEKLRE (SEQ ID |
| | VEKAEKILERLNRVSKAIAEKLKTIVDELNEVSKEIVK | | NO: 27245) |
| | RAEDILRKGKDKETVLRALRTLVKEYADLSKEVLERVE | | |
| | RIVREYVKLSDEVVKSLAEIVEELIRIIEDLLRKGNLD | | |
| | EDVKRNDEELVSRLRKLLEDLAMSCAQESREVERIVRE | | |
| | LAKRSD (SEQ ID NO: 28376) | | |
| degronSwitch_cODC_3plus1_646_t12 | DAEEVVERLADVDRENDETIRKVVEDLVRIAEENDRLW | 3plus1_Key_Cterm_646 | EDVKRALEELVSRLRKLLEDVKKASEDIVREVER |
| | KKLVEDIAEILRRIVELLRRGGVPEELLDRLAKVVKSI | | IVRELAKRSDEILKKEDIVEKLRE (SEQ ID |
| | VEKAEKILERLNRVSKAIAEKLKTIVDELNEVSKEIVK | | NO: 27245) |
| | RAEDILRKGKDKETVLRALRTLVKEYADLSKEVLERVE | | |
| | RIVREYVKLSDEVVKSLAEIVEELIRIIEDLLRKGNLD | | |
| | EDVKRALEELVSRLRKLLEDVKKLAMSCAQESERIVRE | | |
| | LAKRSDEIL (SEQ ID NO: 28377) | | |
| degronSwitch_cODC_3plus1_646_t8 | DAEEVVKRLADVLRENDETIRKVVEDLVRIAEENDRLW | 3plus1_Key_Cterm_646 | EDVNRALEELVSRLRKLLEDVKKASEDIVREVER |
| | KKLVEDIAEILRRIVELLRRGGVPEELLDRLAKVVKSI | | IVRELAKRSDEILKKLEDIVEKLRE (SEQ ID |
| | VEKAEKILERLNRVSKAIAEKLKTIVDELNEVSKEIVK | | NO: 27245) |
| | RAEDILRKGKDKETVLRALRTLVKEYADLSKEVLERVE | | |
| | RIVREYVKLSDEVVKSLAEIVEELIRIIEDLLPKGNLD | | |
| | EDVKRALEELVSRLRKLLEDVKKASEDLVMSCAQESRE | | |
| | LAKRSDEILKKLE (SEQ ID NO: 28378) | | |
| degronSwitch_cODC_3plus1_646_t5 | DAEEVVKRLADVLRENDETIRKVVEDLVRIAEENDRLW | 3plus1_Key_Cterm_646 | EDVKRALEELVSRLRKLLEDVKKASEDIVREVER |
| | KKLVEDIAEILRRIVELLRRGGVPEELLDRLAKVVKSI | | IVRELAKRSDEILKKLEDIVEKLRE (SEQ ID |
| | VEKAEKILERLNRVSKAIAEKLKTIVDELNEVSKEIVK | | NO: 27215) |
| | RAEDILRKGKDKETVLRALRTLVKEYADLSKEVLERVE | | |
| | RIVREYVKLSDEVVKSLAEIVEELIRIIEDLLRKGNLD | | |
| | EDVKRNLEELVSRLRKLLEDVKKASEDIVRLQMSCAQE | | |
| | SAKRSDEILKKLEDIV (SEQ ID NO: 28379) | | |
| degronSwitch_cODC_3plus1_646_t4 | DAEEVVKRLADVLRENDETIRKTVEDLVRIAEENDRLW | 3plus1_Key_Cterm_646 | EDVKRALEELVSRLRKLLEDVKKASEDIVREVER |
| | KKLVEDIAEILRRIVELLRRGGVREELLDRLAKVVKSI | | IVRELAKRSDEILKKLEDIVEKLRE (SEQ ID |
| | VEKAEKILERLNRVSKAIAEKLKTIVDELNEVSKEIVK | | NO: 27245) |
| | PAEDILRKGKDKETVLPALRTLVKEYADLSKEVLERVE | | |
| | RIVREYVKLSDEVVKSLAEIVEELIRIIEDLLRKGNLD | | |
| | EDVKRNLEELVSRLRKLLEDVKKASEDIVRELLMSCAQ | | |
| | ESKRSDEILKKLEDIVE (SEQ ID NO: 28380) | | |
| degronSwitch_cODC_3plus1_646_t1 | DAEEVVKRLADVLRENDETIRKVVEDLVRIAEENDRLW | 3plus1_Key_Cterm_646 | EDVKRNLEELVSRLRKLLEDVKKASEDIVREVER |
| | KKLVEDIAEILRRIVELLRRGGVPEELLDRLAKVVKSI | | IVRELAKRSDEILKKLEDIVEKLRE (SEQ ID |
| | VEKAEKILERLNRVSKAIAEKLKTIVDELNEVSKEIVK | | NO: 27245) |
| | RAEDILRKGKDKETVLRNLRTLVKEYADLSKEVLERVE | | |
| | RIVREYVKLSDEVVKSLNEIVEELIRIIEDLLRKGNLD | | |
| | EDVKRALEELVSRLRKLLEDVKKNSEDIVREVERLVMS | | |
| | CAQESDEILKKLEDIVEKLR (SEQ ID NO: 28381) | | |
| degronSwitch_cODC_3plus1_647_t15 | DAEEVVKRLADVLRENDETIRKTVEDLVRIAEENDRLW | 3plus1_Key_Cterm_647 | EDVKRALEELVSRLRKLLEDVKKASEDIVREVER |
| | KKLVEDIARILRRIVELLRRGGVPEELLDRLAKVVKSI | | IVRELAKRSDEILKKLEDIVEKLRE (SEQ ID |
| | VEKAEKTITRLNRVSKAIAEKLKTIVDELNEVSKEIVK | | NO: 27251) |
| | RAEDILRKGKDKETVLRALRTLVKEYADLSKEVLERVE | | |
| | RIVREYVKLSDEVVKSLAEIVEELIRIIEDLLRKGNLD | | |
| | EDVKRALEELVSRLRKLLEDLRMSCAQESREVERIVRE | | |
| | LAKRSD (SEQ ID NO: 28382) | | |
| degronSwitch_cODC_3plus1_647_t12 | DAEEVVKRLADVLRENDETIRKVVEDLVRIAEENDRLW | 3plus1_Key_Cterm_647 | EDVKRALEELVSRLRKLLEDVKKASEDIVREVER |
| | KKLVEDIAEILRRIVELLRRGGVPEELLDRLAKVVKSI | | IVRELAKRSDEILKKLEDIVEKLRE (SEQ ID |
| | VEKAEKILERLNRVSKAIAEKLKTIVDELNEVSKEIVK | | NO: 27251) |
| | RAEDILRKGKDKETVLRALRTLVKEYADLSKEVLERVE | | |
| | RIVREYVKLSDEVVKSLAEIVEELIRIIEDLLRKGNLD | | |
| | EDVKRALEELVSRLRKLLEDVKKLSMSCAQESERIVRE | | |
| | LNKRSDEIL (SEQ ID NO: 28383) | | |
| degronSwitch_cODC_3plus1_647_t11 | DAEEVVKRLADVLRENDETIRKVVEDLVRIAEENDRLW | 3plus1_Key_Cterm_647 | EDVKRNLEELVSRLRKLLEDVKKNSEDIVREVER |
| | KKLVEDIAEILRRIVELLRRGGVPEELLDRLAKVVKSI | | IVRELAKRSDEILKKLEDIVEKLRE (SEQ ID |
| | VEKAEKILERLNRVSKAIAEKLKTIVDELNEVSKEIVK | | NO: 27251) |
| | RAEDILRKGKDKETVLRALRTLVKEYADLSKEVLERVE | | |
| | RIVREYVKLSDEVVKSLAEIVEELIRIIEDLLPKGNLD | | |
| | EDVKRALEELVSRLRKLLEDVKKALKMSCAQESRIVRE | | |
| | LAKRSDEILK (SEQ ID ND:28384) | | |
| degronSwitch_cODC_3plus1_647_t8 | DAEEVVKRLADVLRENDETIRKVVEDLVRIAEENDRLW | 3plus1_Key_Cterm_647 | EDVKRALEELVSRLRKLLEDVKKASEDIVREVER |
| | KKLVEDIAEILRRIVELLRRGGVPEELLDRLAKVVKSI | | IVRELAKRSDEILKKLEDIVEKLRE (SEQ ID |
| | VEKAEKILERLNRVSKAIAEKLKTIVDELNEVSKEIVK | | NO: 27251) |
| | RAEDILRKGKDKETVLRALRTLVKEYADLSKEVLERVE | | |
| | RIVREYVKISDEVVKSLAEIVEELIRIIEDLLRKGNLD | | |
| | EDVKRALEELVSRLRKLLEDVKKASEDLVMSCAQESRE | | |
| | LAKRSDEILKKLE (SEQ ID NO: 28385) | | |
| degronSwitch_cODC_3plus1_647_t5 | DAEEVVERLADVLRENDETIRKVVEDLVRIAEENDRLW | 3plus1_Key_Cterm_647 | EDVKRALEELVSRLRKLLEDVKKASEDIVREVER |
| | KKLVEDIAEILRRIVELLRRGGVPEELLDRLAKVVKSI | | IVRELAKRSDEILKKLEDIVEKLRE (SEQ ID |
| | VEKAEKILERLNRVSKAIAEKLKTIVDELNEVSKEIVK | | NO: 27251) |
| | RAEDILRKGKDKETVLRALRTLVKEYADLSKEVLERVE | | |
| | RIVREYVKLSDEVVESLAEIVEELIRIIEDLIRKGNLD | | |
| | EDVKRAIEEWSELRKLLEDVKKASEDIVRINMSCAQE | | |
| | SAKESDEILKKLEDIV (SEQ ID NO: 28386) | | |
| degronSwitch_cODC_3plus1_647_t4 | DAEEVVKRLADVLRENDETIRKVVEDLVRIAEEVDRLW | 3pLusl_Key_Cterm_647 | EDVKRALEELVSRLRKLLEDVKKASEDIVREVER |
| | KKLVEDIAEILRRIVELLRRGGVPEELLDRLAKVVKSI | | IVRELAKRSDEILKKLEDIVEKLRE (SEQ ID |
| | VEKAEKILERLNRVSKAIAEKLKTIVDELNEVSKEIVK | | NO: 27251) |
| | RAEDILRKGKDKETVLRALPTLVKEYADLSKEVLERVE | | |
| | RIVREYVKISDEVVKSLNEIVEELIRIIEDLLRKGNLD | | |
| | EDVKRALEELVSRLRKLLEDVKKASEDIVRELQMSCAQ | | |
| | ESKRSDEIDKKLEDIVE (SEQ ID NO: 28387) | | |
| degronSwitch_cODC_3plus1_647_t1 | DAEEVVKRLADVLRENDETIRKVVEDLVRIAEENDRLW | 3plus1_Key_Cterm_647 | EDVKRALEELVSRLRKLLEDVKKASEDIVREVER |
| | KELVEDIAEILRRIVELLRRGGVPEELLDRLAKVVKSI | | IVRELAKRSDEILKKLEDIVEKLRE (SEQ ID |
| | VEKAEKILEPLNRVSKAIAEKLKTIVDELNEVSKEIVK | | NO: 21251) |
| | RAEDILRKGKDKETVLPALPTLVKEYADLSKEVLERVE | | |
| | RIVREYVKLSDEVVKSLAEIVEELIRIIEDLLRKGNLD | | |
| | EDVKRALEELVSRLRKLLEDVKKASEDIVREVERLVMS | | |
| | CAQESDEILKKLEDIVEKLR (SEQ ID NO: 28388) | | |
| degronSwitch_cODC_3plus1_653_t15 | DEEETLRELLERKVELAKEYLDVSKEVIDPTTKLLDEY | 3plus1_Key_Cterm_653 | SREALEEARRRLEELLRELNEITKDLEAKLEKLL |
| | LKTSKRIVDATVELLERGDLGPDELIKRLAEELERSLR | | PDLNELTKALEEELKRLLDELKKRTD (SEQ ID |
| | ELEEEIKRLKRELEESLKKLKEIIDRLAEKAEKLLAVL | | NO: 27257) |
| | KRGEGSEEEALRALASLVRELIEVLRENDERLPDVLRR | | |
| | LIEALRKNNEILERVLRKLVRAAEERGRDESSREALEE | | |
| | ARRRLEELLBELNELAMSCAQESEKLLRDLNELTKALE | | |
| | (SEQ ID NO: 28410) | | |
| degronSwitch_cODC_3plus1_653_t11 | DEEETLRRLLERKVELAKEYLDVSKEVIDRTTYLLDEY | 3plus1_Key_Cterm_653 | SREALEEARRRLEELLRELNEITKDLEAKLEKLL |
| | LKTSKRIVDATVELLERGDLGPDELIKRLAEELERSLR | | RDLNELTKALEEELKRLLDELKKRTD (SEQ ID |
| | ELEEEIKRLKRELEESIKKLKEIIDRLAELAEKLLAVL | | NO: 27257) |
| | KRGEGSEERALRALASLVRELIEVLRENDERLRDVLRR | | |
| | LIEALRKNNEILERVLRKLVRAAEERGRDESSREALEE | | |
| | ARRRLEELLRELNEITKDLEMSCAQESRDLNELTKALE | | |
| | EELK (SEQ TD NO: 28411) | | |
| degronSwitch_cODC_3plus1_653_t8 | DEEETLRRLLERKVELAKEYLDVSKEVIDPTTKLLDEY | 3plus1_Key_Cterm_653 | SREALEEARRRLEELLRELNEITKDLEAKLEKLL |
| | LKTSKRIVDATVELLERGDLGPDELIKRLNEELERSLR | | RDLNELTKALEEELKRLLDELKKRTD (SEQ ID |
| | ELEEEIKRLKRELEESLKKLKEIIDRIAEEAEKLLAVL | | NO: 27257) |
| | KRGEGSEEEALRNLASLVRELIEVLRENDERLRDVLRR | | |
| | LIEALRKNNEILERVLRKLVRAAEEPGRDESSREALEE | | |
| | ARRRLEELIRELNEITKDLEALMMSCAQESNELTKALE | | |
| | EELKRLL (SEQ ID NO: 28412) | | |
| degronSwitch_cODC_3plus1_653_t4 | DEEETLRRLIERKVELAKEYLDVSKEVIDRTTKLLDEY | 3plus1_Key_Cterm_653 | SREALEEARRRLEELLPELNEITKDLEAKLEKLL |
| | LKTSKRIVDATVELLEPGDLGPDELTKRLAEELEPSLR | | RDLNELTKALEEELKRLLDELKKRTD (SEQ ID |
| | ELEEEIKRLKEELEESLKKLKEIIDPLAERAEKLLAVL | | NO: 27257) |
| | KRGEGSEEEALRALASLVRELIEVLRENDERLRDVLRR | | |
| | LIEALRKNNEILERVLRKLVRAAEERGRDESSREALEE | | |
| | ARRRLEELLRELNEITKDLEAKLEKLLMSCAQESKALE | | |
| | EELKRLLDELK (SEQ ID NO: 28413) | | |
| degronSwitch_cODC_3plus1_653_t1 | DEEETLRRLLERKVELAKEYLDVSKEVIDRTTKLLDEY | 3plus1_Key_Cterm_653 | SREALEEARRRLEELLRELNEITKDLEAKLEKLL |
| | LKTSKRIVDATVELLERGDLGPDELIKRLAEELERSLR | | RDLNELTKALEEELKRLLDELKKRTD (SEQ ID |
| | ELEEEIKPIKRELEESLKKLKEIIDPLAEEAEKLLAVL | | NO: 27257) |
| | KRGEGSEERALRALASLVRELIEVLRENDERLPDVLRR | | |
| | LIEALRKNNEILERVLRKLVRAAEERGRDESSREALEE | | |
| | ARRRLEELLKELNEITKDLEAKLEKLLRLLMSCAQESE | | |
| | EELKRLLDELKKRT (SEQ ID NO: 28414) | | |
| degronSwitch_cODC_3plus1_653_t0 | DEEETLRRLLERKVELAKEYLDVSKEVIDRTTKLLDEY | 3plus1_Key_Cterm_653 | SREALEEARRRLEELLRELNEITKELEAKLEKLL |
| | LKTSKRIVDATVELLEPGDLGPDELIKRLAEELERSLR | | RDLNELTKALEEELKRLLDELKKRTD (SEQ ID |
| | ELEEEIKPIKRELEESLKKLKEIIDRDAEEAEKLLAVL | | NO: 27257) |
| | KRGEGSEEEALRALASLVRELIEVLRENDEPLRDVLRR | | |
| | LIEALRKNNEILERVLRKIVRAAEERGRDESSREALEE | | |
| | ARRRLEELLRELNEITKDLEAKLEKLLRDLAMSCAQES | | |
| | EELKRLLDELKKRTD (SEQ ID NO: 28415) | | |
| degronSwitch_cODC_3plus1_658_t15 | DEERIIKTLEDINAKLVEDIKRILDKVAELNERLADAI | 3plus1_Key_Cterm_658 | KDTLRTVEKLVEDVKPRLDKLLEDYKRLIEEVKK |
| | RKILEETKRILEATTRKVRKDGEISEELLPPLEEKLRK | | ELDKLLKEYEDALREIKKRIDE (SEQ ID |
| | LLEDLEPVLAENEDESRRILEEVEPLLKRHADASKELL | | NO: 27259) |
| | DRARSVARGVKSDKELVDPIKKLIDDSLESVRELIERL | | |
| | KELLDPLVKSVEDLIRTIKELLDPLVEVLREGVSDKDT | | |
| | LRTVEKLVEDVKRRLDMSCAQESRLIEEVKKELDKLLK | | |
| | (SEQ ID NO: 28427) | | |
| degronSwitch_cODC_3plus1_658_t12 | DEERIIKTLEDINAKLVEDIKRILDKVAELNERLADAI | 3plus1_Key_Cterm_658 | KDTLRTVEKLVEDVKRRLDKLLEDYKRLIEEVKK |
| | RKILEETKRILEATTPKVRKDOEISEELLRRLEEKLRK | | ELDKLLKEYEDALREIKKRIDE (SEQ ID |
| | LLEDLERVLAENEDESRRILEEVERLLKREADASKELL | | NO: 27259) |
| | DRARSVAROVKSDKELVDRLKKLIDDSLESVRELIERL | | |
| | KELLDRLVKSVEDLIRTIKELLDRLVEVLREGVSDKDT | | |
| | LRTVEKLVEDVKRRLDKLGMSCAQESEEVKKEIDKLLK | | |
| | EYE (SEQ ID NO: 28428) | | |
| degronSwitch_cODC_3plus1_658_t8 | DEERIIKTLEDINAKLVEDIKRILDKVAELNERLADAI | 3plus1_Key_Cterm_658 | KDTLRTVEKLVEDVKRRLDKLLEDYKRLIEEVKK |
| | RKILEETKRILEATTRKVRKDGEISEELLRRLEEKLRK | | ELDKLLKEYEDALREIKKRIDE (SEQ ID |
| | LLEDLERVLAEHEDESRRILEEVERLLKRHADASKELL | | NO: 27259) |
| | DRARSVARGVKSDKELVDRLKKLIDDSLESVRELIERL | | |
| | KELLDRLVKSVEDLIRTIKELLDRLVEVLREGVSDKDT | | |
| | LRTVEKLVEDVKRRIDKLLEDLKMSCAQESKELDKLLK | | |
| | EYEDALR (SEQ ID NO: 28429) | | |
| degronSwitch_cODC_3plus1_658_t5 | DEERIIKTLEDINAKLVEDIKRILDKVAELNERLADAI | 3plus1_Key_Cterm_658 | KDTLRTVEKLVEDVKRRLDKLLEDYKRLIEEVKK |
| | RKILEETKRILEATTRKVRKDGEISEELLRRLEEKLRK | | ELDKLLKEYEDALREIKKRIDE (SEQ ID |
| | LLEDLERVLAEHEDESRRILEEVERLLKRHADASKELL | | NO: 27259) |
| | DRARSVAROVKSDKELVDREKKLIDDSLESVRELIERL | | |
| | KELLDRLVKSVEDLIRTIKELLDRLVEVLREGVSDKDT | | |
| | LRTVEKLVEDVKRRLDKLLEDYKRLMMSCAQESDKLLK | | |
| | EYEDALREIK (SEQ ID NO: 28430) | | |
| degronSwitch_cODC_3plus1_658_t1 | DEERIIKTLEDINAKLVEDIKRILDKVAELNERLADAI | 3plus1_Key_Cterm_658 | KDTLRTVEKIVEDVKRRLDKLLEDYKRLIEEVKK |
| | RKILEETKRILEATTRKVRKDGEISEELLRRLEEKLRK | | ELDKLLKEYEDALREIKKPIDE (SEQ ID |
| | LLEDLERVLAEHEDESRRILEEVERLLKRNADASKELL | | NO: 27259) |
| | DRARSVARGVKSDKELVDRLKKLIDDSLESVRELIERL | | |
| | KELLDRLVKSVEDLIPTIKELLDRLVEVLREGVSDKDT | | |
| | LRTVEKLVEDVKRRLDKLLEDYKRLIEELKMSCAQESK | | |
| | EYEDALREIKKRID (SEQ ID NO: 28431) | | |
| degronSwitch_cODC_3plus1_660_t15 | TEEEVVEDVKRVLDESHDDLRRLIETLTRVLRESLKRI | 3plus1_Key_Cterm_660 | REEVKRVLEEARDELRRLLEEYKAIIEELERELE |
| | KEALEELERVLKKLLDLLEGGRDAREVLDEIRKVLERL | | RLLREHREVIRRIKEEIDKSSK (SEQ ID |
| | REVIEELLRINKEVLRELERVIRELLKKNEDLARRVRS | | NO: 27261) |
| | GVKSRLLEVLERLARESLELNRAILEELRKIVEKSLRA | | |
| | VEKILKRLEEIVRKLLKLVEDGGPREEVKRVLEEARDE | | |
| | LRRLLMSCAESEELERELERLIREHR (SEQ ID | | |
| | NO: 28437) | | |
| degronSwitch_cODC_3plus1_660_t11 | TEEEVVEDVKRVLDESHDDLRRLIETLTRVLRESLKRI | 3plus1_Key_Cterm_660 | REEVKRVLEEARDELRRLLEEYKAIIEELERELE |
| | KEALEELERVLKKLLDLLEGGRDAREVLDEIRKVLERL | | RLLREHREVIRRIKEEIDKSSK (SEQ ID |
| | REVIEELLRINKEVLRELERVIRELLKKNEDLARRVRS | | NO: 27261) |
| | GVKSRLLEVLERLARESLELNRAILEELRKINEKSLRA | | |
| | VEKILKRLEEIVRKLLKLVEDGGPREEVKRVLEEARDE | | |
| | LRRLLEELKMSCAQESRELERLLREHREVIR (SEQ | | |
| | ID NO: 28438) | | |
| degronSwitch_cODC_3plus1_660_t8 | TEEEVVEDVKRVLDESHDDLRRLIETLTRVLRESLKRI | 3plus1_Key_Cterm_660 | REEVKRVLEEARDELRRLLEEYKAIIEELERELE |
| | KEALEELERVLKKLLDLLEGGRDAREVLDEIRKVLERL | | RLLREHREVIRRIKEEIDKSSK (SEQ ID |
| | REVIEELLRINKEVLRELERVIRELLKKNEDLARRVRS | | NO: 27261) |
| | GVKSRLLEVLERLARESLELNRAILEELRKLVEKSLRA | | |
| | VEKILKRLEEIVRKLLKLVEDGGPREEVKRVLEEARDE | | |
| | LRRLLEEYKALVMSCAQESERLLREHREVIRRIK | | |
| | (SEQ ID NO: 28439) | | |
| degronSwitch_cODC_3plus1_660_t1 | TEEEVVEDVKRVLDESHD)LRRLIETLIRVLPESLKRI | 3plus1_Key_Cterm_660 | REEVKRVIEEARDELRRLLEEYKAIIEELERELE |
| | KEALEELERVLKKLLDLLEGGRDAREVLDEIRKVLERL | | RLLREHREVIRRIKEEIDKSSK (SEQ ID |
| | REVIEELLRINKEVLRELERVIRELLKKNEDIARRVRS | | NO: 27261) |
| | GVKSRLLEVLERLARESLELNPAILEELRKLVEKSLRA | | |
| | VEKILKRLEEIVRKLLKINEDGGPREEVKPVLEEARDE | | |
| | LRRLLEEYKAIIEELERLLMSCAQESREVIRRIKEEID | | |
| | KSS (SEQ ID NO: 28440) | | |

In some aspects, the polypeptide of the present disclosure further comprises one or more signaling proteins, e.g., input-receiving member, intermediate member, and/or output producing member, of a signaling pathway. As used herein, the term “signaling protein” generally refers to a protein of a signaling pathway, including natural and synthetic signaling pathways, described in more detail below. Any convenient and appropriate signaling protein of any convenient signaling pathway can be employed. Generally, signaling proteins include proteins that can be activated by an input of the signaling pathway with which the signaling protein is associated. A signaling pathway can generate an output that is dependent upon, or at least influenced by, the function of the signaling protein. Such outputs can be a direct or indirect result of the response of the signaling protein to the input. Useful signaling proteins include members from any convenient and appropriate point a signaling pathway, including input-receiving members, intermediate members, and output-producing members. By “input-receiving members”, as used herein, is generally meant the initial component of a signaling pathway that receives an input to initiate signaling along the pathway. Examples of input-receiving members include but are not limited to e.g., extracellular receptors (e.g., G protein-coupled receptors, protein kinases, integrins, toll-like receptors, ligand-gated ion channels, and the like) and intracellular receptors (e.g., nuclear receptors, cytoplasmic receptors, etc.). In some aspects, an input-receiving member can be a protein that directly binds an input of a signaling pathway, such as a ligand input of a signaling pathway. In some aspects, a signaling protein that includes a degron in a cage polypeptide of the present disclosure can be an input-receiving member. In some aspects, a signaling protein that includes a a degron in a cage polypeptide in a polypeptide of the present disclosure cannot be an input-receiving member, e.g., it can be an intermediate member or an output-producing member.

By “intermediate member”, as used herein, is generally meant a component of a signaling pathway that is required for, or at least involved in, signal transduction but does not directly receive the initial input or directly produce or cause the final output of the signaling pathway. Examples of intermediate members of a signaling pathway include but are not limited to e.g., enzymes, binding partners, protein complex subunits, scaffold proteins, transport proteins, co-activators, co-repressors, and the like. In some aspects, a signaling protein that includes a degron in a cage poly peptide of the present disclosure can be an intermediate member. In some aspects, a signaling protein that includes a degron in a cage polypeptide of the present disclosure cannot be an intermediate member, e.g., it can be an input-receiving member or an output-producing member.

By “output-producing member”, as used herein, is generally meant a component of a. signaling pathway that directly produces an output of the signaling pathway or otherwise causes the output of the signaling pathway to occur. Examples of output-producing members of a signaling pathway include but are not limited to e.g., DNA binding proteins, such as e.g., transcription factors, enzymes, and the like. In some aspects, a signaling protein that includes a degron in a cage polypeptide of the present disclosure can be an output-producing member. In some aspects, a signaling protein that includes a degron in a cage polypeptide of the present disclosure cannot be an output-producing member, e.g., it can be an input-receiving member or an intermediate member.

As summarized above, various signaling pathways, including native and synthetic signaling pathways can be modulated using the herein described molecular circuits. Suitable signaling pathways include those that are modulated (e.g., activated, repressed, etc.) by one or more inputs to produce one or more outputs. Inputs and outputs of signaling pathways can vary and can include endogenous (e.g., native) inputs or outputs of signaling pathways and heterologous (e.g., engineered or synthetic) signaling pathway inputs and outputs. In some aspects, an input of a signaling pathway relevant to a polypeptide of the present disclosure can include an intracellular signal, including e.g., where the output of the pathway can be intracellular or intercellular. In some aspects, an output of a signaling pathway relevant to a polypeptide of the present disclosure can include an intracellular signal, including e.g., where the input of the pathway can be intracellular or intercellular. In some aspects, an input of a signaling pathway relevant to a polypeptide of the present disclosure can include an intercellular signal, including e.g., where the output of the pathway can be intracellular or intercellular. In some aspects, an output of a signaling pathway relevant to a polypeptide of the present disclosure can include an intercellular signal, including e.g., where the input of the pathway can be intracellular or intercellular.

In some aspects, both the input and the output of a signaling pathway relevant to a polypeptide of the present disclosure can include intracellular signals. In some aspects, both the input and the output of a signaling pathway relevant to a polypeptide of the present disclosure can include intercellular signals.

Suitable non-limiting examples of native signaling pathways that can be modulated using a polypeptide of the present disclosure include but are not limited to e.g., the AKT signaling pathway, the Akt/PKB signaling pathway, the AMPK signaling pathway, the apoptosis signaling pathway, the BMP signaling pathway, the cAMP-dependent pathway, the estrogen signaling pathway, the hedgehog signaling pathway, the hippo signaling pathway, an immune activation pathway, an immune suppression pathway, an immune cell differentiation pathway, an insulin signal transduction pathway, the JAK-STAT signaling pathway, the MAPK/ERK signaling pathway, the mTOR signaling pathway, the NF-B signaling pathway, the nodal signaling pathway, the notch signaling pathway, the p53 signaling pathway, the PI3K signaling pathway, the TGF beta signaling pathway, the TLR signaling pathway, the TNF signaling pathway, the VEGF signaling pathway, the Wnt signaling pathway, and the like.

Suitable non-limiting examples of pathways, the components of which can be modified to include a degron in a cage polypeptide as described herein, also include those PANTHER (Protein Analysis THrough Evolutionary Relationships) pathways described as part of the Gene Ontology Phylogenetic Annotation Project, descriptions of which (including descriptions of the components of such pathways) are available online at www(dot)pantherdb(dot)org. Nonlimiting examples include 2-arachidonoylglycerol biosynthesis, the 5HT1 type receptor mediated signaling pathway, the 5HT2 type receptor mediated signaling pathway, the 5HT3 type receptor mediated signaling pathway, the 5HT4 type receptor mediated signaling pathway, 5-Hydroxytryptamine biosynthesis, 5-Hydroxytryptamine degredation, Acetate utilization, the Activin beta signaling pathway, the Adenine and hypoxanthine salvage pathway, Adrenaline and noradrenaline biosynthesis, Alanine biosynthesis, Allantoin degradation, the ALP23B signaling pathway, the Alpha adrenergic receptor signaling pathway, the Alzheimer diseaseamyloid secretase pathway, the Alzheimer disease-presenilin pathway, Aminobutyrate degradation, Anandamide biosynthesis. Anandamide degradation, Androgen/estrogene/progesterone biosynthesis, the Angiogenesis pathway, Angiotensin Ilstimulated signaling through G proteins and beta-arrestin, the Apoptosis signaling pathway, Arginine biosynthesis, Ascorbate degradation, Asparagine and aspartate biosynthesis, ATP synthesis, Axon guidance mediated by netrin, Axon guidance mediated by semaphorins, Axon guidance mediated by Slit/Robo, the B cell activation pathway, the Betal adrenergic receptor signaling pathway, the Beta2 adrenergic receptor signaling pathway, the Beta3 adrenergic receptor signaling pathway, Biotin biosynthesis, Blood coagulation, the BMP/activin signaling pathway. Bupropion degradation, the Cadherin signaling pathway, Coenzyme A linked carnitine metabolism, Carnitine metabolism, CCKR signaling, the Cell cycle, Cholesterol biosynthesis, Chorismate biosynthesis, Circadian clock system, Cobalamin biosynthesis, Coenzyme A biosynthesis, the Cortocotropin releasing factor receptor signaling pathway, Cysteine biosynthesis, Cytoskeletal regulation by Rho GTPase. De novo purine biosynthesis. De novo pyrimidine deoxyribonucleotide biosynthesis, De novo pyrimidine ribonucleotides biosythesis, DNA replication, the Dopamine receptor mediated signaling pathway, the DPP-SCW signaling pathway, the DPP signaling pathway, the EGF receptor signaling pathway, the Endogenous cannabinoid signaling, the Endothelin signaling pathway, Enkephalin release, the FAS signaling pathway, the FGF signaling pathway, Flavin biosynthesis, Tetrahvdrofolate biosynthesis, Formyltetrahydroformate biosynthesis, Fructose galactose metabolism, GABA-B receptor II signaling, Gamma-aminobutyric acid synthesis, the GBB signaling pathway, General transcription by RNA polymerase I, General transcription regulation, Glutamine glutamate conversion, Glycolysis, the Gonadotropin-releasing hormone receptor pathway, the Hedgehog signaling pathway, Heme biosynthesis, the Heterotrimeric G-protein signaling pathway-Gi alpha and Gs alpha mediated pathway, the Heterotrimeric G-protein signaling pathway-Gq alpha and Go alpha mediated pathway, Heterotrimeric G-protein signaling pathway-rod outer segment phototransduction, the Histamine H1 receptor mediated signaling pathway the Histamine H2 receptor mediated signaling pathway, Histamine synthesis, Histidine biosynthesis, the Hinitington disease pathway, Hypoxia response via HIF activation, the Inflammation mediated by chemokine and cytokine signaling pathway, Insulin/IGF pathway-mitogen activated protein kinase kinase/MAP kinase cascade, Insulin/IGF pathway-protein kinase B signaling cascade, the lntegrin signalling pathway, the Interferon-gamma signaling pathway, the Interleukin signaling pathway, the lonotropic glutamate receptor pathway, isoleucine biosynthesis, the JAK/STAT signaling pathway, Leucine biosynthesis, Lipoate_biosynthesis, Lysine biosynthesis, Mannose metabolism, the Metabotropic glutamate receptor group III pathway, the Metabotropic glutamate receptor group II pathway, the Metabotropic glutamate receptor group I pathway, Methionine biosynthesis, Methylcitrate cycle, the Methylmalonvl pathway, mRNA splicing, the Muscarinic acetylcholine receptor 1 and 3 signaling pathway, the Muscarinic acetylcholine receptor 2 and 4 signaling pathway, the MYO signaling pathway, N-acetylglucosamine metabolism, Nicotine degradation, the Nicotine pharmacodynamics pathway, the Nicotinic acetylcholine receptor signaling pathway, the Notch signaling pathway, O-antigen biosynthesis, the Opioid prodynorphin pathway, the Opioid proenkephalin pathway, the Opioid proopiomelanocortin pathway, Omithine degradation, Oxidative stress response, the Oxytocin receptor mediated signaling pathway, the p38 MAPK pathway, the p53 pathway, p53 pathway by glucose deprivation. Pantothenate biosynthesis, Parkinson disease, the PDGF signaling pathway, the Pentose phosphate pathway, Peptidoglycan biosynthesis, Phenylacetate degradation, Phenylalanine biosynthesis, Phenylethylamine degradation, Phenylpropionate degradation, the PI3 kinase pathway, Plasminogen activating cascade, Pyridoxal-5-phosphate biosynthesis, Proline biosynthesis, PRPP biosynthesis, Purine metabolism, the Pyridoxal phosphate salvage pathway, Pyrimidine Metabolism, Pyruvate metabolism, the Ras Pathway. S-adenosylmethionine biosynthesis, Salvage pyrimidine deoxyribonucleotides, Salvage pyrimidine ribonucleotides, the SCW signaling pathway, Serine glycine biosynthesis, Succinate to proprionate conversion, Sulfate assimilation, Synaptic vesicle trafficking, TCA cycle, the T cell activation pathway, the TGF-beta signaling pathway, Thiamin biosynthesis, Thiamin metabolism, Threonine biosynthesis, the Thyrotropin-releasing hormone receptor signaling pathway, the Toll pathway, the Toll receptor signaling pathway, Transcription regulation by bZIP transcription factor, Triacylglycerol metabolism, Tryptophan biosynthesis, Tyrosine biosynthesis, the Ubiquitin proteasome pathway, Valine biosynthesis, Vasopressin synthesis, the VEGF signaling pathway, Vitamin B6 biosynthesis, Vitamin B6 metabolism, the Vitamin D metabolism and pathway, the Wnt signaling pathway, the Xanthine and guanine salvage pathway, and the like.

Further non-limiting examples of signaling pathways, and description thereof, include the following: AKT Signaling Pathway (AKT is a serine/threonine kinase that is involved in mediating various biological responses, such as inhibition of apoptosis), Angiopoietin-TIE2 Signaling (The angiopoietins are a new family of growth factor ligands that bind to TIE2/TEK RTK (Receptor Tyrosine Kinase)), Antigen Processing and Presentation by MHCs (Antigen processing and presentation are the processes that result in association of proteins with major histocompatibility complex (MHC) molecules for recognition by a T-cell), Apoptosis Through Death Receptors (Certain cells have unique sensors, termed death receptors (DRs), which detect the presence of extracellular death signals and rapidly ignite the cell's intrinsic apoptosis machinery), APRIL Pathway (In immune responses, APRIL acts as a co-stimulator for B-cell and T-cell proliferation and supports class switch), B-Cell Development Pathway (The B-cell receptor (BCR) complex usually consists of an antigen-binding subunit that is composed of two Ig heavy chains, two Ig light chains, and a signaling subunit), BMP Pathway (Bone morphogenetic proteins (BMPs) are a large subclass of the transforming growth factor-beta (TGF-beta) superfamily), Cancer Immunoediting (The immune system attempts to constrain tumor growth, but sometimes tumor cells might escape or attenuate this immune pressure), CCR5 Pathway in Macrophages (C-C motif chemokine receptor type 5 (CCR5) is a member of the chemokine receptor subclass of the G protein-coupled receptor (GPCR) superfamily). CD4 and CD8 T-Cell Lineage (Each mature T-cell generally retains expression of the co-receptor molecule (CD4 or CD8) that has a major histocompatibility complex (MHC)-binding property that matches that of its T-cell receptor (TCR)), Cellular Apoptosis Pathway (Apoptosis is a naturally occurring process by which a cell is directed to programmed cell death), CTLMediated Apoptosis (The cytotoxic T lymphocytes (CTLs), also known as killer T-cells, are produced during cell-mediated immunity designed to remove body cells displaying a foreign epitope), CTLA4 Signaling Pathway (The co-stimulatory CTLA4 pathway attenuates or downregulales T-cell activation CTLA4 is designed to remove body cells displaying a foreign epitope), Cytokine Network (Cytokines have been classified on the basis of their biological responses into pro- or anti-inflammatory cytokines, depending on their effects on immunocytes), ErhB Family Pathway (The ErbB family of transmembrane receptor tyrosine kinases (RTKs) plays an important role during the growth and development of organs), Fas Signaling (FAS (also called APO1 or CD95) is a death domain-containing member of the tumor necrosis factor (TNF) receptor superfamily), FGF Pathway (One of the most well characterized modulators of angiogenesis is the heparin-binding fibroblast growth factor (FGF)), Granulocyte Adhesion and Diapedesis (Adhesion and diapedesis of granulocytes have mostly been analyzed in context to non-lymphoid endothelium), Granzyme Pathway (Granzyme A (GzmA) activates a caspaseindependent cell death pathway with morphological features of apoptosis), GSK3 Signaling (GSK3 is a ubiquitously expressed, highly conserved serine/threonine protein kinase found in all eukaryotes), Hematopoiesis from Multipotent Stem Cells (Hematopoietic stem cells are classified into long-term, short-term and multipotent progenitors, based on the extent of their self-renewal abilities), Hematopoiesis from Pluripotent Stern Cells (Pluripotent stem cells are capable of forming virtually all of the possible tissue types found in human beings), IL-2 Gene Expression in Activated and Quiescent T-Cells (IL-2 is a cytokine that stimulates the growth, proliferation, and differentiation of T-cells, B-cells, NK cells, and other immune cells), IL-6 Pathway (IL-6 is a pleiotropic cytokine that affects the immune system and many physiological events in various organs), IL-10 Pathway (IL-10 is a pleiotropic cytokine with important immunoregulatory functions and whose activities influence many immune cell types), IL-22 Pathway (IL-22 is a member of the IL-10 family of cytokines and exerts multiple effects on the immune system), Interferon Pathway (Interferons are pleiotropic cytokines best known for their ability to induce cellular resistance to viral infection), JAK/STAT Pathway (The JAK/STAT pathway is a signaling cascade whose evolutionarily conserved roles include cell proliferation and hematopoiesis), MAPK Family Pathway (Mitogen-activated protein kinases (MAPKs) belong to a large family of serine/threonine protein kinases that are conserved in organisms as diverse as yeast and humans), Nanog in Mammalian ESC Pluripotency (NANOG is a transcription factor transcribed in pluripotent stem cells and is down-regulated upon cell differentiation), p53-Mediated Apoptosis Pathway (Tumor protein p53 is a nuclear transcription factor that regulates the expression of a wide variety of genes involved in apoptosis, growth arrest, or senescence in response to genotoxic or cellular stress), Pathogenesis of Rheumatoid Arthritis (Rheumatoid arthritis (RA) is a chronic symmetric polyarticular joint disease that primarily affects the small joints of the hands and feet), PI3K Signaling in B Lymphocytes (The phosphoinositide 3-kinases (PI3Ks) regulate numerous biological processes, including cell growth, differentiation, survival, proliferation, migration, and metabolism), RANK Pathway (RANKL and its receptor RANK are key regulators of bone remodeling, and are essential for the development and activation of osteoclasts), RANK Signaling in Osteoclasts (RANKL induces the differentiation of osteoclast precursor cells and stimulates the resorption function and survival of mature osteoclasts), TGF-Beta Pathway (Members of the transforming growth factor (TGF)-beta family play an important role in the development, homeostasis, and repair of most tissues), THC Differentiation Pathway (T-helper cells of type 1 (TH1) and type 2 (TH2) are derived from ‘T’-helper cells and provide help to cells of both the innate and adaptive immune systems), TNF Signaling Pathway (Tumor necrosis factor (TNF) is a multifunctional proinflammatory cytokine with effects on lipid metabolism, coagulation, insulin resistance, and endothelial function), TNF Superfamily Pathway (The tumor necrosis factor (TNF) superfamily consists of 19 members that signal through 29 receptors that are members of the TNF receptor (TNFR) superfamily), Transendothelial Migration of Leukocytes (Transport of plasma proteins and solutes across the endothelium involves two different routes: transcellular and paracellular junctions), Tumoricidal Effects of Hepatic NK Cells (The liver is a major site for the formation and metastasis of tumors), TWEAK Pathway (TWEAK is a cell surface-associated protein belonging to the tumor necrosis factor (TNF) superfamily and has multiple biological activities). VEGF Family of Ligands and Receptor Interactions (Vascular endothelial growth factor (VEGF) is a highly-conserved genetic pathway that has evolved from simple to complex systems), and the like.

As summarized above, a component of a signaling pathway, including but not limited to a pathway described herein, can be modified to include a degron in a cage polypeptide such that degradation of the signaling pathway member can be controlled by expression of a key polypeptide. Suitable pathway components that can be employed include e.g., input-receiving members, intermediate members, and output-producing members, including but not limited to e.g., the corresponding member of the pathways listed above.

Similarly, essentially any synthetic pathway can be modulated using a degron in a cage polypeptide as described herein. Suitable non-limiting examples of synthetic signaling pathways that can be modulated using a degron in a cage polypeptide of the present disclosure include, but are not limited to, those pathways controlled by a synthetic or engineered receptor, such as but not limited to e.g., a CAR, an engineered TCR, a synNotch, etc.

In some aspects, a pathway modulated using a degron in a cage polypeptide of the present disclosure can include an immune modulation pathway, such as e.g., an immune activation pathway or an immune suppression pathway. Such immune modulation pathways can be natural or synthetic and can be endogenous to the cell in which the degron in a cage polypeptide is employed or heterologous to the cell in which the degron in a cage polypeptide is employed.

Suitable non-limiting examples of synthetic signaling pathways that can be modulated using a degron in a cage polypeptide of the present disclosure also include biosynthesis and/or bioproduction pathways. Biosynthesis and/or bioproduction pathways can be natural or synthetic and can be employed in cells and/or organisms where the pathway is endogenous or heterologous.

Non-limiting examples of biosynthesis pathways that can be modulated using a degron in a cage polypeptide of the present disclosure include, hut are not limited to, hormone production pathways (e.g., an insulin production pathway, an estrogen/progesterone production pathway, an androgen production pathway, a growth hormone production pathway, and the like), opioid production pathways, isobutanol production pathways, non-ribosomal polyketide synthetase (NRPS) production pathways, antibiotic production pathways, chemotherapeutic production pathways, artemisinic acid production pathways, terpenoid production pathways, polyketide production pathways, and the like.

Non-limiting examples of synthetic biosynthesis pathways include but are not limited to e.g., synthetic hormone production pathways, synthetic opioid production pathways, synthetic antibiotic production pathways, synthetic chemotherapeutic production pathways, synthetic artemisinic acid production pathways, synthetic terpenoid production pathways, synthetic polyketide production pathways, and the like.

As used throughout the present application, the term “polypeptide” is used in its broadest sense to refer to a sequence of subunit amino acids. The polypeptides of the invention may comprise L-amino acids+glycine, D-amino acids+glycine (which are resistant to L-amino acid-specific proteases in vivo), or a combination of D- arid L-amino acids+glycine. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill inthe art.

In a fourth aspect the disclosure provides nucleic acids encoding the polypeptide of any embodiment or combination of embodiments of each aspect disclosed herein. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a fifth aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated vet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In a sixth aspect, the disclosure provides host cells that comprise the nucleic acids or expression vectors (i.e.: episomal or chromosornally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection. In one embodiment, the recombinant host cells comprise:

(a) a first nucleic acid encoding the polypeptide of any embodiment or combination of embodiments of the cage polypeptides of the disclosure, operatively linked to a first promoter; and

(b) a second nucleic acid encoding the polypeptide of any embodiment or combination of embodiments of the key polypeptide of the disclosure, wherein the key poly peptide is capable of binding to a structural region of the cage polypeptide to induce a conformational change in the can polypeptide, wherein the second nucleic acid is operatively linked to a second promoter.

The recombinant host cells may comprise a single cage polypeptide encoding nucleic acid and a single key polypeptide encoding nucleic acid, or may comprise a plurality (i.e.: 2, 3, 4, 5, 6, 7, 8, 9, 10, or more) first and second nucleic acids—in one such embodiment, each second-nucleic acid may encode a key polypeptide capable of binding to a structural region and inducing a conformational change of a different cage polypeptide encoded by the plurality of first nucleic acids. In another embodiment, each second nucleic acid may encode a key polypeptide capable of binding to a structural region and inducing a conformational change of more than one of the cage polypeptides encoded by the plurality of first nucleic acids.

Thus, in one embodiment the first nucleic acid comprises a plurality of first nucleic acids encoding a plurality of different cage polypeptides. In one such embodiment, the second nucleic acid comprises a plurality of second nucleic acids encoding a plurality of different key polypeptides, wherein the plurality of different key polypeptides comprise one or more key polypeptides that are capable of binding to and inducing a conformational change in only a subset of the plurality of different cage polypeptides. In another such embodiment, the second nucleic acid encodes a single key polypeptide that is capable of binding to and inducing a conformational change in each different cage polypeptide.

In another embodiment, the host cells comprise nucleic acids encoding and/or expression vectors capable of expressing the fusion proteins disclosed herein, wherein the host cells comprise:

(a) a first nucleic acid encoding a first fusion protein (i.e.: cage polypeptide fused to key polypeptide) linked to a first promoter; and

(b) a second nucleic acid encoding a second fusion protein operatively linked to a second promoter, wherein:
(i) the cage polypeptide encoded by the first nucleic acid is activated by the key polypeptide encoded by the second nucleic acid;
(ii) the cage polypeptide encoded by the first nucleic acid is not activated by the key polypeptide encoded by the first nucleic acid;
(iii) the cage polypeptide encoded by the second nucleic acid is activated by the key polypeptide encoded by the first nucleic acid; and
(iv) the cage polypeptide encoded by the second nucleic acid is not activated by the key polypeptide encoded by the second nucleic acid.

In all these embodiments, the first and/or second nucleic acids may, for example, be in the form of an expression vector. In other embodiments, the first and/or second nucleic acids may be in the form of nucleic acid integrated into the host cell genome.

A method of producing a polypeptide according to the disclosure is an additional part of the disclosure. In one embodiment, the method comprises the steps of (a) culturing a host according to this aspect of the disclosure under conditions conducive to the expression of the polypeptide, and (b) optionally, recovering the expressed polypeptide. The expressed polypeptide can be recovered from the cell free extract or recovered from the culture medium. In another embodiment, the method comprises chemically synthesizing the polypeptides.

In a seventh aspect, the disclosure provides kits. In one embodiment, the kits comprise:

(a) one or more cage polypeptides of any embodiment or combination of embodiments of the disclosure;

(b) one or more key polypeptides of any embodiment or combination of embodiments of the disclosure; and

(c) optionally, one or more fusion proteins of any embodiment disclosed herein.

In another embodiment, the kits comprise:

(a) a first nucleic acid encoding the cage polypeptide of any embodiment or combination of embodiments of the disclosure;

(b) a second nucleic acid encoding the key polypeptides of any embodiment or combination of embodiments of the disclosure; and

(c) optionally, a third nucleic acid encoding the fusion protein of any embodiment disclosed herein.

In another embodiment, the kit comprises:

(a) a first expression vector comprising a first nucleic acid encoding the cage polypeptide of any embodiment or combination of embodiments of the disclosure, wherein the first nucleic acid is operatively linked to a first promoter; and

(b) a second expression vector comprising a second nucleic acid encoding the key polypeptides of any embodiment or combination of embodiments of the disclosure, wherein the second nucleic acid is operatively linked to a second promoter.

In each of the kit embodiments, the first nucleic acid, the second nucleic acid, the first expression vector, and/or the second expression vector may comprise a single nucleic acid encoding or expression vector capable of expressing the cage or key polypeptide, or may comprise a plurality (i.e.: 2, 3, 4, 5, 6, 7, 8, 9, 10, or more) of first nucleic acids, second nucleic acids, first expression vectors, and/or the second expression vectors. In various such embodiments, each second nucleic. acid may encode, or each second expression vector may be capable of expressing, a key polypeptide capable of binding to a structural region and inducing a conformational change of a different cage polypeptide encoded by the plurality of first nucleic acids or capable of being expressed by the plurality of first expression vectors. In other embodiments, each second nucleic acid may encode, or each second expression vector may be capable of expressing, a key polypeptide capable of binding to a stnictural region and inducing a conformational change of more than one of the cage polypeptides encoded by the plurality of first nucleic acids or capable of being expressed by the plurality of first expression vectors.

In one embodiment, the promoters operatively linked to the cage polypeptide-encoding nucleic acids (first promoters) are different than the promoters operatively linked to the key polypeptide-encoding nucleic acids (second promoters), allowing tunable control of the cage polypeptides and any functional polypeptide domains by controlling expression of the key polypeptide. In other embodiments, the promoters operatively linked to the cage polypeptide-encoding nucleic acids (first promoters) are the same as the promoters operatively linked to the key polypeptide-encoding nucleic acids (second promoters). In other embodiments, the first promoters and/or second promoters may be inducible promoters.

In an eighth aspect, the disclosure provides uses of cage polypeptides, kits, degronLOCKR switches, nucleic acids, expression vectors, or host cells comprising cage polypeptides to sequester a degron in the cage polypeptide until a key is expressed and activates the cage polypeptide, and the degron targets the cage polypeptide and any functional peptide fused to it for degradation. As disclosed in detail herein, when a key is expressed and activates the cage polypeptide by interacting with the structural region, the degron targets the cage polypeptide, and any functional polypeptide domains fused to the cage polypeptide, for degradation. In this way, a functional polypeptide domain of interest fused to the cage polypeptide having a degron can be conditionally degraded in a titratable manner via expression of the key. This is sometimes referred to herein as degron LOCKR. The kits and degron LOCKR switches disclosed herein are activated separate from natural proteins and work in any eukaryote. Furthermore, there are no current methods to modularly tag any functional polypeptide domain of interest with a conditional degron dependent on expression of a control peptide. Thus, the kits and degron LOCKR switches disclosed herein can be used as a modular regulatory hub in a wide variety of biotechnological applications as described herein.

### EXAMPLE 1

### Summary:

We have developed a general approach to design novel protein switches that can sequester bioactive peptides and/or binding domains, holding them in an inactive (“off”) state, until combined with a second designed polypeptide called the Key, which induces a conformational change that activates (“on”) the bioactive peptide or binding domain.

Nomenclature and Structural Features that Define LOCKR Switches:
LOCKR stands for Latching Orthogonal Cage-Key pRotiens; each LOCKR design consists of a Cage protein and a Key protein, which are two separate polypeptide chains.
The Cage encodes the sequestered bioactive peptide or binding domain in a region of the Cage scaffold denoted as the Latch. The general strategy is to optimize the position of the encoded peptide or binding domain for maximum burial of the functional residues that need to be sequestered, simultaneously optimizing for burial of hydrophobic residues, and for solvent exposure/compensatory hydrogen bonds of polar residues.
The Key displaces the Latch through competitive intermolecular binding that induces conformational change, exposing the encoded bioactive peptide or domain and activating the system (FIG. 1).
Orthogonal LOCKR designs (FIG. 3) are denoted by lowercase letter subscripts: LOCKRₐ consists of Cageₐ and Keyₐ, and LOCKR_{b} consists of Cage_{b} and Key_{b}, etc. such that Cageₐ is only activated by Keyₐ, and Cage_{b} is only activated by Key_{b}, etc.
Prefixes denote the functional group that is encoded and controlled by the LOCKR switch. For example, BimLOCKR refers to designed switches that encode the Bim peptide, and GFP11-LOCKR refers to designed switches that encode GFP11 (the 11^{th} strand of GFP).
Toehold: The dynamic, range of LOCKR activation by Key can be tuned by truncating the Latch length, simultaneously weakening the Cage-Latch interaction and opening an exposed region on the Cage that the Key can bind to as a “toehold” (FIG. 2. LOCKR can also be tuned in a similar manner by designing mutations into the Latch that weaken the Cage-Latch interaction (FIGS. 1-2, FIG. 10). The length of the toehold is included as a suffix to the design name: For example “−t0” means no toehold, and “−t9” means a toehold of 9 residues (i.e. Latch truncated by 9 residues).
If the term “Lock” is used in reference to a single polypeptide chain (not in reference to the LOCKR acronym), it is assumed to be synonymous with “Cage”.

These designs comprise the first ever de novo designed proteins that can undergo conformational switching in response to protein binding. They are modular in that they can encode bioactive peptides of all three types of secondary structure in an inactive conformation: alpha helix, beta strand, loop, and are tunable in that their responsiveness can be tuned over a large dynamic range by varying length (length of Cage scaffold and/or Latch Toehold), and/or mutating residues in the Cage-Latch interface. Designed LOCKR switches can be used to control the activity of a wide range of functional peptides. The ability to harness these biological functions with tight, inducible control is useful, for example, in engineering cells (inducible activation of function, engineering complex logic behavior and circuits), developing sensors, developing inducible protein-based therapeutics, and creating new biomaterials.

### Design of LOCKR Switches

We set out to design de novo switchable protein systems guided by the following general considerations. First, the free energy tuning required to achieve maximal dynamic range upon addition of the switch-triggering input is more straightforward in a system governed by competition between inter- and intra-molecular interactions at the same site rather than at distant sites (as is common in allosteric biological systems). Second, a stable protein framework with an extended binding surface available for the competing interactions has advantages over a framework that only becomes ordered upon binding, as the former is more programmable and less likely to engage in off-target interactions. These features are described by the abstract system depicted in Fig l a, which undergoes thermodynamically-driven switching between a binding incompetent and a binding competent state. A latch (blue) contains a peptide sequence (orange) that can bind a target (yellow) unless blocked by intramolecular interactions to a cage (cyan); a more tightly binding key (magenta) outcompetes the latch allowing the peptide to bind target. The behavior of such a system is governed by the binding equilibrium constants for the individual subreactions (FIG. 1a): Kₒₚₑₙ, the dissociation of latch from cage; K_{LT}, the binding of latch to target; and K_{CK}, the binding of key to cage. Solution of this set of equations shows that when the latch-cage interaction is too weak (red and orange curves), the system is leaky and the fold induction by key is low, while when the latch-cage interaction is too strong (purple curve), the system is only partially activated, even at high key concentrations. The latch-cage interaction affinity that gives optimal switching (FIG. 1b, blue curve left, green curve right) is a function of the latch-target binding affinity. We used this model to guide design of an optimally switchable protein system, as described in the following sections.

### LOCKR Design Strategy

To design such a switchable system, we chose structural features amenable to tuning of the affinities of the cage-latch and cage-key interactions over a wide dynamic range. Alpha helices have advantages over beta strands because inter-helical interfaces are dominated by sidechain-sidechain interactions, which can be more readily tuned than the cooperative backbone hydrogen bonding necessary for beta sheets. To allow fine control over the relative affinities of the cage-latch and cage-key interactions, we chose to design interfaces containing buried hydrogen bond networks: as illustrated by Watson Crick base pairing, considerable specificity can be obtained with relatively minor changes in the positions of hydrogen bond donors and acceptors^{4.5}. We selected as a starting point a designed homo-trimer of α-helical hairpins with hydrogen bond network-mediated subunit-subunit interaction specificity (5L6HC3_1)⁵. By designing short unstructured loops connecting the subunits, we generated monomeric protein frameworks with five or six helices and 40 residues per helix (FIG. 1c). In the five-helix framework, there is an open binding site for a sixth helix added in trans, whereas this site is filled by a sixth helix in cis in the six-helix framework.

The five helix (cage) and six helix (cage plus latch) designs were soluble when recombinantly expressed in E. coli, and the purified proteins were largely monomeric by size-exclusion chromatography with multi-angle light scattering, and very thermostable, remaining folded at upwards of 95° C. and 5 M guanidine hydrochloride (FIG. 1d). Small-angle X-ray scattering (SAXS) spectra were in close agreement to the design models and previous of the original trimers (FIG. 1e), suggesting that the structure was not altered by the loops. The five-helix framework, but not the six-helix framework, bound the sixth helix fused to GFP in a pull-down assay (FIG. 1f); the latter result is expected since if the interfaces are otherwise identical, the intramolecular interaction Kₒₚₑₙ, should outcompete its intermolecular counterpart, K_{CK}, because of the reduced entropic cost of formation of intramolecular interactions. To tune Kₒₚₑₙ, we screened destabilizing mutations in the latch (large hydrophobics to alanine or serine, and alanine residues to larger hydrophobics or serine) and using the GFP pull-down assay, identified mutants with a range of affinities for the key. A double mutant, V223S/I238S, bound key as strongly as the five-helix cage without the latch (FIG. 1e, 10); the two serines likely weaken the cage-latch interaction because of the desolvation penalty associated with burying the sidechain hydroxyls, and because they decrease the helical propensity of the latch. SAXS and CD spectra indicate that in the absence of key, V223S, I238S is a well-folded six-helix bundle with structure similar to the original monomer (FIG. 1d). We call this cage-latch-key system LOCKR, for Latching Orthogonal Cage-Key pRoteins.

### Controlling Bim-Bcl2 Binding, and Tuning the Dynamic Range of Activation:

To install function into LOCKR, we selected the Bim-Bcl2 interaction central to apoptosis as a model system, seeking to cage Bim such that binding to Bcl2 only occurred in the presence of key. We designed constructs with two possible Bim-related sequences designed onto the latch: a designed Bcl2-binding peptide (aBcl2LOCKR) or just the Bim residues crucial for Bcl2 binding (pBimLOCKR). Each has a different affinity for Bcl2, allowing us to sample a range of K_{LT} values in the initial series of designs. Bim-related sequences were grafted onto the latch by sampling different helical registers such that residues involved in binding to Bcl2 are sequestered in the cage-latch interface (data not shown), optimizing for the burial of hydrophobic residues and surface exposure of polar residues. Kₒₚₑₙ can be tuned by non-optimal interactions between the cage and Bim residues or by changing the length of the latch (FIG. 2a). Initial designs were tested for binding to Bcl2 by bio-layer interferometry, and were either showed little Bcl2 binding even in the presence of key, or Bcl2 binding even in the absence of key. The range of Kₒₚₑₙ and K_{CK} values accessible with this system was evidently not matched to K_{LT} in this case: the key induced response was far from the ideal curves in FIG. 1b.

We hypothesized that the system could be improved by extending the interface area presented on the cage: extending the latch could increase the affinity for the cage (decrease Kₒₚₑₙ) to make the system more “off” in absence of key, while extending the key to be longer could allow it to outcompete the latch (decrease K_{CK} relative to Kₒₚₑₙ), making the system more inducible. Taking advantage of the modular nature of de novo parametric helical bundles, the cage, latch and key were each extended by 5, 9 or 18 residues. To enable the key to outcompete the latch, the latter was truncated by four to nine residues to access a range of Kₒₚₑₙ values; this creates a “toehold” on the cage for the key to bind). The 18-residue extension with a 9 residue toehold resulted in strongly inducible binding (FIG. 2b,c; the signal on bio-layer interferometry is not due to key binding Bcl2 nor the key adding bulk to inactive LOCKR. The activation of binding by the key is approximately 40-fold (FIG. 2c), comparable to or better than many naturally occurring processes that are regulated by protein interactions.

The range of key concentrations over which BimLOCKR is activated can be controlled by tuning K_{CK} by varying the length of the key since the interaction energy is roughly proportional to the total surface area of interacting residues. The EC50 for the 58-length designed key is 55.6+/−34 nM (FIG. 2c,d), and for a 45 residue key, 230+/−58 nM. Truncating an additional five residues completely negates key activation, indicating the equilibria are very sensitive to small changes in free energy as expected from our model (FIG. 2d). To examine function of BimLOCKR over a three orders of magnitude range of K_{LT} we studied key induced binding to Bcl2 homologs BclB and Bak (which bind Bim with Kd's of 0.17 nM (Bcl2), 20 nM (BclB), and 500 nM (Bak))⁶. Bio-layer interferometry experiments were performed with target immobilized assayed against the switch with or without key in solution, as well as with key immobilized and assayed against the switch alone or with target in solution. From these results, we can obtain the fraction of target or key bound as a function of the concentrations of switch, key, and target. A global fit of the model to these data for Kₒₚₑₙ, K_{CK}, and K_{LT} yields estimates of Kₒₚₑₙ=0.01+/−0.0033, K_{CK}=2.1+/−1.1 nM, K_{LT}(Bcl2)=28+/−7.8 nM, and K_{LT}(BclB)=32+/−22 nM with no estimate for K_{LT} (Bak) as little switch activation was observed. This fit has an RMSE (root-mean-square-error) of 0.072 nm to the observed BLI data. The approximate agreement of these estimates with the Bim binding Kd's (which were not used in the fitting) suggests the thermodynamic model (FIG. 1a) is a good representation of the system while possibly missing small features of the system affecting target binding.

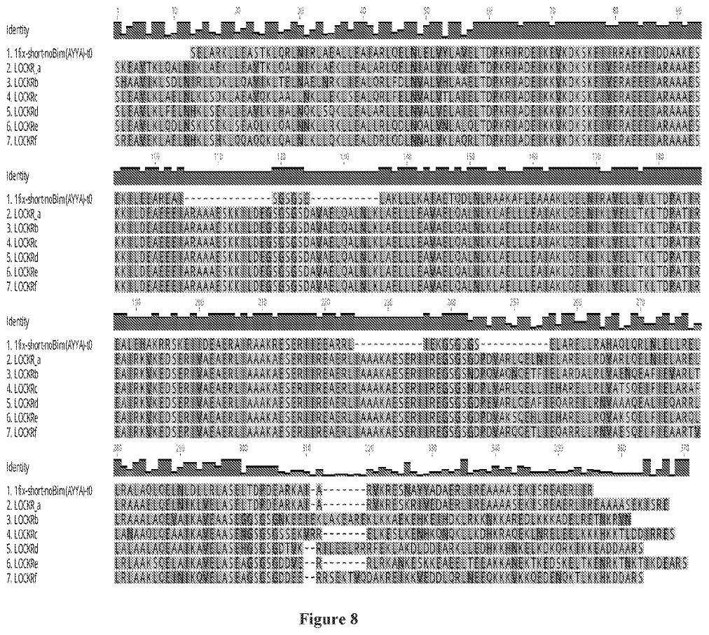

We next sought to design a series of orthogonal LOCKR systems with the goal of engineering multiple switches able to be activated selectively in a heterogeneous mixture. Specificity was designed for using different hydrogen bond networks at the cage-key interface. The latch helix was deleted from the original extended LOCKRₐ model and backbones for a new sixth helix were generated by parametrically sampling the radius, helical phase, and z-offset of the new latch/key helix. The resulting structures were scanned for new hydrogen bond networks spanning the interface between the new sixth helix and cage with all buried polar atoms participating in hydrogen bonds; the remaining interface around the networks was subjected to full sequence and sidechain rotamer optimization using Rosetta™ design. Five designs were selected based on packing quality, sequence dissimilarity, and lack of buried polar atoms not participating in hydrogen bonds. Truncations and toehold variants were assayed for cognate and off target key binding using the GFP pulldown assay from FIG. 1c. Three of the new designs were found to bind their cognate keys (FIG. 11) and did so orthogonally from one another. All bound keyₐ to some extent yet is unknown. The Bim sequence was threaded onto the latches of these three designs as it was for the original design. BimLOCKRₐ, (FIG. 2). BimLOCKR_{b} and BimLOCKR_{c} show 22-fold and 8-fold activation, respectively, from their cognate keys given a nine residue toehold on the latch (FIG. 3a,b). BimLOCKRₐ, BimLOCKR_{b} and BimLOCKR_{c} are also orthogonal; each is activated only by its cognate key at concentrations up to 5 μM (FIG. 3c). The power of the buried hydrogen bond network approach to achieving specificity is illustrated by the fact that of the six designed BimLOCKR proteins, three successfully switch and can be activated orthogonally, a 50% success rate starting from a single scaffold.

### Asymmetrized LOCKR Switches

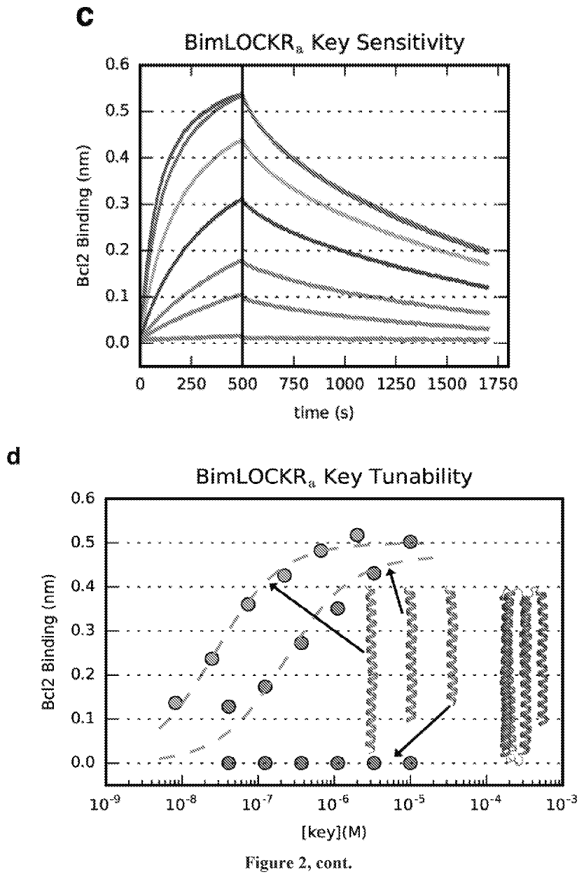

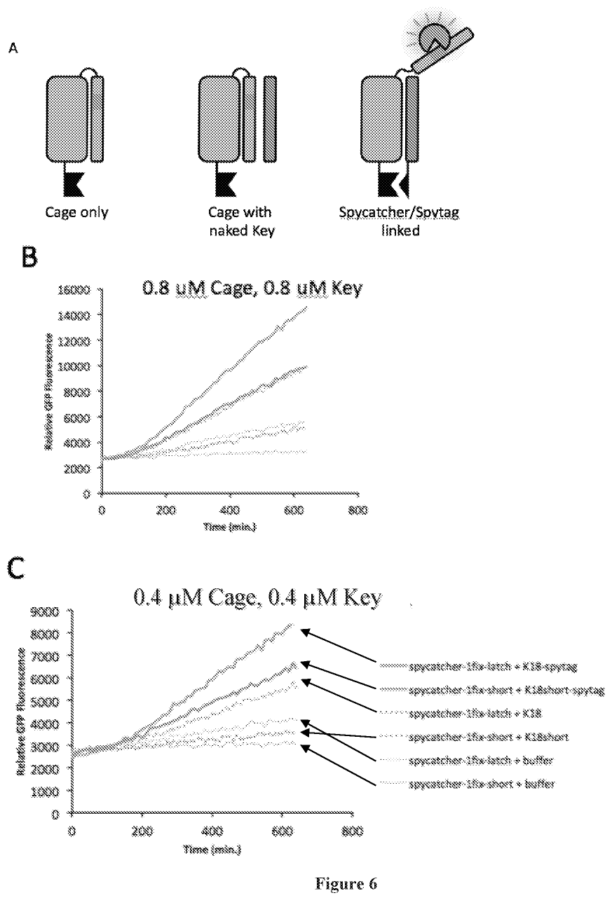

The original LOCKR switch design (FIGS. 1-2) was built starting from a de novo designed symmetric homotrimer, 5L6HC3_1, which contains 6 helices⁵. The symmetric repeating sequence motifs create opportunities for misfolding and aggregation. To mitigate these effects, we redesigned the original LOCKR switch to be asymmetric (sequences listed at the end of this document). The asymmetric designs are better behaved, more monomeric, and we experimentally solved x-ray crystal structures (FIG. 4), both with the encoded BIM peptide (FIG. 4a), and without the BIM peptide (FIG. 4b). The experimental structure without BIM is nearly identical to the computational design model (FIG. 4b), demonstrating atomic-level accuracy of our design strategy. Details of computational design and experimental testing providing in Methods. See FIG. 9 for a superposition of the crystal structure of 1fix-short-noBim(AYYA)-t0 (FIG. 4b) onto the x-ray crystal structure of the base scaffold 5L6HC3_1⁵ (dark) used to make LOCKRa (FIG. 1).

gfpLOCKR (GFP11-LOCKR)

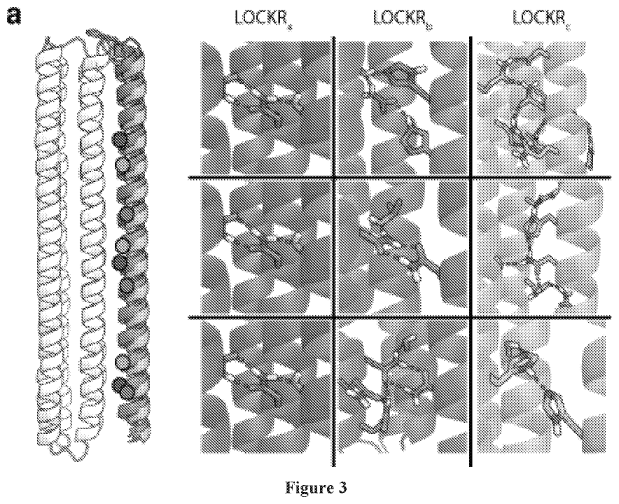

Using the asymmetric designs as a starting point, we successful encoded the 11^{th} strand of GFP into designed LOCKR switches (FIG. 5). A common split GFP consists of two parts: Strands 1-10, and Strand 11; when mixed, 1-10 combines with 11 to yield fluorescence. Here we demonstrate that the 11^{th} strand is sequestered in the absence of Key, unable to combine with GFP-1-10, but readily yields fluorescence when mixed with Key in the presence of GFP-1-10 (FIG. 5). We experimentally determined x-ray crystal structures of the designed protein, which shows that GFP-11 is structurally encoded as an alpha helix, in a nearly identical conformation to that of the computational design model (FIG. 5); this result highlights the power and modularity of the LOCKR system, suggesting that we can encode bioactive peptides with secondary structure propensities that are non-helical.

### Tuning for Co-Localization Dependence

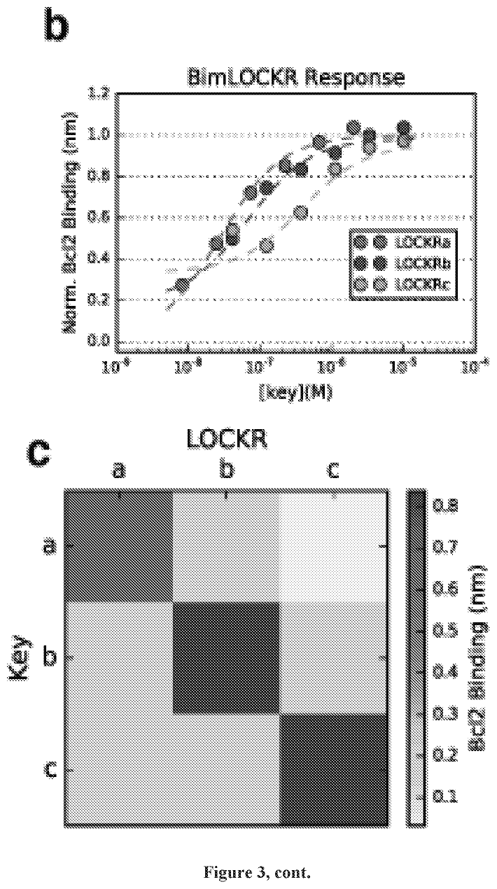

FIGS. 1-2 demonstrate that the dynamic range of LOCKR activation can be predictively tuned, suggesting that the system can be modulated to respond only when the Cage and Key are colocalized, which would be advantageous for a wide range of functions. Using the GFP11-LOCKR from FIG. 4, we demonstrated that this is indeed the case, and that that designed LOCKR switches can be tuned to be colocalization dependent using Spycatcher™/Spytag™ fusions (FIG. 6). Spycatcher™ covalently fuses to Spytag™; when Spycatcher™ fuse Cage was mixed with its Spytag™-fused Key, it showed significantly more fluorescence that when mixed with its Key that was not fused to Spytag™ (FIG. 6).

### Caged Intein LOCKR Switches

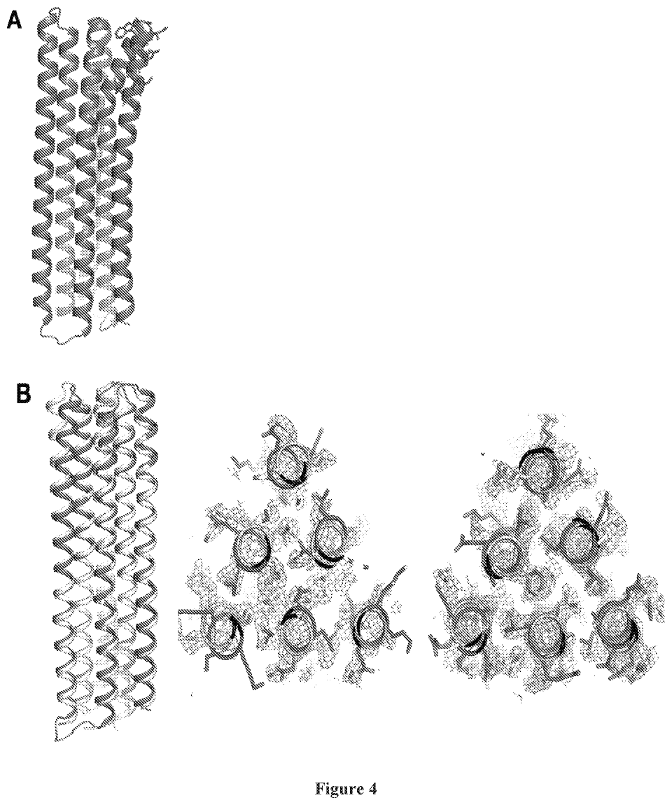

Designed LOCKR switch, with Cage component encoding the VMAc Intein, shows successful activation when mixed with designed Key fused to sfGFP and VMAn Intein (FIG. 7). The SDS-PAGE shows successful VMAc-VMAn reaction, with bands corresponding to the correct molecular weight of the expected spliced protein products (FIG. 7).

### Large-Scale High-Throughput Design of LOCKR Switches

The original LOCKR switch design (FIGS. 1-2) was built starting from a de novo designed symmetric homotrimer, 5L6HC3_1, which contains 6 helices⁵. We reasoned we should be make smaller LOCKR switches, consisting of 3 or 4 helices. Using everything that we learned from the testing and experimental validation of the original LOCKR switch, we created a computational pipeline to automate the design of thousands of new LOCKR switch scaffolds from scratch by exhaustively sampling Crick helical parameters^{4.9}. These 2plus1 and 3plus1 LOCKR switches have smaller payload than the original designs (advantageous for cell engineering efforts), and due to lack of symmetry, are likely to be well-behaved and not aggregation-prone. (See Methods section for details of computational design and experimental testing).

strepLOCKR (STREII-LOCKR)

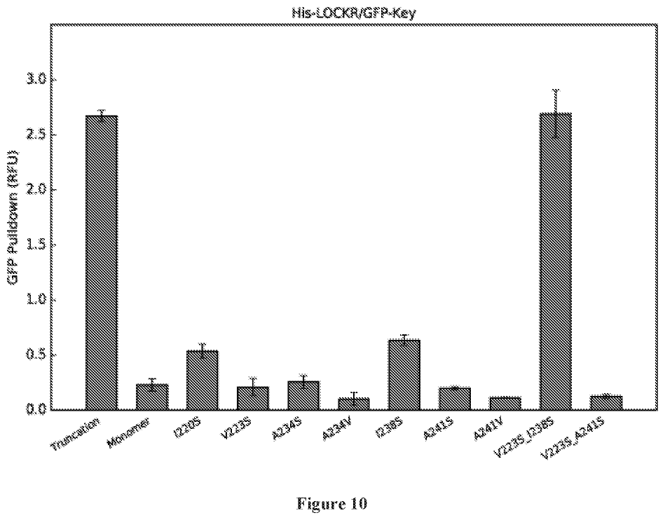

Using the new 2+1 and 3+1 LOCKR scaffolds from the large-scale high-throughput design, we designed and tested new LOCKR scaffolds that encode and control the STREPII sequence. (N)WSHPQFEK (SEQ ID NO: 63) (see Methods section for details). The designs (FIG. 13A) sequester the STREPII tag as compared to a positive control (FIG. 13B) and can be activated in the presence of Key (FIG. 13c-d), as determined by biolayerinterferometry (Octet) data.

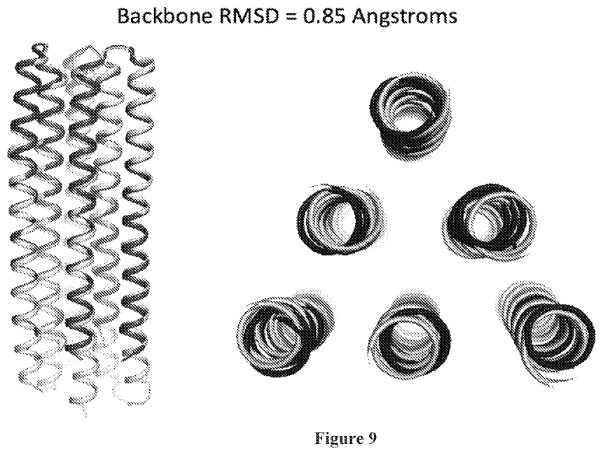

The data in FIG. 12 demonstrate caging of the PAH2 domain of the mSin3A transcriptional repressor. See the figure legend for details.

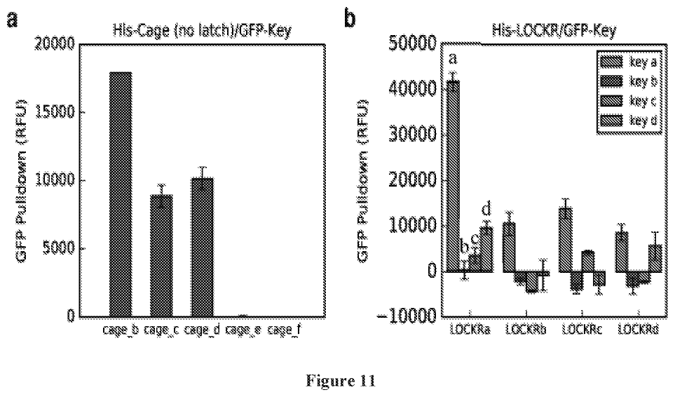

The data in FIG. 14 demonstrate 3plus LOCKR switches activating GFP fluorescence in response to expression of key. See the figure legend for details

### Discussion

Here we demonstrate the power of the LOCKR platform by caging protein-protein interactions that can be inducibly activated by key. We show in vitro data caging the Bim peptide from binding its family members, GFP strand11 from completing the truncated 1-10 construct, and an anti-StrepTag™ II antibody from binding caged StrepTag™II. The modularity and hyperstability of de novo designed proteins enables tuning of switch activation over a broad dynamic range by tuning the strength of the competing cage-key and cage-latch interfaces. Using this approach, we can now design switches beyond these proof-of-concept designs to cage peptides for more complex applications. LOCKR is useful for controlling native signaling networks, and in general for controlling biological function through fully synthetic networks of de novo signaling molecules.

LOCKR brings to proteins the modularity of DNA switching technology, but with advantages of being able to control, and be coupled to, the wide range of biochemical functions that can be carried out by proteins and bioactive peptides (which are much more diverse and wide ranging than nucleic acids).

### Methods

### PCR Mutagenesis and Isothermal Assembly

All primers for mutagenesis were ordered from Integrated. DNA Technologies (IDT). Mutagenic primers were designed to anneal >18 bp on either side of the site for mutagenesis with the desired mutation encoded in the primer. PCR was used to create fragments upstream and downstream of the mutation site with >20 bp overlap with the desired pET vector. The resulting amplicons were isothermally assembled into either pET21b, pET28b, or pET29b restriction digested with Xhol and Ndel and transformed into chemically competent E. coli XL1-Blue cells. Monoclonal colonies were sequenced with Sanger sequencing. Sequence verified plasmid was purified using Qiagen miniprep kit and transformed into chemically competent E. coli BL21(DE3)Star, BL21(DE3)Star-pLysS cells (Invitrogen), or Lemo21(DE3) cells (NEB) for protein expression.

### Synthetic Gene Construction

Synthetic genes were ordered from Genscript Inc. (Piscataway, N.J. USA) and delivered in pET 28 , pET21+, or pET29b+ E coil expression vectors, inserted at the Ndel and XhoI sites of each vector. For pET28b+ constructs, synthesized DNA was cloned in frame with the N-terminal hexahistidine tag and thrombin cleavage site and a stop codon was added at the C-terminus. For pET21b+ constructs, a stop codon was added at the C-terminus such that the protein was expressed with no hexahistidine tag. For pET29b+ constructs, the synthesized DNA was cloned in frame with the C-terminal hexahistidine tag. Plasmids were transformed into chemically competent E. coli BL21(DE3)Star, BL21(DE3)Star-pLysS cells (Invitrogen), or Lemo21(DE3) cells (NEB) for protein expression.

### Bacterial Protein Expression and Purification

Starter cultures were grown in Lysogeny Broth (LB) or Terrific™ Broth II (TBII) overnight in the presence of 50 μg/ml, carbenicillin (pET21b+) or 30 μg/mL (for LB) to 60 μg/mL (for TBII) kanamycin (pET28b+ and pET29b+). Starter cultures were used to inoculate 500 mL of Studier TBM-5052 autoinduction media containing antibiotic and grown at 37° C. for 24 hours. Cells were harvested by centrifugation at 4000 ref for 20 minutes at 4° C. and resuspended in lysis buffer (20 mM Tris. 300 mM NaCl, 20 mM Imidazole, pH 8.0 at room temperature), then lysed by microfluidization in the presence of 1 mM PMSF. Lysates were cleared by centrifugation at 24,000 rcf for at least 30 minutes at 4° C. Supernatant was applied to Ni-NTA (Qiagen) columns pre-equilibrated in lysis buffer. The column was washed twice with 15 column volumes. (CV) of wash buffer (20 mM Tris, 300 mM NaCl, 40 mM Imidazole, pH 8.0 at room temperature), followed by 15 CV of high-salt wash buffer (20 mM Tris, 1M NaCl, 40 mM Imidazole, pH 8.0 at room temperature) then 1.5 CV of wash buffer. Protein was eluted with 20 mM Tris. 300 mM NaCl, 250 mM Imidazole, pH 8.0 at room temperature. Proteins were further purified by gel filtration using FPLC and a Superdex™ 75 Increase 10/300 GL (GE) size exclusion column, pooling fractions containing monomeric protein.

### Size-Exclusion Chromatography, Multi-Angle Light Scattering (SEC-MALS)

SEC-MALS experiments used a Superdex™ 75 Increase 10/300 GL (GE) size exclusion column connected to a miniDAWN™ TREOS multi-angle static light scattering and an Optilab T-rEX™ (refractometer with Extended range) detector (Wyatt Technology Corporation, Santa Barbara Calif., USA). Protein samples were injected at concentrations of 3-5 mg/mL in TBS (pH 8.0). Data was analyzed using ASTRATM™ (Wyatt Technologies) software to estimate the weight average molar mass (Mw) of eluted species, as well as the number average molar mass (Mn) to assess monodispersity by polydispersity index (PDT)=Mw/Mn.

### Circular Dichroism (CD) Measurements

CD wavelength scans (260 to 195 nm) and temperature melts (25 to 95 C) were measured using an AVIV model 420 CD spectrometer. Temperature melts monitored absorption signal at 222 nm and were carried out at a heating rate of 4° C./min. Protein samples were at 0.3 mg/mL in PBS pH 7.4 in a 0.1 cm cuvette. Guanidinium chloride (GdmCl) titrations were performed on the same spectrometer with an automated titration apparatus in PBS pH 7.4 at 25 μ C. monitored at 222 nm with protein sample at 0.03 mg/mL in a 1 cm cuvette with stir bar. Each titration consisted of at least 40 evenly distributed concentration points with one minute mixing time for each step. Titrant solution consisted of the same concentration of protein in PBS+GdmCl. GdmCl concentration was determined by refractive index.

### Small Angle X-ray Scattering (SAXS)

Samples were exchanged into SAXS buffer (20mM Tris, 150mM NaCl, 2% glycerol, pH 8.0 at room temperature) via gel filtration. Scattering measurements were performed at the SIBYLS™ 12.3.1 beamline at the Advanced Light Source. The X-ray wavelength (λ) was 1.27 Å and the sample-to-detector distance of the Mar165 detector was 1.5 m, corresponding to a scattering vector q (q=4π*sin(θ/λ) where 2θ is the scattering angle) range of 0.01 to 0.59 Å⁻¹. Data sets were collected using 34 0.2 second exposures over a period of 7 seconds at 11 keV with protein at a concentration of 6 mg/mL. Data were also collected at a concentration of 3 mg/mL to determine concentration-dependence; all presented data was collected at the higher concentration as no concentration-dependent aggregation was observed. Data from 32 exposures was averaged separately over the Gunier, Parod, and Wide-q regions depending on signal quality over each region and frame. The averages were analyzed using the ScÅtter software package to analyze data and report statistics. FoXS was used to compare design models to experimental scattering profiles and calculate quality of fit (X) values. The hexahistidine tags and thrombin cleavage sites on the N-terminii of LOCKR proteins were modeled using Rosetta Remodel™ so that the design sequence matched that of the experimentally tested protein. To capture conformational flexibility of these residues, 100 independent models were generated, clustered, and the cluster center of the largest cluster was selected as a representative model for FoXS fitting without bias.

### GFP Pulldown Assay

His-tagged LOCKR was expressed per the above protocol from pET28b+ while the key was expressed fused to superfolder CGP (sfGFP) without a his-tag in pET21b+. The his-tagged LOCKR was purified to completion and dialyzed into TBS (20 mM Tris, 150 mM NaCl, pH 8.0 at room temperature); the key-GFP remained as lysate for this assay. 100 μL LOCKR at >1 uM was applied to a 96-well black Pierce® Nickel Coated Plate (ThermoFisher) and incubated at room temperature for 1 hour. Sample was discarded from the plate and washed 3× with 200 μL TBST (TBS+0.05% Tween-20). 100 μL of lysate containing key-GFP was added to each well and incubated at room temperature for 1 hour. Sample was discarded from the plate and washed 3× with 200 μL TBST (TBS+0.05% Tween-20). The plate was washed 1× with TBS, and 100 μL of TBS was added to each well. sfGFP fluorescence was measured on a Molecular Devices SpectraMax™ M5 plate reader or BioTek Synergy Neo2 plate reader; fluorescence was measured at 485 nm excitation and 530 nm emission, with a bandwidth of 20 nm for excitation and emission.

### Bio-Layer Interferometry (BLI)

BLI measurements were made on an Octet® RED96 System (ForteBio) with streptavidin (SA) coated biosensors and all analysis was performed within ForteBio Data Analysis Software version 9.0.0.10. Assays were performed with protein diluted into HBS-EP+ Buffer from GE (10 mM HEPES, 150 mM NaCl, 3 mM EDTA, 0.05% v/v Surfactant P20, 0.5% non-fat dry milk, pH7.4 at room temperature). Biotinylated Bcl2 was loaded onto the SA tips to a threshold of 0.5 nm programmed into the machine's protocol. Baseline was obtained by dipping the loaded biosensors into HBS-EP+ buffer; association kinetics were observed by dipping into wells containing defined concentrations of LOCKR and key, then dissociation kinetics were observed by dipping into the buffer used to obtain the baseline. Kinetic constants and response at equilibrium were computed by fitting a 1:1 binding model.

### Thermodynamic LOCKR Model

The thermodynamic model in FIG. 1a illustrates three free parameters for five equillibrea. This defines three equations that relate the concentrations of all species (open or closed Switch, Key, Target, Switch-Key, Switch-Target, and Switch-Key-Target) at equilibrium.

Kₒₚₑₘ=[Switchₒₚₑₙ]/[Switch_{closed}]

K_{CK}=[Switchₒₚₑₙ][Key]/[Switch−Key]=[Switch−Target][Key]/[Switch−Key−Target]

K_{LT}=[Switch−Key][Target]/[Switch−Key−Target]=[Switchₒₚₑₙ][Target]/[Switch−Target]

The total amount of each component (Switch. Key, and Target) is also constant and constrains the values of each species at equilibrium. This introduces the following equations to the model.

[Switch]ₜₒₜₐₗ=[Switchₒₚₑₙ]+[Switch_{closed}]+[Switch−Key]+[Switch−Target]+[Switch−Key−Target]

[Key]ₜₒₜₐₗ=[Key]+[Switch−Key]+[Switch−Key−Target]

[Target]ₜₒₜₐₗ=[Target]+[Switch−Target]+[Switch−Key−Target]

These six equations were fed into sympy.nsolve( ) to find solutions given the six constants (three equilibrium constants, three total concentrations). Fraction bound was extracted from this solution and plotted for the corresponding figures.

Grafting Functional Sequence onto LOCKR using Rosetta

Models of functional LOCKRs were made by grafting bioactive sequences onto the latch were designed using Rosetta™ XML to sample grafts starting at every helical register on the latch. This protocol uses two Rosetta movers, SimpleThreadingMover to change the amino acid sequence on the latch, and FastRelax™ with default settings to find the lowest energy structure given the functional mutations. Designs were selected by eye in PyMol™ 2.0 and high quality grafts had important binding residues interacting with the cage and minimized the number of buried unsatisfied hydrogen bonding residues.

### Rosetta Design of Orthogonal LOCKR

Redesign of LOCkRₐ to orthogonal cage-key pairs using was carried out using Rosetta with scorefunction bet_nov16. We extracted a model of the five-helix cage from the extended LOCKR model and used the Rosetta™ BundleGridSampler module to generate an ensemble of backbones for new latch geometries. The BundleGridSampler generates backbone geometry based on the Crick mathematical expressions for a coiled-coil, and allows efficient, parallel sampling of a regular grid of coiled-coil expression parameter values, which correspond to a continuum of peptide backbone conformations. For each parametrically-generated latch conformation sampled, Rosetta™ residue selectors specified the interface of the cage and latch for design of hydrogen bonding networks (HBNet) followed by Rosetta™ sidechain design. Residues were selected for design through Rosetta residue selectors by selecting the interface of the latch and cage via the InterfaceByVector residue selector. This residue selection was passed into both HBNet and sidechain design to strictly design the switching interface while leaving the cage with its original LOCKR sequence. Hydrogen Bond networks were designed using HBNetStapleInterface on the residues selected at the interface. The output contained designs with two or three hydrogen bond networks which span the three helices that make up the interface. All output from HBNet was then designed using PackRotamersMover to place residues at the interface while maintaining the hydrogen bond networks. Two rounds of design were done. The first used beta_soft to aggressively pack the interface with potentially clashing rotamers while optimizing the interaction energy at the interface, then the structure was minimized using beta to resolve potential clashing atoms according to the full Rosetta score function. The final round of design placed rotamers with the full beta Rosetta score function to finally optimize the interactions across the cage-latch interface.

Candidate orthogonal LOCKR designs were selected based on lacking unsatisfied buried hydrogen bonding residues, the count of alanine residues as a proxy for packing quality, and sequence dissimilarity as a metric to find polar/hydrophobic patterns dissimilar enough to be orthogonal. Unsatisfied hydrogen bonding atoms were filtered out using the BuriedUnsatHbonds filter allowing no unsatisfied polar atoms according to the filter's metrics. Packing quality was determined by counting alanine residues at the interface because high alanine count means poor interdigitation of residues. A maximum of 15 alanine residues were allowed in the entire three helix interface. Pairwise sequence dissimilarity of every designed latch was scored with BLOSUM62 by aligning sequences using the Bio.pairwise2 package from BioPython as shown in seq_alignment.py. Alignment was performed disallowing gaps within the sequence through large opening and extension penalties which is analogous to a structural alignment of two helices to find the most similar superposition based on hydrophobic-polar patterning. Each score was subtracted from the maximum score to convert scores into a distance metric; the most diverse sequences has the lowest BLOSUM62 score which converts to the largest distance. The sequences were then clustered using HeirClust_fromRMSD.py and clustered with a cutoff of 170, resulting in 13 clusters. The center of each cluster was picked by maximizing distance between the 13 centers selected. The 13 candidates were then filtered by eye in PyMol™ 2.0 for unsatisfied hydrogen bonding atoms and qualitative packing quality. The five best designs by these three metrics were ordered as LOCKR_{b-f}.

### Asymmetrized LOCKR Switches

The original LOCKRₐ switch was redesigned using Rosetta™ with HBNet; residues known to be important for LOCKR function were kept fixed, and remaining residues were optimized to preserve hydrophobic packing while introducing sequence diversity that minimized the number of repeating amino acid sequences and motifs. Synthetic DNA coding for the designs was obtained as described previously and designs were expressed, purified, and biophysically characterized as described previously. Crystallization trials were set up as described in the next section.

### X-Ray Crystallography

### Crystallization of Protein Samples

Purified protein samples were concentrated to 12-50 mg/ml in 20 mM Tris pH 8.0 and 100 mM NaCl. Samples were screened with a 5-position deck Mosquito crystal (ttplabtech) with an active humidity chamber, utilizing the following crystallization screens: JCSG+ (Qiagen), JCSG Core I-IV (Qiagen), PEG/Ion (Hampton Research), and Morpheus (Molecular Dimensions). The optimal conditions for crystallization of the different designs were found as follows:
1-fix-short-BIM-t0: 0.1M Tris pH 8,5, 5% (w/v) PEG 8000, 20% (v/v) PEG 300, 10% (v/v) Glycerol (no cryo needed)
1fix-short-GFP-t0: 0.2M Sodium chloride, 0.1 M Sodium cacodylate pH 6.5, 2.0M Ammonium sulfate (plus 20% glycerol for cryo)
1fix-short-noBim(AYYA)-t0: 0.2M di-Sodium tartrate, 20% (w/v) PEG 3350 (no cryo added)

### X-Ray Data Collection and Structure Determination

The crystals of the designed proteins were looped and placed in the corresponding reservoir solution, containing 20% (v/v) glycerol if the reservoir solution did not contain cryoprotectant, and flash-frozen in liquid nitrogen. The X-ray data sets were collected at the Advanced Light Source at Lawrence Berkeley National Laboratory with beamlines 8.2.1 and 8.2.2. Data sets were indexed and scaled using either XDS³⁵ or HKL2000³⁶. Initial models were generated by the molecular-replacement method with the program PHASER™³⁷ within the Phenix™ software suite³⁸, using the design models as the initial search models. Efforts were made to reduce model bias through refinement with simulated annealing using Phenix.refine, or, if the resolution was sufficient, by using Phenix.autobuild⁴⁰ with rebuild-in-place set to false, simulated annealing and prime-and-switch phasing. Iterative rounds of manual building in COOT and refinement in Phenix™ were used to produce the final models. Due to the high degree of self-similarity inherit in coiled-coil-like proteins, datasets for the reported structures suffered from a high degree of pseudo translational non-crystallographic symmetry, as report by Phenix.Xtriage™, which complicated structure refinement and may explain the higher than expected R values reported. RMSDs of bond lengths, angles and dihedrals from ideal geometries were calculated with Phenix™. The overall quality of all final models was assessed using the program MOLPROBITY™.

gfpLOCKR: (GFP11-LOCKR) Switch Design and Characterization

Using the asymmetrized LOCKRₐ design scaffold, the 11^{th} strand of GFP was encoded into the Latch sequence of the Cage as described in the section above “Grafting Functional Sequence onto LOCKR using Rosetta™”, and synthetic genes coding for the designed proteins obtained as described above. Proteins were purified and biophysically characterized as described above. To test for induction of fluorescence upon addition of Key, the proteins were mixed by pipetting and immediately assayed in a black 96-well plate using a Biotek Synergy Neo2 plate reader to monitor relative GFP fluorescence (Ex: 488, Em: 508, 10 minutes between reads). Cage leakiness was evaluated by measuring GFP fluorescence over time in the absence of Key

In Vitro Co-Localization-Dependent Switching with gfpLOCKR (GFP11-LOCKR)

The GfpLOCKR Cage was cloned with SpyCatcher™ fused to its N-terminus via a floppy linker, the gfpLOCKR Key was cloned with SpyTag™ fused to its C-terminus via a floppy linker, and GFP1-10 was cloned into its own pET21 vector. These proteins were expressed in E. coli Lemo21 cells with Studier's autoinduction media overnight at 18° C. After expression, the producer cells were harvested by centrifugation and lysed by microtluidizer. The desired proteins were purified from clarified lysates by Ni-NTA affinity chromatography and quantitated by A²⁸⁰ on a nanodrop. Proteins were diluted to final concentrations in PBS (GFP1-10: 1.9 uM in all samples; Cage: 1.5 uM. 0.8 uM, 0.4 uM, 0.2 uM, 0.094 uM; Key: 1.5 uM, 0.8 uM, 0.4 uM, 0.2 uM, 0.094 uM) and pooled as follows: SpyCatcher™-Cage alone (no Key), SpyCatcher™-Cage with naked Key (no SpyTag™), and SpyCatcher-Cage with SpyTag-Key. The proteins were mixed by pipetting and immediately assayed in a black 96-well plate using a Biotek Synergy Neo2 plate reader to monitor relative GFP fluorescence (Ex: 488, Em: 508, 10 minutes between reads). Cage leakiness was evaluated by measuring GFP fluorescence over time in the absence of Key. Co-localization dependence was confirmed by showing that SpyTag™-Key activated GFP fluorescence faster than did naked Key.

### Caged Intein LOCKR Switches

The VMAc intein sequence was designed to be encoded into the Latch of LOCKRₐ. The VMAn Intein sequence was fused to Keyₐ. Constructs were cloned and purified as previous LOCKR designs described above. Intein activity (splicing) was assessed by SDS-PAGE.

### Large-Scale High-Throughput Design of LOCKR Switches

The computational pipeline to design of thousands of new LOCKR switch scaffolds from scratch is as follows: backbones were exhaustively sampled using Crick helical parameters for 3-helix bundles (denoted 2plus1 or 2+1 because of a 2-helix scaffold plus 1-helix latch) and 4-helix bundles (denoted 3plus1 or 3+1 because of 3-helices plus 1-helix latch); parameters sampled include z-offset (−1.51, 0 and 1.51), helical phase every 10 degrees between 0 and 100, and superhelical radii for each helix ranging from 5-10 angstroms from the central superhelical axis (z-axis); based on the success of the original LOCKR design, we focused on designs with straight helices and no supercoiling (superhelical twist fixed to 0.0). Each generated helix is 58 residues in length; Rosetta loop closure methods were used to add loops connecting all helices into a single polypeptide chain (Cage scaffold). Sequence and sidechain design was carried out using HBNet, MC-HBNet, and RosettaDesign™. Additional designs were generated by truncating the helical bundles into shorter scaffolds, making versions with the Latch as either the N-terminal or C-terminal helix, and by trying different toehold lengths (truncations of Latch helix that end in a polar residue and remove at least one or two hydrophobic packing residues from the original design). Designs were selected based on computational methods learned from iterative testing and design of previous LOCKR scaffolds and HBNet helical bundles: important metrics include secondary structure shape complementarity (ss_sc)>0.65 (best designs had ss_sc>0.7); RosettaHoles™ filter in regions surrounding hydrogen bond networks to eliminate designs with large cavities adjacent to hydrogen bond networks in the core of the scaffolds; designs were required to have at least 2 distinct hydrogen bond networks that spanned all helices of the design model (i.e. each helix must contribute at least one amino acid sidechain to the network); the number of Ile, Leu, and Val residues, and number of contacts made by these amino acid types, as compared to Ala (smaller amino acid) also serves as a proxy that correlates well with designs that have tight, interdigitated hydrophobic packing, which is important for generating a stable protein scaffold.

strepLOCKR (STREPII-LOCKR) Computational Design:

LOCKR switches encoding the STREPII tag, (N)WSHPQFEK (SEQ ID NO: 63), were designed using the 2plus1 and 3plus1 switches from the large-scale high-throughput LOCKR design set. This sequence is difficult to encode because of the Pro (which kinks alpha helices) and the Trp and His, which if buried must likely participate in hydrogen bonds. To address these issues, rather than sampling all helical residues, the large-scale design set was mined to find LOCKR scaffolds that already contained Trp (W), His (H) already pre-organized into hydrogen bond networks of the designs. Designs with pre-organized Phe (F) were also considered.

strepLOCKR (STREPII-LOCKR) Experimental Testing:

The purified proteins were tested for their ability to sequester the STREPII sequence in absence of Key, and activate in presence of Key using biolayerinterferometry (Octet® RED96 System, PALL ForteBio): THE™ NWSHPQFEK (SEQ ID NO: 63) Tag Antibody (mAb mouse, Genscript A01732-200) was loaded onto Anti-Mouse IgG Fc Capture (AMC) Biosensors (PALL ForteBio); tips were preconditioned by cycling between Glycine pH 1.65 and Octet assay buffer: HBS-EP+Buffer from GE (10 mM HEPES, 150 mM NaCl, 3 mM EDTA, 0.05% v/v Surfactant P20, 0.5% non-fat dry milk, pH 7.4 at room temperature). Protein samples were diluted into Octet assay buffer, keeping dilution factors consistent as to minimize noise. The antibody-loaded tips were reused up to 8 times using the recommended regeneration protocol of cycling between Glycine pH 1,65 and Octet assay buffer (minimal loss in loading was observed when the tips were preconditions, and a signal threshold was set to ensure consistent loading of the tips each time).

The THE™ NWSHPQFEK (SEQ ID NO: 63) Tag Antibody (mAb mouse, Genscript A01732-200) was used at a concentration of 5 ug/mL in Octet assay buffer; stocks of antibody were made up to 0.5 mg/mL with 400 ul mqH2O, aliquoted and stored at −80° C., thawed immediately before use.

Purification of Proteins from Bacterial Preps Not Already Described Above:

Starter cultures were grown at 37° C. in either Luria-Bertani (LB) medium overnight, or in Terrific Broth for 8 hours, in the presence of 50 μg/ml carbenicillin (pET21-NESG) or 50 μg/ml kanamycin (pET-28b+). Starter cultures were used to inoculate 500mL of LB (induced with 0.2 mM IPTG at OD600 of ˜0.6-0.9) or Studier auto-induction media containing antibiotic. Cultures were expressed overnight at 18° C. (many designs were also later expressed at 37° C. for 4 hours with no noticeable difference in yield). Cells were harvested by centrifugation for 15 minutes at 5000 ref 4° C. and resuspended in lysis buffer (20 mM Tris, 300 mM NaCl, 20 mM Imidazole, pH 8.0 at room temperature), then lysed by sonication in presence of lysozyme, DNAse, and EDTA-free cocktail protease inhibitor (Roche) or 1 mM PMSF. Lysates were cleared by centrifugation at 4° C. 18,000 rpm for at least 30 minutes and applied to Ni-NTA (Qiagen) columns pre-equilibrated in lysis buffer. The column was washed three times with 5 column volumes (CV) of wash buffer (20 mM Tris, 300 mM NaCl, 40 mM Imidazole, pH 8.0 at room temperature), followed by 3-5 CV of high-salt wash buffer (20 mM Tris, 1 M NaCl, 40 mM lmidazole, pH 8.0 at room temperature), and then 5 CV of wash buffer. Protein was eluted with 20 mM Tris, 300 mM NaCl, 250 mM Imidazole, pH 8.0 at room temperature. No reducing agents were added, as none of the designed proteins contained cysteines.

### REFERENCES

1. Huang, P.-S., Boyken, S. E. & Baker, D. The coming of age of de novo protein design. Nature 537, 320-327 (2016).
2. Joh, N. H. et al. De novo design of a transmembrane Zn²⁺-transporting four-helix bundle. Science 346, 1520-1524 (2014).
3. Davey, J. A., Damry, A. M., Goto, N. K. & Chica, R. A. Rational design of proteins that exchange on functional timescales. Nat. Chem. Biol. 13, 1280-1285 (2017).
4. Huang, P.-S. et al. High thermodynamic stability of parametrically designed helical bundles. Science 346, 481-485 (2014).
5. Boyken, S. E. et al. De novo design of protein homo-oligomers with modular hydrogen-bond network-mediated specificity. Science 352, 680-687 (2016).
6. Berger, S. el al. Computationally designed high specificity inhibitors delineate the roles of BCL2 family proteins in cancer. Elife 5, 1422 (2016).
7. Leaver-Fay, A. et al. ROSETTA3: an object-oriented software suite for the simulation and design of macromolecules. Meth Enzymol 487, 545-574 (2011).
8. Kuhlman, B. & Baker, D. Native protein sequences are close to optimal for their structures. Proc Natl Acad Sci USA 97, 10383--10388 (2000).
9. Crick, F. H. C. The packing of [alpha]-helices: simple coiled-coils. Acta Cyst (1953). Q6, 689-697 [doi: 10.1107/S0365110X53001964]6, 1-9 (1953).
10. Glantz, S. T. et al. Functional and topological diversity of LOV domain photoreceptors. Proc Natl Acad Sci USA 113, E1442-51 (2016).
11. Dueber, J. E., Mirsky, E. A. & Lim, W. A. Engineering synthetic signaling proteins with ultrasensitive input/output control. 25, 660-662 (2007).
12. Dueber, J. E., Yeh, B. J., Chak, K. & Lim, W. A. Reprogramming control of an allosteric signaling switch through modular recombination. Science 301, 1904-1908 (2003).
13. Huang, P.-S. et al. RosettaRemodel: A Generalized Framework for Flexible Backbone Protein Design. PLoS ONE 6, e24109 (2011).
14. Schneidman-Duhovny, D., Hammel, M. & Sali, A. FoXS: a web server for rapid computation and fitting of SAXS profiles. Nucleic Acids Res 38, W540-4 (2010).
15. Maguire, J. B., Boyken, S. E., Baker, D. & Kuhlman, B. Rapid Sampling of Hydrogen Bond Networks for Computational Protein Design. J. Chem. Theory Comput 14, 2751-2760 (2018).

### EXAMPLE 2

### degronLOCKR for Tunable Control of Protein Degradation

### Summary:

This example builds on the Latching Orthogonal Cage Key pRoteins (LOCKR) technology, disclosed above, which are de novo designed protein switches. Degron LOCKR is a specific embodiment of LOCKR for use (for example) in cellular engineering with a modified version of a degron (including but not limited to the C-terminus of the murine omithine decarboxylase (cODC)) caged. When a key is expressed and activates the switch, the degron targets the switch and any protein (hereafter “cargo”) fused to it for degradation. In this way, a cargo of interest that has the degron Switch fused to its C-terminus can be conditionally degraded in a titratable manner via expression of the key. This is sometimes referred to as degronLOCKR. This embodiment provides significant benefits, including but not limited to the following:
degronLOCKR is activated separate from natural proteins and works in any eukaryote.
Natural proteins cannot be tuned to the extent LOCKR can. We have enabled tighter control over the dynamic range and specificity of protein degradation in vivo.
No current method to use orthogonally acting degrons in a single cell.
No current method to modularly tag any cellular protein with a conditional degron dependent on expression of the control peptide
Can tune responsiveness over a large dynamic range by varying length, changing residues within the cODC degron, and or mutating residues in interface
degronLOCKR can be used as a modular regulatory hub in biotechnological applications. By tagging degronLOCKR to (for example) metabolic enzymes, transcription factors, kinases, or phosphatases, flux through different biological pathways can be controlled.
Tagged to functional modules of engineered cell types, such as CAR, SynNotch, and kinases, this invention can improve the fidelity and modulate the function of therapeutically relevant engineered cells.
dCas9 has known off-target effects so degrading it selectively under control of degronLOCKR is a means of controlling gene therapies that rely on precise gene editing.
   degronLOCKR Design

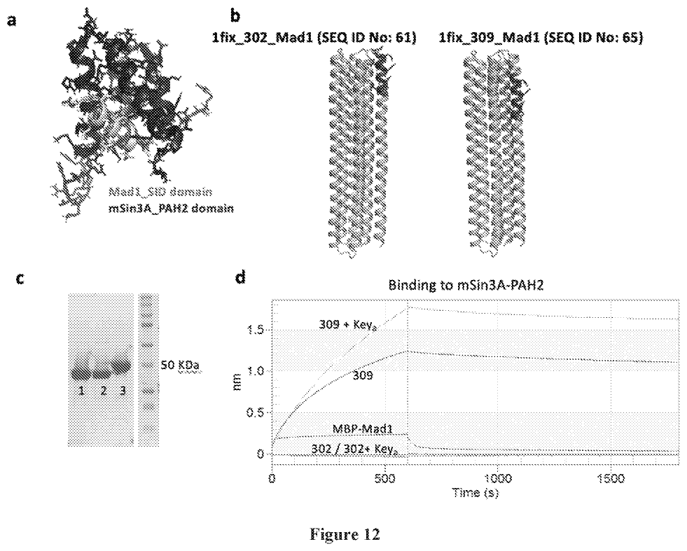

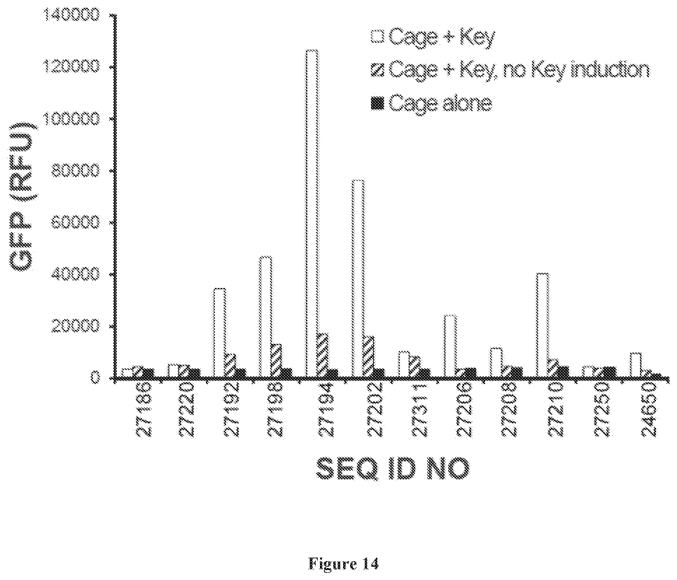

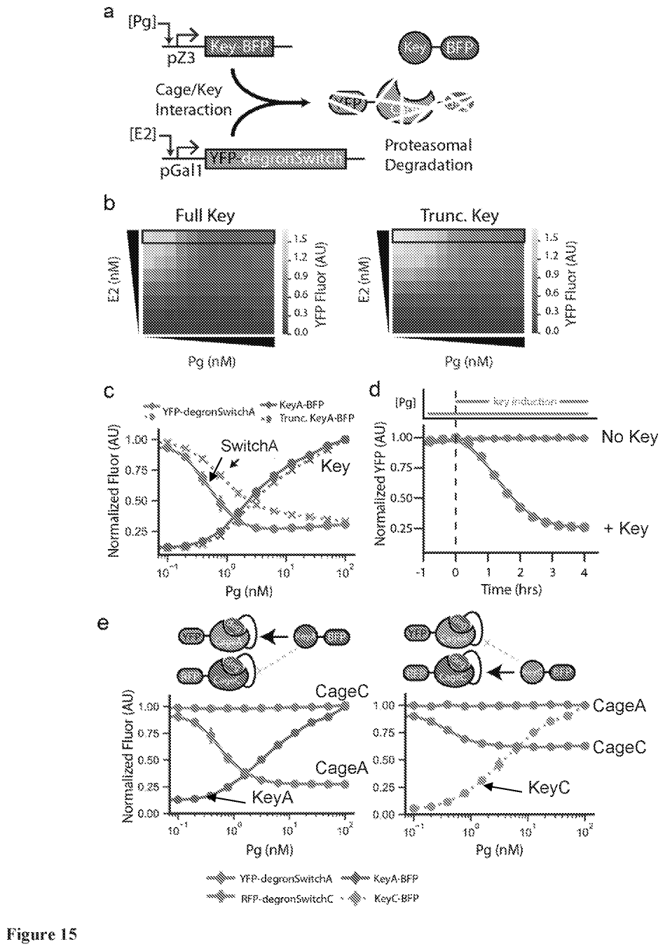

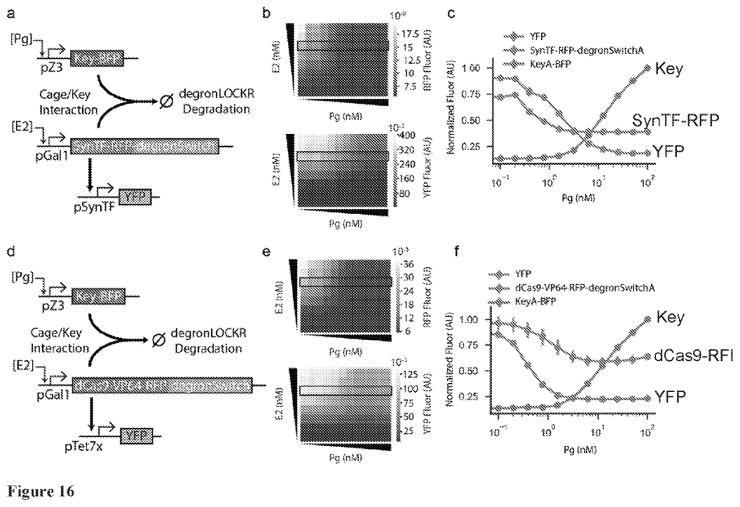

We caged the cODC degron, a ubiquitin-independent degradation signal from the C-terminus of murine ornithine decarboxylase as a strategy for controlling arbitrary protein stability in a living cell. Our goal was to have degradation of the switch, and any protein fused to it, be inducible by Key. The caging strategy employed for Bim was used to embed three variants of cODC into Switchₐ: the wild-type sequence, wild-type with a proline removed (since proline destabilizes alpha helices), and the dipeptide sequence CA, believed to be the minimal functional residues of the degron (FIG. 17). We tested each switch variant in budding yeast S. cerevisiae, using a dual inducible system² to independently titrate the concentration of the switch with a yellow fluorescent protein (YFP) N-terminal fusion and the Key with a blue fluorescent protein (BFP) C-terminal fusion (FIG. 15a). To assess the dynamic range of switch activation we titrated in different amounts of Key using a range of progesterone (Pg) concentrations at a fixed amount of YFP-degronSwitchₐ (at a single concentration of estradiol (E2)) and measured steady-state fluorescence using flow cytometry. Key induced degradation observed for these initial constructs was dependent on the presence of the cODC degron in the switch, and was not observed when YFP was fused to either BimSwitchₐ or Switchₐ (FIG. 18). In order to optimize the amount of inducible degradation, we varied the switch toehold length to tune Kₒₚₑₙ. The switch with the largest dynamic range was the proline-removed cODC and a 12-residue toehold (hereafter referred to as degronSwitchₐ). Using this variant, YFP fluorescence fused to degronSwitchₐ was reduced up to 73% upon full induction of Keyₐ (FIG. 19).

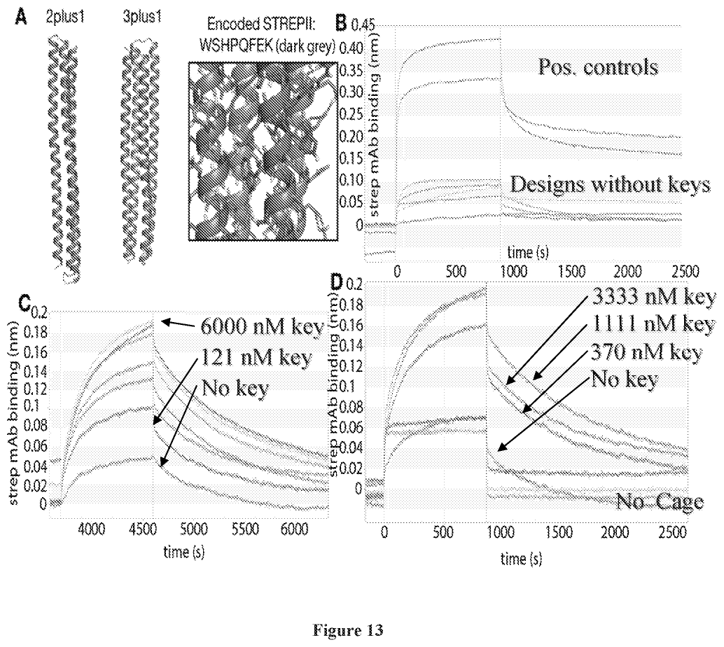

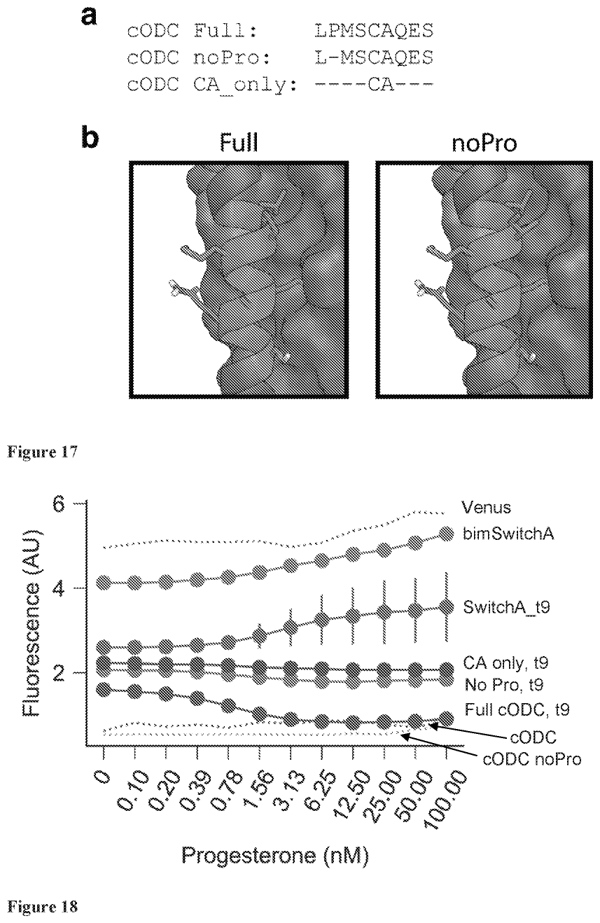

We explored the dynamic range of degronLOCKRₐ at different concentrations of YFP-degronSwitchₐ and Keyₐ-BFP for two different Key lengths (FIG. 15b) by testing the full range of E2 and Pg combinations. The extent of Keyₐ-induced degradation of degronSwitchₐ varied as a function of the concentration of both proteins. Key, fluorescence was stable as a function of degronSwitchₐ concentration (FIG. 20), suggesting the Key is not co-degraded with the degronSwitch. With a truncated Keyₐ (43 residues versus 55 residues), the same dynamic range of switch activation was observed, but a higher Key concentration was required for maximal activation (FIG. 15c). This is similar to the behavior observed with BimLOCKR (FIG. 16d), and suggests our model of Cage/Key interaction holds true within living cells. To assess the dynamics of degronLOCKRₐ activation, we used an automated flow cytometry platform to measure YFP fluorescence as a function of time. Cells were grown at a constant concentration of E2 until YFP-degronSwitchₐ reached steady-state and then induced with Pg to activate production of Keyₐ-BFP. We found that the in viva half-life for active degronLOCKRₐ is 24 minutes, which is very similar to the reported half-life of 11-30 minutes for the constitutive cODC degron.

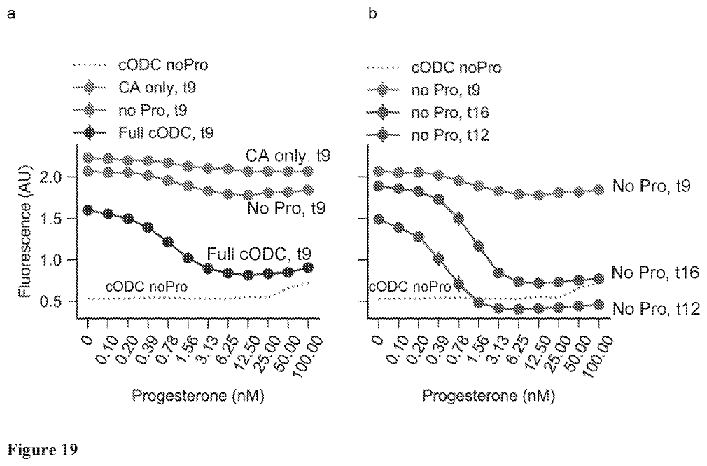

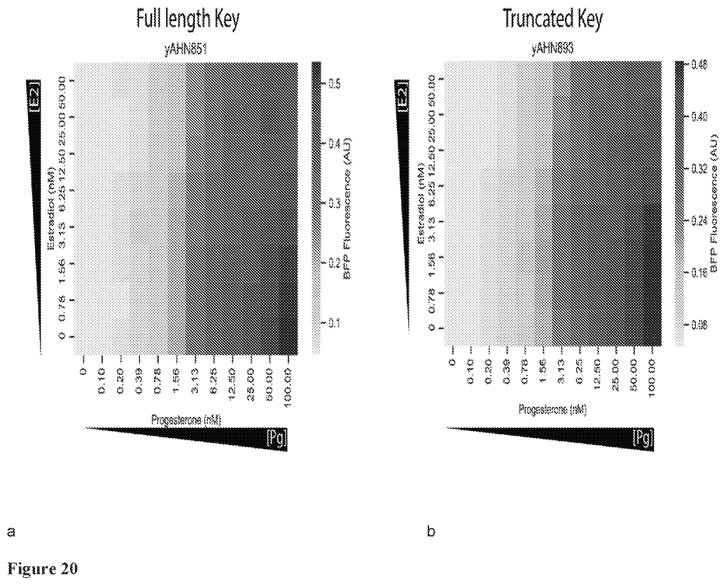

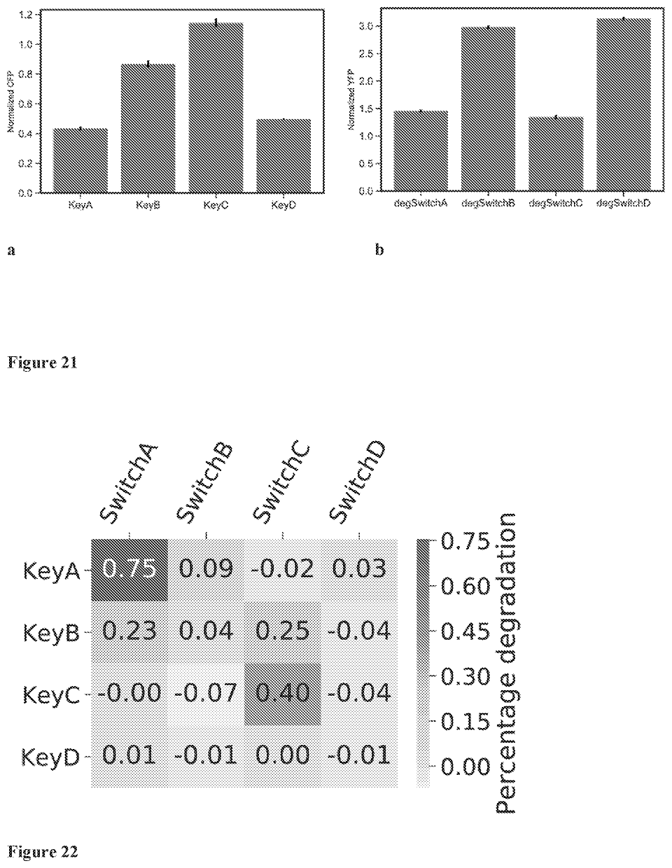

We next sought to enhance the functionality of degronLOCKR to trigger orthogonal degradation of different proteins in the same cell by installing the proline removed cODC degron in LOCKR_{b}, LOCKR_{c}, and LOCKR_{d}. We constitutively expressed each orthogonal switch variant fused to YFP (FIG. 21) and measured the degradation of YFP with constitutive expression of each Key variant fused to cyan fluorescent protein (CFP). DegronLOCKRₐ and degronLOCKR_{c} were strongly activated by their cognate Keys, but not by each other's Key (other constructs did not activate in vivo; FIG. 22). To test the orthogonality of the degronLOCKRs, we constitutively coexpressed degronLOCKRₐ and degronLOCKR_{c} in the same cell fused to YFP and red fluorescent protein (RFP), respectively, and used the Pg inducible system to titrate expression of each Key variant in separate strains. Expression of Keyₐ led to selective degradation of YFP but not RFP, and expression of Key_{c} led to selective degradation of RFP but not YFP (FIG. 15d). This demonstrates that the dual degronLOCKR system can function orthogonally and simultaneously in living cells.

degronLOCKR Control of Gene Expression In Vivo

To demonstrate the utility of degronLOCKR, we used it as a tool to modulate the intracellular concentration of a synthetic transcription factor and dCas9. We first placed a zinc-finger based synthetic transcription factor (SvnTF) fused to both RFP and degronSwitchₐ under the control of the E2 inducible promoter. and Keyₐ-BFP-NLS under the control of the Pg inducible promoter. To monitor SynTF activity, we measured pSynTF-YFP fluorescence in the same cell (FIG. 16a). An increase in expression of SynTF-RFP-degronSwitchₐ increased both RFP and YFP fluorescence, while an increase in Key expression decreased both outputs (FIG. 16b). For example, at 31.25 nM E2 (FIG. 16b), maximal Key induction caused a 61% reduction of RFP and 82% reduction of YFP (FIG. 16c). Notably, degronLOCKR caused a graded change in YFP fluorescence as a function of Key concentration, which contrasts with the more digital behavior of transcription factors typically used in synthetic biology applications⁵. To further establish degronLOCKR as a general method of transcriptional control, we next tested degradation of an activating dCas9-VP64 fusion. dCas9 was targeted to the tet operator site of the pTet7x with a constitutively expressed sgRNA to induce expression of YFP (FIG. 16d), and Key expression was titrated at different concentrations of dCas9 (FIG. 16e). We observed a 78% reduction of RFP and 41% reduction of YFP upon induction of Key at 31.25 nM E2 (FIG. 16t). Together, these results demonstrate the modularity and functionality of degronLOCKR as a tool to control the stability of proteins in vivo.

### Methods

### Construction of DNA Circuits

Hierarchical golden gate assembly was used to assemble plasmids for yeast strain construction using the method in Lee et al.⁷. Individual parts had their Bsal, BsmBI, and NotI cut sites removed to facilitate downstream assembly and linearization. Parts were either generated via PCR or purchased as gBlocks from IDT. These parts were assembled into transcriptional units (promoter-gene-terminator) on cassette plasmids. These cassettes were then assembled together to form multi-gene plasmids for insertion into the yeast genome.

### Yeast Strains and Growth Media

The base S. cerevisiae strain used in all experiments was BY4741 (MATa his3Δ1 leu2Δ0 met 15Δ0 ura3Δ0). All yeast cultures were grown in YPD media (10 g/L Bacto Yeast Extract, 20 g/L. Bacto peptone, 20 g/L dextrose) or synthetic complete medium (SDC) (6,7 g/L Bacto-yeast nitrogen base without amino acids, 2 g/L complete supplement amino acid mix, 20 g/L dextrose). Selection of auxotrophic markers (URA3, LEU2, and/or HIS3) was performed on synthetic complete medium with the appropriate dropout amino acid mix.

### Estradiol and Progesterone Induction

Yeast strains were grown overnight by picking a single colony from a plate into YPD media. Saturated culture was diluted 1:500 in fresh SDC and aliquoted into individual wells of a 2 mL 96 well storage block (Corning) for a three hour outgrowth at 30° C. and 900 RPM in a Multitron shaker (Infors HT). Estradiol (Sigma-Aldrich) and progesterone (Fisher Scientific) were prepared at a 10× concentration by making the appropriate dilutions into SDC from a 3.6 mM estradiol and 3.2 mM progesterone stock solution. After the three hour outgrowth, 50 ul of estradiol and progesterone inducer were added to the 96 well block in the appropriate combinations and the block was returned to the shaker.

### Description of Automated Flow Cytometry and Continuous Culture System Hardware

We adapted an existing automated experimental platform³ to perform variable concentration small molecule induction and long-term culturing. Yeast cultures were grown in 50 mL optically clear conical tubes (Falcon) that were held in eight custom temperature-controlled, magnetically stirred chambers. Liquid handling was accomplished using a 14 position stream selector (VICI Cheminert) and two syringe pumps (Cavro XCalibur Pump, TECAN) of a BD High-Throughput Sampler. Commands to the HTS were controlled using LABVIEW 2013. This setup allowed for periodic sampling and dilution of individual cultures. Each sampling period consisted of three main steps: 1) send sample to flow cytometer for measurement, 2) extract culture and send to waste, and 3) replenish culture with fresh media at desired hormone concentration. Each sampling period can be designated to either induce cultures to a new higher hormone concentration or to maintain desired hormone concentration. A sampling frequency of 24 minutes and a dilution volume of 3 mL were used.

### Yeast Culture

Yeast strains were grown overnight by picking a single colony from a plate into YPD media. Saturated culture was diluted 1:200 into fresh SDC. Cultures were grown for 2 hours in glass tubes at 30 C and 250 RPM in a Innova 44 shaker (New Brunswick). Cultures were then diluted to 0.01 OD600 in fresh SDC and aliquoted into individual 50 mL optically clear conical tubes (Falcon) at a total volume of 30 mL YPD. Another one hour outgrowth was performed in bioreactors with magnetically-controlled stir bars at 30 C. All SDC media was supplemented with 5,000 U/mL Penicillin Streptomycin (Thermo-Fisher).

Estradiol and Progesterone Induction to Test degronLOCKR Dynamics

A 1× concentration was determined by the highest desired hormone concentration at which to test strains (30 nM E2 and 50 nM Pg. respectively). A solution of E2 and SDC media was created at a 10× concentration to bring pre-induced cultures to a desired concentration in one sampling period. A second solution of Pg and SDC media was created at a 10× concentration to induce Key expression after degSwitch-YFP expression reached steady-state. SDC media was prepared at three different concentrations of hormone: (1) 10× E2/no Pg, (2) 1X E2/no Pg, (3) 1X OX Pg, and (4) 1X E2/1X Pg. After a one hour outgrowth in bioreactors (t=−6 hr), the first induction was performed to achieve E2 concentration by extracting 3 mL from all cultures and replenishing with (1). After E2 induction, sampling proceeded as described above (see Hardware). All sampling periods following the first induction time point included sending a sample to the cytometer for measurement, extracting 3 mL from all cultures, and replenishing cultures with (2). During the second induction time point (t=0 hr), cultures were induced with (3) to activate Key expression. This induction was followed by the same procedure as the first induction, except that hormone concentrations were maintained by (4). Controls (no activated Key expression) did not undergo a second induction and, instead, continued to be replenished by (2).

### Flow Cytometry

Analysis of fluorescent protein expression was performed using a BD LSR11 flow cytometer (BD Biosciences) equipped with a high-throughput sampler. Cultures were diluted in TE before running through the instrument to obtain an acceptable density of cells. YFP (Venus) fluorescence was measured using the FITC channel, RFP (mScarlet) was measured using the PE-Texas Red channel, and BFP (mTagBFP2) was measured using the DAPI channel. For steady-state measurements, 5,000-10,000 events were collected per sample. For dynamic measurements, 2,000-10,000 events were collected per sample. Fluorescence values were calculated as the height (H) measurement for the appropriate channel and normalized to cell size by dividing by side scatter (SSC-H). All analysis of flow cytornetry data was performed in Python 2.7 using the package FlowCytomenyTools and custom scripts.

Appendix
Amino Acid Sequence of cODC degrons threaded: An “X” represents a sequence
position that can be any amino acid
>cODC
(SEQ ID NO: 28, 466)
LPMSCAQES
>cODC_noPro
(SEQ ID NO: 28, 467)
LXMSCAQES
>cODC_CA_only
----CA---
Amino Acid Sequences of degronLOCKR Switches
Degron sequence underlined
Latch indicated by [brackets]
>degonLOCKR_a_327
(SEQ ID NO: 27359)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDP[DEARKAIARVKRESKRIVEDAERLPMSCAQESEKISREAERLIREAA]
>degonLOCKR_a_327_noPro
(SEQ ID NO: 27360)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDP[DEARKAIARVKRESKRIVEDAERLAMSCAQESEKISREAERLIREAA]
>degonLOCKR_a_CAonly
(SEQ ID NO: 27361)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDP[DEARKAIARVKRESKRIVEDAERLIRECAAASEKISREAERLIREAA]
>degonLOCKR_a_324_t12
(SEQ ID NO: 27362)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDP[DEARKAIARVKRESKRIVEDLIMSCAQESAASEKISREAERLIR]
>degonLOCKR_a_320_t16
(SEQ ID NO: 27363)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDP[DEARKAIARVKRESKRLVMSCAQESREAAAASEKISREAE]
>degron-miniLOCKRa_1_t9
(SEQ ID NO: 27364)
NKEDATEAQKKAIRAAEELLKDVTRIQERAIREAEKALERLARVQEEAIRRVYEAVESKNKEELKKVKEEIEELL
RRLKRELDELEREIRELLKEIKEKADRLEKEIRDLIERIRRDRNASDEVVTRLARLNEELIRELREDVRRLAELN
KELLRELERAARELARLNEKLLELADRVETE[EEARKAIARVKRESKRIVEDAERLAMSCAQESEKISREAERLI
REAA]
>degron-miniLOCKRa_1_t12
(SEQ ID NO: 27365)
NKEDATEAQKKAIRAAEELLKDVTRIQERAIREAEKALERLARVQEEAIRRVYEAVESKNKEELKKVKEEIEELL
RRLKRELDELEREIRELLKEIKEKADRLEKEIRDLIERIRRDRNASDEVVTRLARLNEELIRELREDVRRLAELN
KELLRELERAARELARLNEKLLELADRVETE[EEARKAIARVKRESKRIVEDLIMSCAQESAASEKISREAERLI
R]
>degron-miniLOCKRa_2_t9
(SEQ ID NO: 27366)
DERLKRLNERLADELDKDLERLLRLNEELARELTRAAEELRELNEKLVELAKKLQGGRSREVAERAEKEREKIRR
KLEEIKKEIKEDADRIKKRADELRRRLEKTLEDAARELEKLKREPRTEELKRKATELQKEAIRRAEELLKEVTDV
QRRAIERAEELLEKLARLQEEAIRTVYLLVELNKV[DRARKAIARVKRESKRIVEDAERLAMSCAQESEKISREA
ERLIREAA]
>degron-miniLOCKRa_t12
(SEQ ID NO: 27367)
DERLKRLNERLADELDKDLERLLRLNEELARELTRAAEELRELNEKLVELAKKLQGGRSREVAERAEKEREKIRR
KLEEIKKEIKEDADRIKKRADELRRRLEKTLEDAARELEKLKREPRTEELKRKATELQKENIRRAEELLKEVTDV
QRRNIERAEELLEKLARLQEENIRTVYLLVELNKV[DRARKAIARVKRESKRIVEDLIMSCAQESAASEKISREA
ERLIR]
>1fix-short_cODC_mut
(SEQ ID NO: 27368)
SKEAAKKLQDLNIELARKLLEASTKLQRLNIRLAEALLEAIARLQELNLELVYLAVELTDPKRIRDEIKEVKDKS
KEIIRRAEKEIDDAAKESKKILEEARKAIRDAAEESRKILEEGSGSGSDALDELQKLNLELAKLLLKAIAETQDL
NLRAAKAFLEAAAKLQELNIRAVELLVKLTDPATIRRALEHAKRRSKEIIDEAERAIRAAKRESERIIEEARRLI
EKAKEESERIIREGSGSGDPDIKKLQDLNIELARELLRAHAQLQRLNLELLRELLRALAQLQELNLDLLRLASEL
TDPDEARKAIARVKRESKRIVEDAERLSREAAALSMSCAQESERSIREAAAASEKISRE]
>1fix-short_cODC_mut_t6
(SEQ ID NO: 27369)
SKEAAKKLQDLNIELARKLLEASTKLQRLNIRLAEALLEAIARLQELNLELVYLAVELTDPKRIRDEIKEVKDKS
KEIIRRAEKEIDDAAKESKKILEEARKAIRDAAEESRKILEEGSGSGSDALDELQKLNLELAKLLLKAIAETQDL
NLRAAKAFLEAAAKLQELNIRAVELLVKLTDPATIRRALEHAKRRSKEIIDEAERAIRAAKRESERIIEEARRLI
EKAKEESERIIREGSGSGDPDIKKLQDLNIELARELLRAHAQLQRLNLELLRELLRALAQLQELNLDLLRLASEL
TDP[DEARKAIARVKRESKRIVEDAERLSMSCAQESEKISREAERSIREAAAAS]
>1fix-short_cODC
(SEQ ID NO: 27370)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAIARVKRESKRIVEDLEMSCAQESAASEKISREAERLIR]
>1fix-short_cODC_t5
(SEQ ID NO: 27371)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAIARVKRESKRLVMSCAQESREAAAASEKISREA]
>1fix-short_cODC_t8
(SEQ ID NO: 27372)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAIARVKRLSMSCAQESERLIREAAAASEKIK]
>1fix-short_cODC_t11
(SEQ ID NO: 27373)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAIARLKMSCAQESEDAERLIREAAAASE]
>degonLOCKRb
(SEQ ID NO: 27374)
SHAAVIKLSDLNIRLLDKLLQAVIKLTELNAELNRKLIEALQRLFDLNVALVHLAAELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSNDPQVAQNQETFIELARDALRLVAENQEAFIEVARLTLRAAALAQEVAIKAVEAASEG
GSGSGP[NKEEIEKLAKEAREKLKKAEKEHKMSCAQERKKNKKAREDLKKKADK]
>degonLOCKRb_t13
(SEQ ID NO: 27375)
SHAAVIKLSDLNIRLLDKLLQAVIKLTELNAELNRKLIEALQRLFDLNVALVHLAAELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSNDPQVAQNQETFIELARDALRLVAENQEAFIEVARLTLRAAALAQEVAIKAVEAASEG
GSGSGP[NKEEIEKLAKEAREKLKKAEMSCAQEHDKLRKKNKKAREDLKK]
>degonLOCKRc_t9
(SEQ ID NO: 27376)
SLEAVLKLAELNLKLSDKLAEAVQKLAALLNKLLEKLSEALQRLFELNVALVTLAIELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSNDPLVARLQELLIEHARELLRLVATSQEIFIELARAFLANAAQLQEAAIKAVEAASEN
GSGSGP[SSEKVRRELKESLKENHKQNQKLLMSCAQEQEKLNRELEELKKKHKK]
>degonLOCKRc_t13
(SEQ ID NO: 27377)
SLEAVLKLAELNLKLSDKLAEAVQKLAALLNKLLEKLSEALQRLFELNVALVTLAIELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSNDPLVARLQELLIEHARELLRLVATSQEIFIELARAFLANAAQLQEAAIKAVEAASEN
GSGSGP[SSEKVRRELKESLKENHKQNMSCAQEHKRAQEKLNRELEELKK]
>degron-miniLOCKRc_1_t9
(SEQ ID NO: 27378)
LIERLTRLEKEHVRELKRLLDTSLEILRRLVEAFETNLRQLKEALKRALEAANLHNEEVEEVLRKLEEDLRRLEE
ELRKTLDDVRKEVKRLKEELDKRIKEVEDELRKIKEKLKKGDKNEKRVLEEILRLAEDVLKKSDKLAKDVQERAR
ELNEILEELSRKLQELFERVVEEVTRNVPTTE[RIEKVRRELKESLKENHKQNQKLLMSCAQEQEKLNRELEELK
KKHKK]
>degron-miniLOCKRc_1_t13
(SEQ ID NO: 27379)
LIERLTRLEKEHVRELKRLLDTSLEILRRLVEAFETNLRQLKEALKRALEAANLHNEEVEEVLRKLEEDLRRLEE
ELRKTLDDVRKEVKRLKEELDKRIKEVEDELRKIKEKLKKGDKNEKRVLEEILRLAEDVLKKSDKLAKDVQERAR
ELNEILEELSRKLQELFERVVEEVTRNVPTTE[RIEKVRRELKESLKENHKLNMSCAQEHKRAQEKLNRELEELK
K]
>degron-miniLOCKRc_2_t9
(SEQ ID NO: 27380)
SEERVLELAEEALRLSDEAAKEIQELARRLNEELEKLSKELQDLFERIVETVTRLIDADEETLKRAAEEIKKRLE
DARKKAKEAADKAREELDRARKKLKELVDEIRKKAKDALEKAGADEELVARLLRLLEEHARELERLLRTSARIIE
RLLDAFRRNLEQLKEAADKAVEAAEEAVRRVEDVRV[WSEKVRRELKESLKENHKQNQKLLMSCAQEQEKLNREL
EELKKKHKK]
>degron-miniLOCKRc_t13
(SEQ ID NO: 27381)
SEERVLELAEEALRLSDEAAKEIQELARRLNEELEKLSKELQDLFERIVETVTRLIDADEETLKRAAEEIKKRLE
DARKKAKEAADKAREELDRARKKLKELVDEIRKKAKDALEKAGADEELVARLLRLLEEHARELERLLRTSARIIE
RLLDAFRRNLEQLKEAADKAVEAAEEAVRRVEDVRV[WSEKVRRELKESLKENHKLNMSCAQEHKRAQEKLNREL
EELKK]
>degronLOCKRc_1fix_t13
(SEQ ID NO: 27382)
SLEAALKLAELNLKLSDKLAEASQKLAALLNKLLEKLSEAIQRLFELNLALVTLAIELTDPKRIADEIKKVKDKS
KEIIERAEEEIARAAAESKKILDEAEEEIARAAAESKKILDEGSGSGSDALAELQALNLKLAELLLEAIAETQAL
NLKAAEAFLEAAAKLQELNIKAVELLVKLTDPATIREALRKAKEDSERIIAEAERAIAAAKAESERIIREAERLI
AAAKAESERIIREGSGSNDPLIARLQELLIEHARELLRLHATSQEIFVELLRAFLANLAQLQEAALKALEAASEN
GSGSGP[SSEKVRRELKESLKENHKQNQKLLMSCAQEQEKLNRELEELKKKHKK]
>degonLOCKRd
(SEQ ID NO: 27383)
SLEAVLKLFELNHKLSEKLLEAVLKLHALNQKLSQKLLEALARLLELNVALVELAIELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPEVARLQEAFIEQAREILRNVAAAQEALIEQARRLLALAALAQEAAIKAVELASEH
GSGSGP[DTVKRILEELRRRFEKLAKDLDDIAMSCAQEHKKHNKELKDKQRKIK]
Amino Acid Sequences of keys for degronKOCKRs
>key_a
(SEQ ID NO: 28477)
EARKAIARVKRESKRIVEDAERLIREAAAASEKISREAERLIREAAAASEKISRE
>key_a_m4
(SEQ ID NO: 28478)
DEARKAIARVKRESKRIVEDAERLIREAAAASEKISREAERLIREAAAASEK
>key_a_m9
(SEQ ID NO: 28479)
DEARKAIARVKRESKRIVEDAERLIREAAAASEKISREAERLIREAAK
>key_a_m12
(SEQ ID NO: 28480)
DEARKAIARVKRESKRIVEDAERLIREAAAASEKISREAERLIR
>key_a_m15
(SEQ ID NO: 28481)
DEARKAIARVKRESKRIVEDAERLIREAAAASEKISREAER
>key_b
(SEQ ID NO: 28482)
NKEEIEKLAKEAREKLKKAEKEHKEIHDKLRKKNKKAREDLKKKADELRETNKRVN
>key_c
(SEQ ID NO: 28483)
SSEKVRRELKESLKENHKQNQKLLKDHKRAQEKLNRELEELKKKHKKTLDDIRRES
>key_d
(SEQ ID NO: 28484)
DTVKRILEELRRRFEKLAKDLDDIARKLLEDHKKHNKELKDKQRKIKKEADDAARS
>key_d_m4
(SEQ ID NO: 28485)
RILEELRRRFEKLAKDLDDIARKLLEDHKKHNKELKDKQRKIKKEADDAARS
>key_d_m7
(SEQ ID NO: 28486)
EELRRRFEKLAKDLDDIARKLLEDHKKHNKELKDKQRKIKKEADDAARS

Mapping Switch to Key that Activates It: See Table 6 Above
cODC degon Sequences

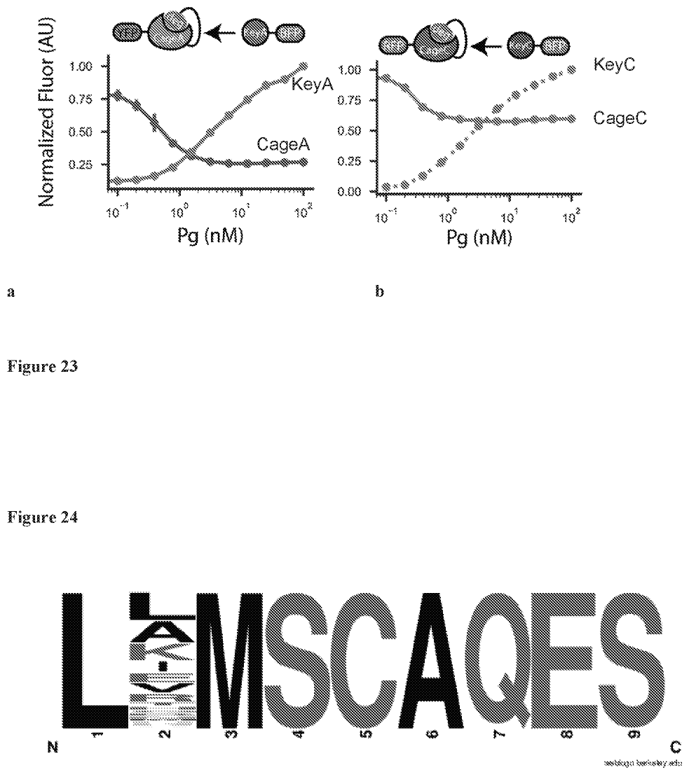

All cODC Variants encoded into the Latch summarized in this sequence logo shown in FIG. 24. The minimal motif for degradation activity is CA in the fifth and sixth positions, which are between 10-30 residues from the C-terminus. Multiple designs have residues 3-8 fixed at MSCAQE, except for the CA_only design. Diversity in the first and last position is due to structural considerations in LOCKR_{b}, LOCKR_{c}, and LOCKR_{d} where the residue from the base scaffold was chosen over the cODC sequence. Diversity in the second position is due to the proline destabilizing the helical conformation, as described in the above text. In that case, the residue from the base scaffold was chosen at that position.

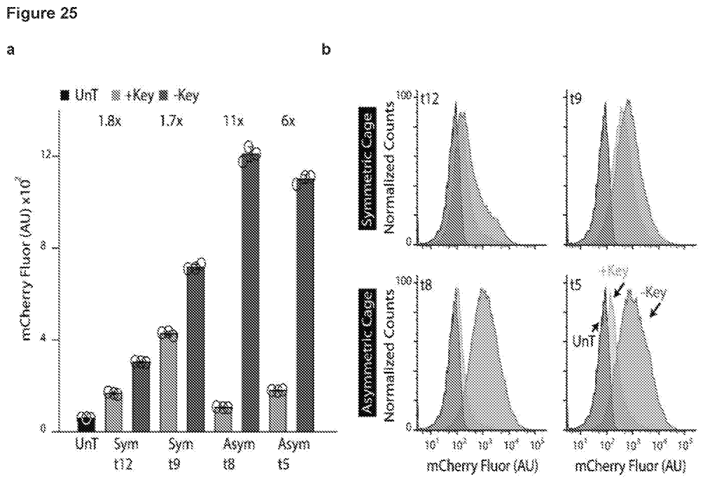

To evaluate degronLOCKR function in mammalian cells, degronSwitcha fused to mCherry™ RFP was expressed in human HEK293T cells, and RFP fluorescence was measured in the presence and absence of Key. A redesigned asymmetric degronSwitcha with an 8-residue toehold (1fix-short_cODC_t8 (SEQ ID NO: 27,372)) (see FIG. 25) triggered a 11-fold reduction in mean RFP fluorescence in the presence of Key. These data demonstrate the functionality of the degronLOCKR system in mammalian cells.

The ability of degronLOCKR to function in human primary T cells was demonstrated by inducibly degrading a mCherry™ fluorescent protein. Lentiviral transfer constructs were constructed containing mCherry™ fused to the asymmetric short scaffold degronSwitch with a t8 toehold and cODC degron embedded in the latch (1fix-short_cODC_t8 (SEQ ID NO: 27,372)). The mCherry™-degronSwitch fusion was expressed using pPGK constitutive promoter. In a second lentiviral construct a fusion of Key to tagBFP was expressed using four different constitutive promoters (pPGK, pSFFV, pCMV(G), pCMV(D)).

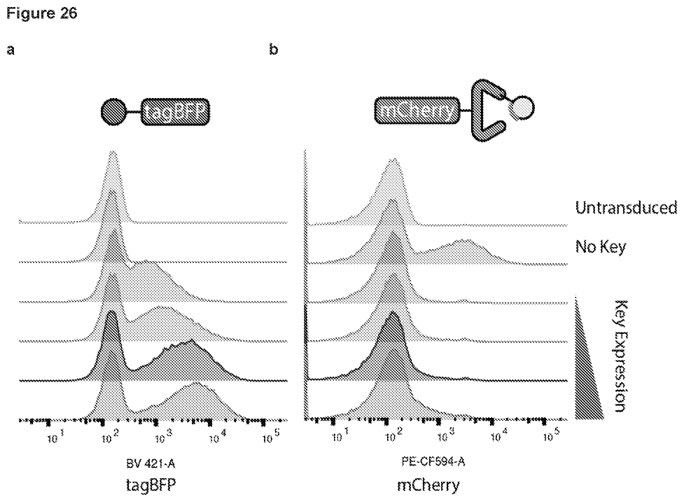

Experiments were performed in human primary CD4+ T cells. Cells were transduced with different combinations of the aforementioned lentiviruses. In one instance, cells were transduced with only mCherry™-degronSwitch. In others, cells received both the mCherry™-degronSwitch virus in addition to a virus expressing Key-tagBFP. After lentiviral transduction, fluorescence was measured using flow cytometry. Distributions are shown in FIG. 26. We observed that mCherry™ fluorescence was nearly completely abolished when cells were co-transduced with a virus containing any amount of Key production (Key production was quantified using tagBFP fluorescence). This data indicates that the Key is able to trigger the degronSwitch and activate degradation of mCherry™.

### REFERENCES

1. Takeuchi, J., Chen, H., Hoyt, M. A. & Coffino, P. Structural elements of the ubiquitin-independent proteasome degron of ornithine decarboxylase. Biochem. J.1410, 401-407 (2008).
2. Aranda-Diaz, A., Mace, K., Zuleta, I., Harrigan, P. & El-Samad, H. Robust Synthetic Circuits for Two-Dimensional Control of Gene Expression in Yeast. ACS Synth. Biol. 6, 545-554 (2017).
3. Harrigan, P., Madhani, H. & El-Samad, H. Real time genetic compensation operationally defines the dynamic demands of feedback control. bioRxiv 244020 (2018). doi:10.1101/244020
4. Khalil, A. S. et al. A synthetic biology framework for programming eukaryotic transcription functions. Cell 150, 647-658 (2012).
5. Nielsen, A. A. K. et al. Genetic circuit design. automation. Science 352, aac7341 (2016).
6. Perez-Pinera, P. et al. RNA-guided gene activation by CRISPR-Cas9-based transcription factors. Nat. Methods 10, 973-976 (2013).
7. Lee, M. E., DeLoache, W. C., Cervantes, B. & Dueber, J. E. A highly characterized yeast toolkit for modular, multipart assembly. AGS Synth. Biol. 4,975-986 (2015).

We claim:

1. A non-naturally occurring cage polypeptide comprising:
(a) a helical bundle, comprising between 2 and 7 alpha-helices, wherein the helical bundle comprises:
(i) a structural region; and
(ii) a latch region, wherein the latch region comprises a degron located within the latch region, wherein the structural region interacts with the latch region to prevent activity of the degron; and
(b) amino acid linkers connecting each alpha helix.

2. The cage polypeptide of claim 1, wherein the latch region is C-terminal to the structural region, and wherein the degron is located within about 100, 95, 90, 85, 80, 75, 70, 65, 60, 55, 50, 45, 40, 35, 30, 25, 20, 15, 10, 5, 4, 3, 2, 1, or 0 amino acid residues of the C-terminus of the latch region, and/or within about 100, 95, 90, 85, 80, 75, 70, 65, 60, 55, 50, 45, 40, 35, 30, 25, 20, 15, 10, 5, 4, 3, 2, 1, or 0 amino acid residues of the C-terminus of the cage polypeptide.

3. The cage polypeptide of claim 2, wherein the degron comprises a CA dipeptide located between 10-30 residues from the C-terminus of the cage polypeptide.

4. The cage polypeptide of claim 3 wherein the degron comprises the peptide MSCAQES (SEQ ID NO: 28468).

5. The cage polypeptide of claim 3 wherein the degron comprises the peptide L(X)MSCAQES (SEQ ID NO: 28467), wherein X can be any amino acid residue, wherein X is optionally not praline.

6. The cage polypeptide of claim 1 or 2, wherein the degron comprises an amino acid residue or peptide selected from the group consisting of
(a) GG; RG; KG; QG; WG; PG; AG; RxxG; EE; R; Rxx; Vx; Ax; A, wherein “x” can be any amino acid residue, and wherein the degron is within 10-30 amino acids of a terminus of the latch region, and/or within 10-30 amino acids of a terminus of the cage polypeptide;
(b) an amino acid residue or peptide that recruits an ubiquitin ligase that ubiquitilates the cage polypeptide and/or the operably linked functional polypeptide;
(c) a proteolysis-targeting chimeric molecule (PROTAC); and
(d) any other degron described herein.

7. The cage polypeptide of claim 1, wherein the latch region is N-terminal to the structural region, and wherein the degron is located within about 100, 95, 90, 85, 80, 75, 70, 65, 60, 55, 50, 45, 40, 35, 30, 25, 20, 15, 10, 5, 4, 3, 2, 1, or 0 amino acid residues of the N-terminus of the latch region, and/or within about 100, 95, 90, 85, 80, 75, 70, 65, 60, 55, 50, 45, 40, 35, 30, 25, 20, 15, 10, 5, 4, 3, 2, 1, or 0 amino acid residues of the N-terminus of the cage polypeptide.

8. The cage polypeptide of claim 1 or 2, wherein the degron comprises a peptide selected from the group consisting of the following (residues within brackets are optional):
(SEQ ID NO: 28469)
[[KTRGVEEVAEGVVLL]]RRRG [[NK(FAM)KKK]],
(SEQ ID NO: 28470)
[[KPFLNGGPY]] HSREQSTDSG [[LGLGSYK(FAM)KKK]],
(SEQ ID NO: 28471)
ASADLDLEALAPYIPADDDFQLRK(FAM)KKK,
(SEQ ID NO: 28472)
[[K-(PEG)-KEEK]] DINN [[VKKTK(FAM)KKK]],
(SEQ ID NO: 28487)
[[K-(PEG)]] DVQKADVSST [[GQGIDSK(FAM)KKK]],
(SEQ ID NO: 28473)
KAAEEEEVSLASTPTDVRDVDIK(FAM)KKK,
(SEQ ID NO: 28474)
[[KKYSSQTSQ]] DSGNYS [[NK(FAM)KKK]],
(SEQ ID NO: 28475)
KPLSSSVPSQKTYQGSYGFRLGK(FAM)KKK,
(SEQ ID NO: 28476)
[[KAWQQQSYL]] DSGIHSG [[ATTTAPK(FAM)KKK]];
(SEQ ID NO: 28488)
[[KTRGVEEVAEGVVLL]]RRRG [[NKKKK]],
(SEQ ID NO: 28489)
[[KPFLNGGPY]] HSREQSTDSG [[LGLGSYKKKK]],
(SEQ ID NO: 28490)
ASADLDLEALAPYIPADDDFQLRKKKK
(SEQ ID NO: 28491)
[[KKEEK]] DINNN [[VKKTKKKK]],
(SEQ ID NO: 28492)
[[K]] DVQKADVSST [[GQGIDSKKKK]],
(SEQ ID NO: 28493)
KAAEEEEVSLASTPTDVRDVDIKKKK,
(SEQ ID NO: 28494)
[[KKYSSQTSQ]] DSGNYS [[NKKKK]],
(SEQ ID NO: 28495)
KPLSSSVPSQKTYQGSYGFRLGKKKK,
(SEQ ID NO: 28496)
[[KAWQQQSYL]] DSGIHSG [[ATTTAPKKKK]];
(SEQ ID NO: 28497)
RRRG;
(SEQ ID NO: 28498)
HSREQSTDSG;
(SEQ ID NO: 28499)
DINNN;
(SEQ ID NO: 28500)
DVQKADVSST;
(SEQ ID NO: 28501)
DSGNYS;
and
(SEQ ID NO: 28502
DSGIHSG.

9. The cage polypeptide of any one of claims 1-8, further comprising one or more functional polypeptide domains.

10. The cage polypeptide of claim 9, wherein the one or more functional polypeptide domains are located at the N-terminus of the cage polypeptide and the latch region is located C-terminal to the structural region, or wherein the one or more functional polypeptide domains are located at the C-terminus of the cage polypeptide and the latch region is located N-terminal to the structural region.

11. The cage polypeptide of claim 9, wherein the one or more functional polypeptide domains are located at the same terminus or of the cage polypeptide as the latch region.

12. The cage polypeptide of any one of claims 9-11, wherein the one or more functional polypeptide domains include, but are not limited to metabolic enzymes, transcription factors, kinases, phosphatases, Chimeric Antigen Receptor (CAR), T Cell Receptor (TCR), SynNotch, TCR mimics, cytokine receptors, G-protein coupled receptors (GPCR), co-stimulatory receptors (including but not limited to CD28, CTLA-4, ICOS), co-inhibitory receptors (e.g. PD-1), endogenous signaling domains (including but not limited to Pleckstrin Homology (PH), Src Homology 2 (SH2), Src Homology 3 (SH3), WW, C1, PDZ, CARD, phosphotyrosine-binding, proline-rich region, coiled-coil, and pseudokinase domains), synthetic receptors or synthetic signaling proteins comprising-one or more signaling domain (including but not limited to Pleckstrin Homology (PH), Src Homology 2 (SH2), Src Homology 3 (SH3), WW, C1, PDZ, CARD, phosphotyrosine-binding, proline-rich region, coiled-coil, and pseudokinase domains), engineered or endogenous receptors containing ITAM or ITIM motifs, jAK/STAT binding motifs, DNA binding domains (including but not limited to Cas9, dCas9, TALEs, and Zinc Fingers), vesicular trafficking domains, protein degradation domains (including but not limited to ubiqui tin recruitment domains and proteasomal-targeting domains), cell death domains (including but not limited to those involved in the apoptosis, necroptosis, and pyroptosis), fluorescent proteins, de novo designed proteins, a second cage polypeptide described herein, such as one that binds to a key polypeptide different than a key polypeptide bound by the cage polypeptide), a key polypeptide described herein, such as one that does not bind to the cage polypeptide, and active domains thereof.

13. The cage polypeptide of any one of claims 1-12, wherein the latch region comprises one or more additional bioactive peptides besides the degron, wherein the structural region interacts with the latch region to prevent activity of the one or more additional bioactive peptides.

14. The cage polypeptide of claim 13, wherein the one or more additional bioactive peptides may comprise one or more additional bioactive peptide comprising the amino acid sequence selected from the non-limiting group consisting of SEQ ID NO: 50, 60, 62-64, 66, 27052-27093, and 27118-27119.

15. The cage polypeptide of any one of claims 1-14. wherein each helix is independently 18-60, 18-55, 18-50, 18-45, 22-60, 22-55, 22-50, 22-45, 25-60, 25-55, 25-50, 25-45, 28-60, 28-55, 28-50, 28-45, 32-60, 32-55, 32-50, 32-45, 35-60, 35-55, 35-50, 35-45, 38-60, 38-55, 38-50, 38-45, 40-60, 40-58, 40-55. 40-50, or 40-45 amino acids in length.

16. The cage polypeptide of any one of claims 1-15, wherein each amino acid linker is independently between 3-10, 4-10, 5-10, 6-10, 7-10, 8-10, 9-10, 2-9, 3-9, 4-9, 5-9, 6-9, 7-9, 8-9, 2-8. 3-8, 4-8, 5-8, 6-8, 7-8, 2-7, 3-7. 4-7, 5-7, 6-7, 2-6, 3-6, 4-6, 5-6, 2-5, 3-5, 4-5, 2-4, 3-4, 2-3, or 2, 3, 4. 5, 6, 7, 8, 9, or 10 amino acids in length, not including any functional polypeptide domain fused to the linker.

17. The cage polypeptide of any one of claims 1-16, wherein the cage polypeptide comprises the amino acid sequence having at least 45%, 50%, 55%, 60%, 65%, 70%, 75%, 80%, 85%, 90%, 91%, 92%, 93%, 94%, 95%, 96%, 97%, 98%, 99%, or 100% sequence identity, not including optional residues, along the full length of the amino acid sequence of a cage polypeptide selected from the group consisting of (a) SEQ ID NO: 27359-28465 or a cage polypeptide listed in Table 7 (in (a) embodiments, the degron is included within the polypeptide sequence), and (b) SEQ ID NOS:1-49, 51-52, 54-59, 61, 65, 67-91, 92 -2033, SEQ ID NOS:2034-14317, 27094-27117, 27120-27125, 27278-27321, and cage polypeptides listed in Table 2 (polypeptides with an even-numbered SEQ ID NO between SEQ ID NOS: 27126 and 27276), Table 3, and/or Table 4 ((in (b) embodiments, the degron is not included in the amino acid sequence and would be added within the latch region, including but not limited to those degron amino acid sequences disclosed herein.

18. A kit comprising:
(a) the cage polypeptide of any one of claims 1-17; and
(b) a key polypeptide capable of binding to the cage polypeptide structural region, thereby displacing the latch region and activating the one or more degron.

19. A degron LOCKR switch comprising:
(a) the cage polypeptide of any one of claims 1-17; and
(b) a key polypeptide capable of binding to the cage polypeptide structural region, thereby displacing the latch region and activating the one or more degron.

20. The kit of claim 18 or the degron LOCKR switch of claim 19, wherein the key polypeptide comprises an amino acid sequence having at least 45%, 50%, 55%, 60%, 65%, 70%, 75%, 80%, 85%, 90%, 91%, 92%, 93%, 94%, 95%, 96%, 97%, 98%, 99%, or 100% sequence identity, not including optional residues, along the full length of the amino acid sequence of a key protein disclosed herein, or a key polypeptide selected from the group consisting of SEQ ID NOS: 26602-27050, and 27,322 to 27,358. and 28477-28486, or a key polypeptide listed in Table 7.

21. The kit or degron LOCKR switch of any one of claims 18-20, wherein the cage polypeptide comprises an amino acid sequence having at least 45%, 50%, 55%, 60%, 65%, 70%, 75%, 80%, 85%, 90%, 91%, 92%. 93%, 94%, 95%, 96%, 97%, 98%, 99%, or 100% sequence identity, not including optional residues, along the full length of the amino acid sequence selected from the group consisting of SEQ ID NO: 27359-28465 or a cage polypeptide listed in Table 7.

22. The kit or degron LOCKR switch of any one of claims 18-21, wherein the one or more cage polypeptide and the one or more key polypeptide comprise at least one cave polypeptide and at least one key polypeptide comprising an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity, not including optional residues, along the full length of a cage polypeptide and a key polypeptide, respectively, the in the same row of Table 2, 3, 4, 5, 6, or 7, in particular Table 6 or Table 7.

23. A nucleic acid encoding the cage polypeptide of any one of claims 1-17.

24. An expression vector comprising the nucleic acid of claim 23 operatively linked to a promoter.

25. A kit comprising:
(a) one or more nucleic acids encoding the cage polypeptide of any one of claims 1-17: and
(b) one or more nucleic acids encoding one or more key polypeptides capable of binding to the cage polypeptide structural region.

26. A kit comprising:
(a) one or more expression vectors according to claim 24; and
(b) one or more expression vectors comprising one or more nucleic acids encoding one or more key polypeptides capable of binding to the cage polypeptide structural region, wherein the one or more nucleic acids encoding one or more key polypeptides are operatively linked to a promoter.

27. A host cell comprising one or more nucleic acids encoding the cage polypeptide of any one of claims 1-17, and/or one or more of the expression vectors of claim 24.

28. The host cell of claim 27, further comprising one or more nucleic acids encoding one or more key polypeptides capable of binding to the cage polypeptide structural region, wherein the one or more nucleic acids encoding one or more key polypeptides capable of binding to the cage polypeptide structural region are operatively linked to a promoter, and/or one or more expression vectors comprising one or more nucleic acids encoding one or more key polypeptides capable of binding to the cage polypeptide structural region, wherein the one or more nucleic acids encoding one or more key polypeptides are operatively linked to a. promoter.

29. The host cell of claim 28, wherein the one or more expression vectors of claim 24 are operatively linked to a first promoter, and the one or more expression vectors comprising one or more nucleic acids encoding one or more key polypeptides are operatively linked to a second promoter, where the first promoter and the second promoter are different.

30. The kit of claim 25 or 26, or the host cell of any one of claims 28 and 29, wherein the one or more nucleic acids encoding the one or more key polypeptides comprise an amino acid sequence having at least 45%, 50%, 55%, 60%, 65%, 70%, 75%, 80%, 85%, 90%, 91%, 92%, 93%, 94%, 95%, 96%, 97%, 98%, 99%, or 100% sequence identity, not including optional residues, along the full length of the amino acid sequence of a key protein disclosed herein, or a key polypeptide selected from SEQ ID NOS: 26602-27050, 27,322-27,358, and 28477-28486.

31. The kit of claim 25 or 26, or the host cell of any one of claims 28 and 29, wherein the one or more nucleic acids encoding the one or more key polypeptides comprise an amino acid sequence having at least 45%, 50%, 55%, 60%, 65%, 70%, 75%, 80%, 85%, 90%, 91%, 92%, 93%, 94%, 95%, 96%, 97%, 98%, 99%, or 100% sequence identity, not including optional residues, along the full length of the amino acid sequence of a key polypeptide selected from the group consisting of SEQ ID NOS: 28477-28486.

32. The kit of claim 25 or 26, or the host cell of any one of claims 28 and 29, wherein the one or more nucleic acids encoding the polypeptide of any one of claims 1-17, encodes a polypeptide that comprise an amino acid sequence having at least 45%, 50%, 55%, 60%, 65%, 70%, 75%, 80%, 85%, 90%, 91%, 92%, 93%, 94%, 95%, 96%, 97%, 98%, 99%, or 100% sequence identity, not including optional residues, along the full length of the amino acid sequence of a cage polypeptide selected from the group consisting of SEQ ID NOS: 27359-28465 or a cage polypeptide listed in Table 7.

33. The kit of claim 24, 25, 31, or 32, or the host cell of any one of claims 27 and 28, wherein the one or more cage polypeptide and the one or more key polypeptide comprise at least one cage polypeptide and at least one key polypeptide comprising an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity, not including optional residues, along the full length of a cage polypeptide and a key polypeptide, respectively, the in the same row of Table 2, 3, 4, 5, 6, or 7, in particular Table 6 or Table 7.

34. Use of the polypeptides, LOCKR switches, nucleic acids, expression vectors, or host cells disclosed herein to sequester a degron in the cage polypeptide until a key is expressed and activates the cage polypeptide, and the degron targets the cage polypeptide and any functional peptide fused to it for degradation.
